(19) United States
(12) Patent Application Publication
(10) Pub. No.: US 2025/0223299 A1
(43) Pub. Date: Jul. 10, 2025

(54) MAYTANSINOID DERIVATIVES, CONJUGATES THEREOF, AND METHODS OF USE

(71) Applicant: Regeneron Pharmaceuticals, Inc., Tarrytown, NY (US)

(72) Inventor: Thomas Nittoli, Pearl River, NY (US)

(21) Appl. No.: 19/093,440

(22) Filed: Mar. 28, 2025

Related U.S. Application Data
(62) Division of application No. 17/528,144, filed on Nov. 16, 2021, now Pat. No. 12,297,207.
Application No. 17/528,144 is a continuation of application No. 16/712,941, filed on Dec. 12, 2019, now abandoned.
Application No. 16/712,941 is a continuation of application No. 15/081,759, filed on Mar. 25, 2016, now abandoned.
(60) Provisional application No. 62/139,044, filed on Mar. 27, 2015.
(60) Provisional application No. 62/252,239, filed on Nov. 6, 2015.

(51) Int. Cl. C07D 498/18; A61K 47/68

(52) U.S. Cl. CPC C07D 498/18; A61K 47/68033; A61K 47/6849; A61K 47/6889

(57) ABSTRACT

Provided herein are maytansinoid derivatives, conjugates thereof, and methods of treating or preventing proliferative diseases with the same.

### MAYTANSINOID DERIVATIVES, CONJUGATES THEREOF, AND METHODS OF USE

This application claims priority to, and the benefit of, U.S. Provisional Patent Application No. 62/139,044, entitled MAYTANSINOID DERIVATIVES, CONJUGATES THEREOF AND METHODS OF TREATING PROLIFERATIVE DISEASES USING THE SAME, which was filed Mar. 27, 2015, and also claims priority to, and the benefit of U.S. Provisional Patent Application No. 62/252,239, entitled MAYTANSINOID DERIVATIVES, CONJUGATES THEREOF AND METHODS OF TREATING PROLIFERATIVE DISEASES USING THE SAME, which was filed Nov. 6, 2015. The contents of each of these provisional patent applications are herein incorporated by reference in their entirety for all purposes.

### FIELD

The present disclosure concerns maytansinoid derivatives, conjugates thereof, and methods of treating or preventing proliferative diseases with the same.

### BACKGROUND

Proliferative diseases, for example cancer, are characterized by the uncontrolled growth of abnormal cells. Current treatments of proliferative diseases include surgery, radiation, chemotherapy, hormone-based therapy and/or immunotherapy. A number of these treatments, particularly chemotherapy, utilize anti-proliferative drugs that limit the spread of the abnormal cells. However, these drugs are typically indiscriminate in their ability to kill cells, affecting both normal and abnormal cells. To address this problem, various approaches to targeted drug delivery have been explored, including the use of conjugates of tumor-targeted probes (such as antibodies or growth factors) with toxins, to selectively target abnormal cells. Antibody drug conjugates (ADCs) are compounds composed of an antibody that is linked, via a chemical linker, to a cytotoxic agent. Such compounds leverage the antibody's binding specificity for its target to deliver a cytotoxic agent to an abnormal cell. Thus, there is a need for anti-proliferative compounds and their conjugates.

### SUMMARY

Provided herein are compounds of Formula (I):

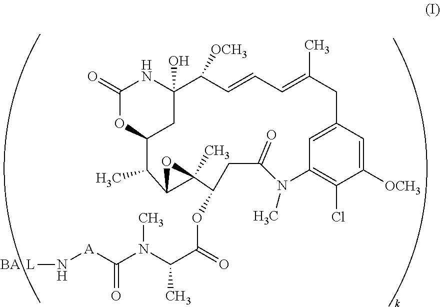

or a pharmaceutically acceptable salt thereof,
wherein:
A is arylene or heteroarylene;
L is a linker;
BA is a binding agent; and
k is an integer from 1 to 30. Also provided herein are stereoisomers of compounds of Formula (I).

Provided herein are also compounds of Formula (II):

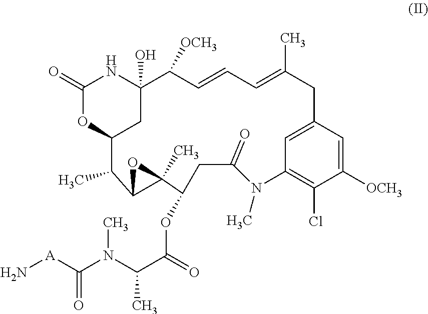

or a pharmaceutically acceptable salt thereof, wherein A is arylene or heteroarylene. Also provided herein are stereoisomers of compounds of Formula (II).

Provided herein are also compounds of Formula PP5:

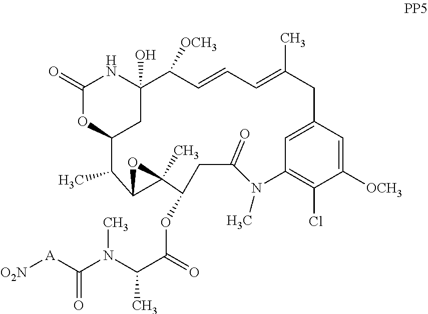

or a salt thereof, wherein A is arylene or heteroarylene. Also provided herein are stereoisomers of compounds of Formula PP5.

Provided herein are also compounds of Formula PT1:

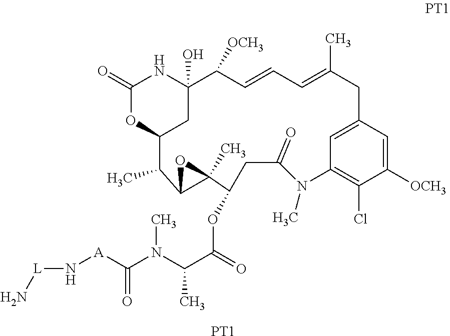

or a salt thereof, wherein A is arylene or heteroarylene and L is a linker. Also provided herein are stereoisomers of compounds of Formula PT1.

Furthermore, provided herein are methods of treating proliferative diseases comprising administering the compounds described herein.

Furthermore, provided herein are methods of treating proliferative diseases comprising administering the conjugates described herein.

Furthermore, provided herein are of methods of preparing compounds of Formula (I) comprising reacting a deglycosylated antibody or aglycosylated antibody with a compound of Formula (PT1) in the presence of transglutaminase.

### BRIEF DESCRIPTIONS OF THE DRAWINGS

FIG. 1 depicts a synthetic sequence for preparing maytansin-N-methyl-L-alanine-4-aminobenzamido-citrulline-valine-caprolyl-6-maleimidyl.

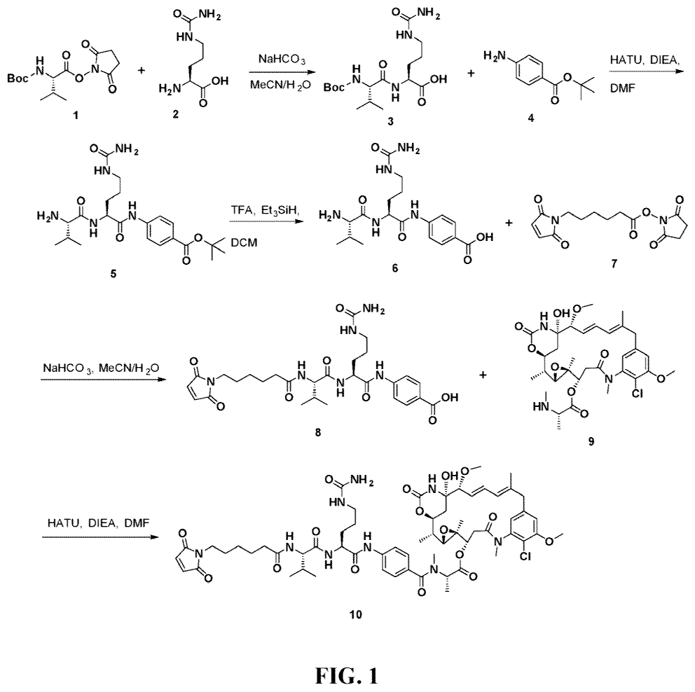

FIG. 2 depicts the plot of % Cell Viability vs. Log₁₀[M] of certain compounds tested in EXAMPLE 41.

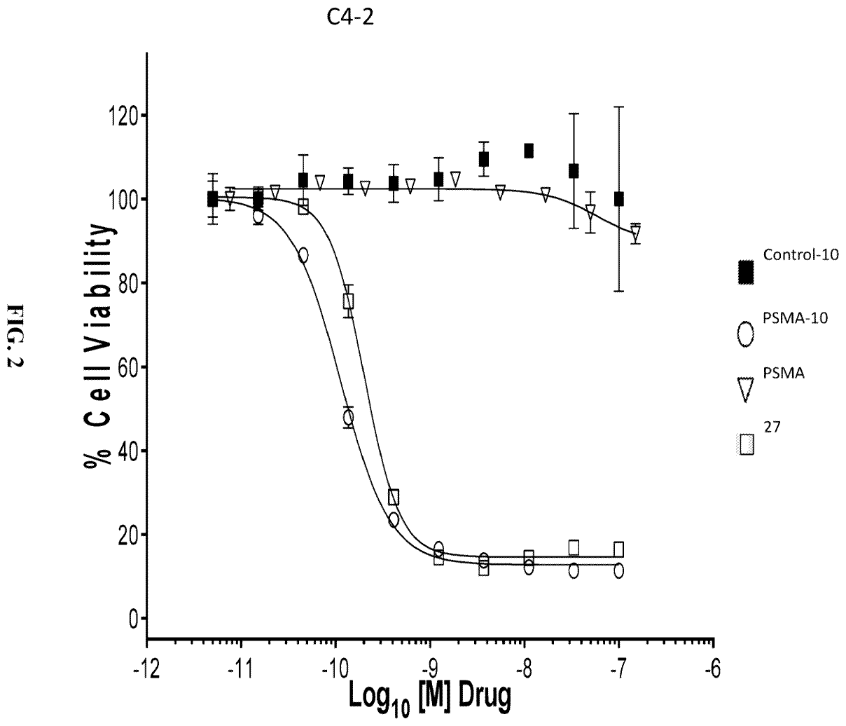

FIG. 3 depicts the plot of % Cell Viability vs. Log₁₀[M] of certain compounds tested in EXAMPLE 41.

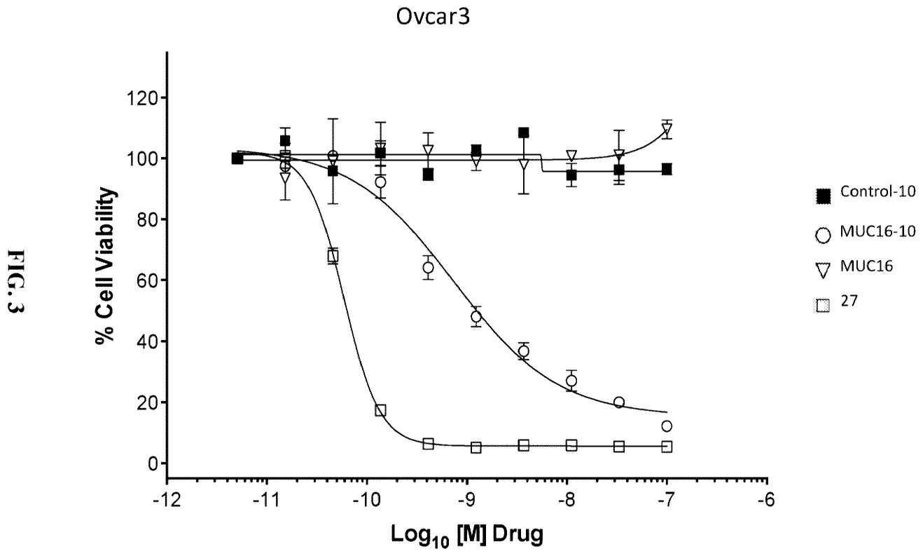

FIG. 4 depicts the plot of % Cell Viability vs. Log₁₀[M] of certain compounds tested in EXAMPLE 41.

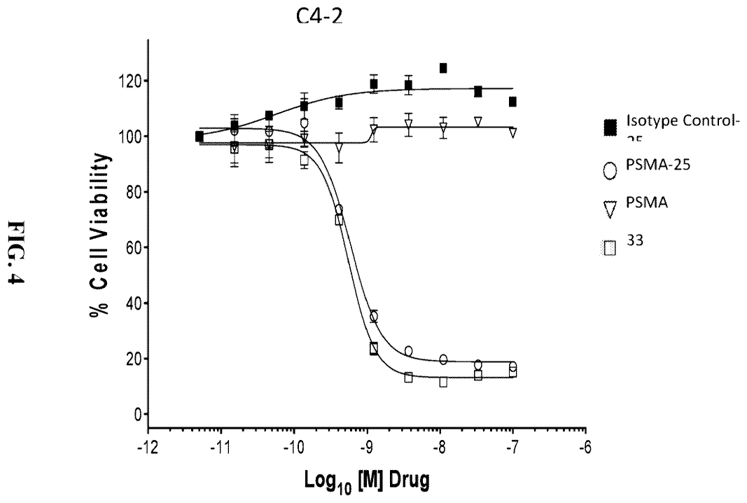

FIG. 5 depicts the plot of % Cell Viability vs. Log₁₀[M] of certain compounds tested in EXAMPLE 41.

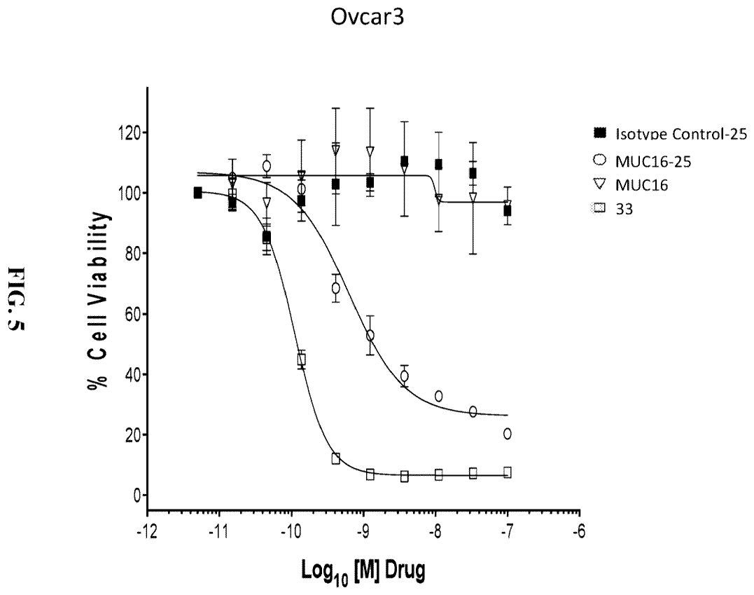

FIG. 6 depicts a synthetic sequence for preparing maytansin-N-methyl-L-alanine-(4-amino-2-fluoro)benzamido-Cit-Val-Cap-Mal.

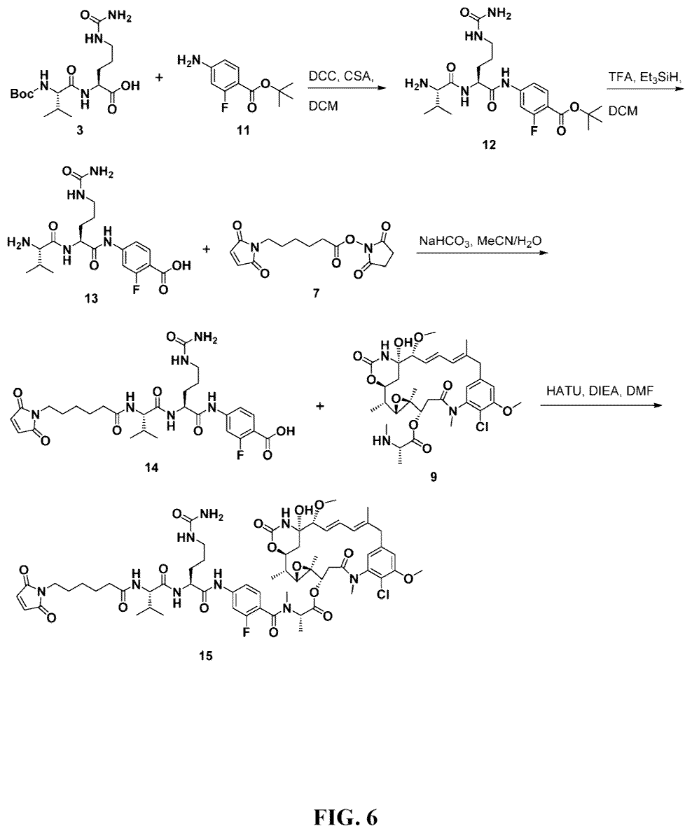

FIG. 7 depicts a synthetic sequence for preparing maytansin-N-methyl-L-alanine-(4-amino-2-trifluoromethyl)benzamido-Cit-Val-Cap-Mal.

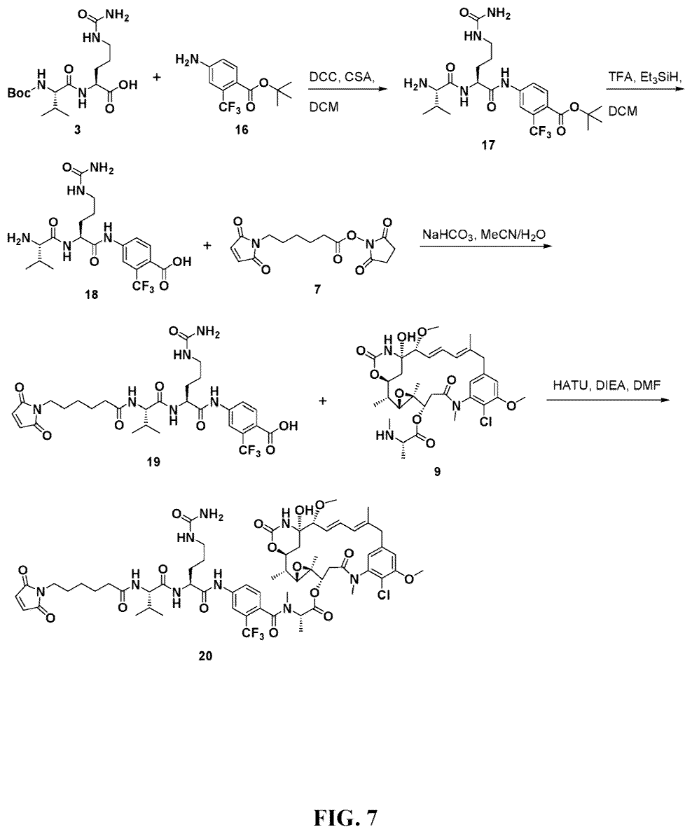

FIG. 8 depicts a synthetic sequence for preparing maytansin-N-methyl-L-alanine-(4-amino-2-methoxy)benzamido-Cit-Val-Cap-Mal.

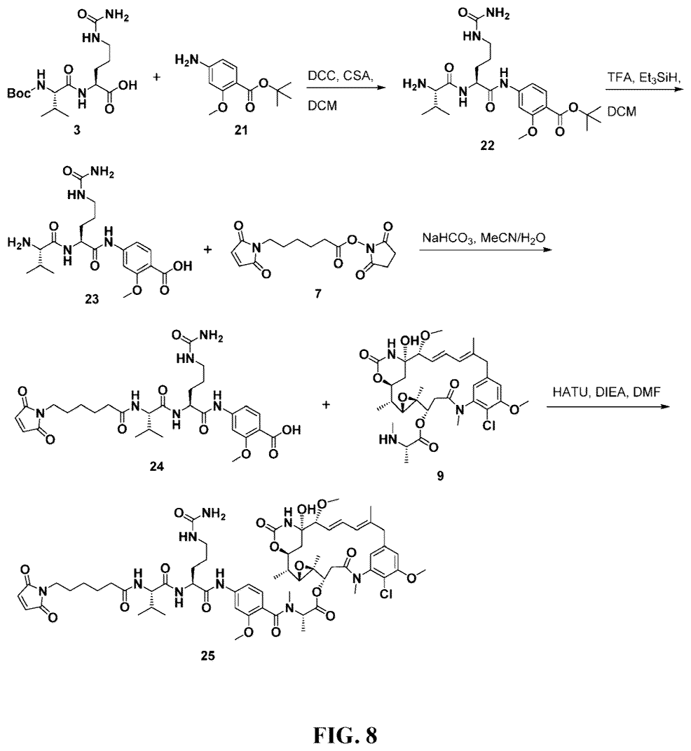

FIG. 9 depicts a synthetic sequence for preparing maytansin-N-methyl-L-alanine-4-aminobenzamide.

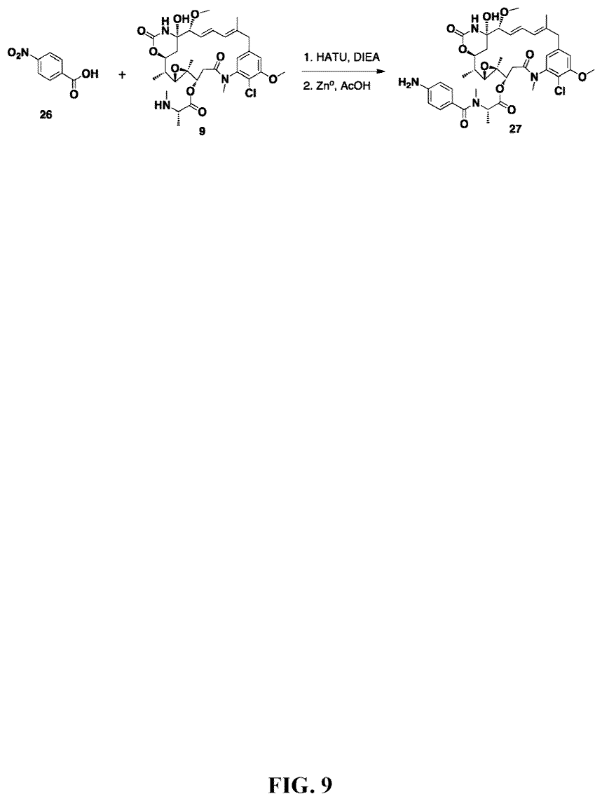

FIG. 10 depicts a synthetic sequence for preparing maytansin-N-methyl-L-alanine-(2-fluoro-4-amino)benzamide

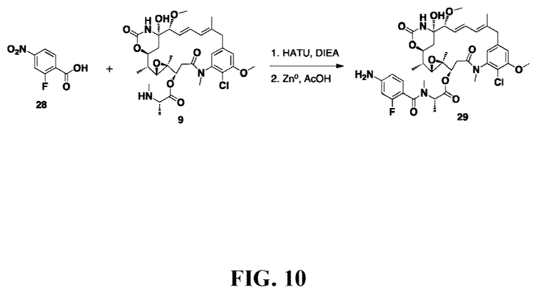

FIG. 11 depicts a synthetic sequence for preparing maytansin-N-methyl-L-alanine-(2-trifluoromethyl-4-amino)benzamide.

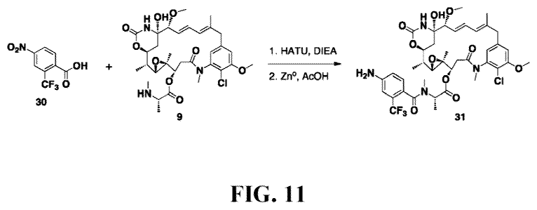

FIG. 12 depicts a synthetic sequence for preparing maytansin-N-methyl-L-alanine-(2-methoxy-4-amino)benzamide.

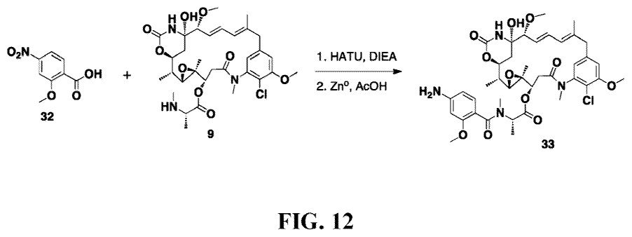

FIG. 13 depicts a synthetic sequence for preparing maytansin-N-methyl-L-alanine-N-(3-trifluoromethyl-4-amino)benzamide.

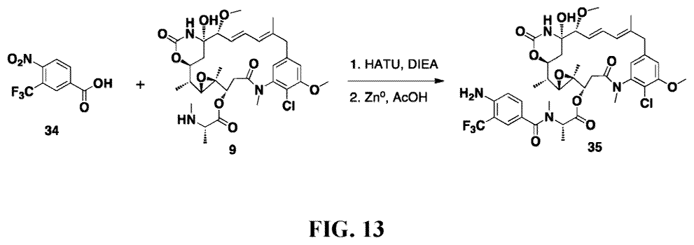

FIG. 14 depicts a synthetic sequence for preparing maytansin-N-methyl-L-alanine-N-(2-chloro-4-amino-5-fluoro)benzamide.

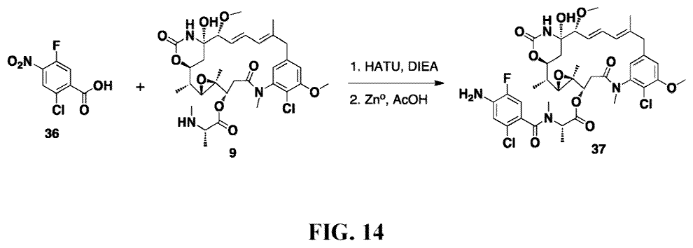

FIG. 15 depicts a general synthetic sequence for preparing compounds of Formula (II) wherein substituent R is defined herein and below.

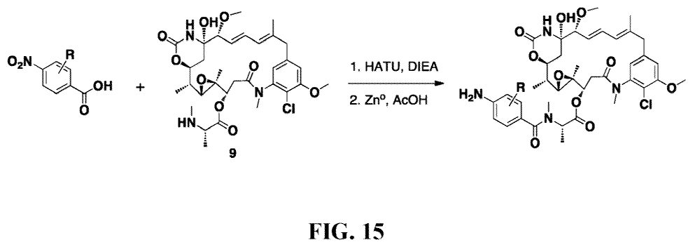

FIG. 16 depicts a synthetic sequence for preparing maytansin-N-methyl-L-alanine-N-(2,5-difluoro-4-amino)benzamide.

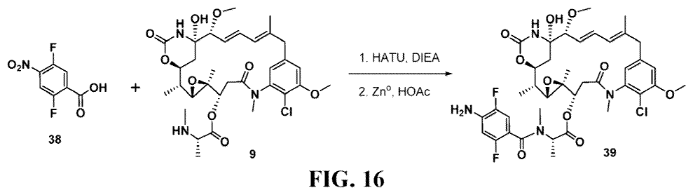

FIG. 17 depicts a synthetic sequence for preparing maytansin-N-methyl-L-alanine-(3-fluoro-4-amino)benzamide

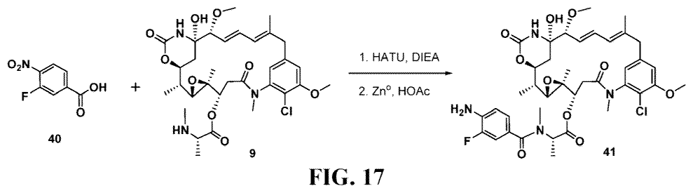

FIG. 18 depicts a synthetic sequence for preparing maytansin-N-methyl-L-alanine-(3-chloro-4-amino)benzamide.

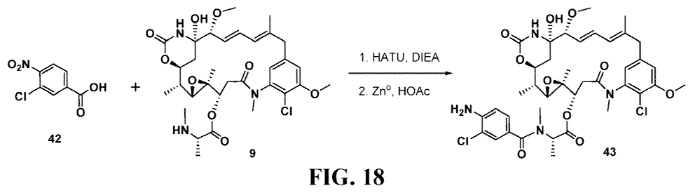

FIG. 19 depicts a synthetic sequence for preparing maytansin-N-methyl-L-alanine-(5-amino-8-carboxyquinoline)carboxamide.

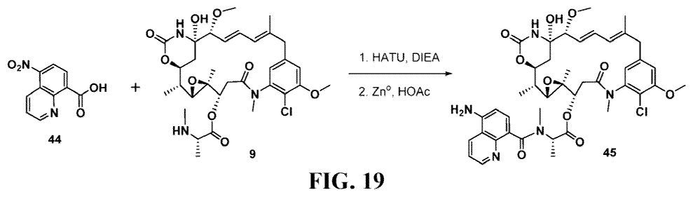

FIG. 20 depicts a synthetic sequence for preparing maytansin-N-methyl-L-alanine-(3-bromo-4-amino)benzamide.

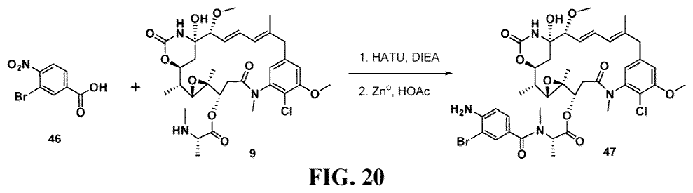

FIG. 21 depicts a synthetic sequence for preparing maytansin-N-methyl-L-alanine-(3-methoxy-4-amino)benzamide.

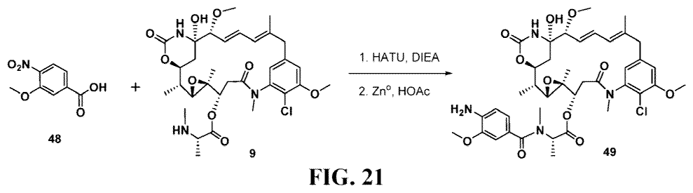

FIG. 22 depicts a synthetic sequence for preparing maytansin-N-methyl-L-alanine-(2-methyl-4-amino)benzamide.

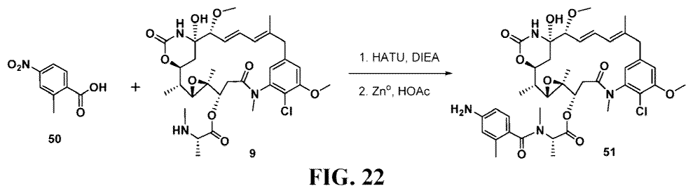

FIG. 23 depicts a synthetic sequence for preparing maytansin-N-methyl-L-alanine-(3-methyl-4-amino)benzamide.

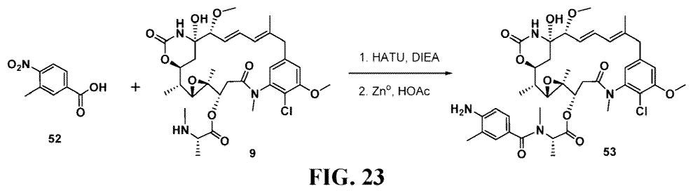

FIG. 24 depicts a synthetic sequence for preparing maytansin-N-methyl-L-alanine-(8-amino-5-carboxyquinoline)carboxamide.

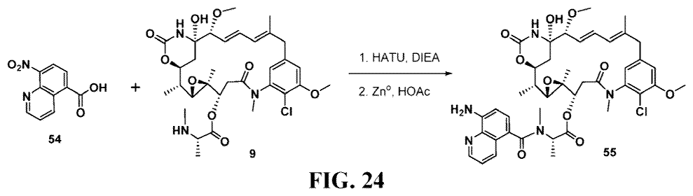

FIG. 25 depicts a synthetic sequence for preparing maytansin-N-methyl-L-alanine-(3-methoxy-4-amino)benzamido-Cit-Val-Cap-Mal.

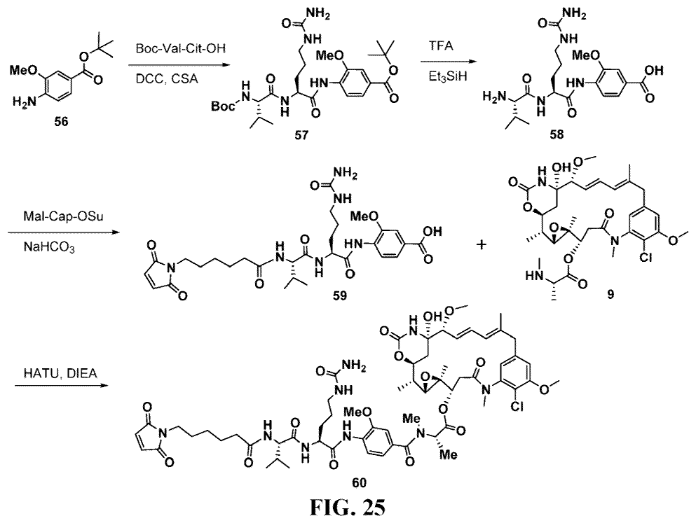

FIG. 26 depicts a synthetic sequence for preparing maytansin-N-methyl-L-alanine-(2-fluoro-4-amino)benzamido-Cit-Val-Cap-6-amine.

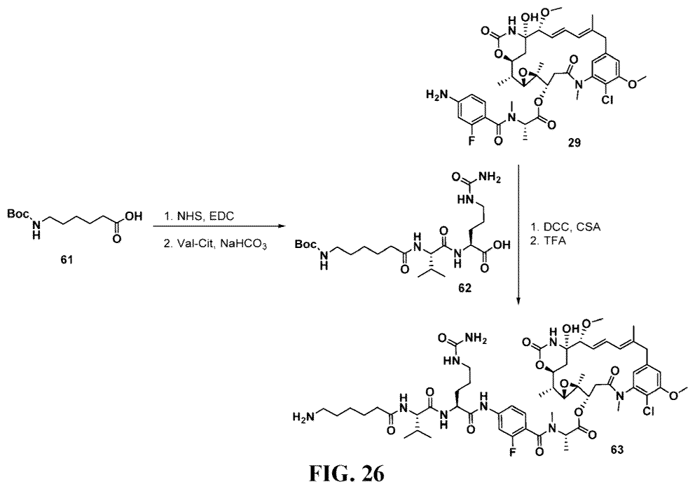

FIG. 27 depicts a synthetic sequence for preparing maytansin-N-methyl-L-alanine-N-(2-methoxy-5-amino)benzamide.

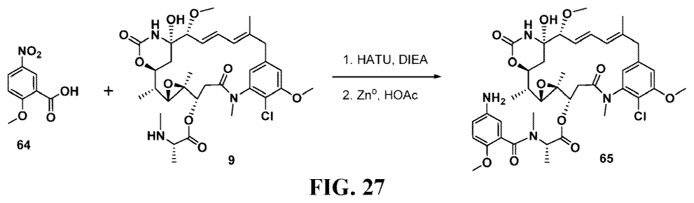

FIG. 28 depicts a synthetic sequence for preparing maytansin-N-methyl-L-alanine-N-(3-amino-4-methoxy)benzamide.

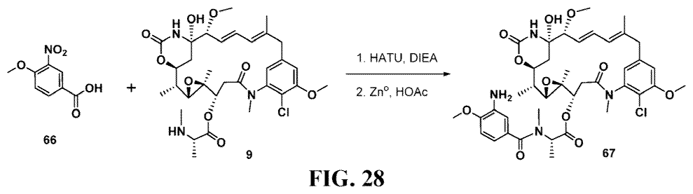

FIG. 29 depicts a synthetic sequence for preparing maytansin-N-methyl-L-alanine-N-(3-amino-5-fluoro)benzamide.

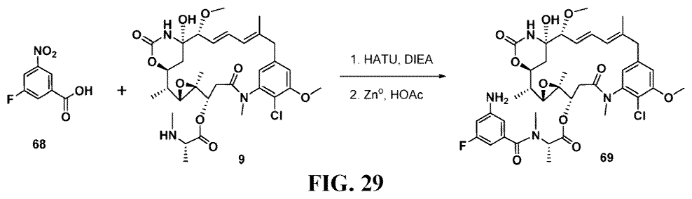

FIG. 30 depicts a synthetic sequence for preparing maytansin-N-methyl-L-alanine-N-(2-fluoro-5-amino)benzamide.

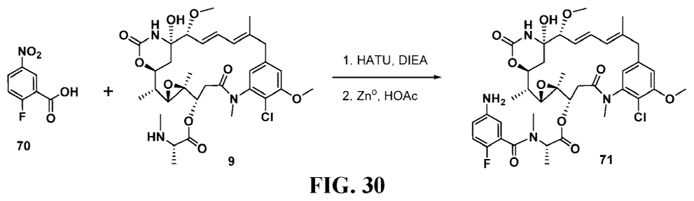

FIG. 31 depicts a synthetic sequence for preparing maytansin-N-methyl-L-alanine-N-(3-amino)benzamide.

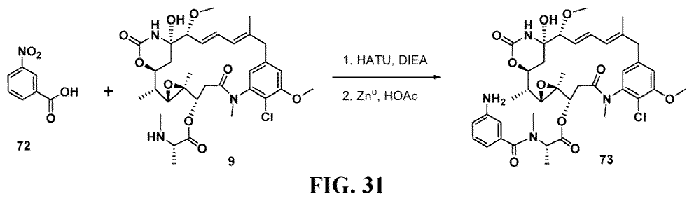

FIG. 32 depicts a synthetic sequence for preparing maytansin-N-methyl-L-alanine-N-(3-amino-4-fluoro)benzamide.

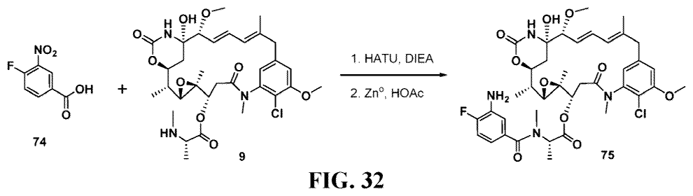

FIG. 33 depicts a synthetic sequence for preparing maytansin-N-methyl-L-alanine-N-4-aminobenzamide-adipic-NHS.

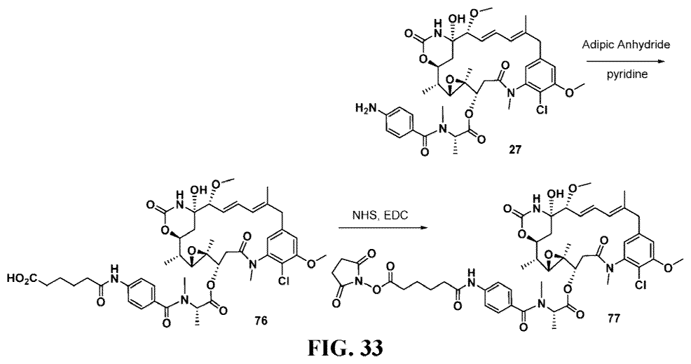

FIG. 34 depicts a synthetic sequence for preparing maytansin-N-methyl-L-alanine-4-aminobenzamide-Cap-Mal.

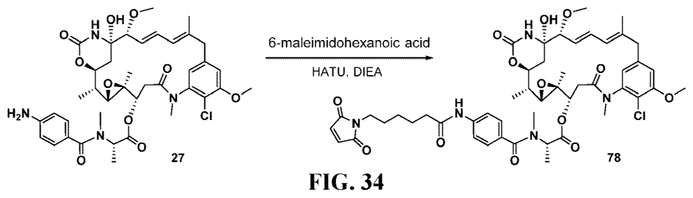

FIG. 35 depicts a synthetic sequence for preparing maytansin-N-methyl-L-alanine-N-(3-methylsulfonyl-4-amino)benzamide.

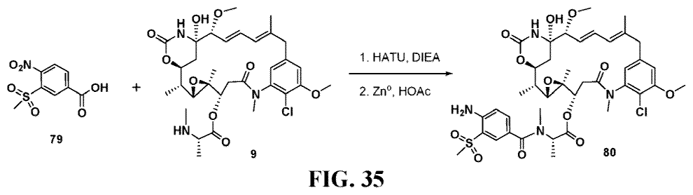

FIG. 36 depicts a synthetic sequence for preparing maytansin-N-methyl-L-alanine-N-(3-hydroxy-4-amino)benzamide.

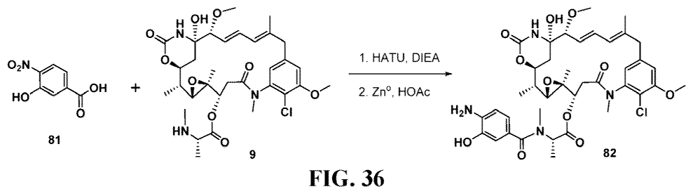

FIG. 37 depicts a synthetic sequence for preparing maytansin-N-methyl-L-alanine-N-(2-amino)benzamide.

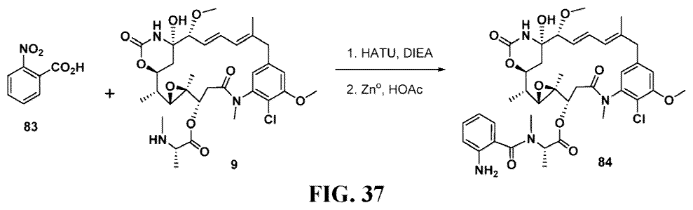

FIG. 38 depicts a synthetic sequence for preparing maytansin-N-methyl-L-alanine-N-(4-methoxy-2-amino)benzamide.

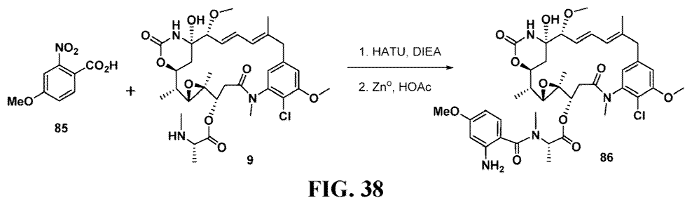

FIG. 39 depicts a synthetic sequence for preparing maytansin-N-methyl-L-alanine-N-(3-morpholino-4-amino)benzamide.

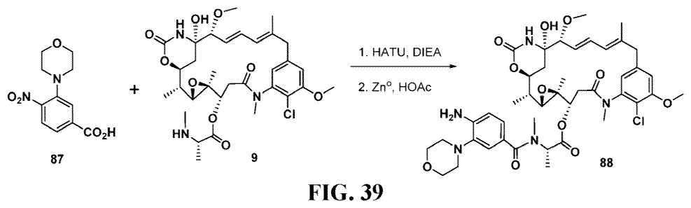

FIG. 40 depicts a synthetic sequence for preparing maytansin-N-methyl-L-alanine-N-(3-acetamido-4-amino)benzamide.

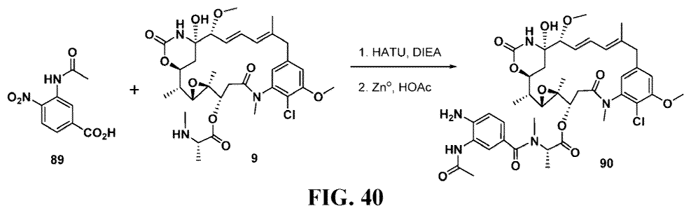

FIG. 41 depicts a synthetic sequence for preparing maytansin-N-methyl-L-alanine-N-4-aminobenzamide-Cit-Val-cap-diBromomethylacryl.

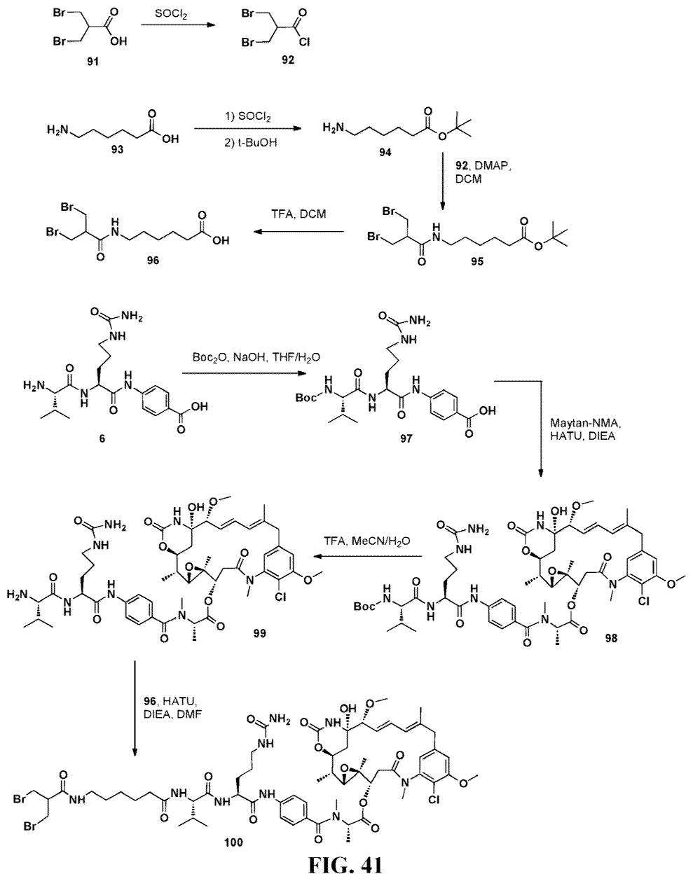

FIG. 42 depicts the deconvoluted mass spectroscopy (MS) spectrum of the antibody drug conjugate, PRLR-Q-63 conjugate from EXAMPLE 43.

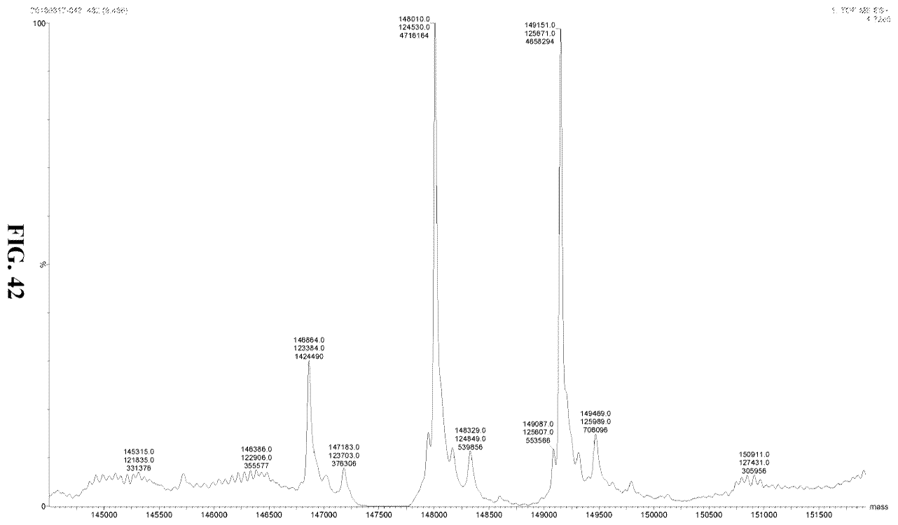

FIG. 43 depicts the deconvoluted MS spectrum of the Isotype Control-Q-63 conjugate from EXAMPLE 43.

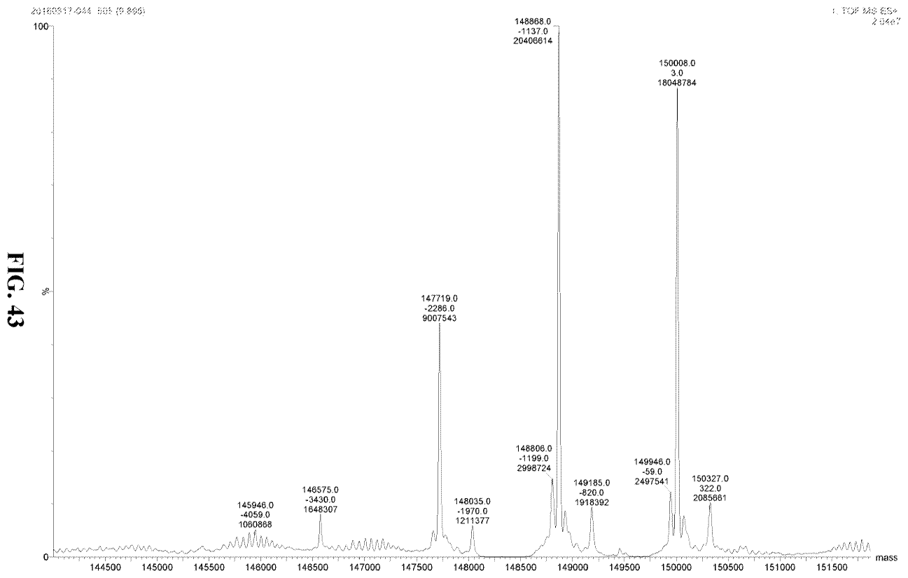

FIG. 44 depicts the plot of % Cell Viability vs. Log₁₀[M] of certain compounds tested in EXAMPLE 45.

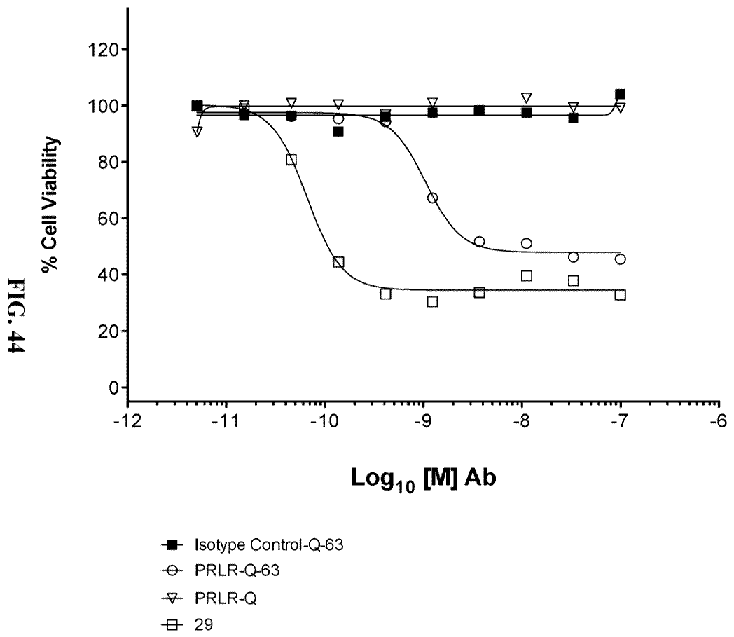

FIG. 45 depicts the plot of % Cell Viability vs. Log₁₀[M] of certain compounds tested in EXAMPLE 45.

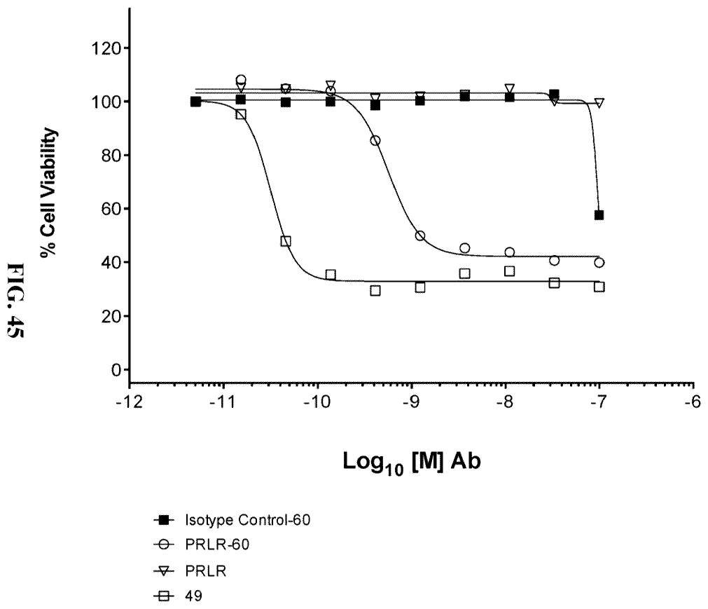

FIG. 46 depicts the plot of % Cell Viability vs. Log 10 [M] of certain compounds tested in EXAMPLE 45.

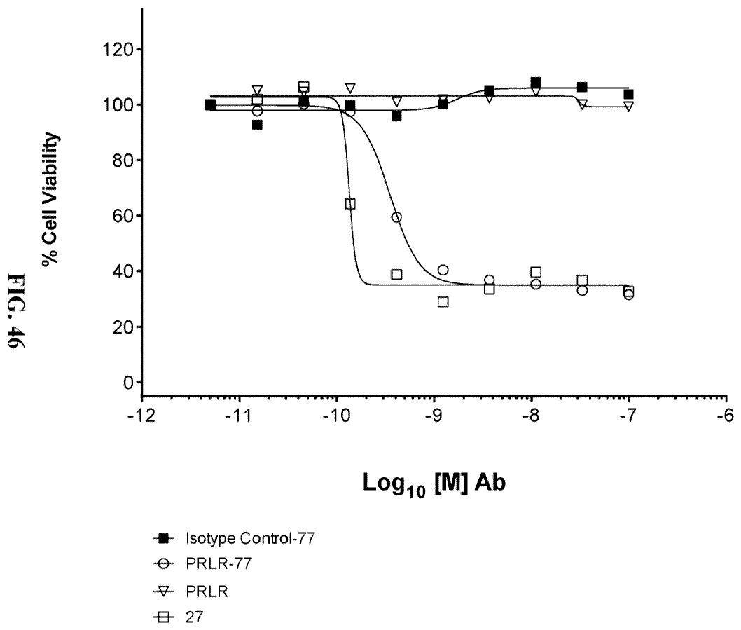

FIG. 47 depicts the plot of % Cell Viability vs. Log 10 [M] of certain compounds tested in EXAMPLE 45.

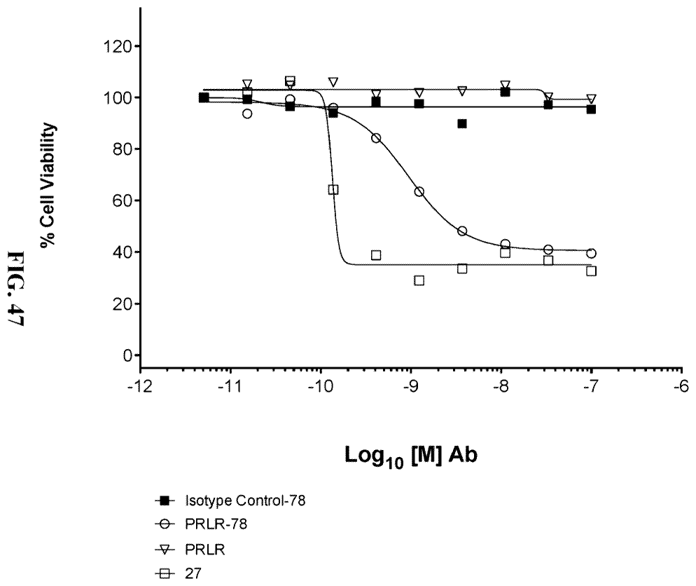

### DETAILED DESCRIPTION

### A. Definitions

As used herein, “alkyl” refers to a monovalent and saturated hydrocarbon radical moiety. Alkyl is optionally substituted and can be linear, branched, or cyclic, i.e., cycloalkyl. Alkyl includes, but is not limited to, those having 1-20 carbon atoms, i.e., C₁₋₂₀ alkyl; 1-12 carbon atoms, i.e., C₁₋₁₂ alkyl; 1-8 carbon atoms, i.e., C₁₋₈ alkyl; 1-6 carbon atoms, i.e., C₁₋₆ alkyl; and 1-3 carbon atoms, i.e., C₁₋₃ alkyl. Examples of alkyl moieties include, but are not limited to methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl.

As used herein, “haloalkyl” refers to alkyl, as defined above, wherein the alkyl includes at least one substituent selected from a halogen, e.g., F, Cl, Br, or I.

As used herein, “alkenyl” refers to a monovalent hydrocarbon radical moiety containing at least two carbon atoms and one or more non-aromatic carbon-carbon double bonds. Alkenyl is optionally substituted and can be linear, branched, or cyclic. Alkenyl includes, but is not limited to, those having 2-20 carbon atoms, i.e., C₂₋₂₀ alkenyl; 2-12 carbon atoms, i.e., C₂₋₁₂ alkenyl; 2-8 carbon atoms, i.e., C₂₋₈ alkenyl; 2-6 carbon atoms, i.e., C₂₋₆ alkenyl; and 2-4 carbon atoms, i.e., C₂₋₄ alkenyl. Examples of alkenyl moieties include, but are not limited to vinyl, propenyl, butenyl, and cyclohexenyl.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “alkoxy” refers to a monovalent and saturated hydrocarbon radical moiety wherein the hydrocarbon includes a single bond to an oxygen atom and wherein the radical is localized on the oxygen atom, e.g., CH₃CH₂—O· for ethoxy. Alkoxy substituents bond to the compound which they substitute through this oxygen atom of the alkoxy substituent. Alkoxy is optionally substituted and can be linear, branched, or cyclic, i.e., cycloalkoxy. Alkoxy includes, but is not limited to, those having 1-20 carbon atoms, i.e., C₁₋₂₀ alkoxy; 1-12 carbon atoms, i.e., C₁₋₁₂ alkoxy; 1-8 carbon atoms, i.e., C₁₋₈ alkoxy; 1-6 carbon atoms, i.e., C₁₋₆ alkoxy; and 1-3 carbon atoms, i.e., C₁₋₃ alkoxy. Examples of alkoxy moieties include, but are not limited to methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy.

As used herein, “haloalkoxy” refers to alkoxy, as defined above, wherein the alkoxy includes at least one substituent selected from a halogen, e.g., F, Cl, Br, or I.

As used herein, “aryl” refers to a monovalent moiety that is a radical of an aromatic compound wherein the ring atoms are carbon atoms. Aryl is optionally substituted and can be monocyclic or polycyclic, e.g., bicyclic or tricyclic. Examples of aryl moieties include, but are not limited to those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to phenyl, naphthyl, fluorenyl, azulenyl, anthryl, phenanthryl, and pyrenyl.

As used herein, “arylene” refers to a divalent moiety of an aromatic compound wherein the ring atoms are only carbon atoms. Arylene is optionally substituted and can be monocyclic or polycyclic, e.g., bicyclic or tricyclic. Examples of aryl moieties include, but are not limited to those having 6 to 20 ring carbon atoms, i.e., C₆₋₂₀ arylene; 6 to 15 ring carbon atoms, i.e., C₆₋₁₅ arylene, and 6 to 10 ring carbon atoms, i.e., C₆₋₁₀ arylene.

As used herein, “alkaryl” refers to an aryl that is substituted with at least one alkyl. Alkaryl is optionally substituted.

As used herein, “heteroalkyl” refers to an alkyl in which one or more carbon atoms are replaced by heteroatoms. As used herein, “heteroalkenyl” refers to an alkenyl in which one or more carbon atoms are replaced by heteroatoms. As used herein, “heteroalkynyl” refers to an alkenyl in which one or more carbon atoms are replaced by heteroatoms. Suitable heteroatoms include, but are not limited to, nitrogen, oxygen, and sulfur atoms. Heteroalkyl is optionally substituted. Examples of heteroalkyl moieties include, but are not limited to, aminoalkyl, sulfonylalkyl, sulfinylalkyl. Examples of heteroalkyl moieties also include, but are not limited to, methylamino, methylsulfonyl, and methylsulfinyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted.

As used herein, “heteroarylene” refers to an arylene in which one or more ring atoms of the aromatic ring are replaced with an oxygen, sulfur, nitrogen, or phosphorus atom. Heteroarylene is optionally substituted.

As used herein, “heterocycloalkyl” refers to a cycloalkyl in which one or more carbon atoms are replaced by heteroatoms. Suitable heteroatoms include, but are not limited to, nitrogen, oxygen, and sulfur atoms. Heterocycloalkyl is optionally substituted. Examples of heterocycloalkyl moieties include, but are not limited to, morpholinyl, piperidinyl, tetrahydropyranyl, pyrrolidinyl, imidazolidinyl, oxazolidinyl, thiazolidinyl, dioxolanyl, dithiolanyl, oxanyl, or thianyl.

As used herein, “optionally substituted,” when used to describe a radical moiety, e.g., optionally substituted alkyl, means that such moiety is optionally bonded to one or more substituents. Examples of such substituents include, but are not limited to halo, cyano, nitro, haloalkyl, azido, epoxy, optionally substituted heteroaryl, optionally substituted heterocycloalkyl,

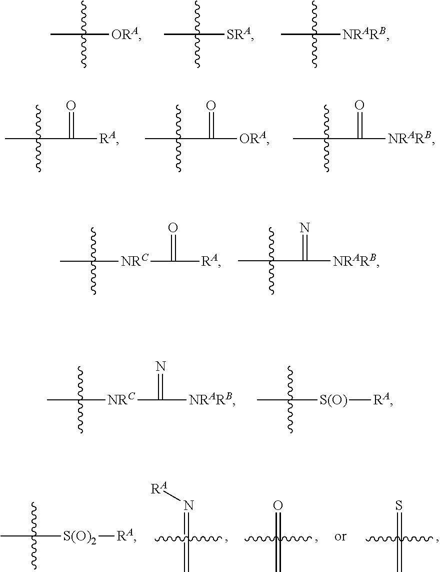

wherein R^{A}, R^{B}, and R^{C} are, independently at each occurrence, a hydrogen atom, alkyl, alkenyl, alkynyl, aryl, alkaryl, aralkyl, heteroalkyl, heteroaryl, or heterocycloalkyl, or R^{A} and R^{B}, together with the atoms to which they are bonded, form a saturated or unsaturated carbocyclic ring, wherein the ring is optionally substituted and wherein one or more ring atoms is optionally replaced with a heteroatom. In some embodiments, R^{A}, R^{B}, and R^{C} are not hydrogen atoms. In some examples, R^{A} is methyl. In some examples, R^{A} is methylamino, methylsulfonyl, and methylsulfinyl. In some examples, R^{A} is methylamino. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

As used herein, “binding agent” refers to any molecule capable of binding with specificity to a given binding partner.

As used herein, “linker” refers to a divalent moiety that covalently links the binding agent to the maytansinoid derivatives described herein.

As used herein, “amide synthesis conditions” refers to reaction conditions suitable to effect the formation of an amide, e.g., by the reaction of a carboxylic acid, activated carboxylic acid, or acyl halide with an amine. In some examples, amide synthesis conditions refers to reaction conditions suitable to effect the formation of an amide bond between a carboxylic acid and an amine. In some of these examples, the carboxylic acid is first converted to an activated carboxylic acid before the activated carboxylic acid reacts with an amine to form an amide. Suitable conditions to effect the formation of an amide include, but are not limited to, those utilizing reagents to effect the reaction between a carboxylic acid an amine, including, but not limited to, dicyclohexylcarbodiimide (DCC), diisopropylcarbodiimide (DIC), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (7-azabenzotriazol-1-yloxy)tripyrrolidinophosphonium hexafluorophosphate (PyAOP), bromotripyrrolidinophosphonium hexafluorophosphate (PyBrOP), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), 1-[Bis(dimethylamino)methylene]-1H-1,2,3-triazolo[4,5-b]pyridinium 3-oxide hexafluorophosphate (HATU), 2-Ethoxy-1-ethoxycarbonyl-1,2-dihydroquinoline (EEDQ), 1-Ethyl-3-(3-dimethylaminopropyl)carbodiimide (EDC), 2-Chloro-1,3-dimethylimidazolidinium hexafluorophosphate (CIP), 2-chloro-4,6-dimethoxy-1,3,5-triazine (CDMT), and carbonyldiimidazole (CDI). In some examples, a carboxylic acid is first converted to an activated carboxylic ester before reacting with an amine to form an amide bond. In certain embodiments, the carboxylic acid is reacted with a reagent. The reagent activates the carboxylic acid by deprotonating the carboxylic acid and then forming a product complex with the deprotonated carboxylic acid as a result of nucleophilic attack by the deprotonated carboxylic acid onto the protonated reagent. For certain carboxylic acids, this activated ester is more susceptible subsequently to nucleophilic attack by an amine than the carboxylic acid is before it is converted. This results in amide bond formation. As such, the carboxylic acid is described as activated. Exemplary reagents include DCC and DIC.

As used herein, “therapeutically effective amount” refers to an amount (of a compound) that is sufficient to provide a therapeutic benefit to a patient in the treatment or management of a disease or disorder, or to delay or minimize one or more symptoms associated with the disease or disorder.

Certain groups, moieties, substituents, and atoms are depicted with a wiggly line that intersects a bond or bonds to indicate the atom through which the groups, moieties, substituents, atoms are bonded. For example, a phenyl group that is substituted with a propyl group depicted as:

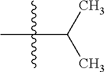 [SMILES: *C(C)C]

has the following structure:

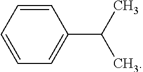

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

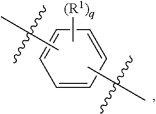

wherein subscript q is an integer from 0 to 4 and in which the positions of substituent R¹ are described generically, includes the following groups in which the positions of substituent R¹ are described specifically:

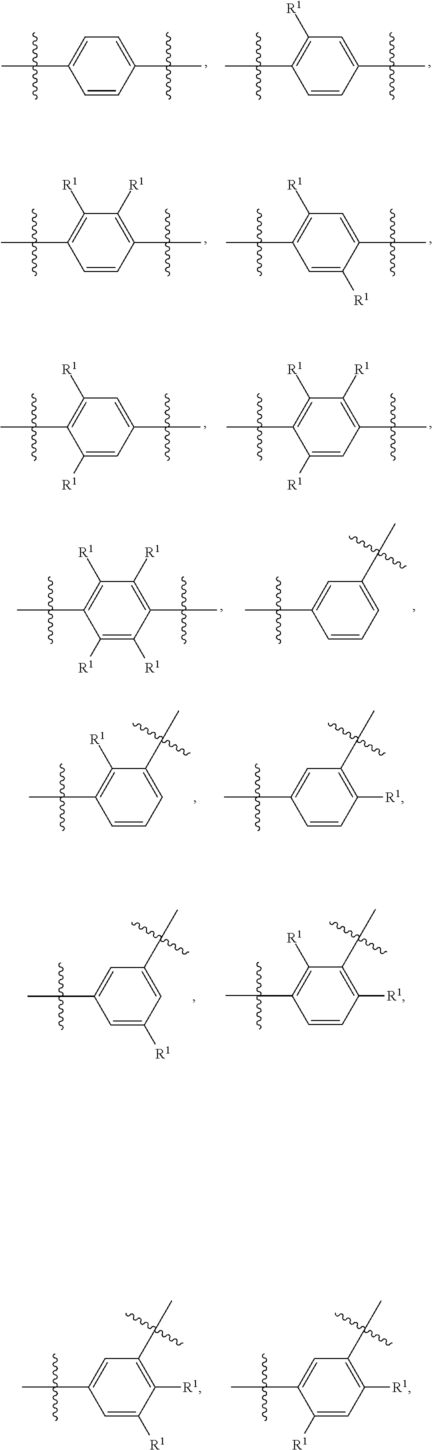

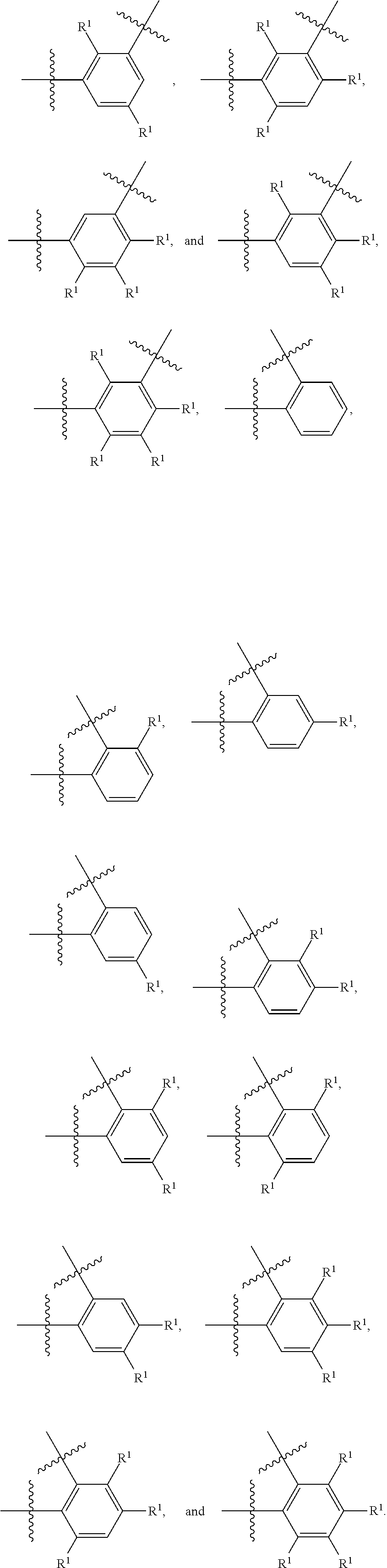

In addition and for example, the group,

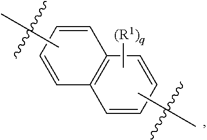

in which the positions of substituents other than R¹ which are bonded to the cyclic group through a bond between ring atoms are described generically, includes the following groups in which the positions of these substituents other than R¹ are described specifically:

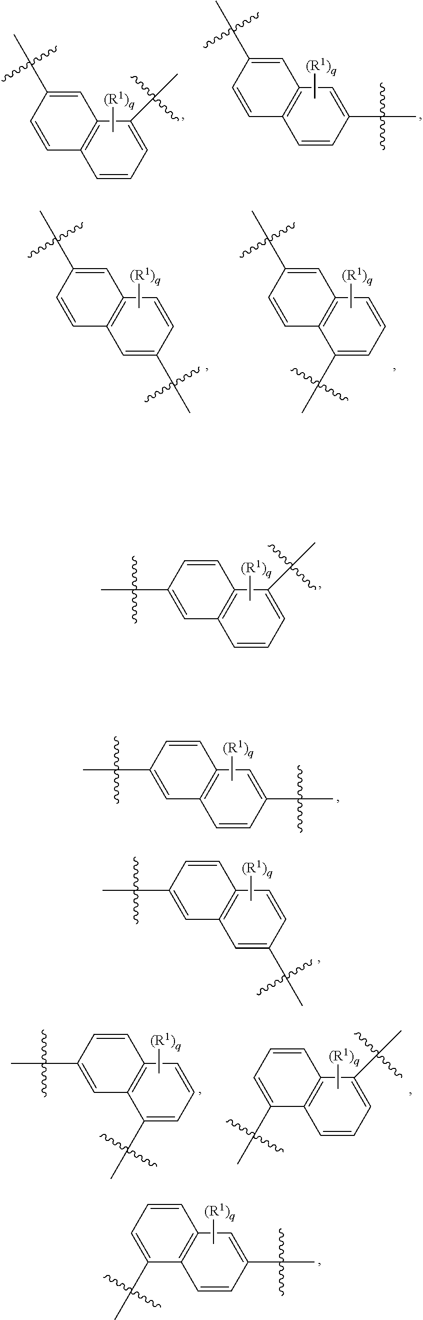

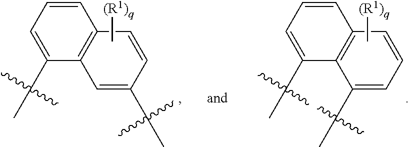

Also, for example, the group,

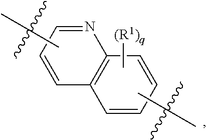

in which the positions of substituents other than R¹ which are bonded to the cyclic group through a bond between ring atoms are described generically, includes the following groups in which the positions of these substituents other than R¹ are described specifically:

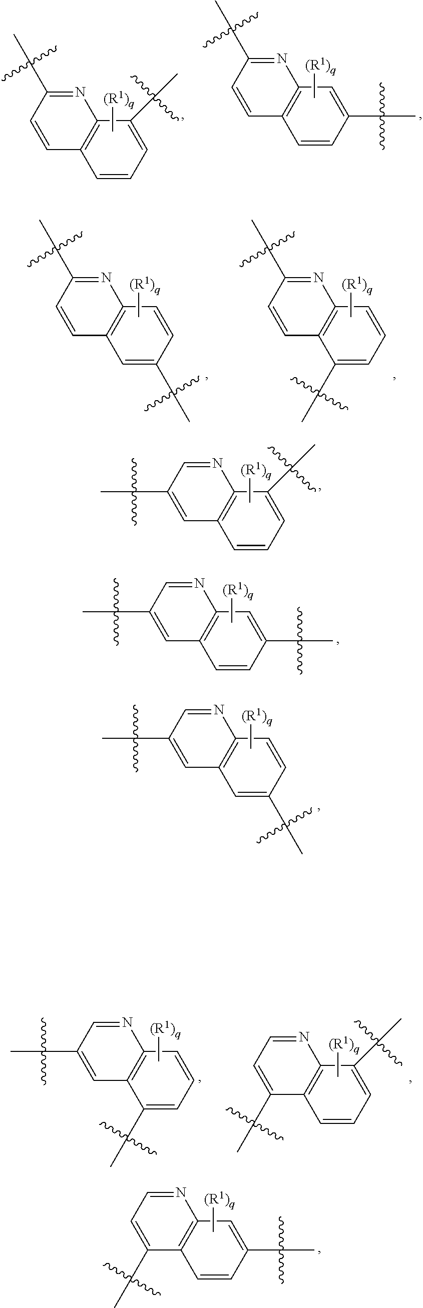

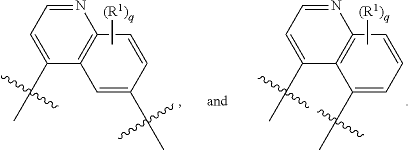

In each of these structures in which the positions of the substituents other than R¹ are described specifically, the substituent R¹ may be bonded to any ring position in the cyclic group or on any ring in the fused ring group which is not occupied by one of these substituents other than R¹. The following non-limiting representative illustrations indicate that the cyclic group can be substituted with the indicated substituent at any ring position or on either ring in the fused ring group:

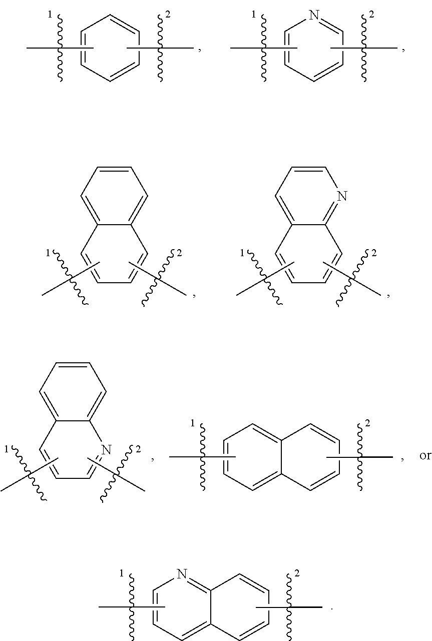

### B. Conjugates

Provided herein are compounds of Formula (I):

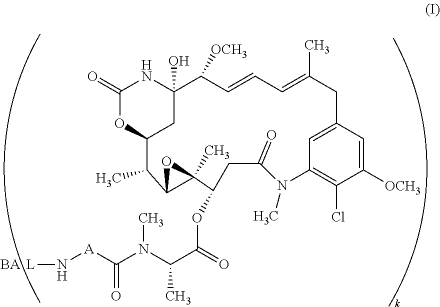

or a pharmaceutically acceptable salt thereof,
wherein:
A is arylene or heteroarylene;
L is a linker;
BA is a binding agent; and
k is an integer from 1 to 30.

### 1. “A” Moieties

In some embodiments, A is arylene. In some embodiments, A is heteroarylene. In some embodiments, the arylene or heteroarylene is substituted with one or more electron withdrawing groups and/or one or more electron donating groups.

In some embodiments, A is a divalent radical of benzene, of pyridine, of naphthalene, or of quinolone, which are optionally substituted.

In some embodiments, A is a divalent radical of benzene which is optionally substituted with a member selected from the group consisting of amino, amido, alkyl, halo, haloalkyl, alkoxy, and haloalkoxy.

In some embodiments, A is:

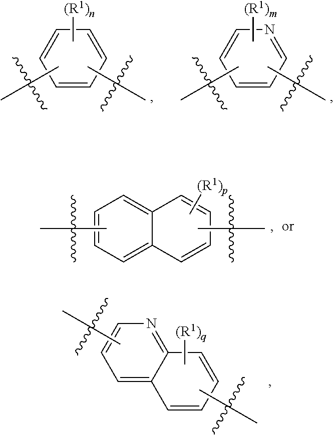

wherein:
R¹, independently at each occurrence, is alkyl, alkenyl, alkynyl, aryl, alkaryl, aralkyl, halo, heteroaryl, heterocycloalkyl, hydroxyl, cyano, nitro,

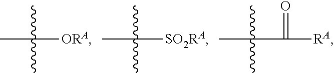

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, A is:

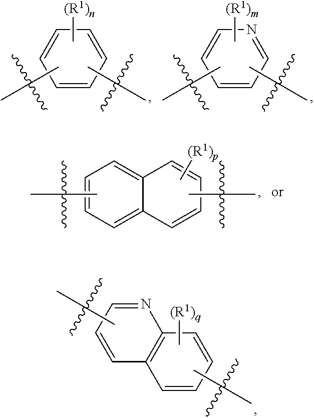

wherein:
R¹ is, independently at each occurrence, halo, haloalkyl, haloalkoxy, hydroxyl, alkyl, alkenyl, alkynyl, alkoxy, haloalkoxy, aryl, alkaryl, aralkylheteroaryl, heteroalkyl, heterocycloalkyl, cyano, nitro,

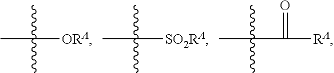

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, A is:

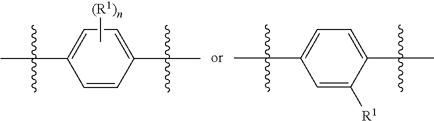

wherein:
R¹ is, independently at each occurrence, halo, haloalkyl, hydroxyl, alkyl, alkenyl, alkynyl, alkoxy, aryl, alkaryl, aralkyl, heteroaryl, heteroalkyl, heterocycloalkyl, cyano, nitro,

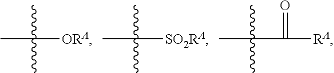

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, R¹ is C₁₋₆ alkyl or C₁₋₆ alkoxy. In some of these embodiments, R¹ is methyl, ethyl, methoxy, or ethoxy. In some of these embodiments, R¹ is methoxy.

In some embodiments, R¹ is, independently, alkyl or halo. In some embodiments, R¹ is, independently, C₁₋₆ alkyl, C₁₋₆ haloalkyl, or halo. In some embodiments, R¹ is, independently, halo. In some embodiments, R¹ is, independently, fluoro, chloro, bromo, iodo, or trifluoromethyl. In some embodiments, n, m, p, or q is 0, 1 or 2. In some embodiments, n, m, p, or q is 0 or 1. In some embodiments, n, m, p, or q is 0.

In some embodiments, R¹ is

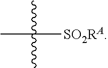

In some embodiments, R¹ is

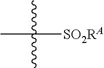

wherein R^{A} is methyl. In some embodiments, R¹ is hydroxyl. In some embodiments, R¹ is N-methylformamide. In some embodiments, R¹ is morpholinyl.

In some embodiments, R¹ is, independently, alkyl, alkoxy, or halo. In some embodiments, R¹ is, independently, C₁₋₆ alkyl, C₁₋₆ alkoxy, C₁₋₆ haloalkyl, C₁₋₆ haloalkoxy, or halo. In some embodiments, R¹ is, independently, halo. In some embodiments, R¹ is, independently, fluoro, chloro, bromo, iodo, or trifluoromethyl. In some embodiments, n, m, p, or q is 0, 1 or 2. In some embodiments, n, m, p, or q is 0 or 1. In some embodiments, n, m, p, or q is 0.

In some embodiments, A is:

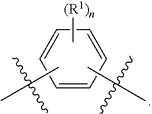

In some embodiments, A is:

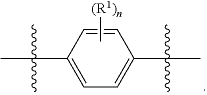

In some embodiments, A is:

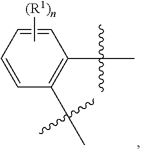

In some embodiments, A is:

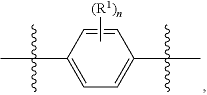

wherein n is 0, 1 or 2.

In some embodiments, A is:

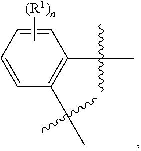

wherein n is 0, 1 or 2.

In some embodiments, A is:

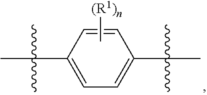

wherein n is 0 or 1; and R¹ is alkoxy, halo, or haloalkyl.

In some embodiments, A is:

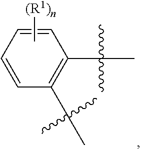

wherein n is 0 or 1; and R¹ is alkoxy, halo, or haloalkyl. In some examples, R¹ is alkoxy.

In some embodiments, A is:

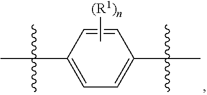

wherein n is 0 or 1; and R¹ is C₁₋₆ alkoxy, halo, or C₁₋₆ haloalkyl.

In some embodiments, A is:

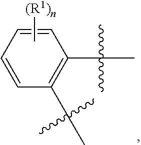

wherein n is 0 or 1; and R¹ is C₁₋₆ alkoxy, halo, or C₁₋₆ haloalkyl. In some examples, R¹ is C₁₋₆ alkoxy.

In some embodiments, A is:

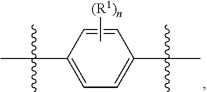

wherein n is 0 or 1; R¹ is C₁₋₆ alkoxy, halo, or C₁₋₆ haloalkyl; and L is

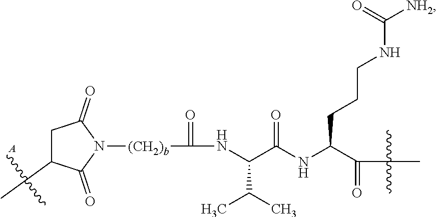

wherein b is an integer from 2 to 8 and

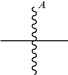

is a bond to the binding agent.

In some embodiments, A is:

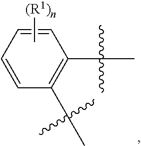

wherein n is 0 or 1; R¹ is C₁₋₆ alkoxy, halo, or C₁₋₆ haloalkyl; and L is

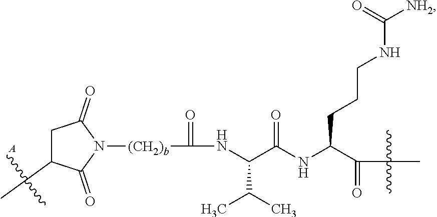

wherein b is an integer from 2 to 8 and

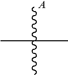

is a bond to the binding agent.

In some embodiments, A is:

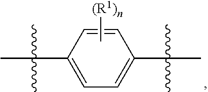

wherein n is 0, 1, 2, 3, or 4.

In some embodiments, A is:

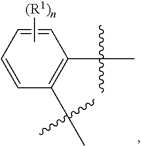

wherein n is 0, 1, 2, 3, or 4.

In some embodiments, A is:

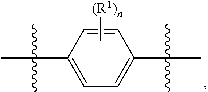

wherein:
R¹ is C₁₋₆ alkyl, halo, or C₁₋₆ haloalkyl; and
n is 0, 1 or 2.

In some embodiments, A is:

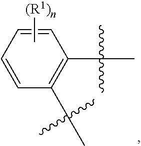

wherein:
   R¹ is C₁₋₆ alkyl, halo, or C₁₋₆ haloalkyl; and
   n is 0, 1 or 2.

In some embodiments, A is:

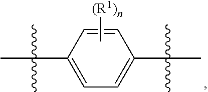

wherein:
R¹ is C₁₋₆ alkyl, C₁₋₆ alkoxy, halo, C₁₋₆ haloalkyl, or C₁₋₆ haloalkoxy; and
n is 0, 1, 2, 3, or 4.

In some embodiments, A is:

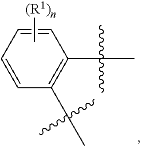

wherein:
R¹ is C₁₋₆ alkyl, C₁₋₆ alkoxy, halo, C₁₋₆ haloalkyl, or C₁₋₆ haloalkoxy; and
n is 0, 1, 2, 3, or 4.

In some embodiments, A is:

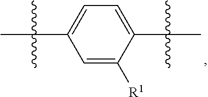

wherein R¹ is C₁₋₆ alkyl, C₁₋₆ alkoxy, halo, or C₁₋₆ haloalkyl. In certain of these embodiments, R¹ is methoxy or methyl. In some specific embodiments, R¹ is methoxy.

In some embodiments, A is:

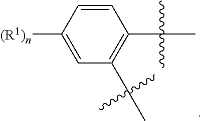

In some embodiments, A is:

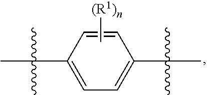

wherein:
R¹ is halo or trifluoromethyl; and
n is 0, 1 or 2.

In some embodiments, A is:

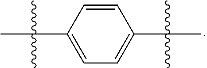 [SMILES: *c1ccc(*)cc1]

In some embodiments, A is:

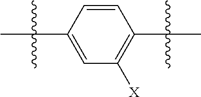

wherein:
X is a hydrogen atom, halo, or trifluoromethyl.

In some embodiments, A is:

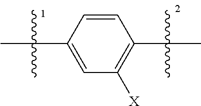

wherein:
X is a hydrogen atom, halo, or trifluoromethyl;

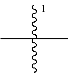

is the bond to the nitrogen atom; and

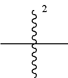

is the bond to the carbonyl.

In some embodiments, A is:

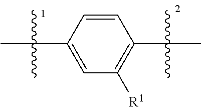

wherein:
R¹ is, independently at each occurrence, a hydrogen atom, alkyl, alkoxy, aryl, heteroalkyl, halo, haloalkyl, haloalkoxy or hydroxyl;

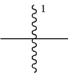

is the bond to the nitrogen atom; and

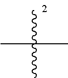

is the bond to the carbonyl. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen. In some embodiments, A is:

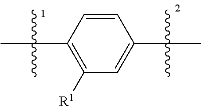

wherein:
R¹ is, independently at each occurrence, a hydrogen atom, alkyl, alkoxy, aryl, heteroalkyl, halo, haloalkyl, haloalkoxy, or hydroxyl;

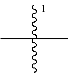

is the bond to the nitrogen atom; and

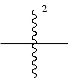

is the bond to the carbonyl. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen.

In some embodiments, R¹ is sulfonyl. In some embodiments, R¹ is N-methylformamide. In some embodiments, R¹ is hydroxyl. In some embodiments, R¹ is morpholinyl.

In some embodiments, A is:

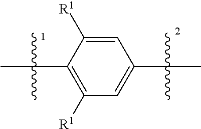

wherein:
R¹ is, independently at each occurrence, a hydrogen atom, alkyl, alkoxy, aryl, heteroalkyl, halo, haloalkyl, or haloalkoxy;

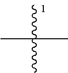

is the bond to the nitrogen atom; and

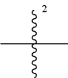

is the bond to the carbonyl. In some embodiments, R¹ is alkyl or alkoxy. In some specific embodiments, R¹ is propylamino, difluoro-methoxy, phenyl, 2-fluorophenyl. In some embodiments, R¹ is trifluoromethyl. In some embodiments, R¹ is methoxy. In some embodiments, R¹ is fluoro. In some embodiments, R¹ is hydrogen.

In some embodiments, A is:

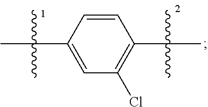 [SMILES: *c1ccc(*)c(Cl)c1]

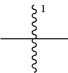

is the bond to the nitrogen atom; and

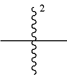

is the bond to the carbonyl.

In some embodiments, A is:

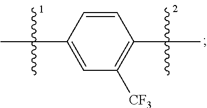 [SMILES: *c1ccc(*)c(C(F)(F)F)c1]

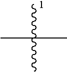

is the bond to the nitrogen atom; and

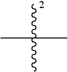

is the bond to the carbonyl.

In some embodiments, A is:

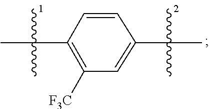 [SMILES: *c1ccc(*)c(C(F)(F)F)c1]

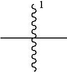

is the bond to the nitrogen atom; and

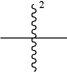

is the bond to the carbonyl.

In some embodiments, A is:

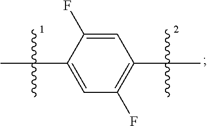 [SMILES: *c1cc(F)c(*)cc1F]

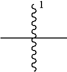

is the bond to the nitrogen atom; and

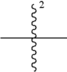

is the bond to the carbonyl.

In some embodiments, A is:

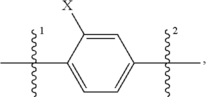

wherein X is F, Cl, Br, CN, methoxy, dimethylamino or cyclopropyl;

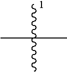

is the bond to the nitrogen atom; and

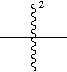

is the bond to the carbonyl.

In some embodiments, A is:

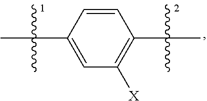

wherein X is F, Cl, Br, CN, methoxy, dimethylamino, 1-methyl-ethyl-thio or cyclopropyl;

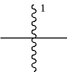

is the bond to the nitrogen atom; and

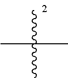

is the bond to the carbonyl.

In some embodiments, A is:

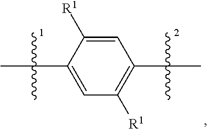

wherein each R¹ is independently, at each occurrence, a hydrogen atom, alkyl, alkoxy, halo, haloalkyl, or haloalkoxy;

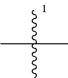

is the bond to the nitrogen atom; and

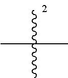

is the bond to the carbonyl. In some embodiments, R¹ is hydrogen, fluoro, trifluoromethyl, or methoxy. In some embodiments, R¹ is fluoro, chloro, bromo, or iodo.

In some embodiments, A is:

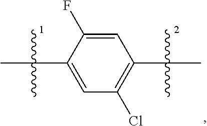 [SMILES: *c1cc(Cl)c(*)cc1F]

wherein

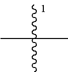

is the bond to the nitrogen atom; and

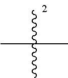

is the bond to the carbonyl.

In some embodiments, A is:

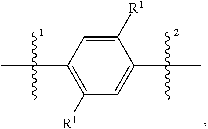

wherein each R¹ is independently, at each occurrence, a hydrogen atom, alkyl, alkoxy, halo, haloalkyl, or haloalkoxy,

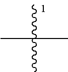

is the bond to the nitrogen atom; and

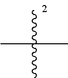

is the bond to the carbonyl.

In some embodiments, A is:

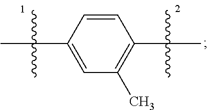 [SMILES: *c1ccc(*)c(C)c1]

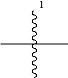

is the bond to the nitrogen atom; and

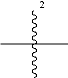

is the bond to the carbonyl.

In some embodiments, A is:

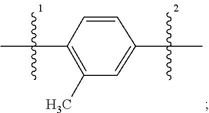 [SMILES: *c1ccc(*)c(C)c1]

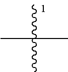

is the bond to the nitrogen atom; and

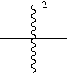

is the bond to the carbonyl.

In some embodiments, A is:

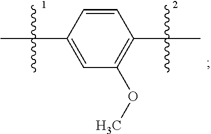

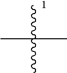

is the bond to the nitrogen atom; and

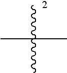

is the bond to the carbonyl.

In some embodiments, A is:

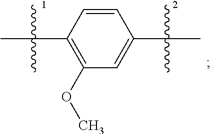

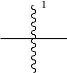

is the bond to the nitrogen atom; and

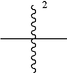

is the bond to the carbonyl.

In some embodiments, A is:

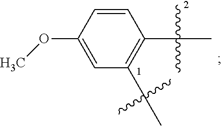

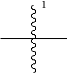

is the bond to the nitrogen atom; and

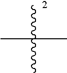

is the bond to the carbonyl.

In some embodiments, A is:

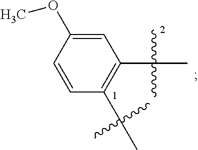

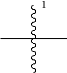

is the bond to the nitrogen atom; and

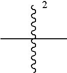

is the bond to the carbonyl.

In some embodiments, A is:

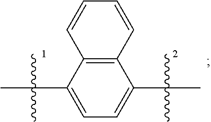

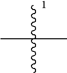

is the bond to the nitrogen atom; and

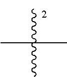

is the bond to the carbonyl.

In some embodiments, A is:

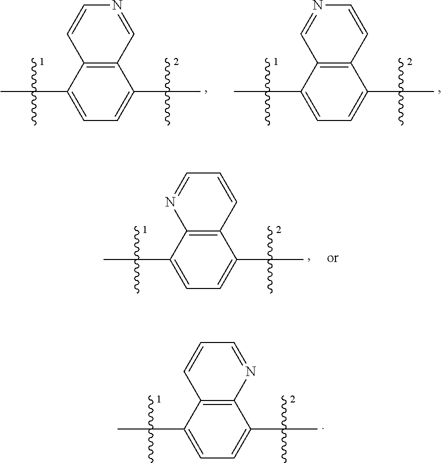 [SMILES: *c1ccc(*)c2cnccc12 ; *c1ccc(*)c2cnccc12 ; *c1ccc(*)c2ncccc12 ; *c1ccc(*)c2ncccc12]

In some embodiments, A is:

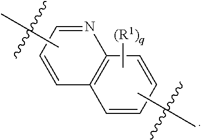

In some embodiments, A is:

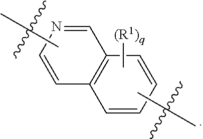

In some embodiments, A is:

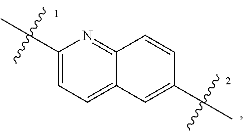 [SMILES: *c1ccc2nc(*)ccc2c1]

wherein:

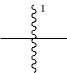

is the bond to the nitrogen atom; and

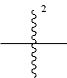

is the bond to the carbonyl.

In some embodiments, A is:

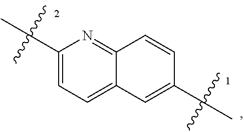 [SMILES: *c1ccc2nc(*)ccc2c1]

wherein:

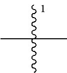

is the bond to the nitrogen atom; and

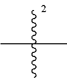

is the bond to the carbonyl.

In some embodiments, A is:

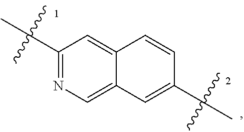 [SMILES: *c1ccc2cc(*)ncc2c1]

wherein:

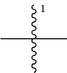

is the bond to the nitrogen atom; and

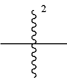

is the bond to the carbonyl.

In some embodiments, A is:

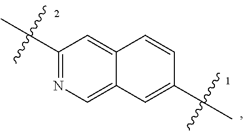 [SMILES: *c1ccc2cc(*)ncc2c1]

wherein:

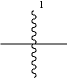

is the bond to the nitrogen atom; and

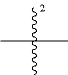

is the bond to the carbonyl.

In some embodiments, A is:

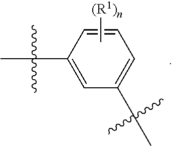

In some embodiments, A is:

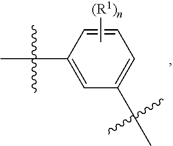

wherein n is 0, 1 2, or 3.

In some embodiments, A is:

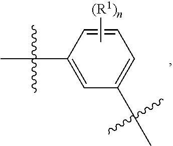

wherein:
R¹ is C₁₋₆ alkyl, C₁₋₆ alkoxy, halo, C₁₋₆ haloalkyl, C₁₋₆ haloalkoxy, C₁₋₆ heteroalkyl; and
n is 0, 12, 3 or 4.

In some embodiments, A is:

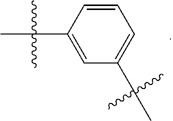 [SMILES: *c1cccc(*)c1]

In some embodiments, A is:

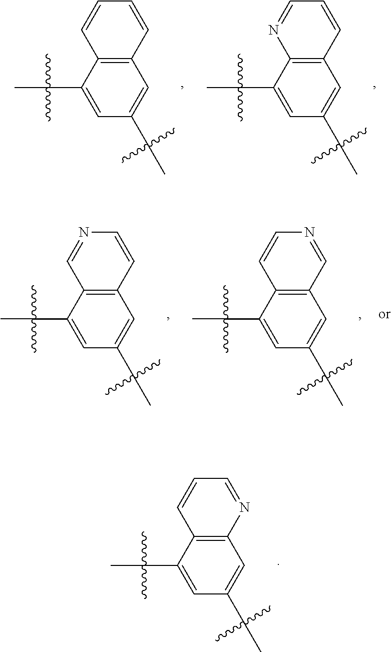 [SMILES: *c1cc(*)c2ccccc2c1 ; *c1cc(*)c2ncccc2c1 ; *c1cc(*)c2cnccc2c1 ; *c1cc(*)c2ccncc2c1 ; *c1cc(*)c2cccnc2c1]

In some embodiments, A is:

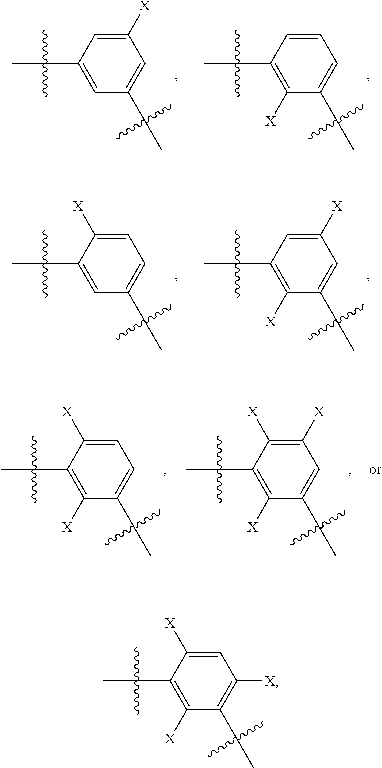

wherein:
X is, independently at each occurrence, a hydrogen atom, alkyl, alkoxy, halo, haloalkoxy, haloalkyl, heteroalkyl. In some embodiments, X is fluoro, chloro, bromo, iodo, dimethylamino, methylamino, methoxy, ethoxy, or trifluoromethyl.

In some embodiments, A is:

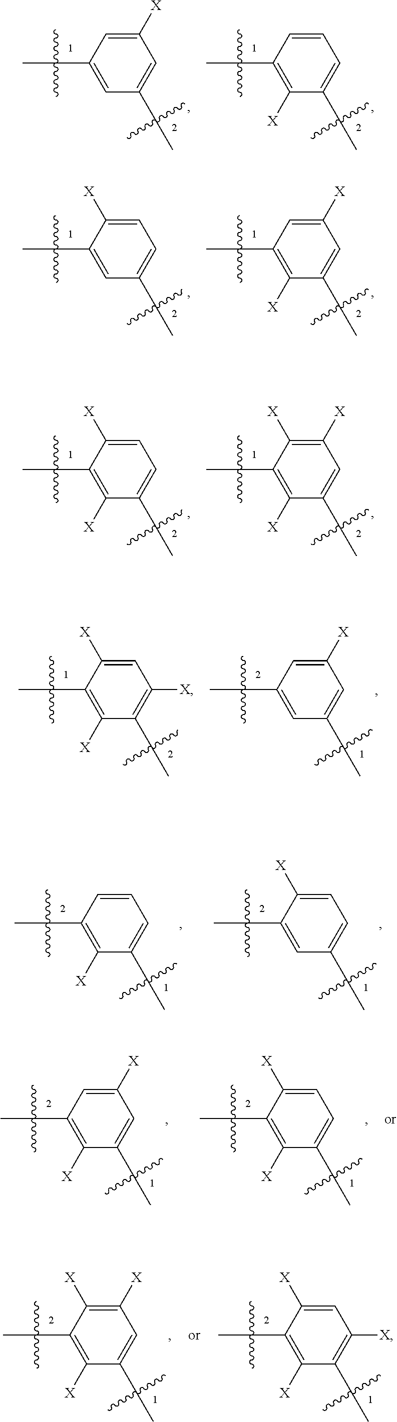

wherein:
X is, independently at each occurrence, a hydrogen atom, alkyl, alkoxy, halo, haloalkoxy, haloalkyl, heteroalkyl. In some embodiments, X is fluoro, chloro, bromo, iodo, dimethylamino, methylamino, methoxy, ethoxy, trifluoromethyl or methoxy;

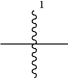

is the bond to the nitrogen atom; and

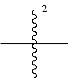

is the bond to the carbonyl.

### 2. Linkers

The linker portion of the conjugates described herein is a divalent moiety that covalently links the binding agent to the maytansinoid derivatives described herein. Suitable linkers include those that release the maytansinoid portion in the presence of an enzyme or at a particular pH range or value.

In some embodiments, the linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the linker comprises a non-cleavable moiety. In some embodiments, the non-cleavable linker is

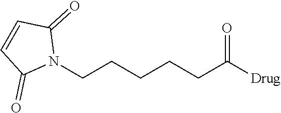

or a residue thereof. In some embodiments, the non-cleavable linker is

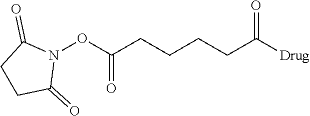

or a residue thereof.

Suitable linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the linker comprises one or more amino acids. Suitable amino acids include natural, non-natural, standard, non-standard, proteinogenic, non-proteinogenic, and L-, or D-α-amino acids. In some embodiments, the linker comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, or derivative thereof.

In some embodiments, the linker comprises valine and citrulline.

In some embodiments, the linker is:

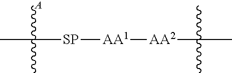

wherein:
SP is a spacer;

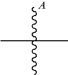

is one or more bonds to the binding agent;
AA¹ is an amino acid; and
AA² is an amino acid.

The spacer is a divalent moiety that connects the AA¹-AA² moiety to the binding agent (BA). Suitable spacers include, but are not limited to, those comprising alkylene or polyethylene glycol. The ends of the spacers, i.e., the portion of the spacer directly bonded to the binding agent or AA¹, can be moieties derived from reactive moieties that are used for purposes of coupling the naked antibody or AA¹ to the spacer during the chemical synthesis of the conjugate.

In some examples, suitable spacers include, but are not limited to, a primary amine-terminated alkylene or a primary amine-terminated polyethylene glycol. The primary amine-terminating end of the spacer can be directly bonded to a deglycosylated antibody or aglycosylated antibody in the presence of transglutaminase.

In some embodiments, the spacer comprises an alkylene. In some embodiments, the spacer comprises a C₅₋₇ alkylene. In some embodiments, the spacer is:

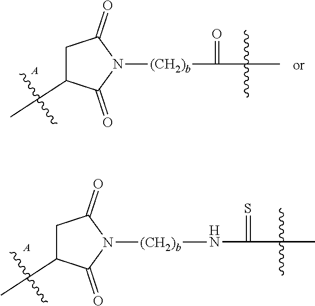

wherein:

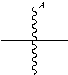

is a bond to the binding agent; and
b is an integer from 2 to 8.

In some embodiments, the spacer comprises a primary amine-terminated alkylene. In some embodiments, the spacer comprises a NH₂—C₅₋₇ alkylene. In some embodiments, the spacer is:

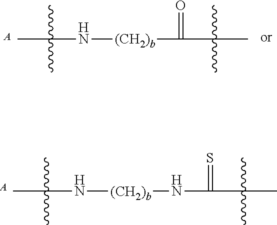

wherein:

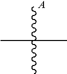

is a bond to the binding agent; and
b is an integer from 2 to 8.

In some embodiments, the spacer is:

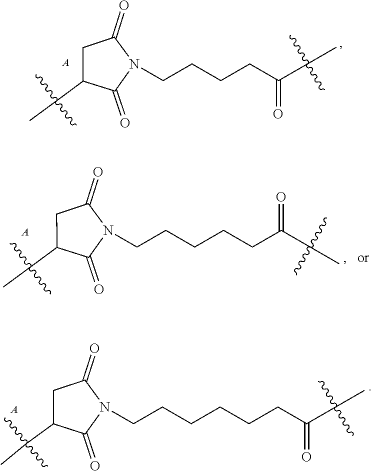

wherein:

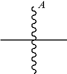

is a bond to the binding agent.

In some embodiments, the spacer is:

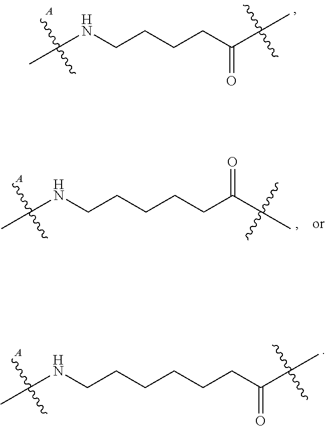

wherein:

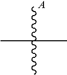

is a bond to the binding agent.

In some embodiments, the spacer is:

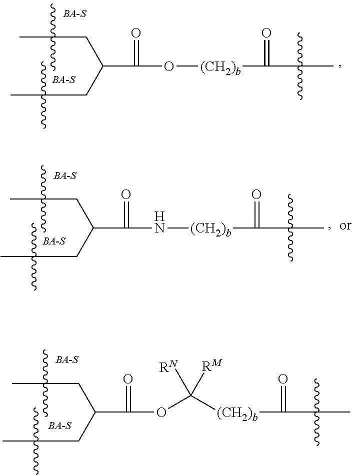

wherein:
R^{N} is a hydrogen atom or alkyl;
R^{M} is alkyl;
the two bonds represented by

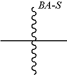

are bonds to cysteines of a binding agent; and
b is an integer from 2 to 8.

In some embodiments, the spacer is:

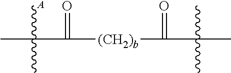

wherein:

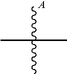

is a bond to the binding agent; and
b is an integer from 2 to 8.

In some embodiments, the spacer is:

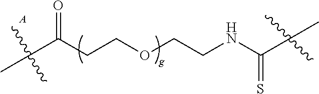

wherein:

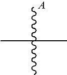

is a bond to the binding agent; and
g is an integer from 2 to 20. In some embodiments, g is 2-8. In some embodiments, g is 2, 4, 6, or 8.

In some embodiments, the spacer is

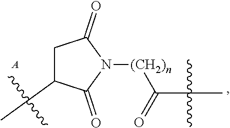

wherein n is an integer from 4 to 10. In some embodiments, n is 4, 5, 6, 7, 8, 9, or 10.

In some embodiments, the spacer is:

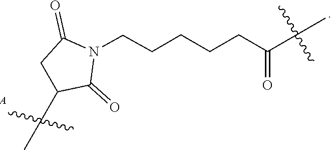

In some embodiments, the spacer is

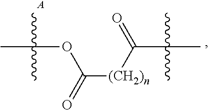

wherein n is an integer from 4 to 10. In some embodiments, n is 4, 5, 6, 7, 8, 9, or 10.

In some embodiments, the spacer is

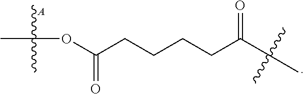

In some embodiments, the spacer is:

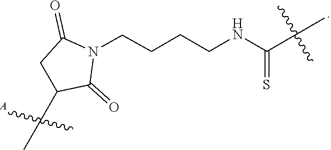

In some embodiments, the spacer is:

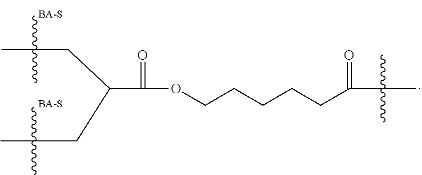 [SMILES: *CC(C*)C(=O)OCCCCCC(*)=O]

In some embodiments, the spacer is:

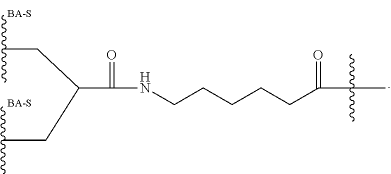 [SMILES: *CC(C*)C(=O)NCCCCCC(*)=O]

In some embodiments, the spacer is:

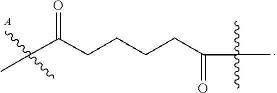

In some embodiments, the spacer is:

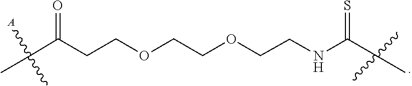

In some embodiments, the spacer is:

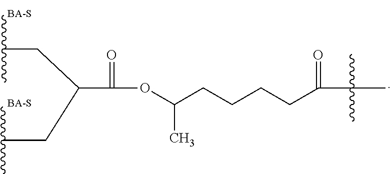 [SMILES: *CC(C*)C(=O)OC(C)CCCCC(*)=O]

In some embodiments, the spacer is:

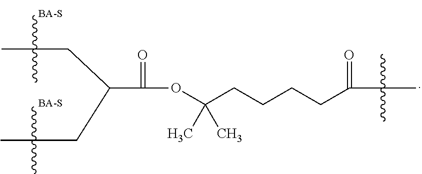 [SMILES: *CC(C*)C(=O)OC(C)(C)CCCCC(*)=O]

In some embodiments, the spacer is:

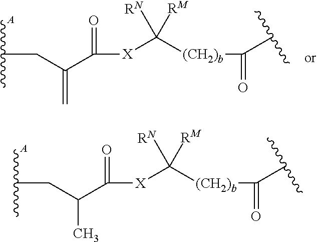

wherein [symbol] is a bond to the binding agent;
   X is N or O; R^{N} and R^{M} are each, independently, hydrogen or alkyl; and b is an integer from 1 to 8.

In some embodiments, AA¹-AA² is: valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some embodiments, AA¹-AA² is: valine-citrulline or citrulline-valine. In some embodiments, AA¹-AA² is: valine-citrulline.

In some embodiments, the linker is:

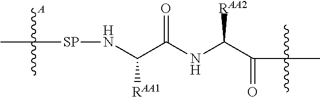

wherein:
SP is a spacer

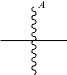

is one or more bonds to the binding agent;
R^{AA1} is an amino acid side chain; and
R^{AA2} is an amino acid side chain.

As used herein, “amino acid side chain” refers the monovalent non-hydrogen substituent bonded to the α-carbon of an α-amino acid, including natural and non-natural amino acids. Exemplary amino acid side chains include, but are not limited to, the α-carbon substituent of alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, and citrulline.

In some embodiments, the linker is:

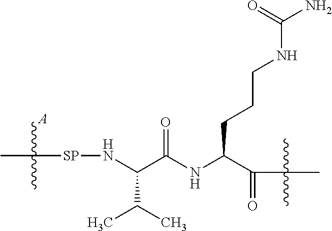

wherein:
SP is a spacer; and

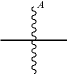

is one or more bonds to the binding agent.

In some embodiments, the linker is:

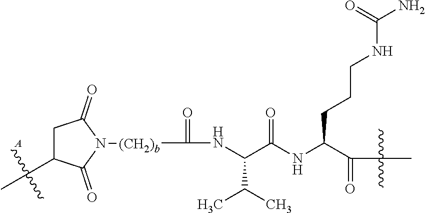

wherein:

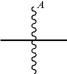

is a bond to the binding agent; and
b is an integer from 2 to 8.

In some embodiments, the linker is:

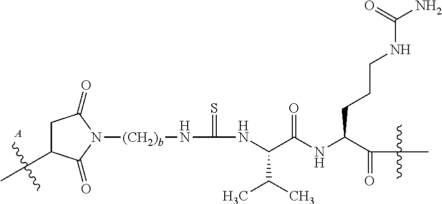

wherein:

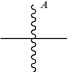

is a bond to the binding agent; and
b is an integer from 2 to 8.

In some embodiments, BA is an antibody and the linker is:

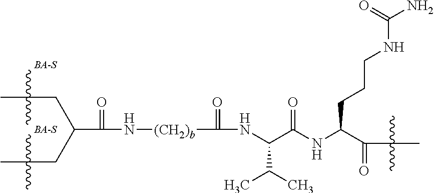

wherein:
the two bonds represented by

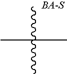

are bonds to cysteines of the antibody; and
b is an integer from 2 to 8.

In some embodiments, BA is an antibody and the linker is:

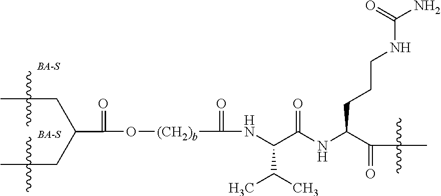

wherein:
the two bonds represented by

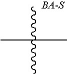

are bonds to cysteines of the antibody; and
b is an integer from 2 to 8.

In some embodiments, BA is an antibody and the linker is:

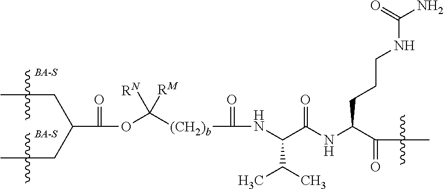

wherein:
R^{N} is a hydrogen atom or alkyl;
R^{M} is alkyl;
the two bonds represented by

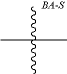

are bonds to cysteines of the antibody; and
b is an integer from 2 to 8.

In some embodiments, the linker is:

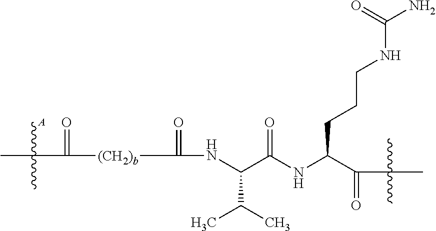

wherein:

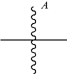

is a bond to the binding agent; and
b is an integer from 2 to 8.

In some embodiments, the linker is:

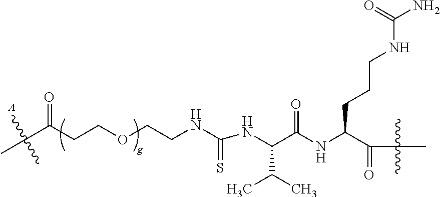

wherein:

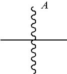

is a bond to the binding agent; and
g is an integer from 2 to 20. In some embodiments, g is 2 to 8. In some embodiments, g is 2, 4, 6, or 8.

In some embodiments, the linker is:

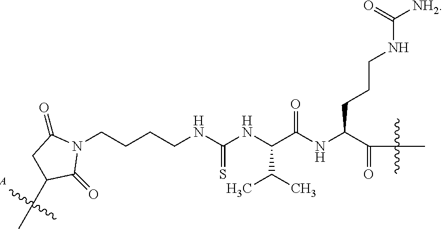

In some embodiments, the linker is:

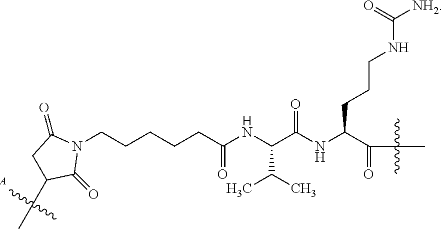

In some embodiments, the linker is:

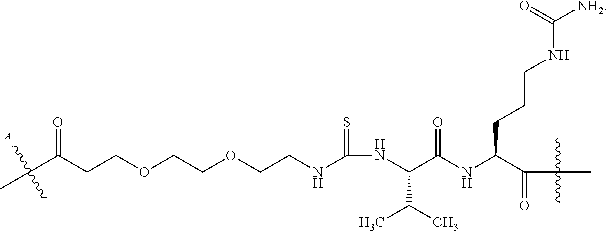

In some embodiments, the linker is:

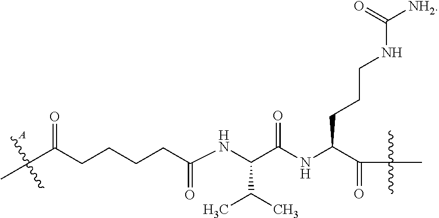

In some embodiments, the linker is:

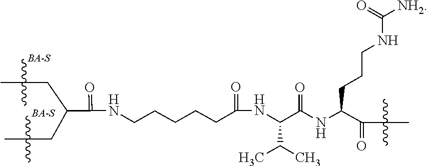 [SMILES: *CC(C*)C(=O)NCCCCCC(=O)N[C@H](C(=O)N[C@@H](CCCNC(N)=O)C(*)=O)C(C)C]

In some embodiments, the linker is:

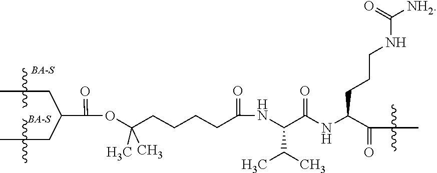 [SMILES: *CC(C*)C(=O)OC(C)(C)CCCCC(=O)N[C@H](C(=O)N[C@@H](CCCNC(N)=O)C(*)=O)C(C)C]

In some embodiments, the linker is:

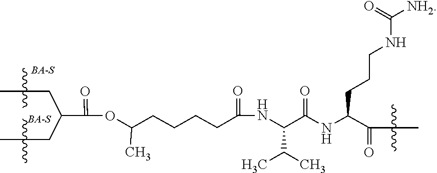 [SMILES: *CC(C*)C(=O)OC(C)CCCCC(=O)N[C@H](C(=O)N[C@@H](CCCNC(N)=O)C(*)=O)C(C)C]

In some embodiments, the linker is:

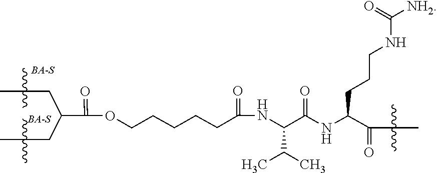 [SMILES: *CC(C*)C(=O)OCCCCCC(=O)N[C@H](C(=O)N[C@@H](CCCNC(N)=O)C(*)=O)C(C)C]

### 3. Binding Agents

Suitable binding agents include, but are not limited to, antibodies, lymphokines, hormones, growth factors, viral receptors, interleukins, or any other cell binding or peptide binding molecules or substances.

In some embodiments, the binding agent is an antibody. In some embodiments, the antibody is a monoclonal antibody, polyclonal antibody, antibody fragment (Fab, Fab′, and F(ab)2, minibody, diabody, tribody, and the like), or bispecific antibody. Antibodies herein can be humanized using methods described in U.S. Pat. No. 6,596,541 and US Publication No. 2012/0096572, each incorporated by reference in their entirety.

Where the binding agent is an antibody, it binds to an antigen binding partner that is a polypeptide and may be a transmembrane molecule (e.g., receptor) or a growth factor that might be glycosylated or phosphorylated. Exemplary antigens include, but are not limited to, molecules such as renin; a growth hormone, including human growth hormone and bovine growth hormone; growth hormone releasing factor; parathyroid hormone; thyroid stimulating hormone; lipoproteins; alpha1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-I-alpha); a serum albumin, such as human serum albumin; Muellerian-inhibiting substance; relaxin A-chain; relaxin B-chain; prorelaxin; mouse gonadotropin-associated peptide; a microbial protein, such as betalactamase; DNase; 19E; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-β1, TGF-β2, TGF-β3, TGF-β4, or TGF-β5; insulin-like growth factor-1 and -II (IGF-1 and IGF-II); des(I-3)-IGF-1 (brain IGF-1), insulin-like growth factor binding proteins, EpCAM, GD3, FLT3, PSMA, PSCA, MUC1, MUC16, STEAP, CEA, TENB2, EphA receptors, EphB receptors, folate receptor, FOLRI, mesothelin, cripto, alphavbeta6, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044 and incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as AFP, ALK, B7H4, BAGE proteins, 0-catenin, brc-abl, BRCA1, BORIS, CA9 (carbonic anhydrase IX), caspase-8, CD20, CD40, CD123, CDK4, CEA, CLEC12A, c-kit, cMET, CTLA4, cyclin-B1, CYP1B1, EGFR, EGFRvIII, endoglin, Epcam, EphA2, ErbB2/Her2, ErbB3/Her3, ErbB4/Her4, ETV6-AML, Fra-1, FOLR1, GAGE proteins (e.g., GAGE-1, -2), GD2, GD3, GloboH, glypican-3, GM3, gp100, Her2, HLA/B-raf, HLA/EBNA1, HLA/k-ras, HLA/MAGE-A3, hTERT, IGF1R, LGR5, LMP2, MAGE proteins (e.g., MAGE-1, -2, -3, -4, -6, and -12), MART-1, mesothelin, ML-IAP, Mucd, Muc16 (CA-125), MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSCA, PSGR, PSMA (FOLH1), RAGE proteins, Ras, RGS5, Rho, SART-1, SART-3, Steap-1, Steap-2, STn, survivin, TAG-72, TGF-β, TMPRSS2, Tn, TNFRSF17, TRP-1, TRP-2, tyrosinase, and uroplakin-3, and fragments of any of the above-listed polypeptides.

Exemplary antigens also include, but are not limited to, BCMA, SLAMF7, B7H4, GPNMB, UPK3A, and LGR5.

In some embodiments, the antigens include prolactin receptor (PRLR) or prostate-specific membrane antigen (PSMA).

Binding agents also include, but are not limited to, ankyrin repeat proteins, interferons, lymphokines such as IL-2 or IL-3, hormones like insulin and glucocorticoids, growth factors such as EGF, transferrin and fibronectin type III.

In some embodiments, the binding agents interact with or bind to tumor antigens, including antigens specific for a type of tumor or antigens that are shared, overexpressed or modified on a particular type of tumor. Examples include, but are not limited to: alpha-actinin-4 with lung cancer, ARTC1 with melanoma, BCR-ABL fusion protein with chronic myeloid leukemia, B-RAF, CLPP or Cdc27 with melanoma, CASP-8 with squamous cell carcinoma, and hsp70-2 with renal cell carcinoma as well as the following shared tumor-specific antigens, for example: BAGE-1, GAGE, GnTV, KK-LC-1, MAGE-A2, NA88-A, TRP2-INT2.

In some embodiments, the binding agent is an antibody. In some embodiments, the binding agent is a monoclonal antibody. In some embodiments, the binding agent is a polyclonal antibody. In some embodiments, the antibody is an anti-PSMA, anti-MUC16, or anti-EGFRvIII, or anti-STEAP-2 antibody.

The linkers can be bonded to the binding agent, e.g., antibody or antigen-binding molecule, through an attachment at a particular amino acid within the antibody or antigen-binding molecule. Exemplary amino acid attachments that can be used in the context of this aspect of the disclosure include, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenocysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Linkers can be conjugated via glutamine via transglutaminase-based chemo-enzymatic conjugation (see, e.g., Dennler et al., Bioconjugate Chem. 2014, 25, 569-578). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, WO 2014/197854, and Shaunak et al., Nat. Chem. Biol., 2006, 2:312-313).

In some embodiments, the binding agent is an antibody, and the antibody is bonded to the linker through a lysine residue. In some embodiments, the antibody is bonded to the linker through a cysteine residue.

### 4. Illustrative Embodiments

In some embodiments,
A is:

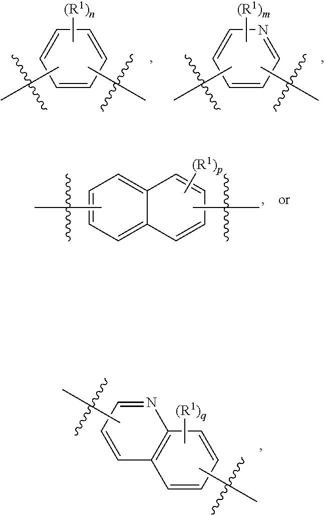

wherein:
   R¹, independently at each occurrence, is alkyl, alkenyl, alkynyl, aryl, alkaryl, aralkyl, halo, heteroaryl, heterocycloalkyl, hydroxyl, cyano, nitro,

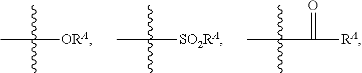

or azido,
   wherein R^{A} is alkyl or heteroalkyl;
n is an integer from 0 to 4;
m is an integer from 0 to 3;
p is an integer from 0 to 6; and
q is an integer from 0 to 5; and
L is:

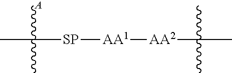

wherein:
   SP is a spacer;

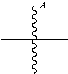

is one or more bonds to the binding agent;
AA¹ is an amino acid; and
AA² is an amino acid.

In some embodiments,
A is:

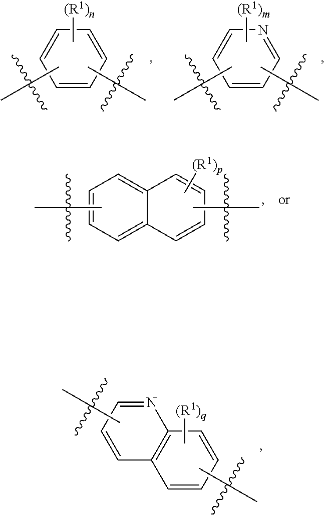

wherein:
   R¹, independently at each occurrence, is C₁₋₆ alkyl, C₁₋₆ haloalkyl, or halo;
   n is an integer from 0 to 4;
   m is an integer from 0 to 3;
   p is an integer from 0 to 6; and
   q is an integer from 0 to 5; and
L is:

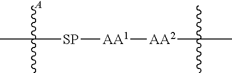

wherein:
   SP is a spacer;

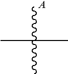

is one or more bonds to the binding agent;
AA¹ is an amino acid; and
AA² is an amino acid.

In some embodiments, A is:

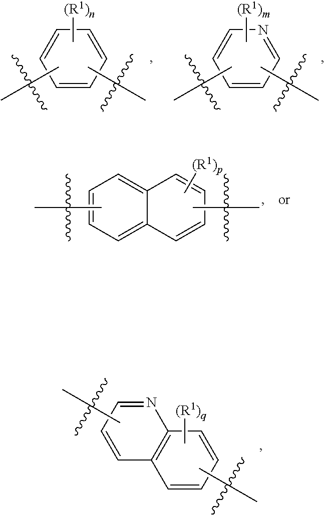

wherein:
   R¹, independently at each occurrence, is alkyl, alkenyl, alkynyl, alkoxy, aryl, alkaryl, aralkyl, halo, haloalkoxy, haloalkyl, haloalkoxy, heteroaryl, heteroalkyl, heterocycloalkyl, hydroxyl, cyano, nitro,

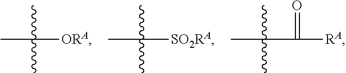

or azido,
wherein R^{A} is alkyl or heteroalkyl;
n is an integer from 0 to 4;
m is an integer from 0 to 3;
p is an integer from 0 to 6; and
q is an integer from 0 to 5; and
L is:

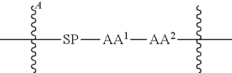

wherein:
   SP is a spacer;

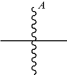

is one or more bonds to the binding agent;
AA¹ is an amino acid; and AA² is an amino acid.

In some embodiments,
A is:

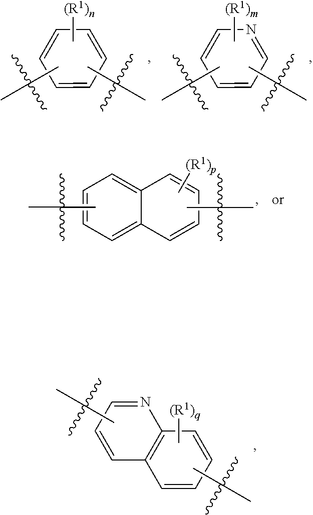

wherein:
   R¹, independently at each occurrence, is C₁₋₆ alkyl, C₁₋₆ haloalkyl, or halo;
   n is an integer from 0 to 4;
   m is an integer from 0 to 3;
   p is an integer from 0 to 6; and
   q is an integer from 0 to 5; and
L is:

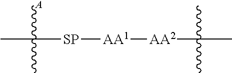

wherein:
   SP is:

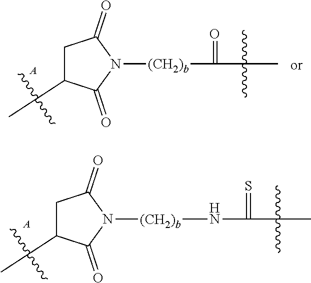

wherein:

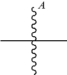

is a bond to the binding agent; and
b is an integer from 2 to 8; and
AA¹ is an amino acid; and
AA² is an amino acid.

In some embodiments, A is:

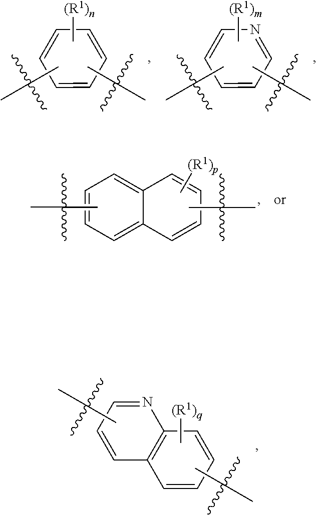

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

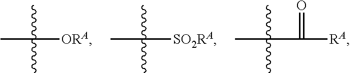

or azido,
   wherein R^{A} is alkyl or heteroalkyl;
   n is an integer from 0 to 4;
   m is an integer from 0 to 3;
   p is an integer from 0 to 6; and
   q is an integer from 0 to 5; and
L is:

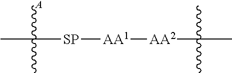

wherein:
   SP is:

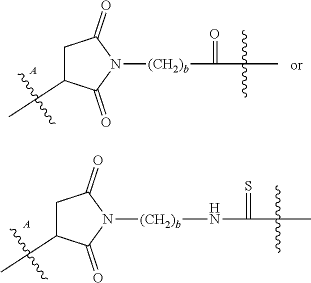

wherein:

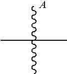

is a bond to the binding agent; and
b is an integer from 2 to 8; and
AA¹ is an amino acid; and
AA² is an amino acid.

In some embodiments,
A is:

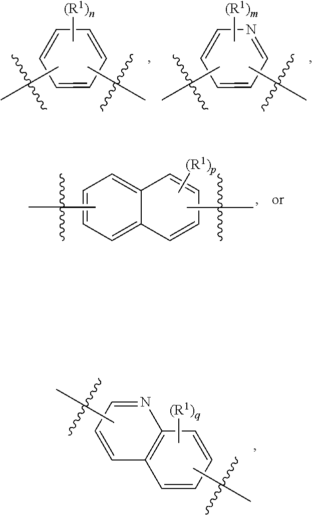

wherein:
R¹, independently at each occurrence, is C₁₋₆ alkyl, C₁₋₆ haloalkyl, or halo;
n is an integer from 0 to 4;
m is an integer from 0 to 3;
p is an integer from 0 to 6; and
q is an integer from 0 to 5; and
L is:

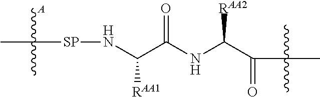

wherein:
   SP is a spacer;

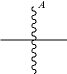

is one or more bonds to the binding agent;
R^{AA1} is an amino acid side chain; and
R^{AA2} is an amino acid side chain.

In some embodiments, A is:

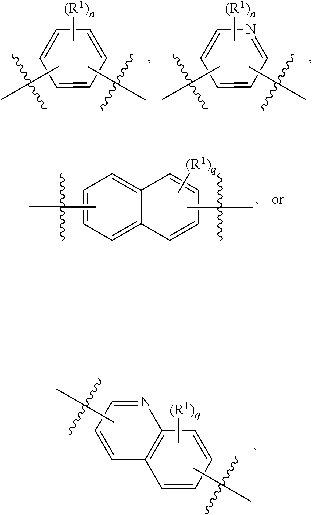

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

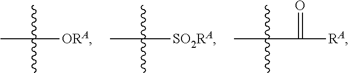

or azido,
wherein R^{A}, is alkyl or heteroalkyl n is an integer from 0 to 4;
m is an integer from 0 to 3;
p is an integer from 0 to 6; and
q is an integer from 0 to 5; and
L is:

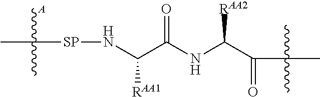

wherein:
   SP is a spacer;

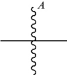

is one or more bonds to the binding agent;
R^{AA1} is an amino acid side chain; and
R^{AA2} is an amino acid side chain.

In some embodiments,
A is:

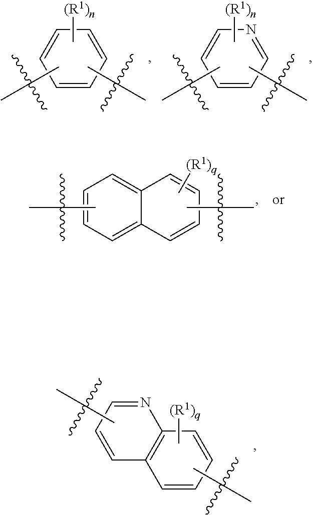

wherein:
R¹, independently at each occurrence, is C₁₋₆ alkyl, C₁₋₆ haloalkyl, or halo;
n is an integer from 0 to 4;
m is an integer from 0 to 3;
p is an integer from 0 to 6; and
q is an integer from 0 to 5; and
L is:

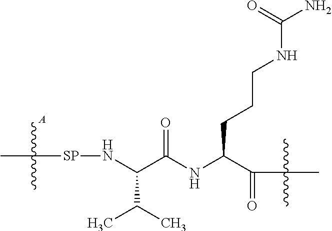

wherein:
   SP is a spacer; and

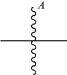

is the one or more bonds to the binding agent.

In some embodiments, A is:

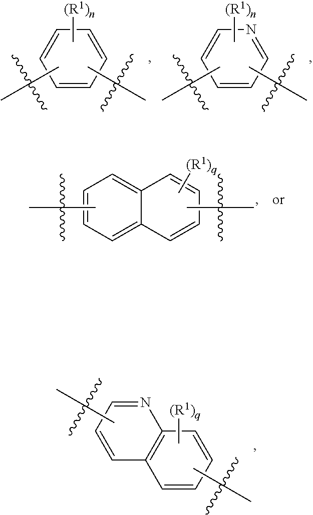

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

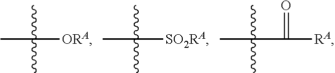

or azido,
   wherein R^{A} is alkyl;
   n is an integer from 0 to 4;
   m is an integer from 0 to 3;
   p is an integer from 0 to 6; and
q is an integer from 0 to 5; and
L is:

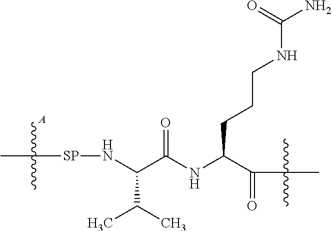

wherein:
   SP is a spacer; and

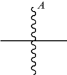

is the one or more bonds to the binding agent.

In some embodiments,
A is:

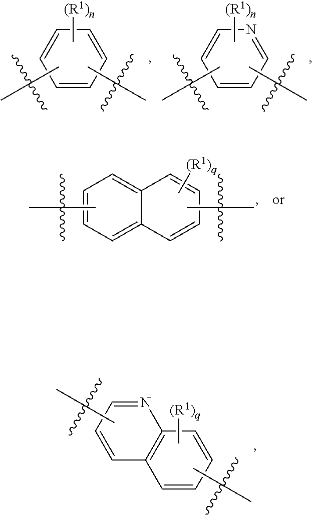

wherein:
R¹, independently at each occurrence, is C₁₋₆ alkyl, C₁₋₆ haloalkyl, or halo;
n is an integer from 0 to 4;
m is an integer from 0 to 3;
p is an integer from 0 to 6; and
q is an integer from 0 to 5; and
L is:

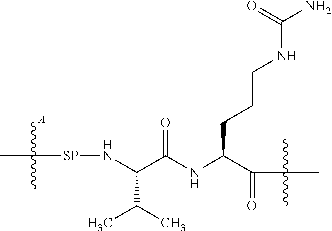

wherein:
   SP is:

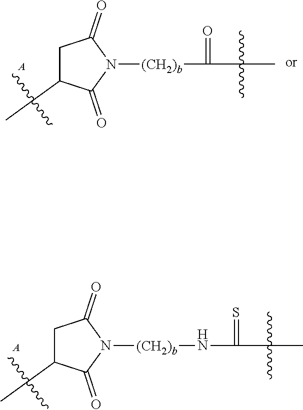

wherein:

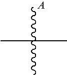

is a bond to the binding agent; and
b is an integer from 2 to 8.

In some embodiments, A is:

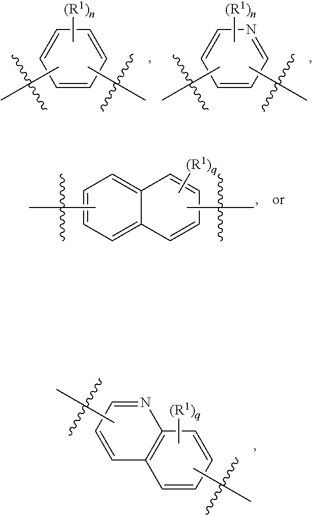

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

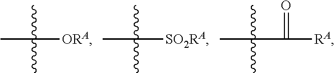

or azido,
   wherein R^{A} is alkyl or heteroalkyl;
   n is an integer from 0 to 4;
   m is an integer from 0 to 3;
   p is an integer from 0 to 6; and
q is an integer from 0 to 5; and
L is:

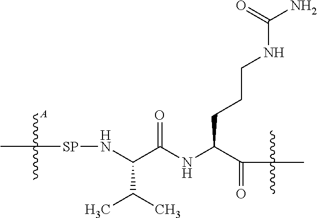

wherein:
   SP is:

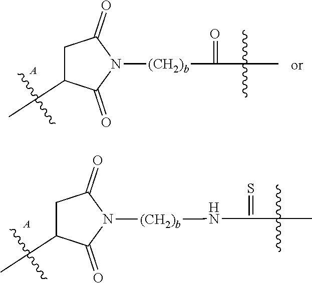

wherein:

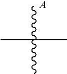

is a bond to the binding agent; and
b is an integer from 2 to 8.

In some embodiments,
A is:

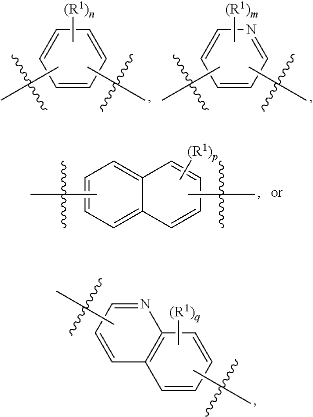

wherein:
   R¹, independently at each occurrence, is C₁₋₆ alkyl, C₁₋₆ haloalkyl, or halo; and n, m, p, and q are 0, 1, or 2; and
L is

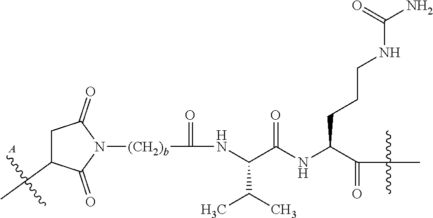

wherein:

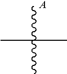

is a bond to the binding agent; and
b is an integer from 2 to 8.

In some embodiments, A is:

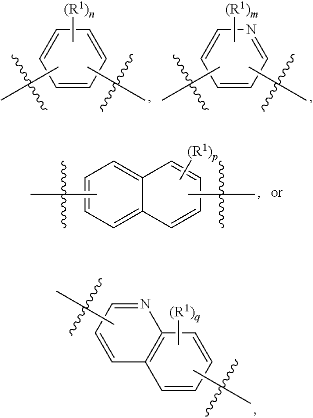

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

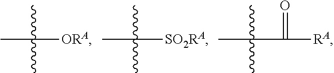

or azido,
   wherein R^{A} is alkyl;
   n is an integer from 0 to 4;
   m is an integer from 0 to 3;
   p is an integer from 0 to 6; and
q is an integer from 0 to 5; and
L is

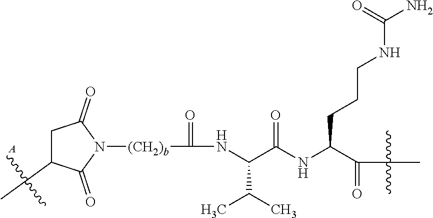

wherein:

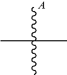

is a bond to the binding agent; and
b is an integer from 2 to 8.

In some embodiments,
A is:

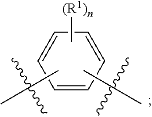

wherein
   R¹ is, independently at each occurrence, is C₁₋₆ haloalkyl, or halo; and
   n is 0, 1, or 2; and
L is:

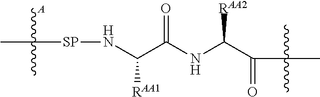

wherein:
   SP is a spacer;

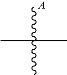

is the one or more bonds to the binding agent;
R^{AA1} is an amino acid side chain; and
R^{AA2} is an amino acid side chain.

In some embodiments, A is:

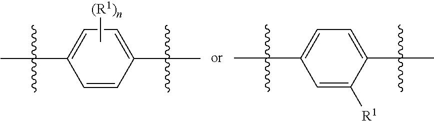

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

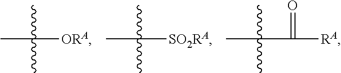

or azido,
   wherein R^{A} is alkyl or heteroalkyl;
wherein n is an integer from 0 to 4;
L is:

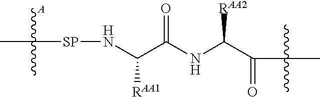

wherein:
   SP is a spacer;

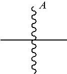

is the one or more bonds to the binding agent;
R^{A1} is an amino acid side chain; and
R^{A2} is an amino acid side chain.

In some embodiments,
A is:

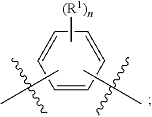

wherein
   R¹ is, independently at each occurrence, is halo; and
   n is 0, 1, or 2; and
L is:

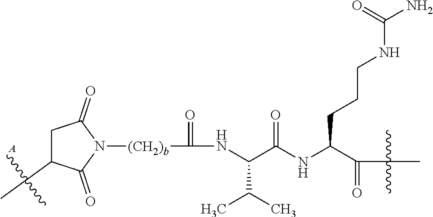

wherein:

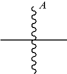

is a bond to the binding agent; and
b is an integer from 2 to 8.

In some embodiments, A is:

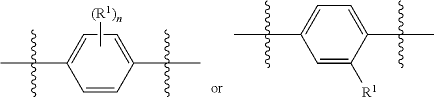

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

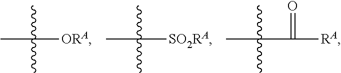

or azido,
   wherein R^{A} is alkyl or heteroalkyl;
L is:

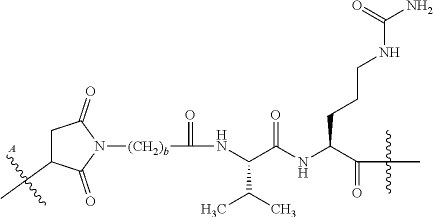

wherein:

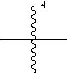

is a bond to the binding agent;
wherein n is an integer from 0 to 4; and
b is an integer from 2 to 8.

In some embodiments,
A is:

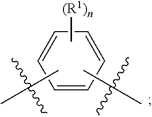

wherein
   R¹ is, independently at each occurrence, is halo; and
   n is 0, 1, or 2; and
L is:

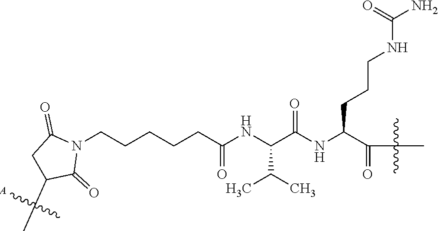

wherein

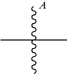

is a bond to the binding agent.

In some embodiments, A is:

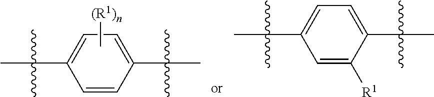

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, hydroxyl, cyano, nitro,

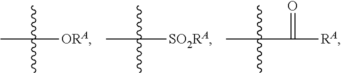

or azido,
   wherein R^{A} is alkyl or heteroalkyl;
wherein n is an integer from 0 to 4;
L is:

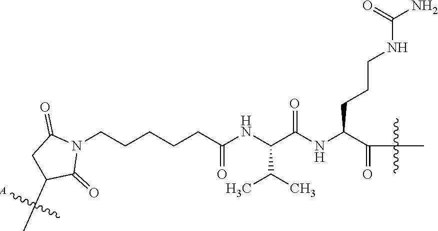

wherein

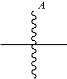

is a bond to the binding agent.

In some embodiments,
A is:

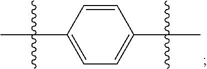 [SMILES: *c1ccc(*)cc1]

and
L is

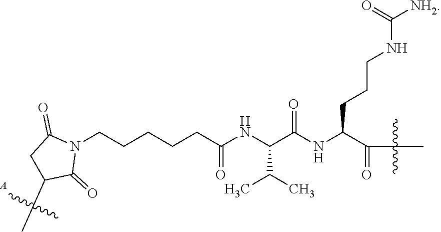

In some embodiments, A is:

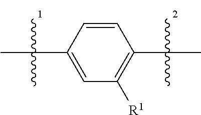

wherein:
   R¹ is, independently at each occurrence, a hydrogen atom, alkyl, alkoxy, aryl, heteroalkyl, halo, haloalkoxy, haloalkyl, or haloalkoxy;

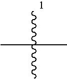

is the bond to the nitrogen atom; and

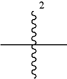

is the bond to the carbonyl. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen.

In some embodiments, A is:

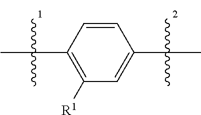

wherein:
   R¹ is, independently at each occurrence, a hydrogen atom, alkyl, alkoxy, aryl, heteroalkyl, halo, haloalkyl, haloalkoxy;

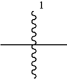

is the bond to the nitrogen atom; and

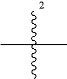

is the bond to the carbonyl. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen.

In some embodiments,
A is:

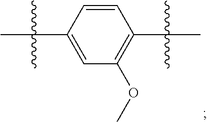 [SMILES: *c1ccc(*)c(OC)c1]

and
L is

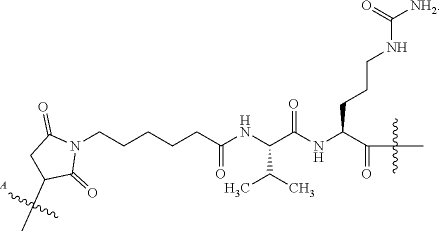

In some embodiments,
BA is an antibody,
A is:

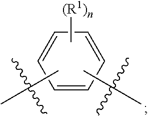

wherein
   R¹ is, independently at each occurrence, is halo; and
   n is 0, 1, or 2; and
L is:

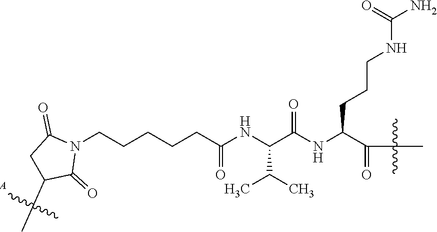

wherein

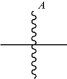

is a bond to the binding agent.

In some embodiments,
A is:

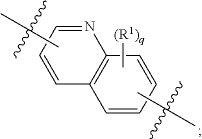

wherein
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

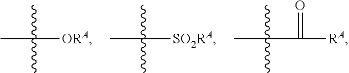

or azido; and
q is an integer from 0 to 5; and
L is:

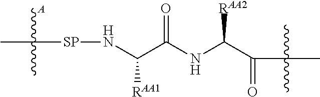

wherein:
   SP is a spacer;

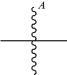

is the one or more bonds to the binding agent;
R^{AA1} is an amino acid side chain; and
R^{AA2} is an amino acid side chain.

In some embodiments, A is:

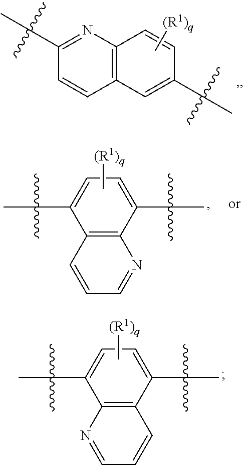

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

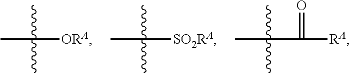

or azido;
wherein q is an integer from 0 to 5;
L is:

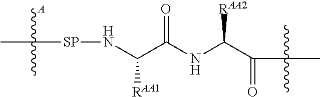

wherein:
   SP is a spacer;

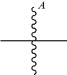

is the one or more bonds to the binding agent;
R^{AA1} is an amino acid side chain; and
R^{AA2} is an amino acid side chain.

In some embodiments,
A is:

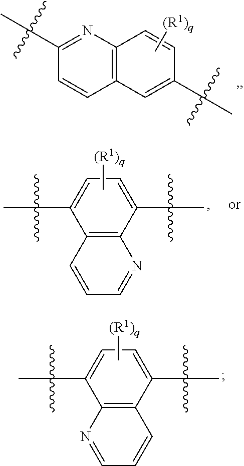

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

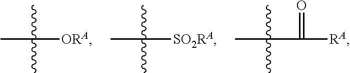

or azido;
wherein q is an integer from 0 to 5; and
L is:

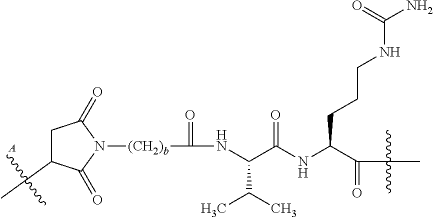

wherein:

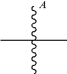

is a bond to the binding agent; and
b is an integer from 2 to 8.

In some embodiments,
A is:

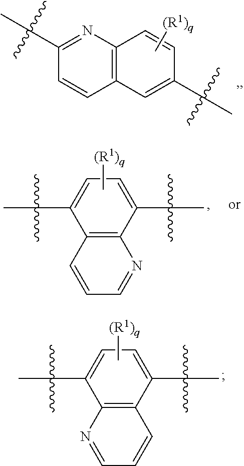

wherein:
   R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

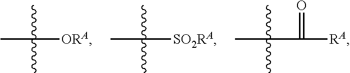

or azido; and
   q is an integer from 0 to 5; and
L is:

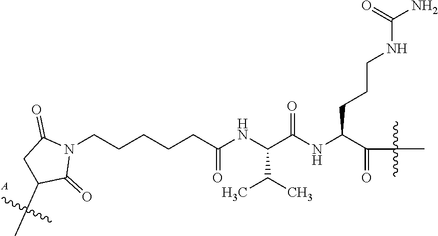

wherein

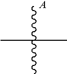

is a bond to the binding agent.

In some embodiments, A is:

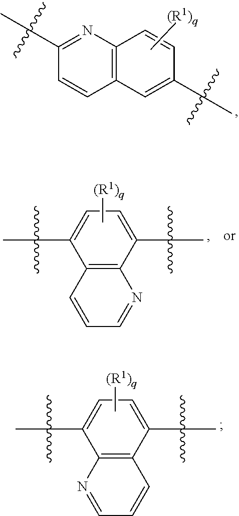

wherein:
R¹, independently at each occurrence, is alkyl, alkenyl, alkynyl, alkoxy, aryl, alkaryl, aralkyl, halo, haloalkoxy, haloalkyl, heteroaryl, heteroalkyl, heterocycloalkyl, cyano, nitro,

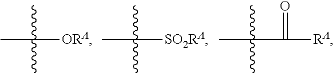

or azido;
   wherein q is an integer from 0 to 5;
L is:

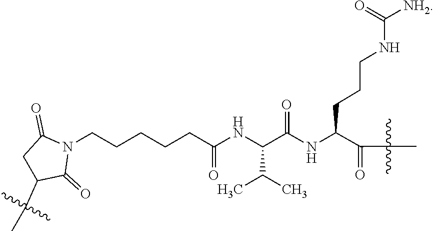 [SMILES: *C(=O)[C@H](CCCNC(N)=O)NC(=O)[C@@H](NC(=O)CCCCCN1C(=O)CC(*)C1=O)C(C)C]

wherein

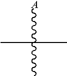

is a bond to the binding agent.

In some embodiments,
A is:

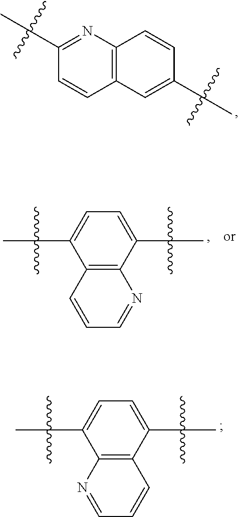 [SMILES: *c1ccc2nc(*)ccc2c1 ; *c1ccc(*)c2ncccc12 ; *c1ccc(*)c2ncccc12]

and
L is

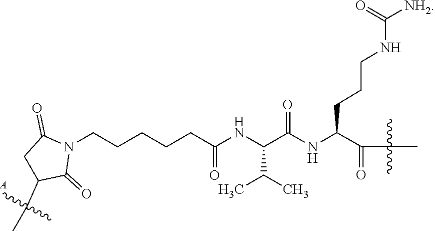

In some embodiments, the compound of Formula I is:

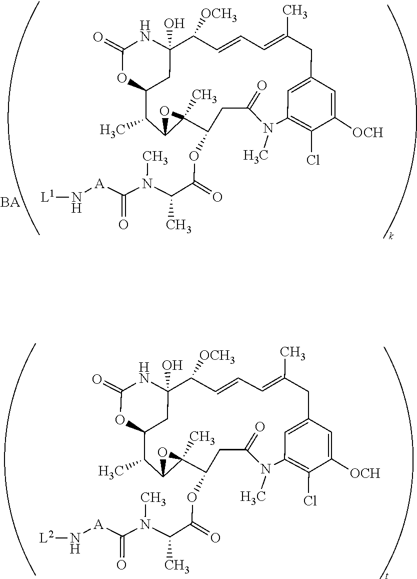

wherein A is arylene or heteroarylene, L¹ and L² are linkers, BA is a binding agent, k is an integer from 0 to 30, and t is an integer from 0 to 8. In some of these embodiments, L¹ is a linker which binds to the BA through a lysine residue. In some of these embodiments, the subscript, k, represents the number of linkers, L¹, bonded to the BA through lysine residues on the BA. In some of these embodiments, L² is a linker which binds to the BA through a cysteine residue. In some of these embodiments, the subscript, t, represents the number of linkers, L², bonded to the BA through cysteine residues on the BA. In some embodiments, when the linker, L², is a monodentate linker, t is an integer from 0 to 8. In some embodiments, when the linker, L², is a bidentate linker, t is an integer from 0 to 4. In some of these examples, the sum of k+t is equal to 1-8.

In some embodiments, the compound of Formula I is:

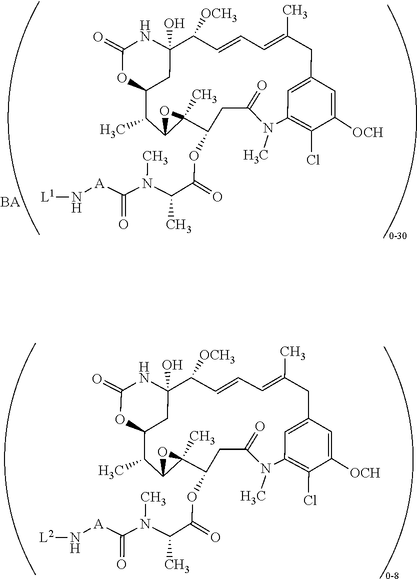

wherein A is arylene or heteroarylene, L¹ and L² are linkers, and BA is a binding agent. In some of these embodiments, L¹ is a linker which binds to the BA through a lysine residue. In some of these embodiments, L² is a linker which binds to the BA through a cysteine residue.

In some embodiments, the compound of Formula I is:

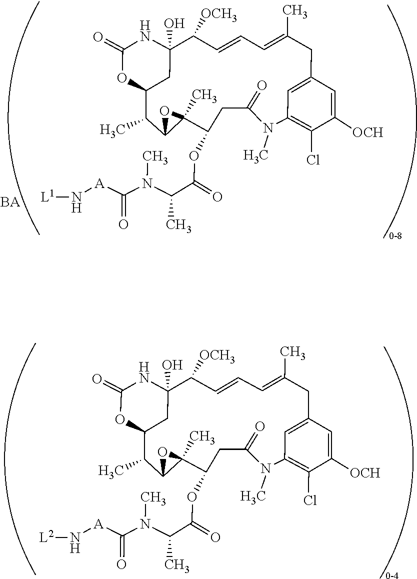

wherein A is arylene or heteroarylene, L¹ and L² are linkers, and BA is a binding agent. In some of these embodiments, L¹ is a linker which binds to the BA through a lysine residue. In some of these embodiments, L² is a linker which binds to the BA through a cysteine residue.

In some embodiments, A is:

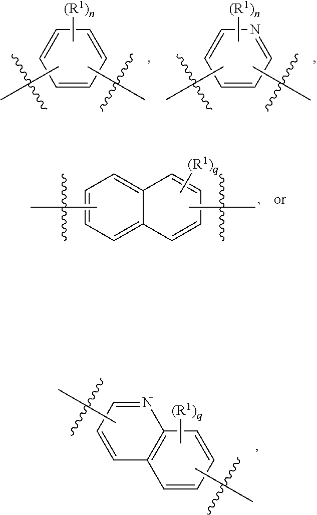

wherein:
   R¹ is, independently at each occurrence, halo, haloalkyl, haloalkoxy, hydroxyl, alkyl, alkenyl, alkynyl, alkoxy, haloalkoxy, aryl, alkaryl, aralkyl, heteroaryl, heteroalkyl, heterocycloalkyl, cyano, nitro

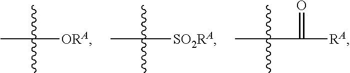

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, the linker is:

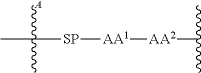

wherein:
   SP is a spacer;

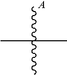

is one or more bonds to the binding agent;
AA¹ is an amino acid; and
AA² is an amino acid.

The spacer is a divalent moiety that connects the AA¹-AA² moiety to the binding agent (BA). Suitable spacers include, but are not limited to, those comprising alkylene or polyethylene glycol. The ends of the spacers, i.e., the portion of the spacer directly bonded to the binding agent or AA¹, can be moieties derived from reactive moieties that are used for purposes of coupling the antibody or AA¹ to the spacer during the chemical synthesis of the conjugate.

In some embodiments, the spacer comprises an alkylene. In some embodiments, the spacer comprises a C₅₋₇ alkylene. In some embodiments, the spacer is:

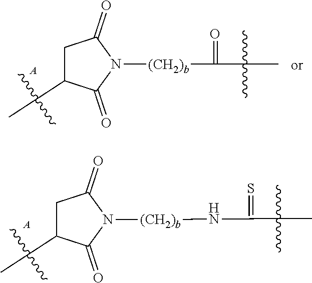

wherein:

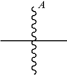

is a bond to the binding agent; and
b is an integer from 2 to 8.

In some embodiments, the spacer is:

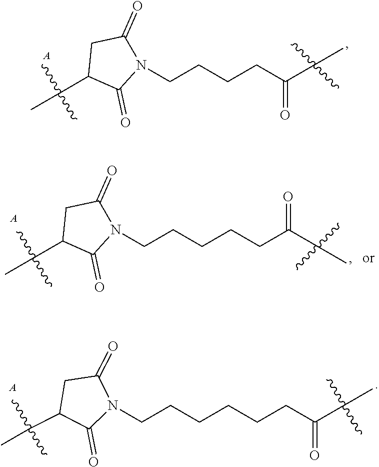

wherein:

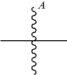

is a bond to the binding agent.

In some embodiments, the spacer is:

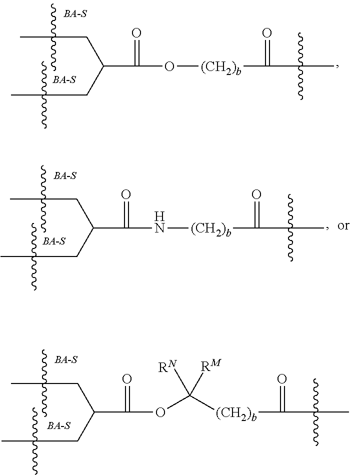

wherein:
   R^{N} is a hydrogen atom or alkyl;
   R^{M} is alkyl;
   the two bonds represented by

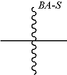

are bonds to cysteines of a binding agent; and
b is an integer from 2 to 8.

In some embodiments, the spacer is:

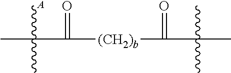

wherein:

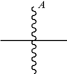

is a bond to the binding agent; and
b is an integer from 2 to 8.

In some embodiments, the spacer is:

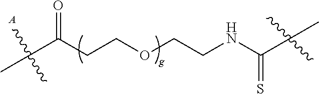

wherein:

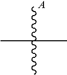

is a bond to the binding agent; and
g is an integer from 2 to 20. In some embodiments, g is 2-8. In some embodiments, g is 2, 4, 6, or 8.

In some embodiments, the spacer is:

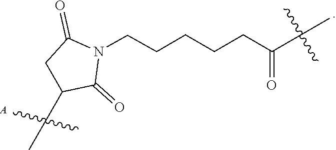

In some embodiments, the spacer is:

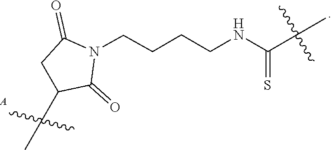

In some embodiments, the spacer is:

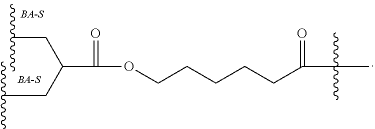 [SMILES: *CC(C*)C(=O)OCCCCCC(*)=O]

In some embodiments, the spacer is:

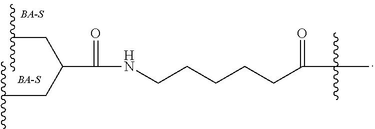 [SMILES: *CC(C*)C(=O)NCCCCCC(*)=O]

In some embodiments, the spacer is:

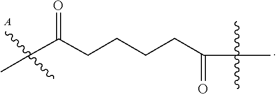

In some embodiments, the spacer is:

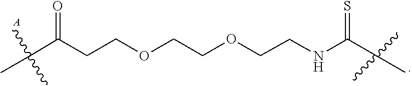

In some embodiments, the spacer is:

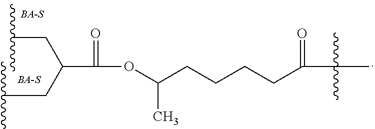 [SMILES: *CC(C*)C(=O)OC(C)CCCCC(*)=O]

In some embodiments, the spacer is:

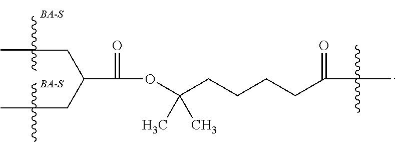 [SMILES: *CC(C*)C(=O)OC(C)(C)CCCCC(*)=O]

In some embodiments, the spacer is:

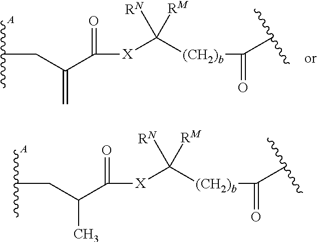

wherein [symbol] is a bond to the binding agent;
   X is N or O; R^{N} and R^{M} are each, independently, hydrogen or alkyl; and b is an integer from 1 to 8.

In some embodiments, AA¹-AA² is: valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some embodiments, AA¹-AA² is: valine-citrulline or citrulline-valine. In some embodiments, AA¹-AA² is: valine-citrulline.

In some embodiments, the compound of Formula I is:

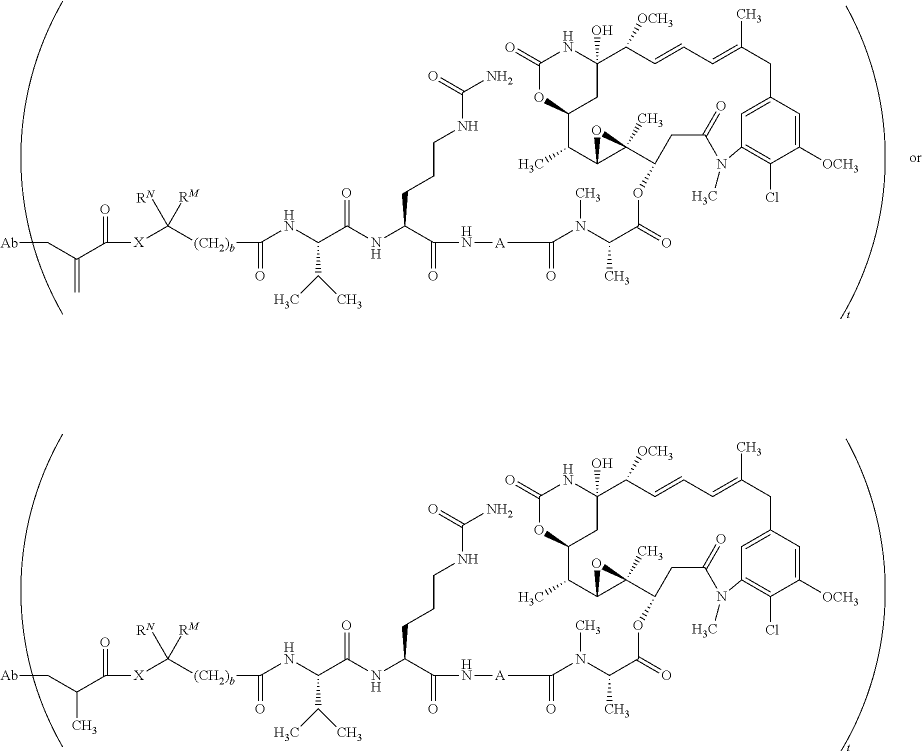

wherein X is N or 0,
R^{N} and R^{M} are each, independently, hydrogen or aryl,
b is an integer from 1 to 8,
A is aryl or heteroaryl, and
   t is an integer from 1-8.

In some embodiments, the compound of Formula I is:

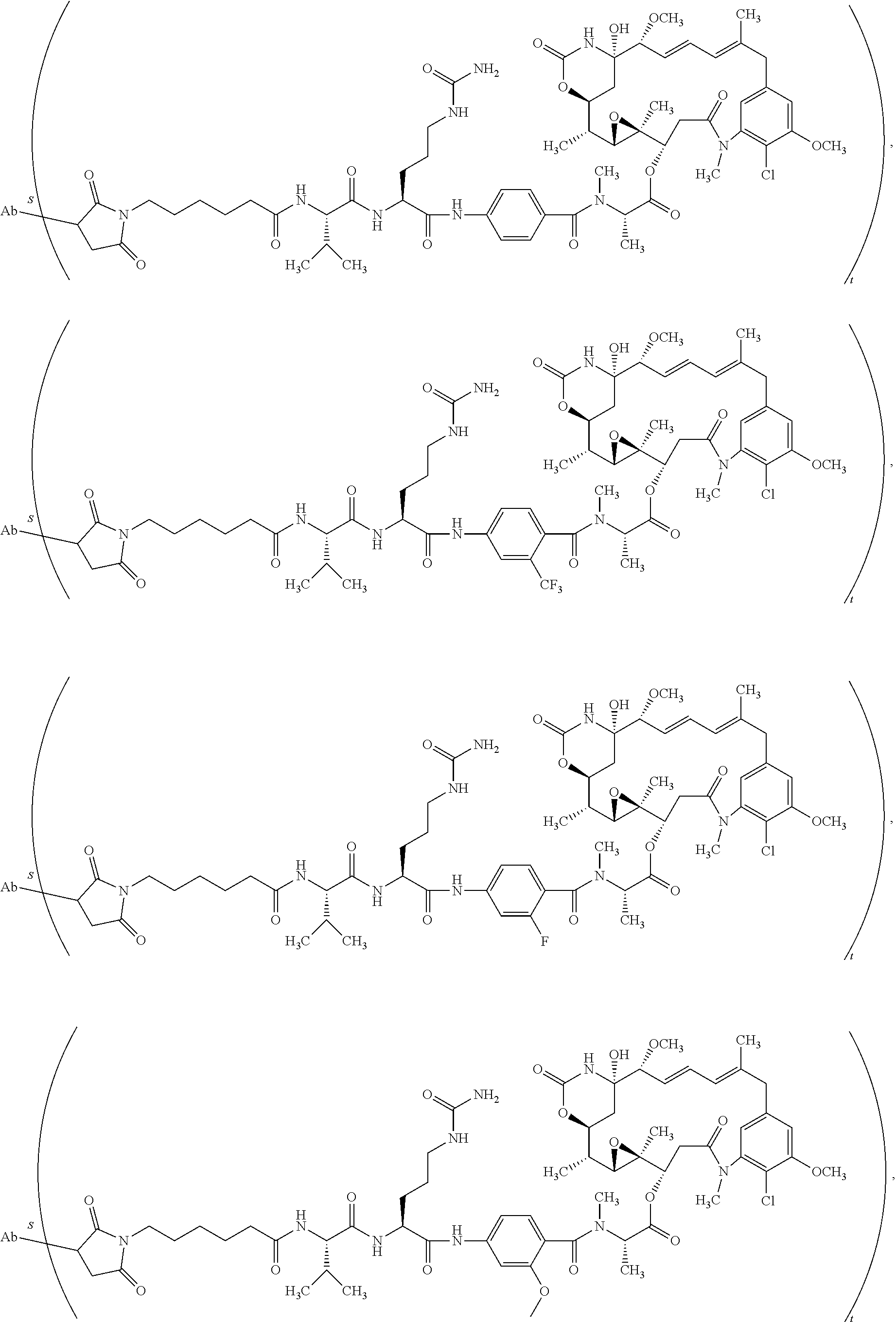

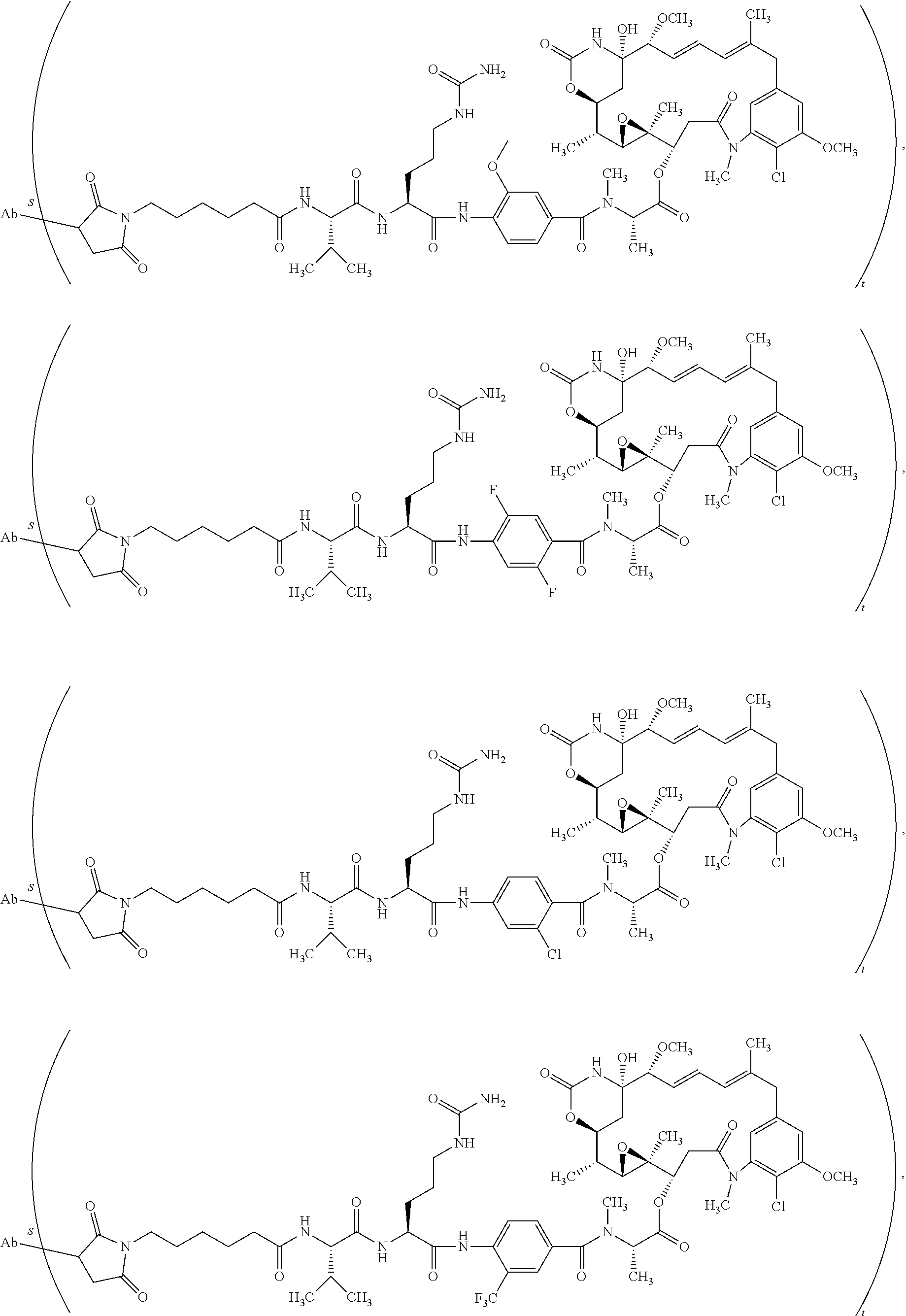

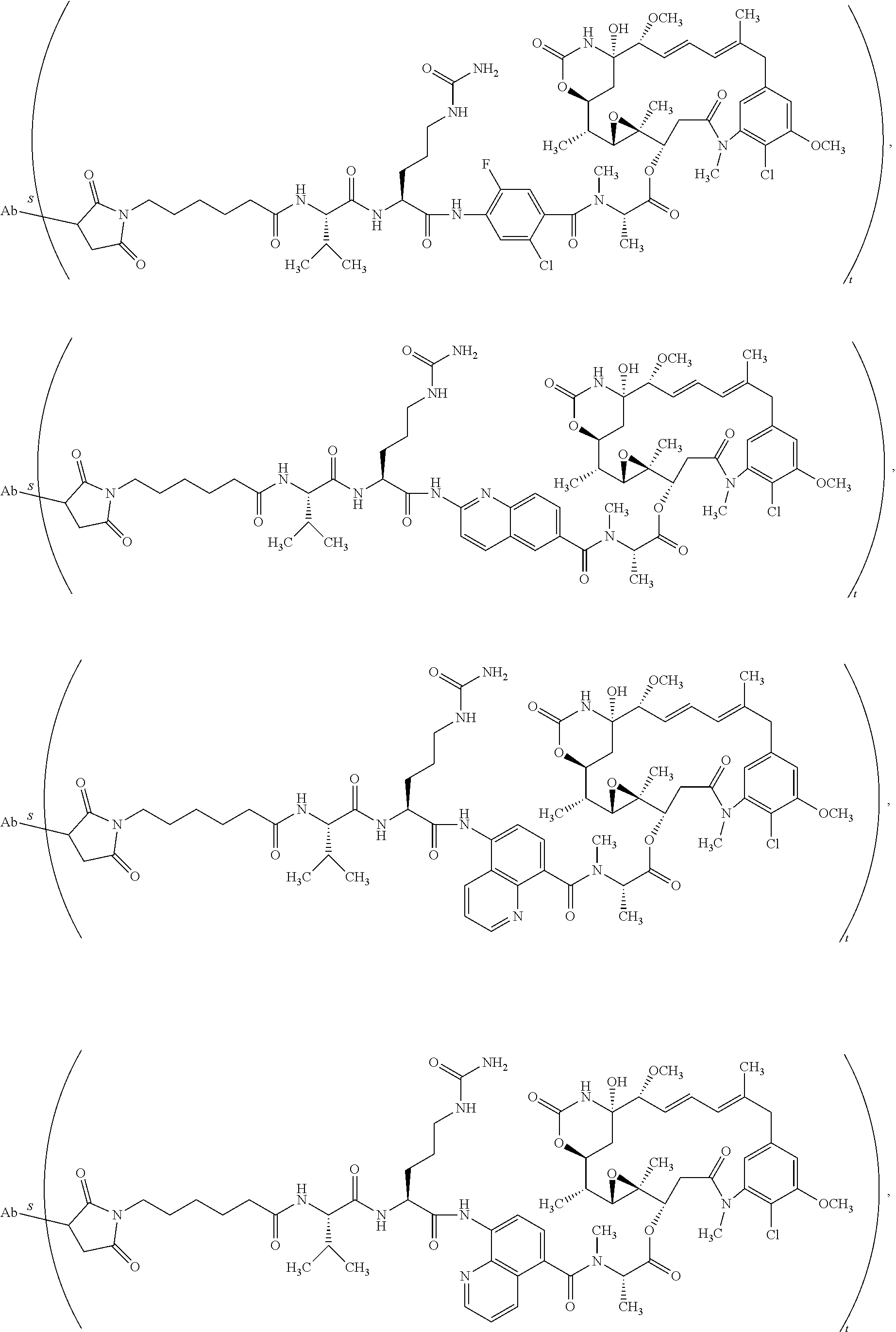

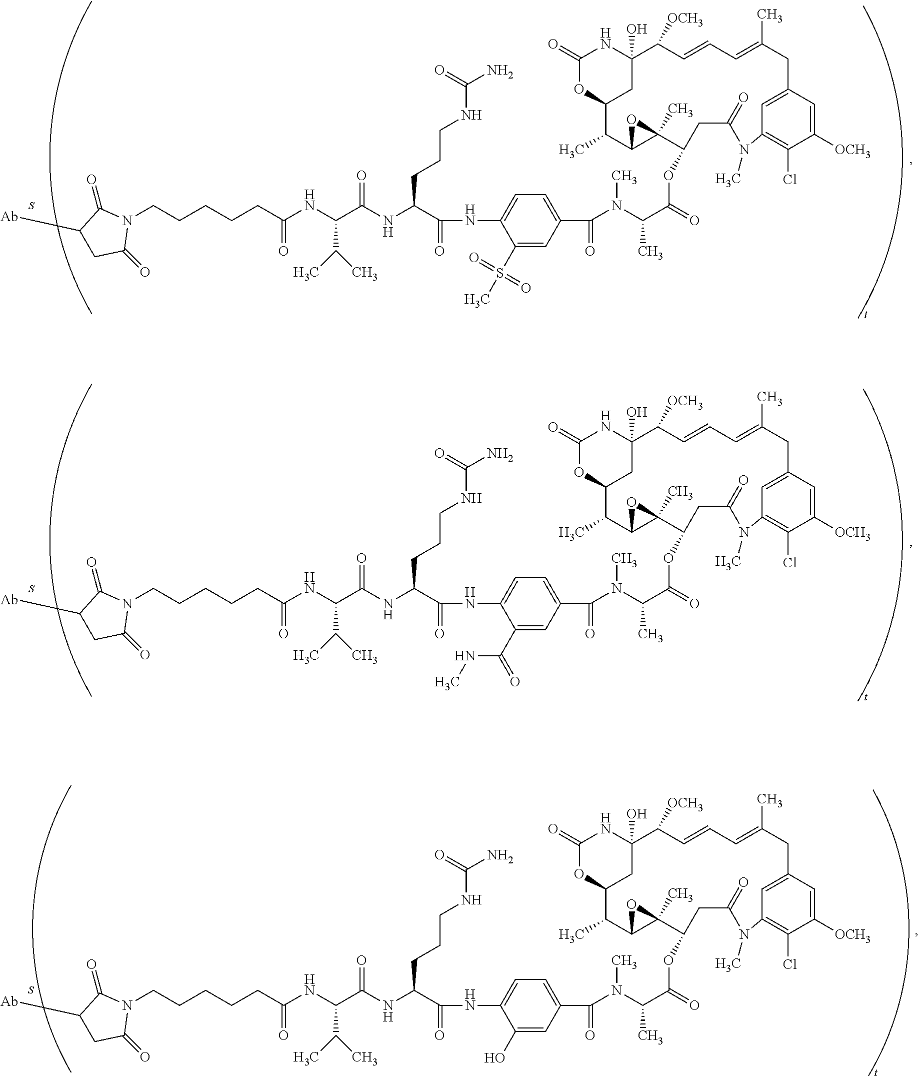

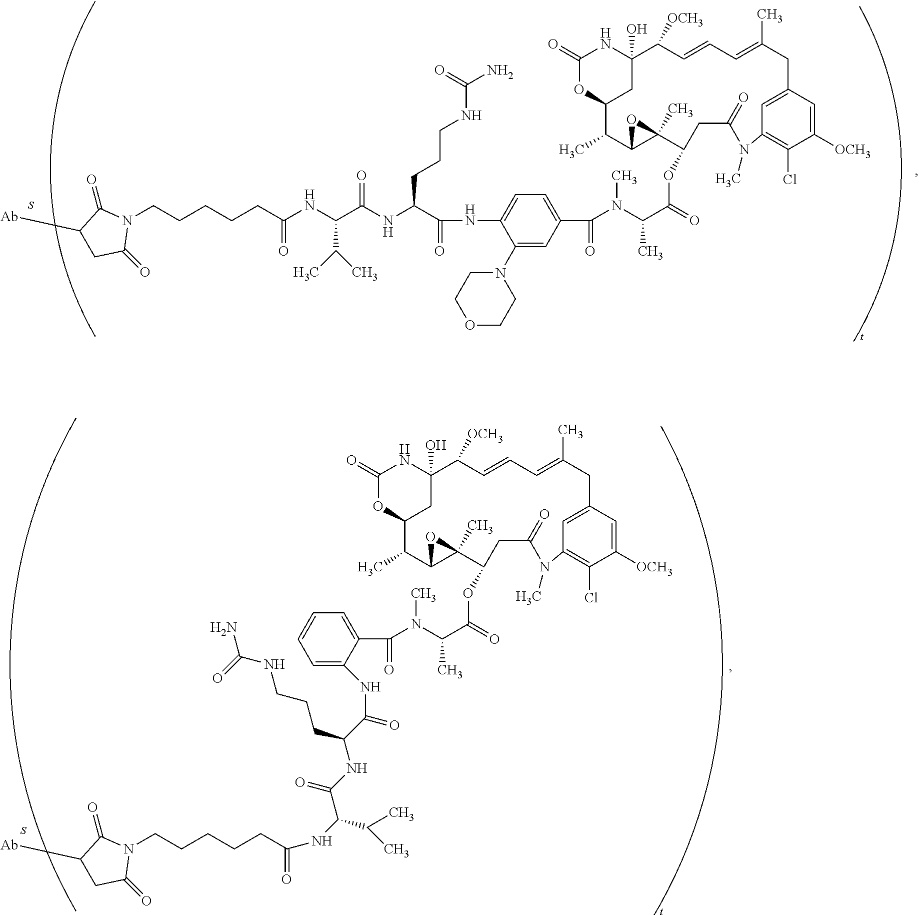

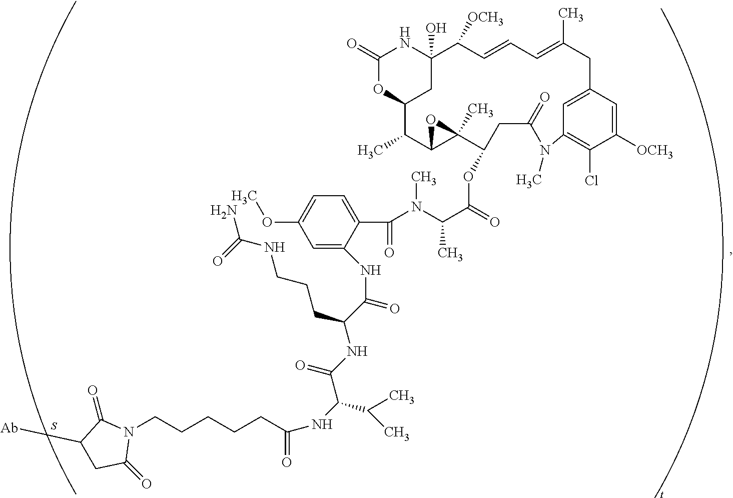

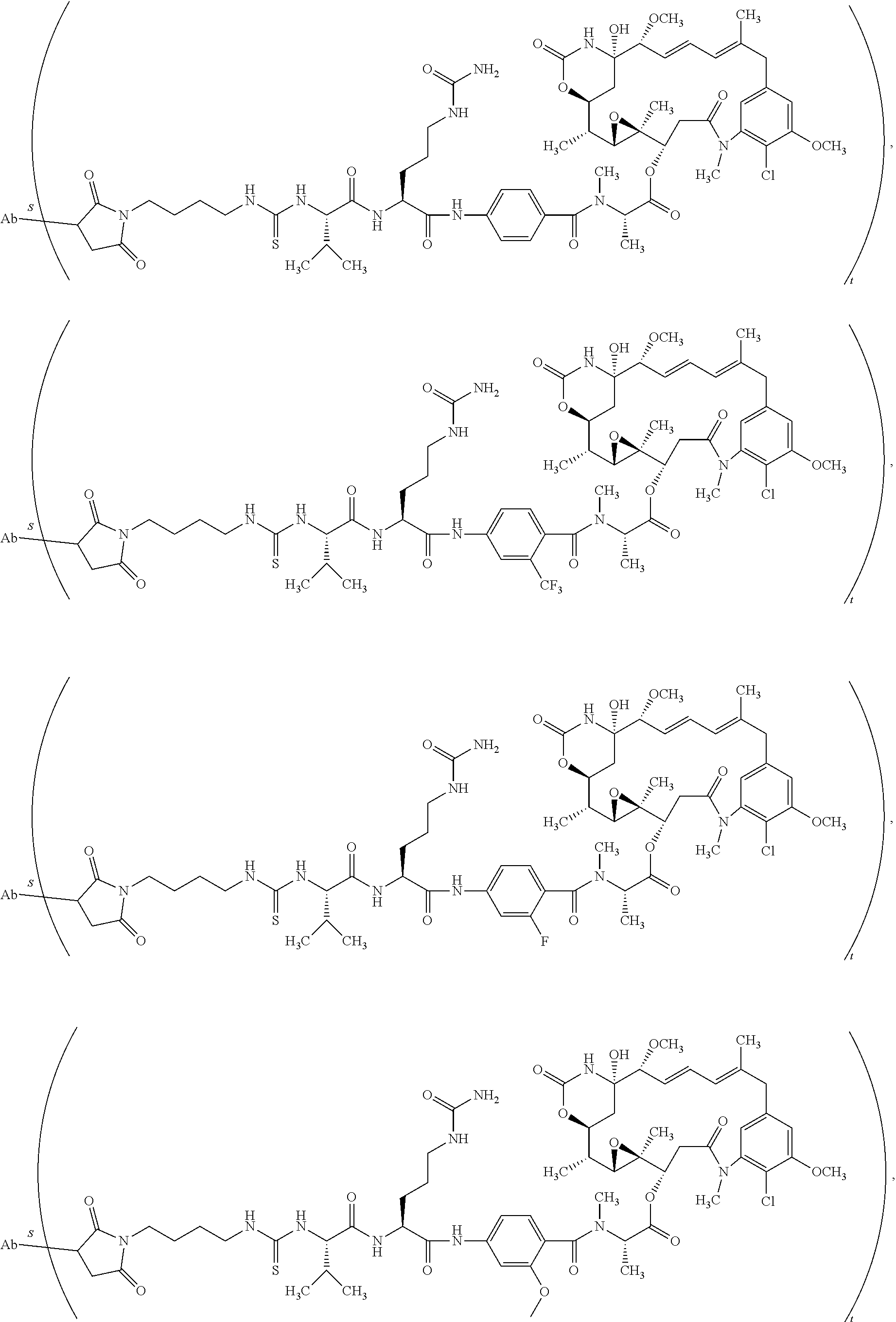

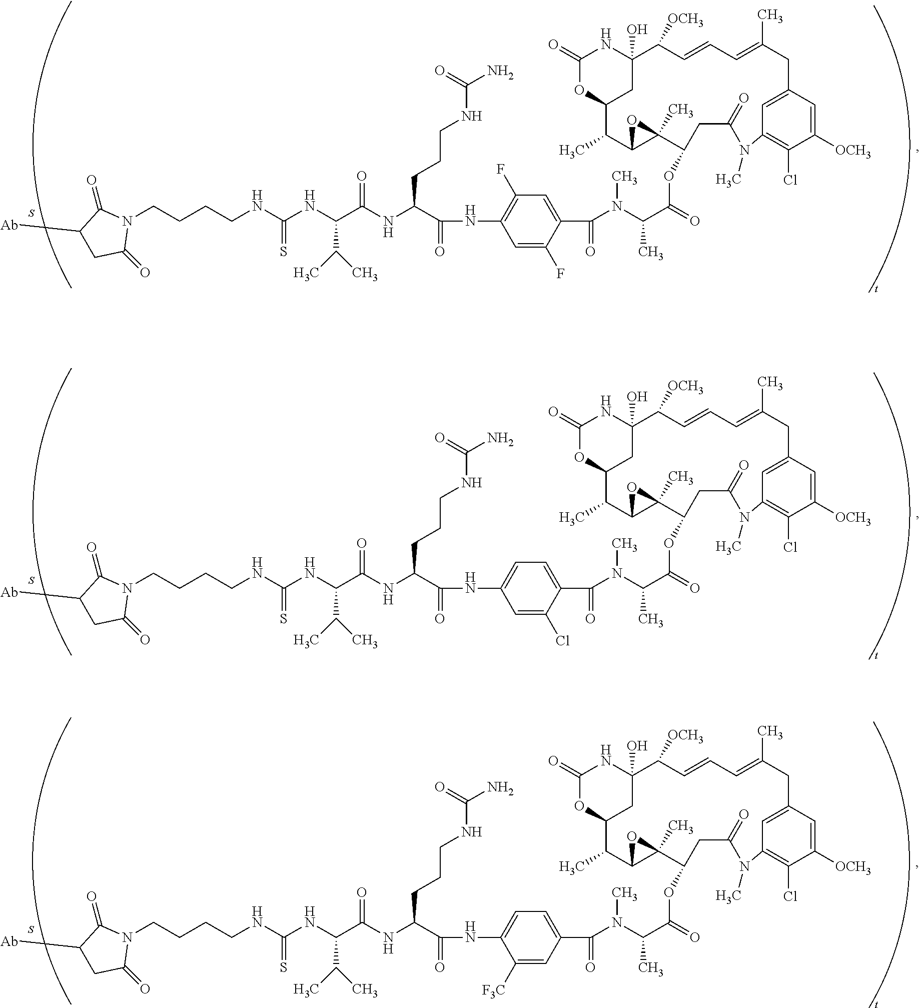

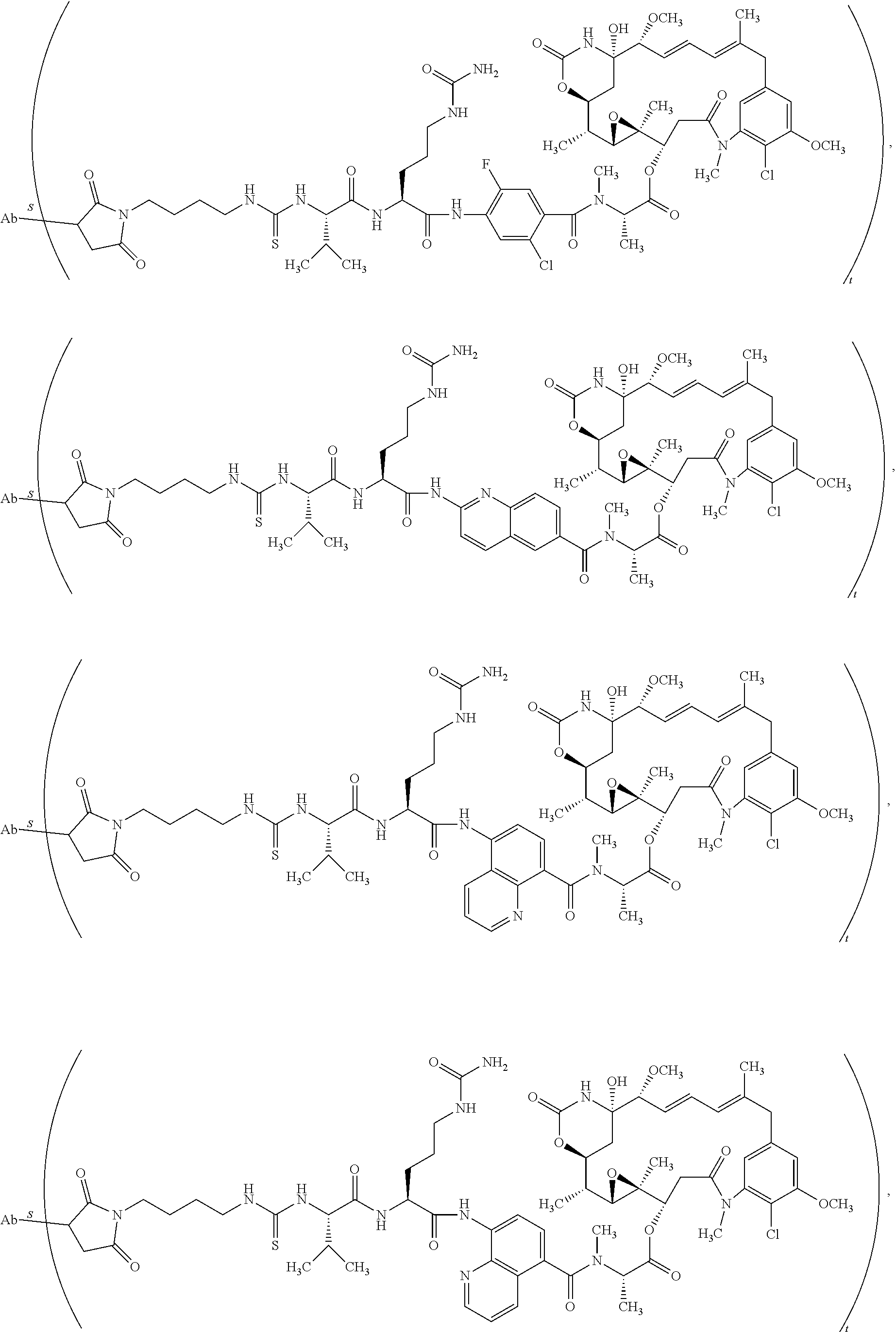

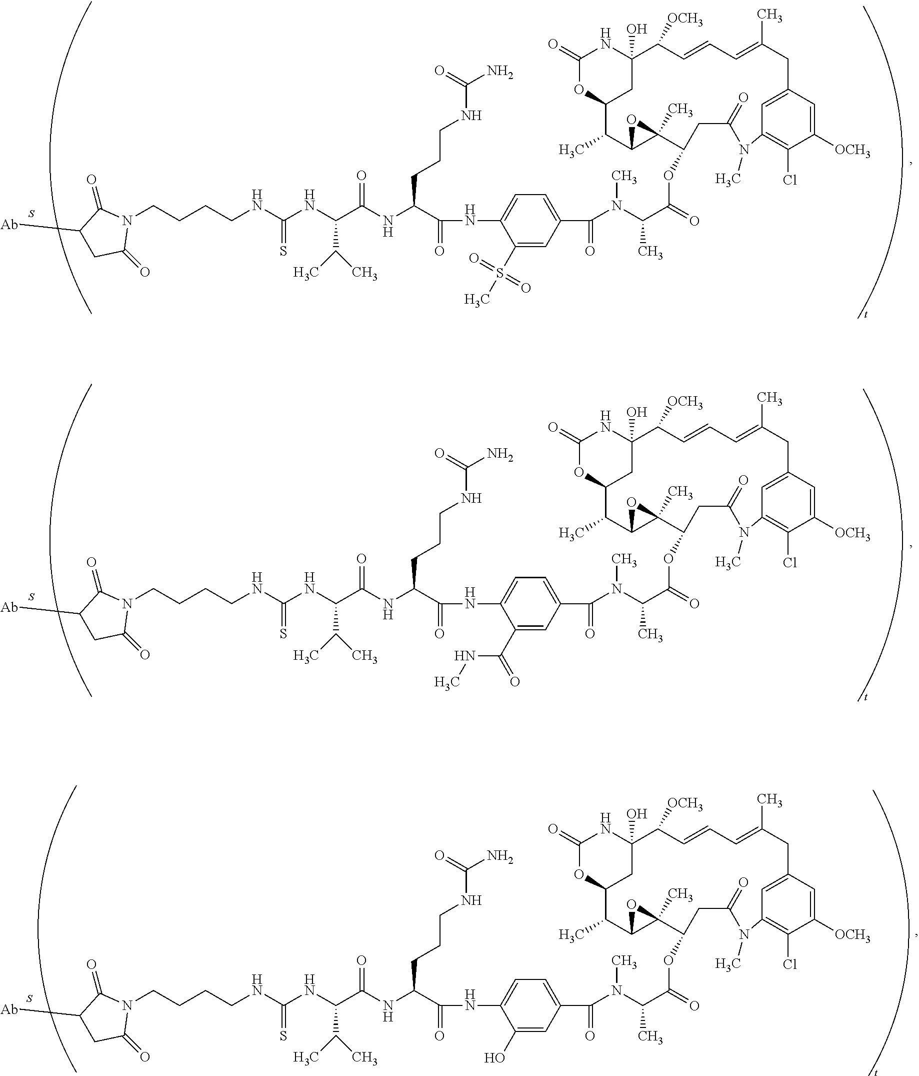

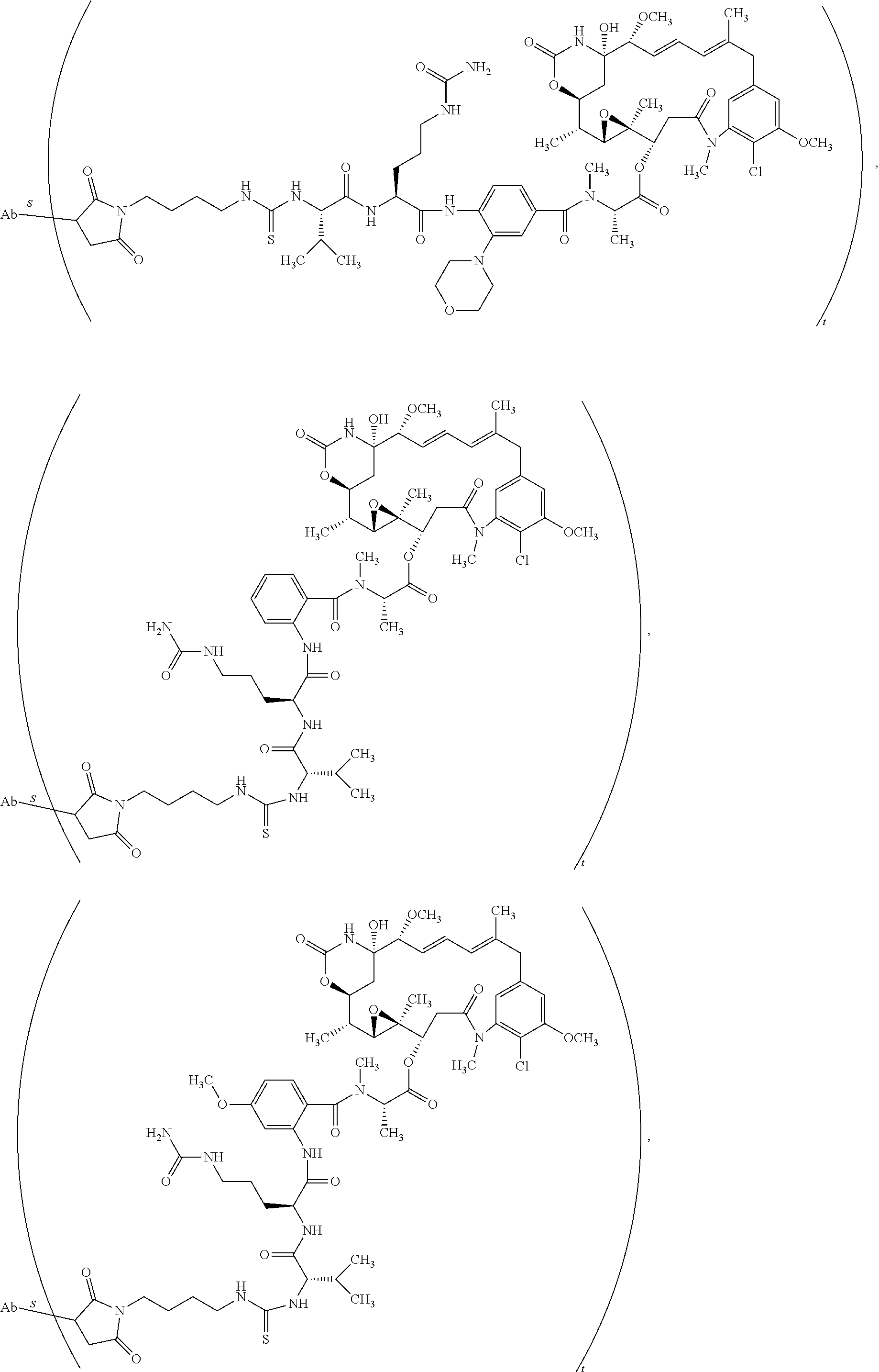

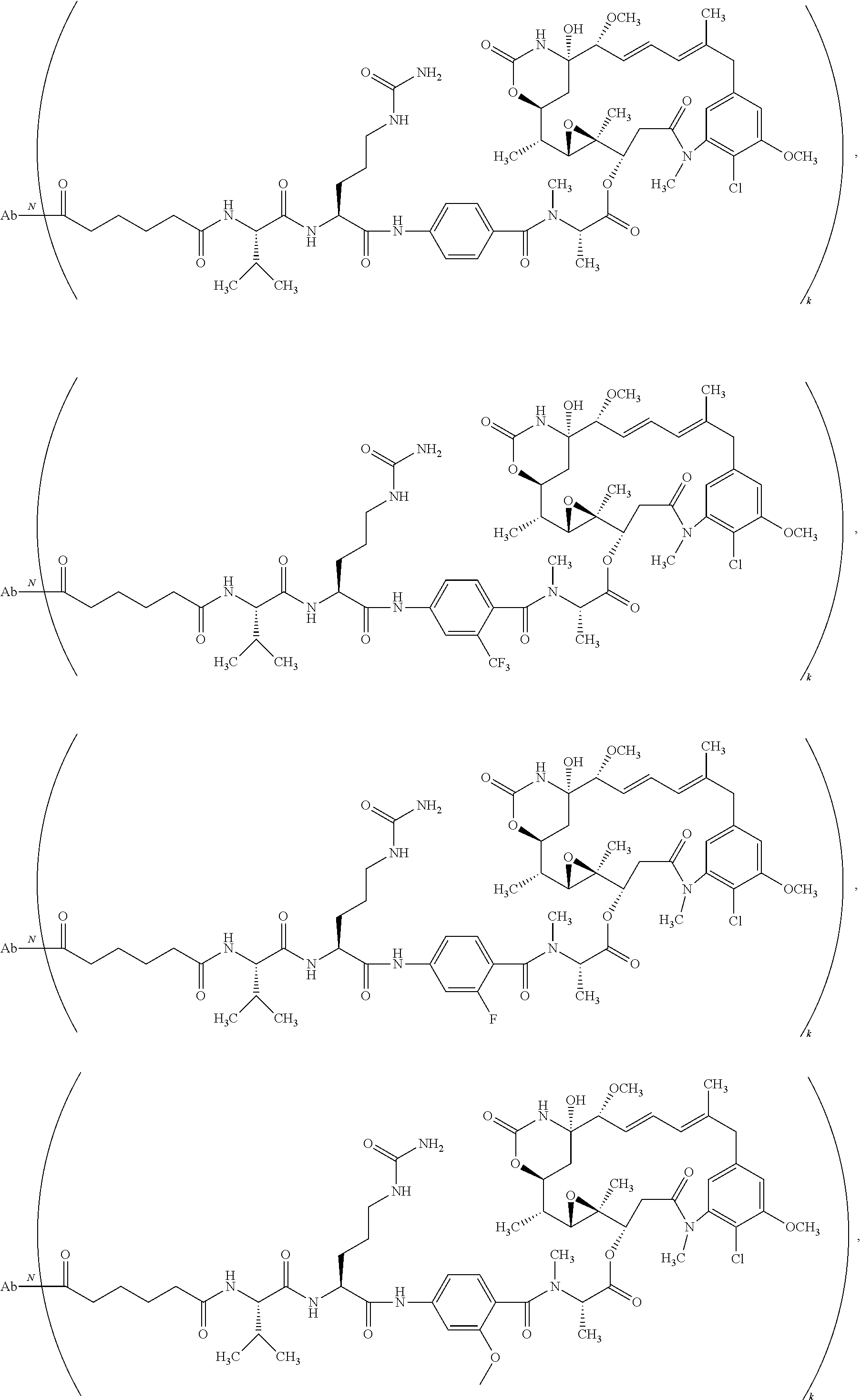

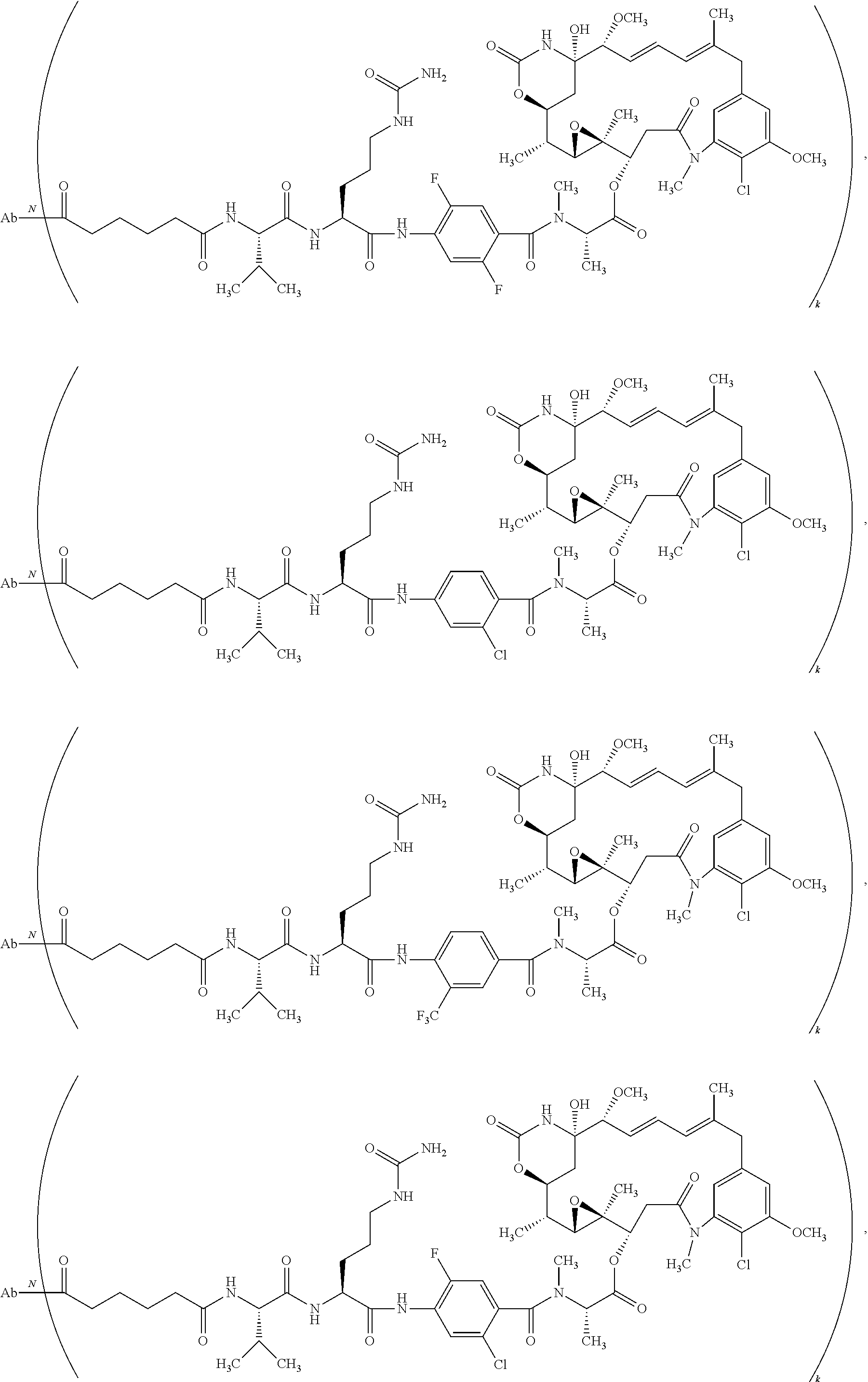

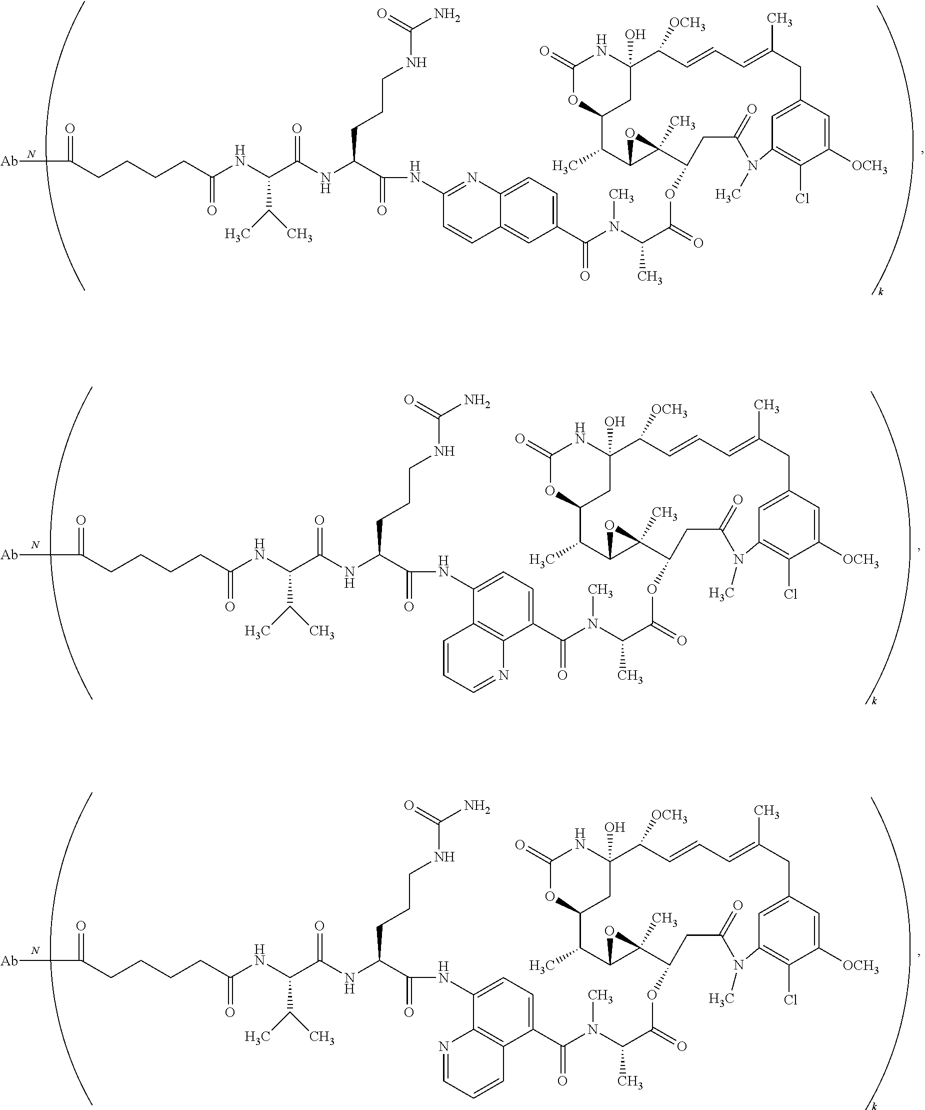

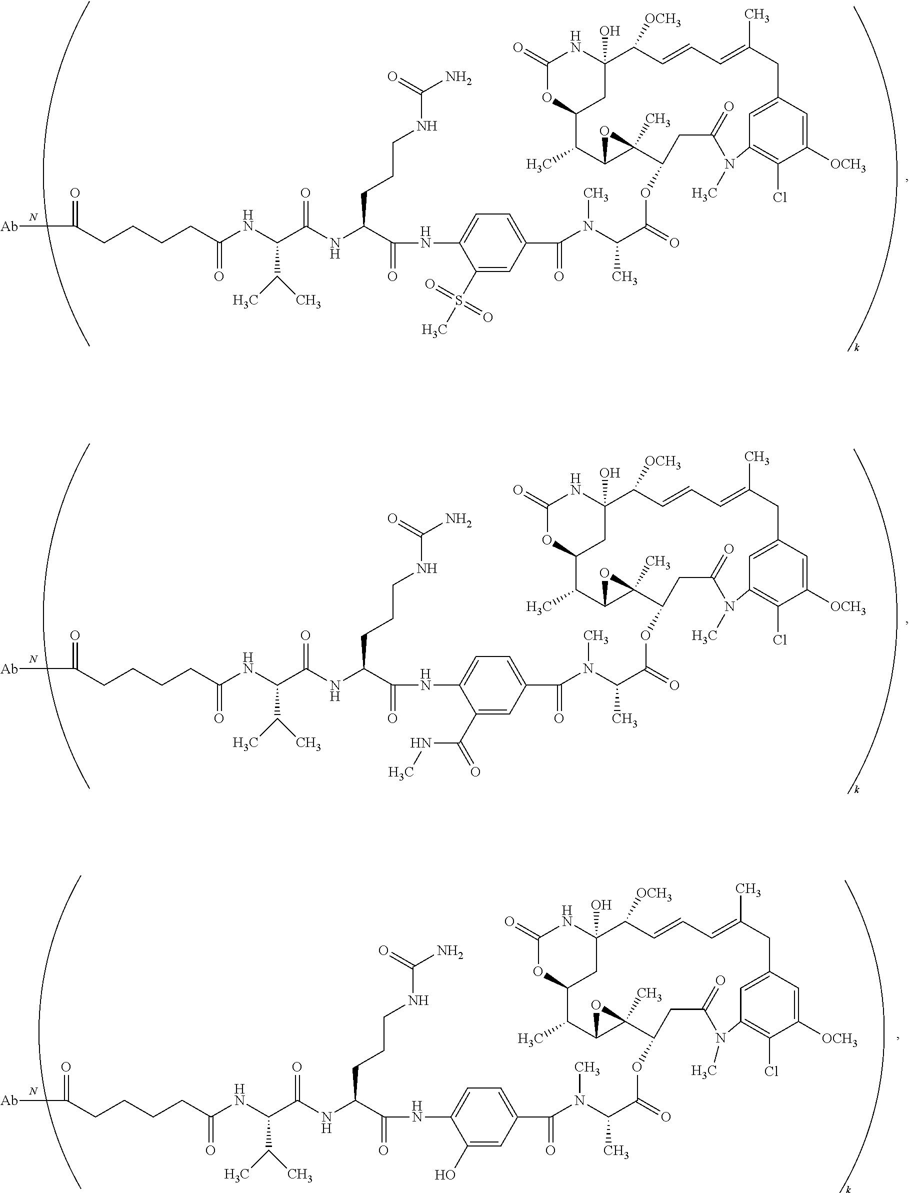

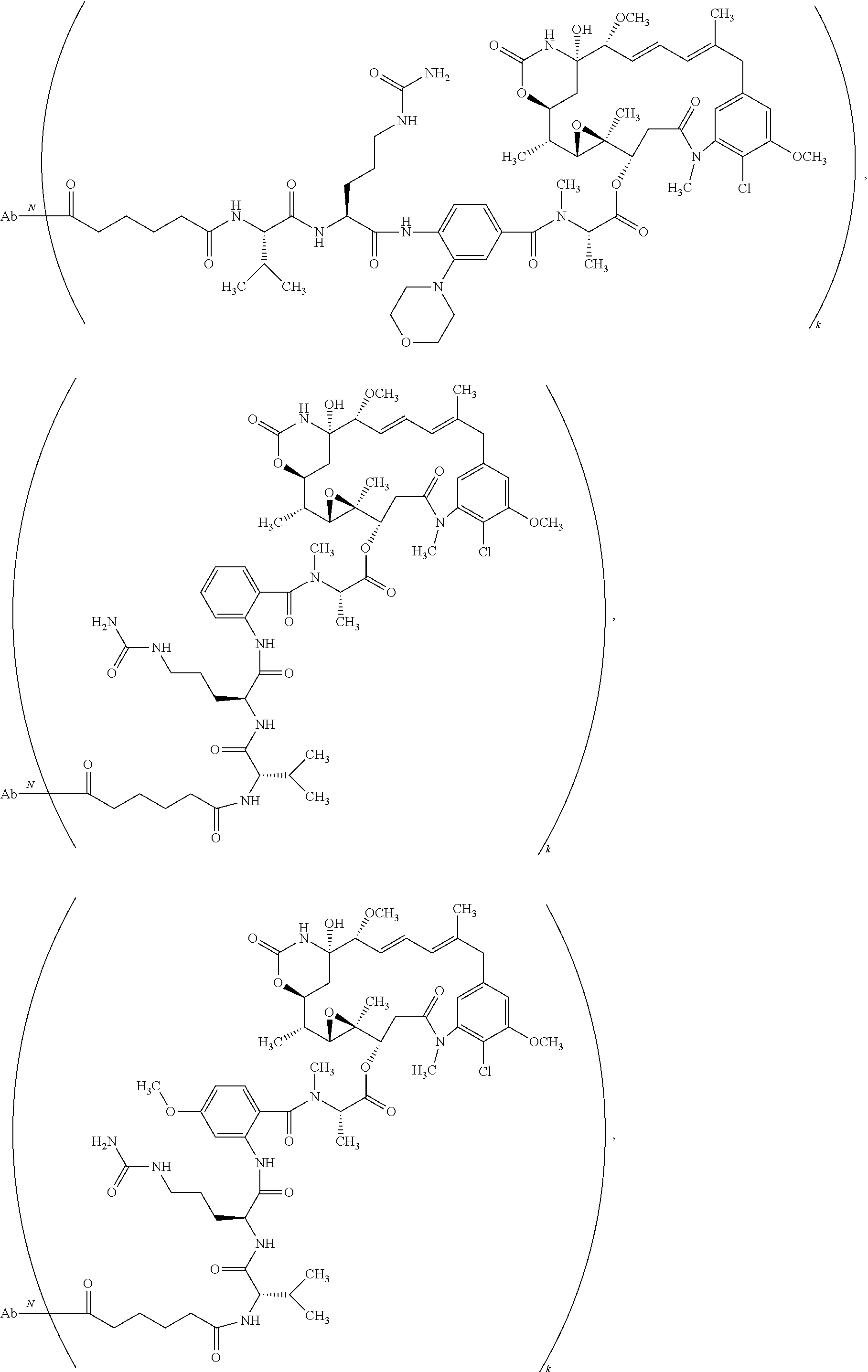

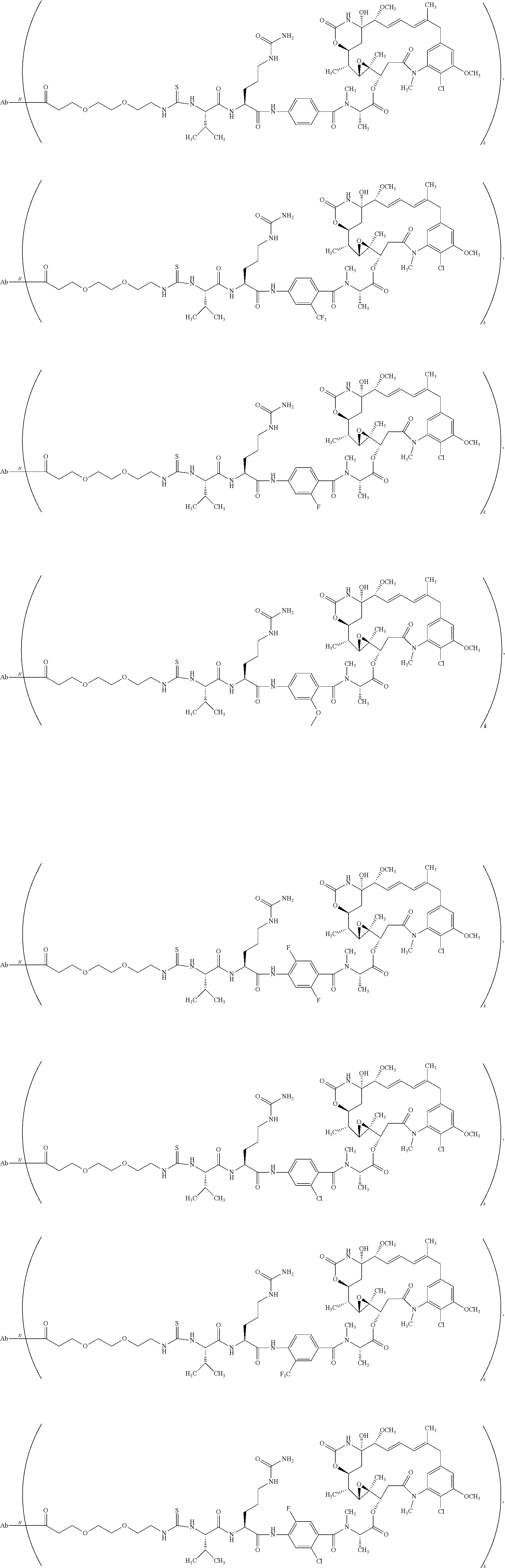

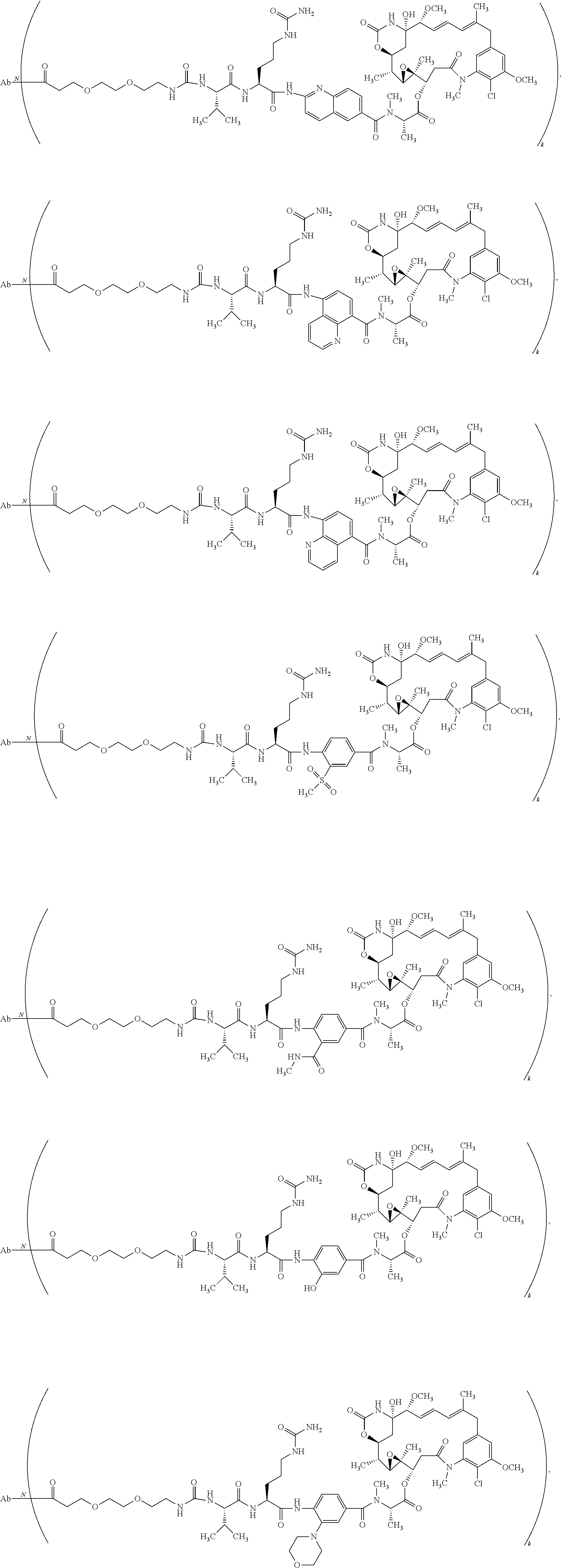

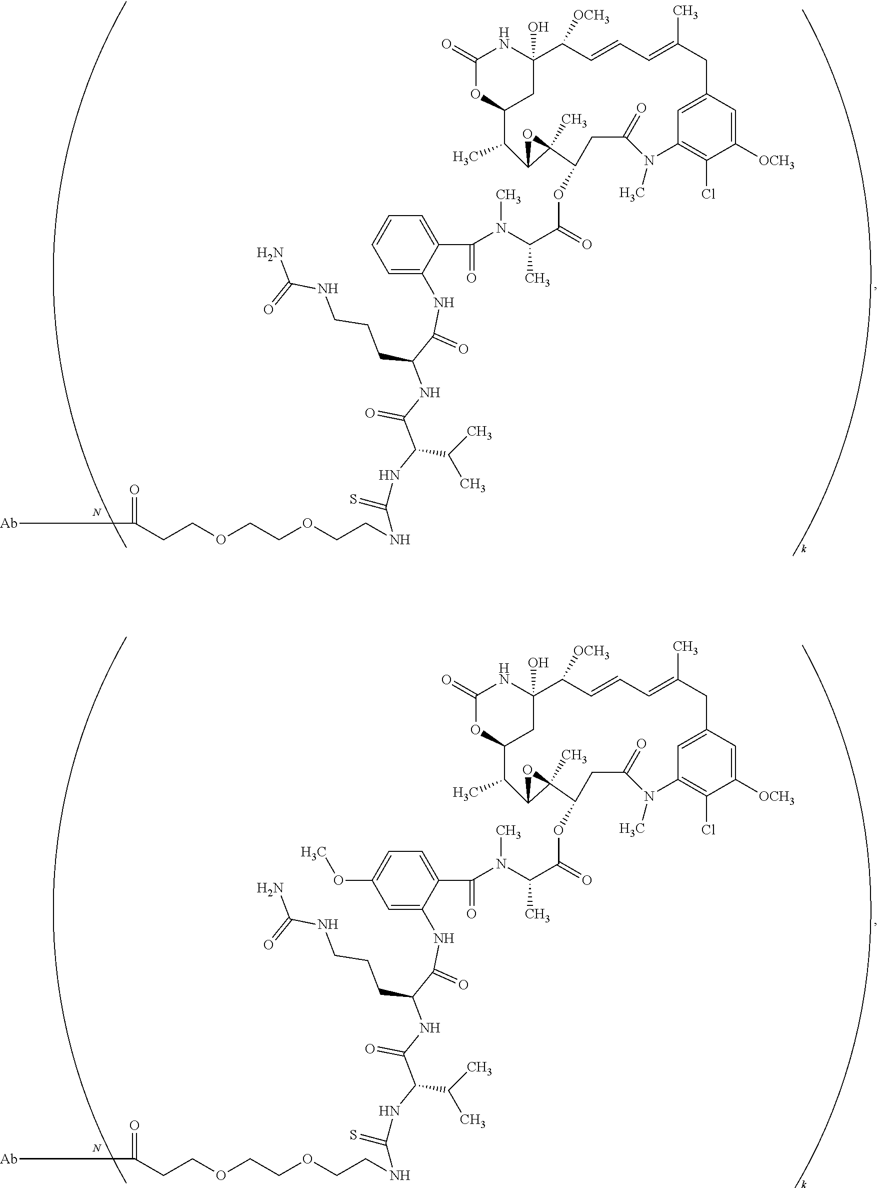

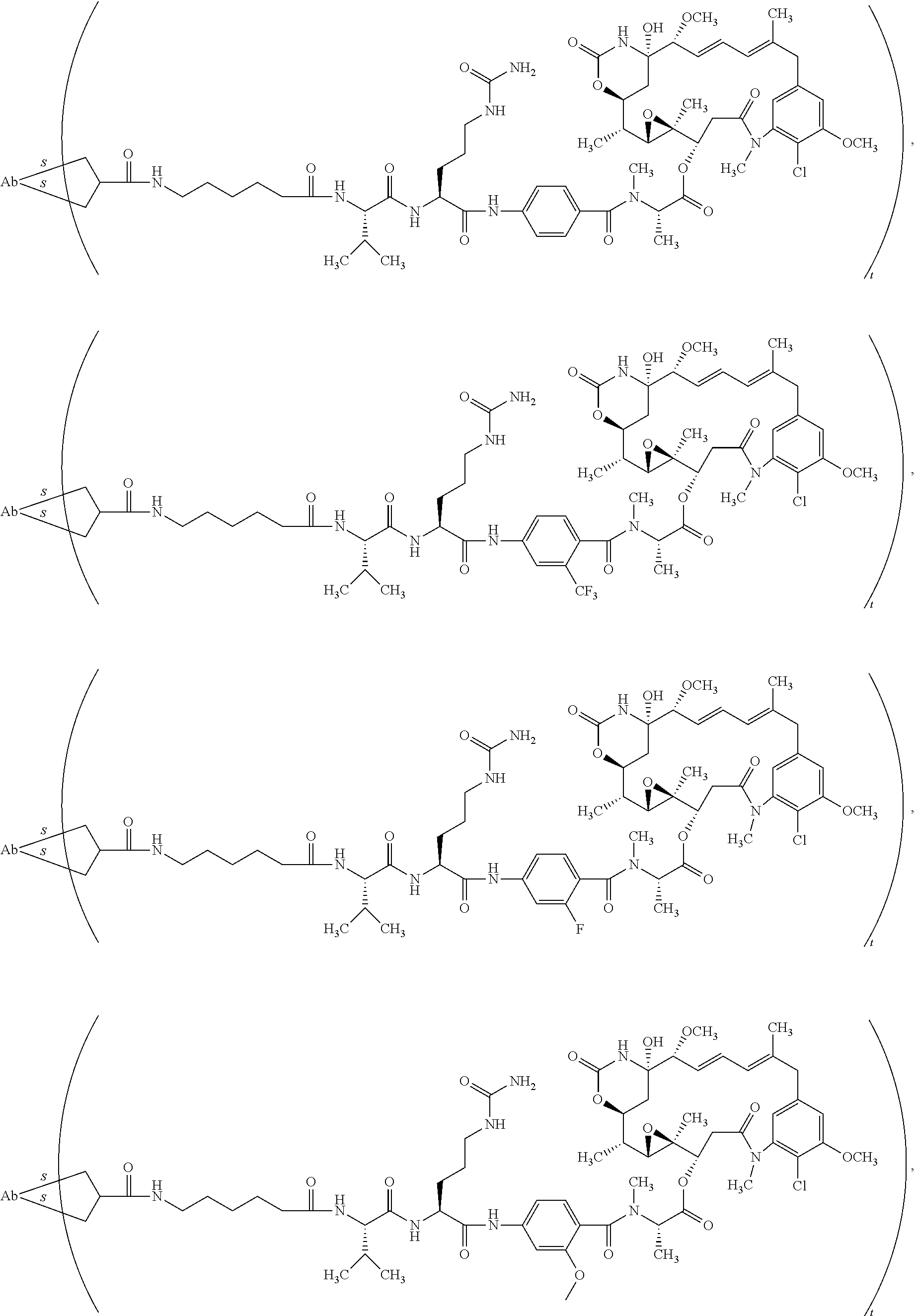

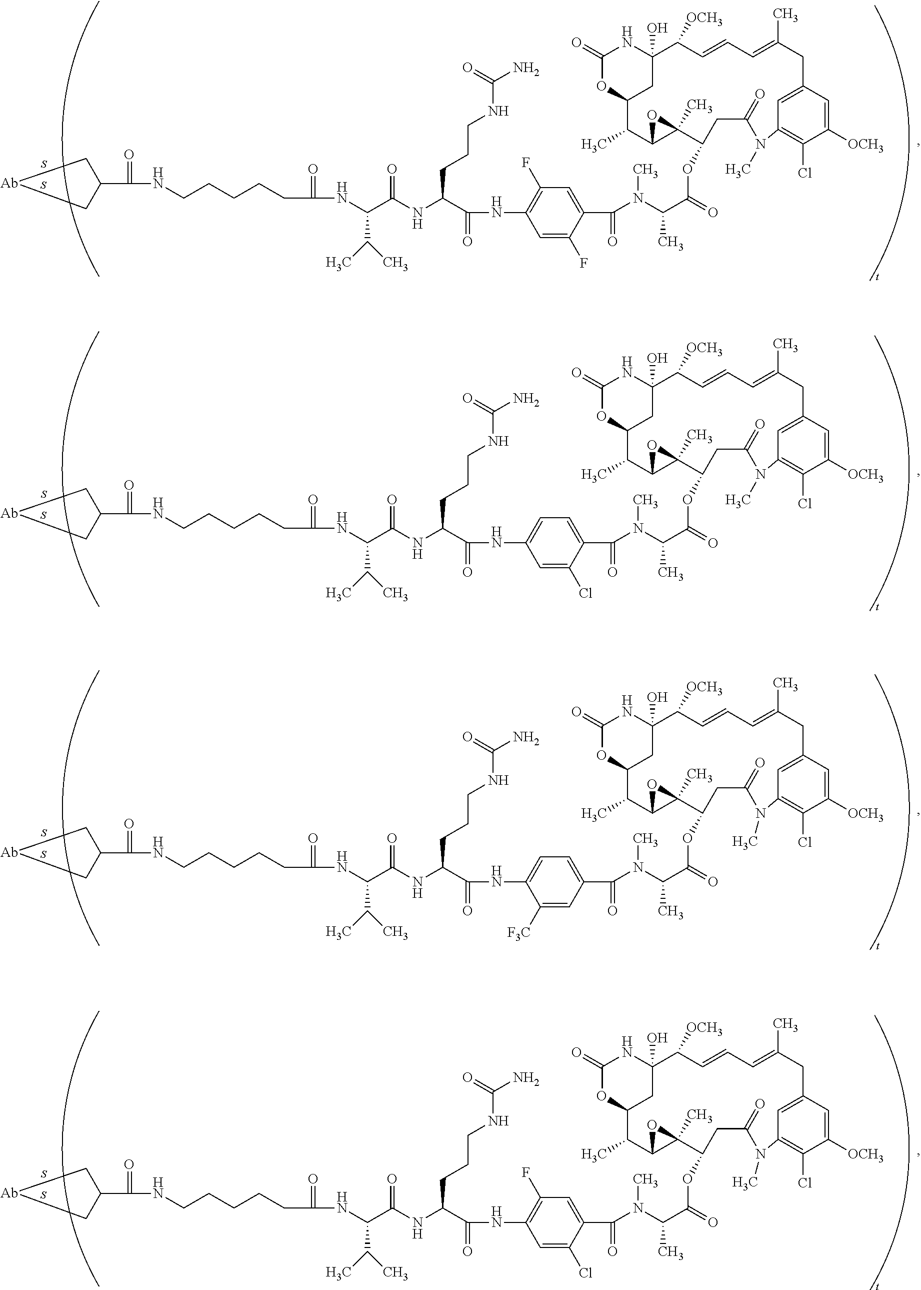

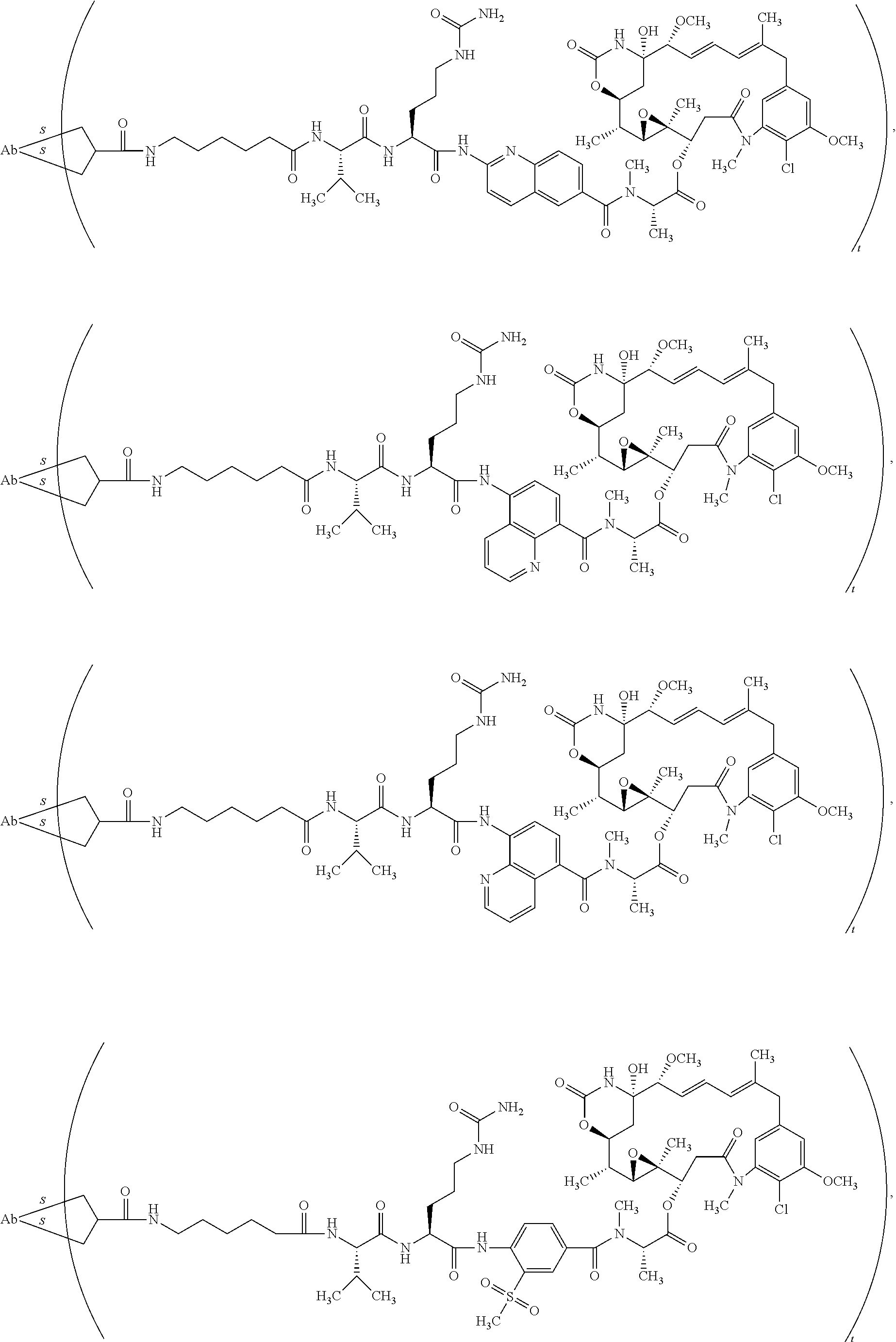

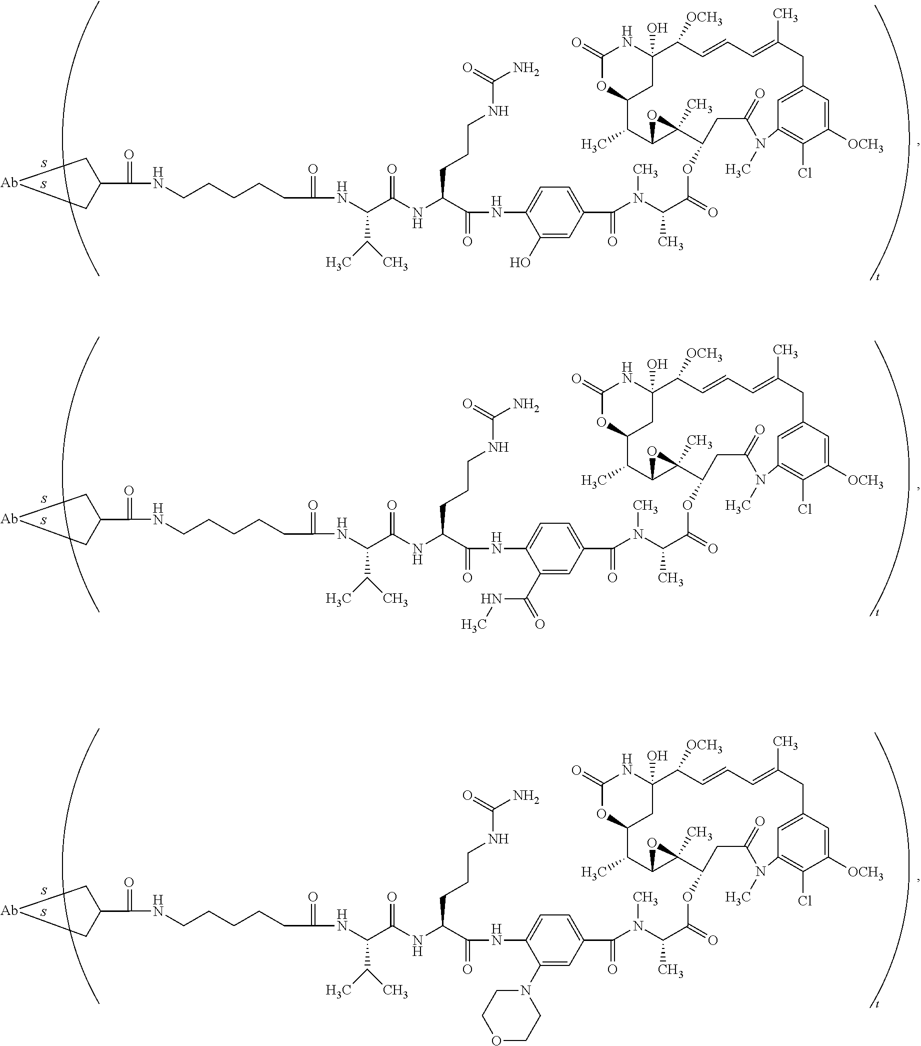

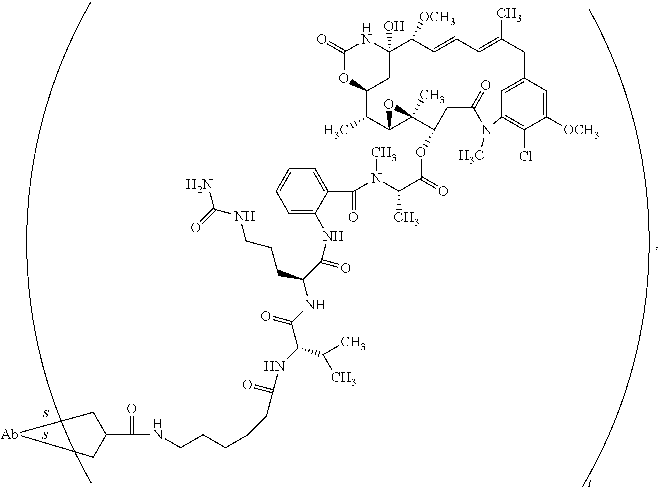

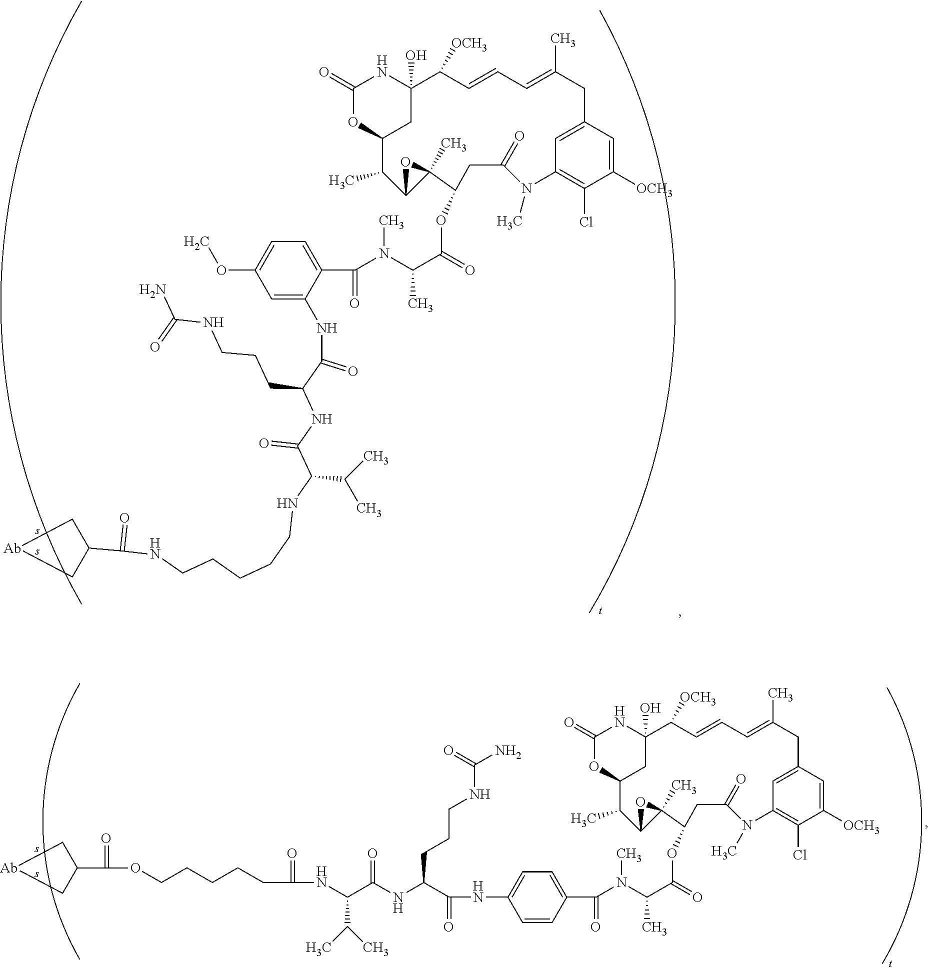

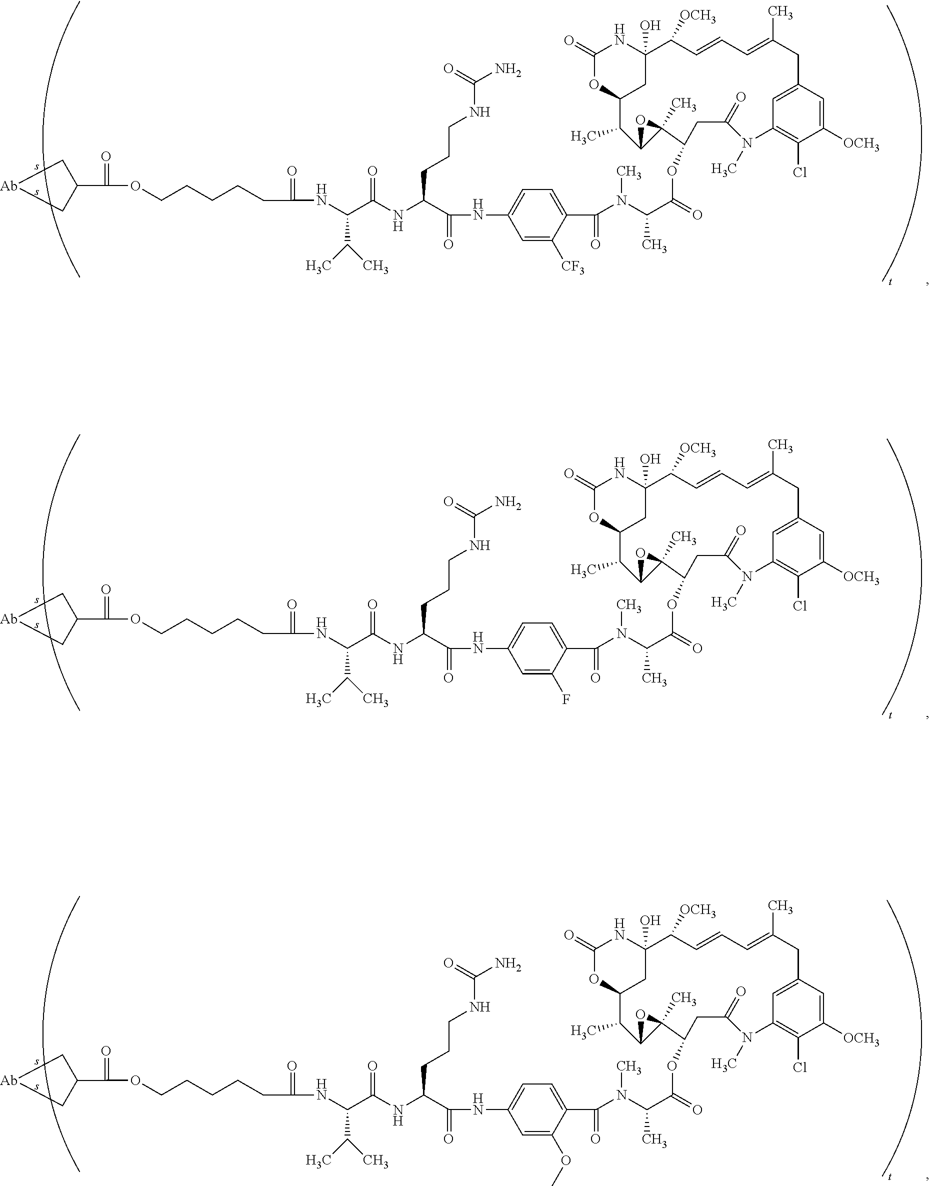

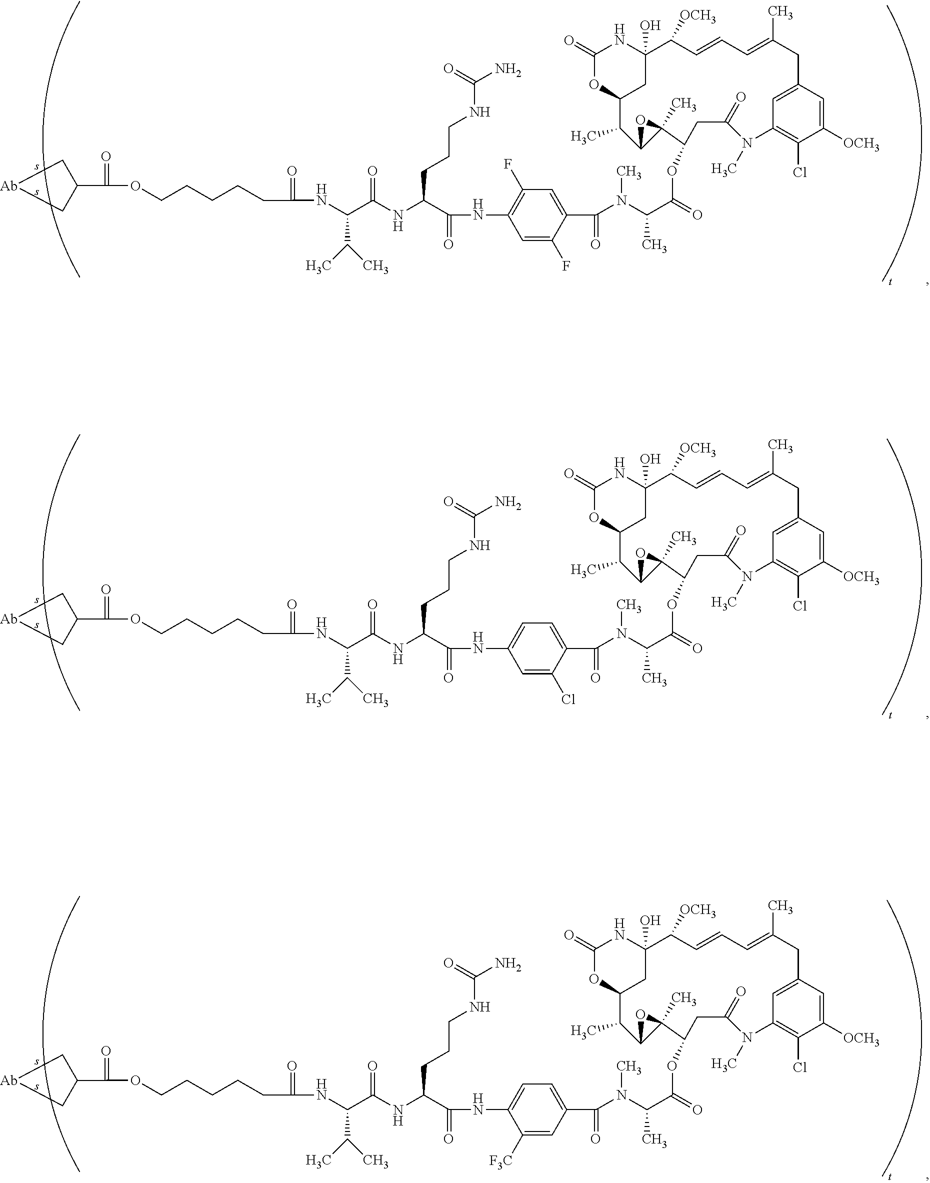

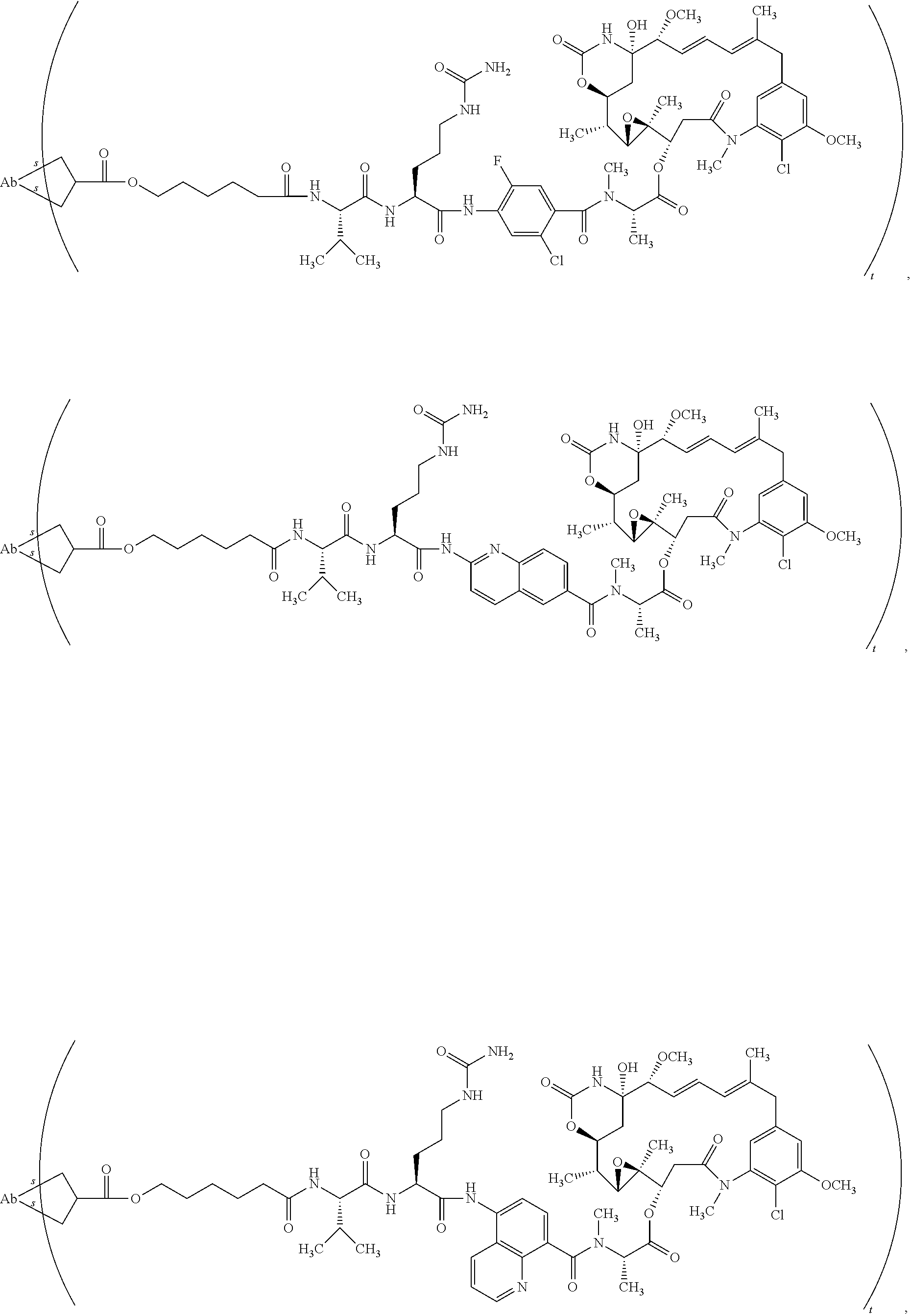

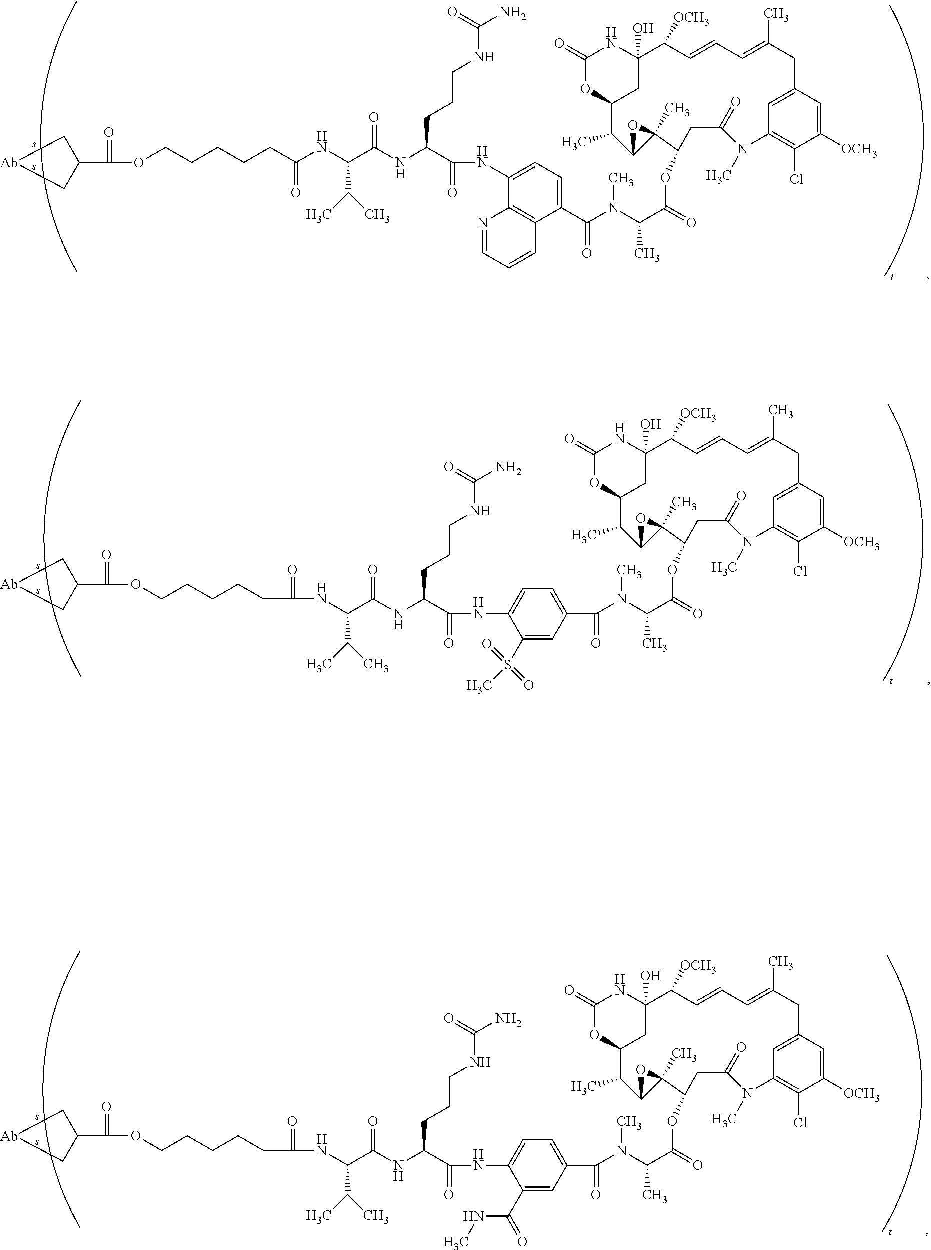

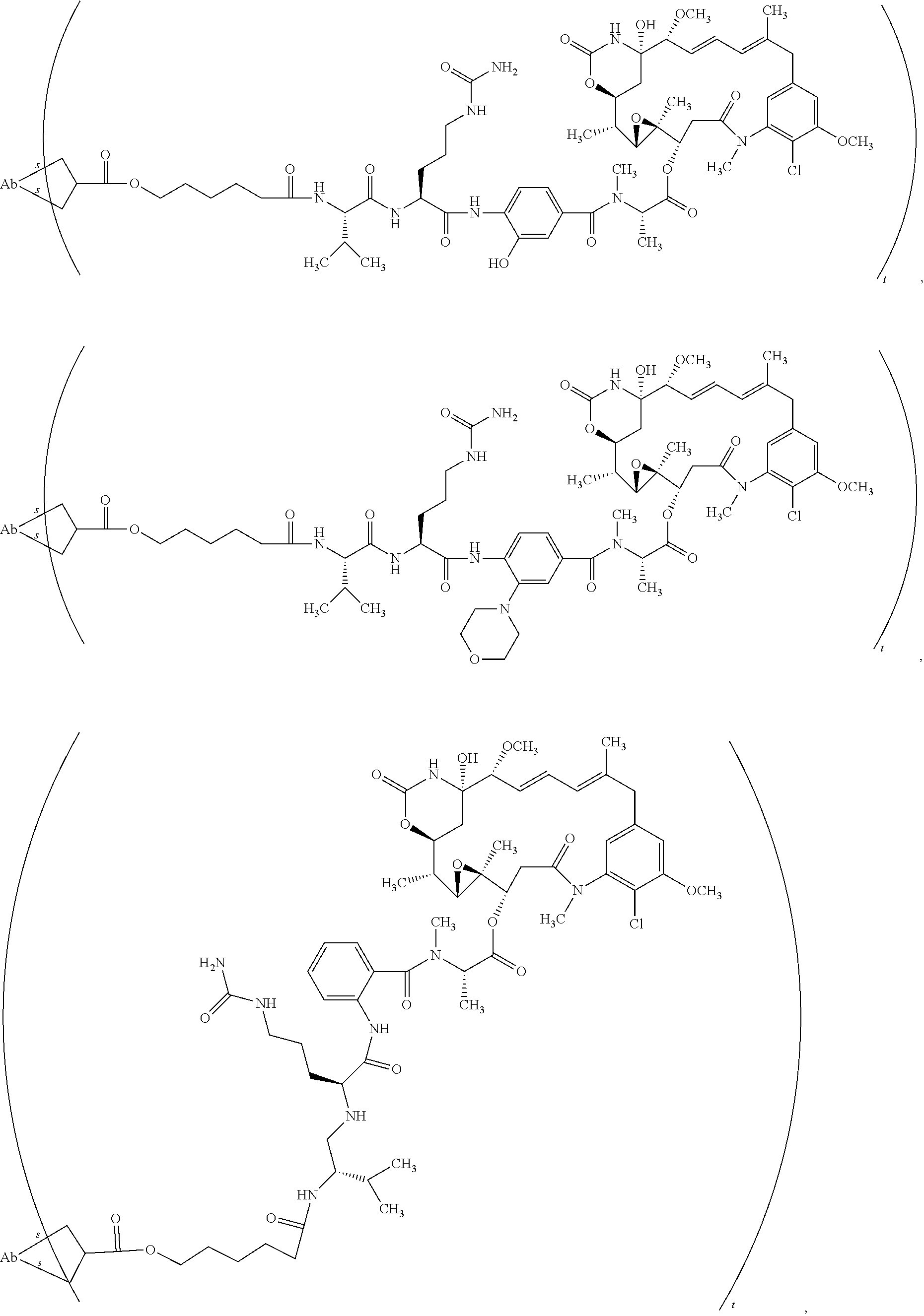

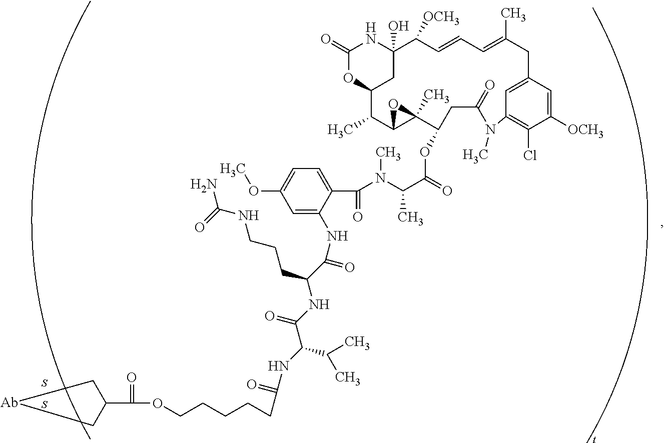

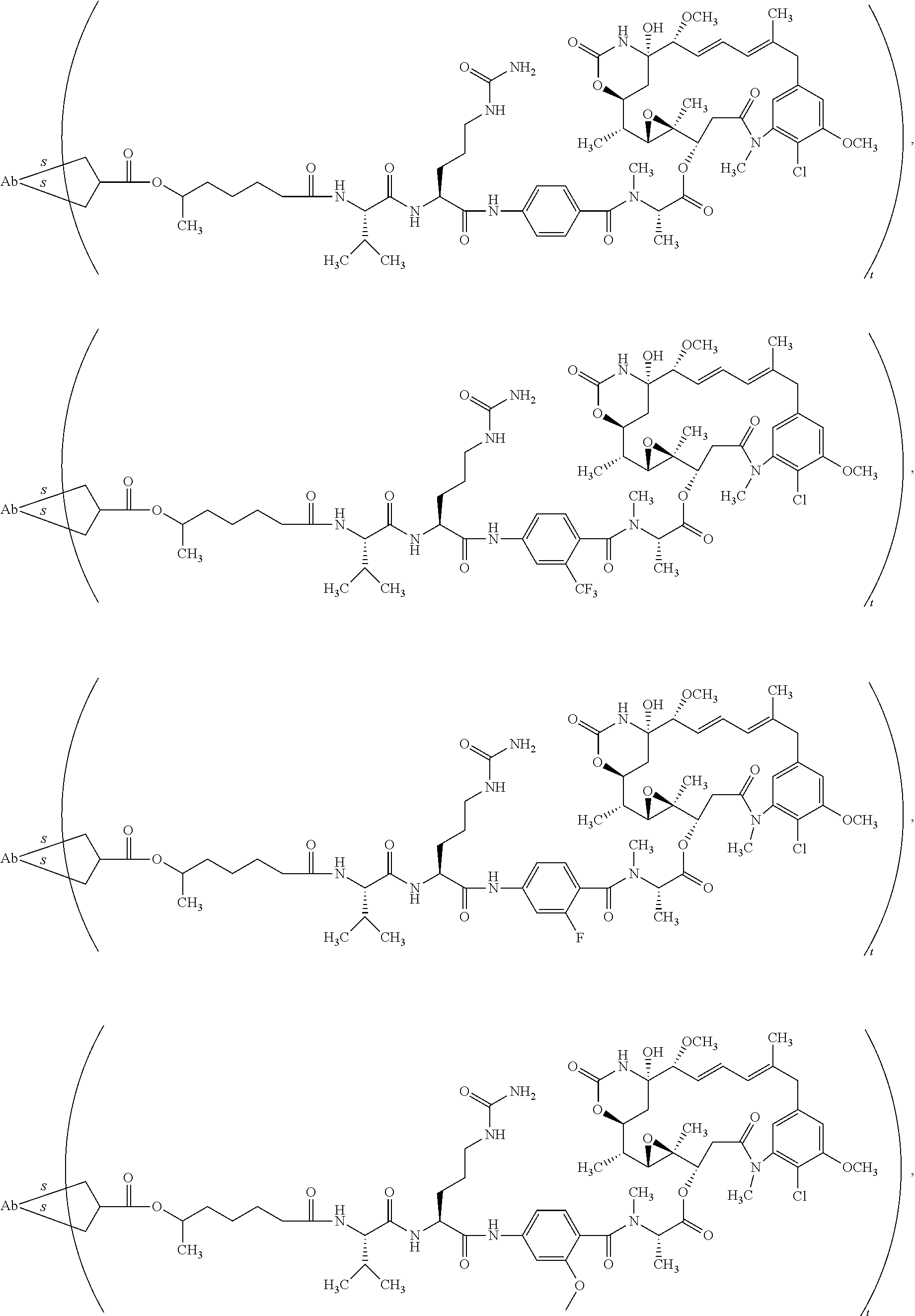

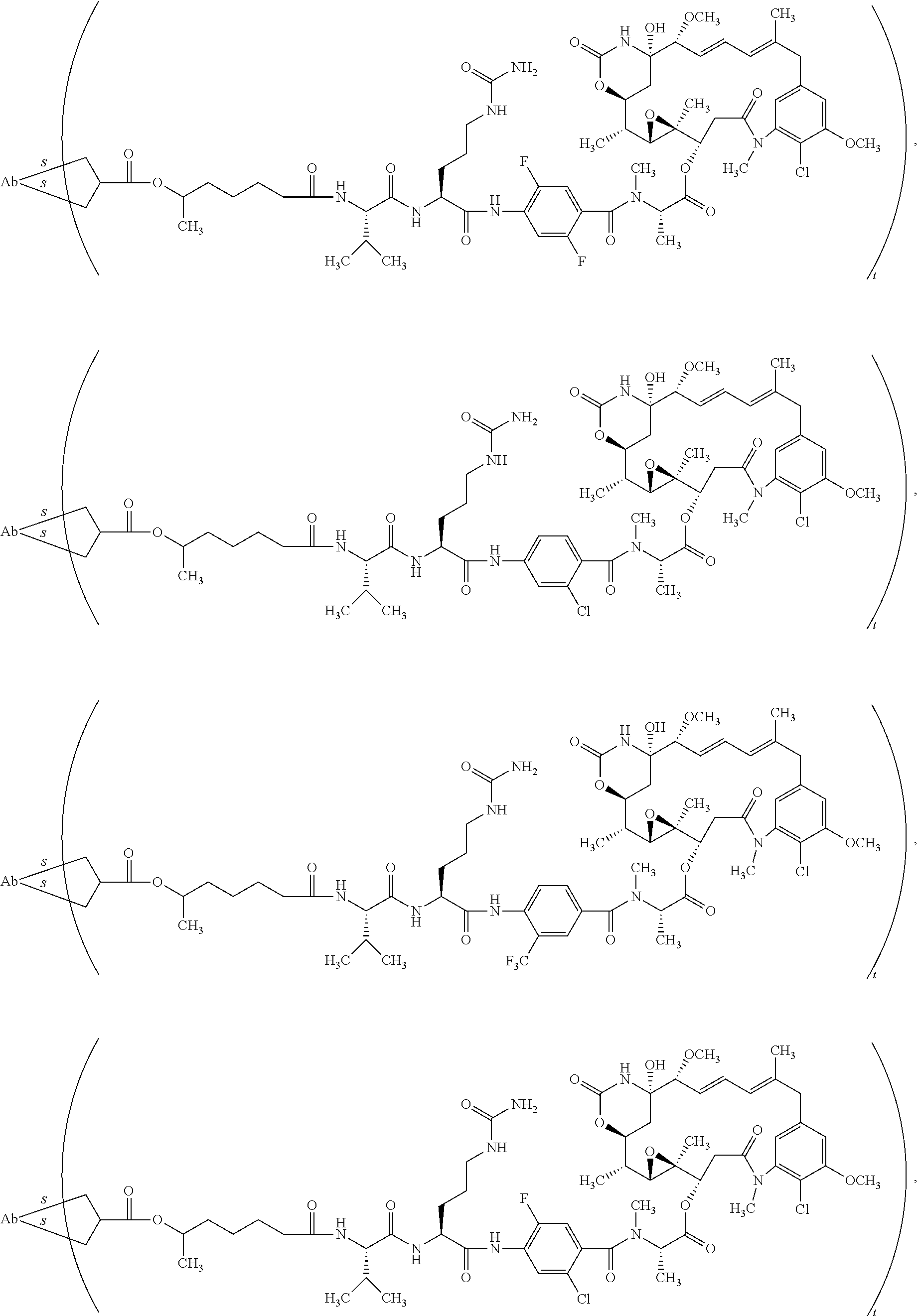

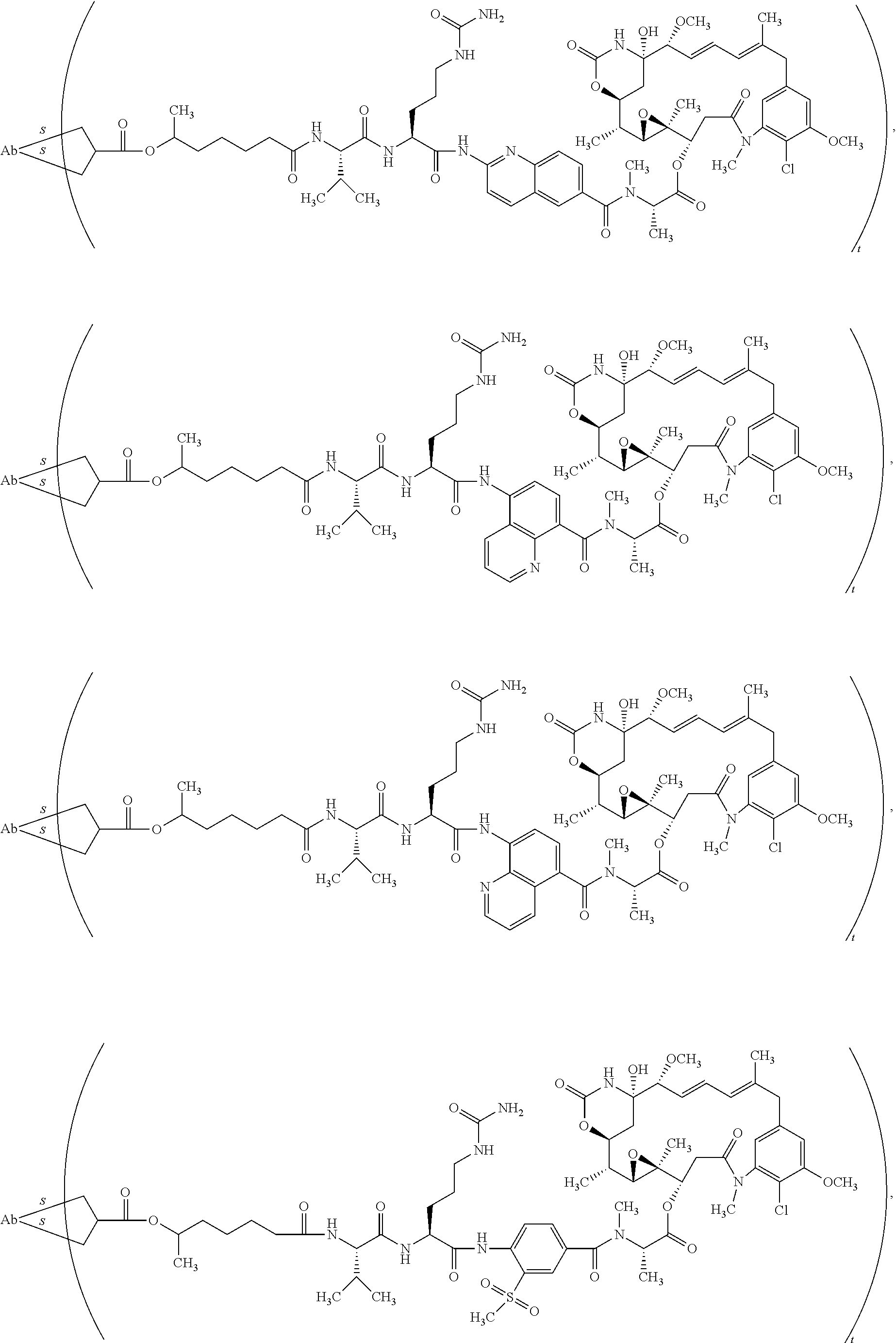

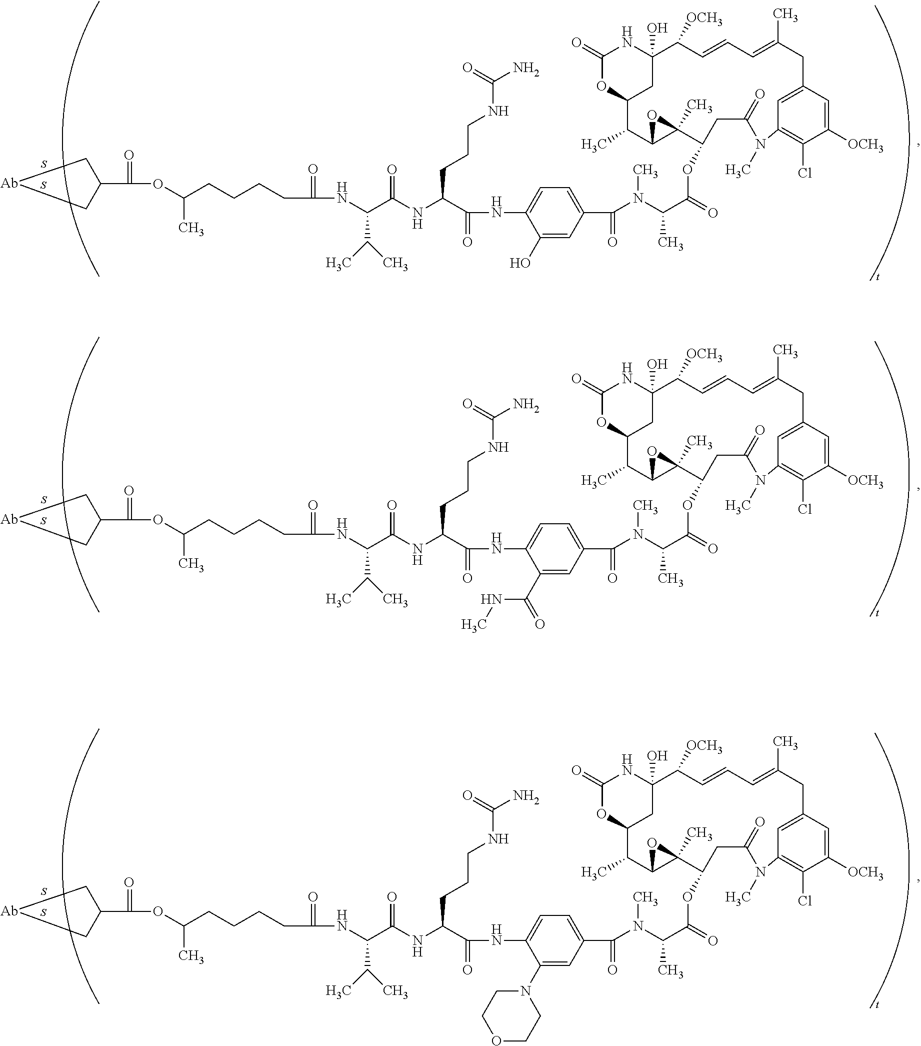

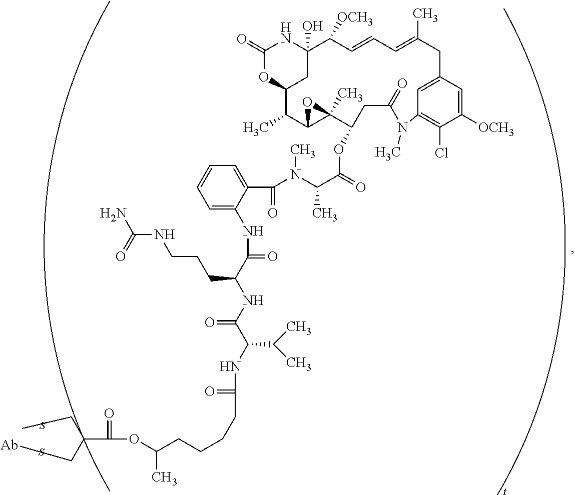

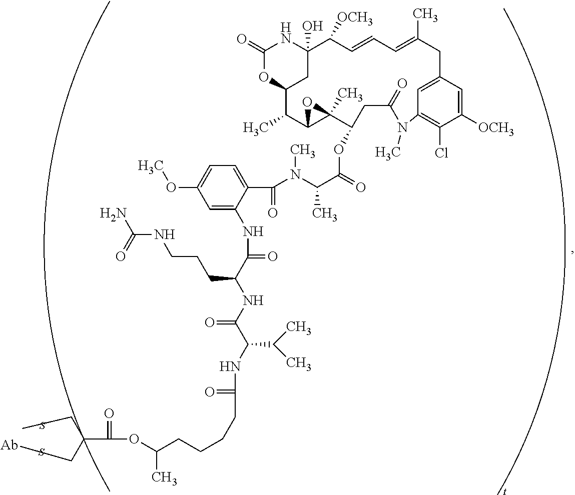

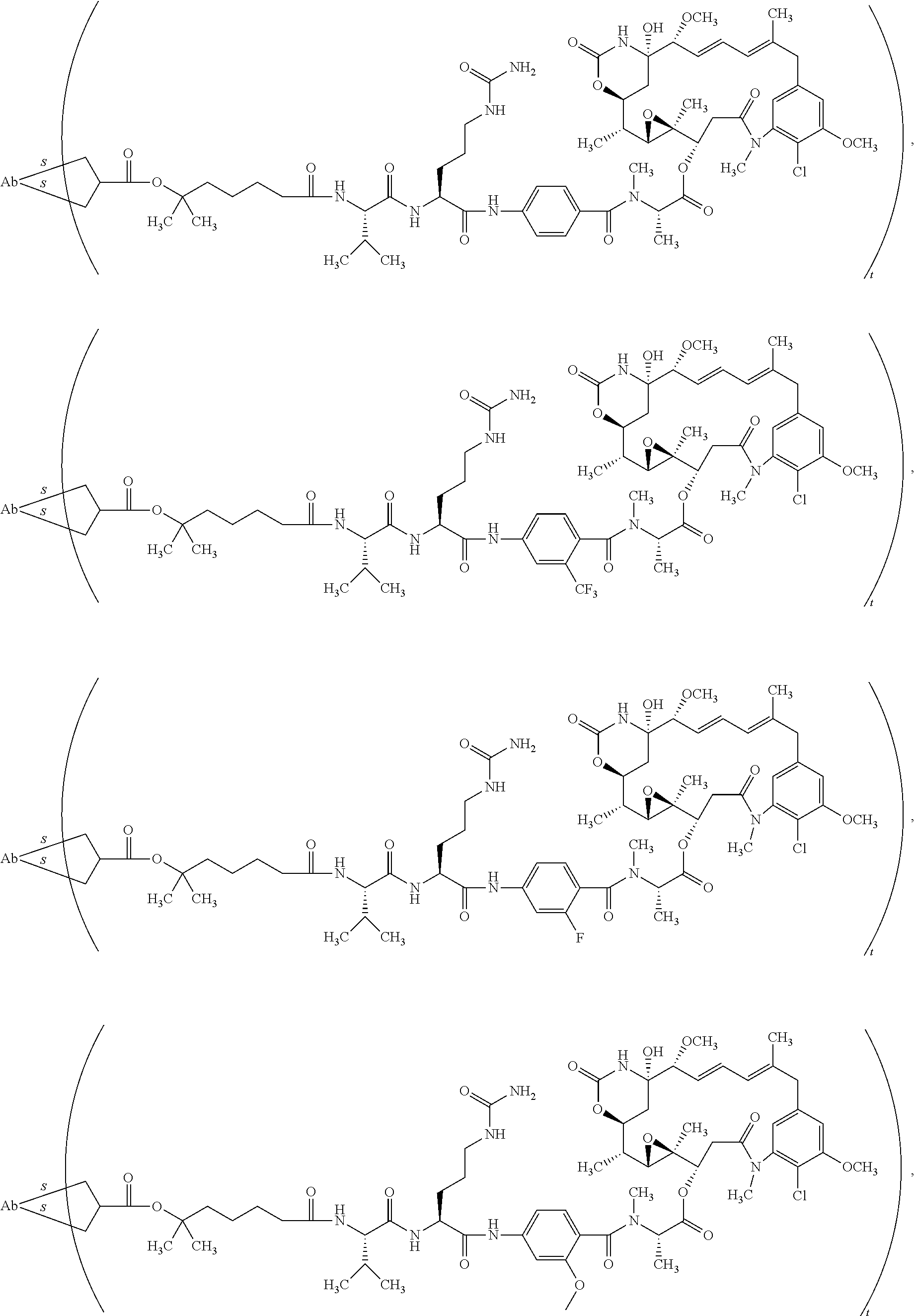

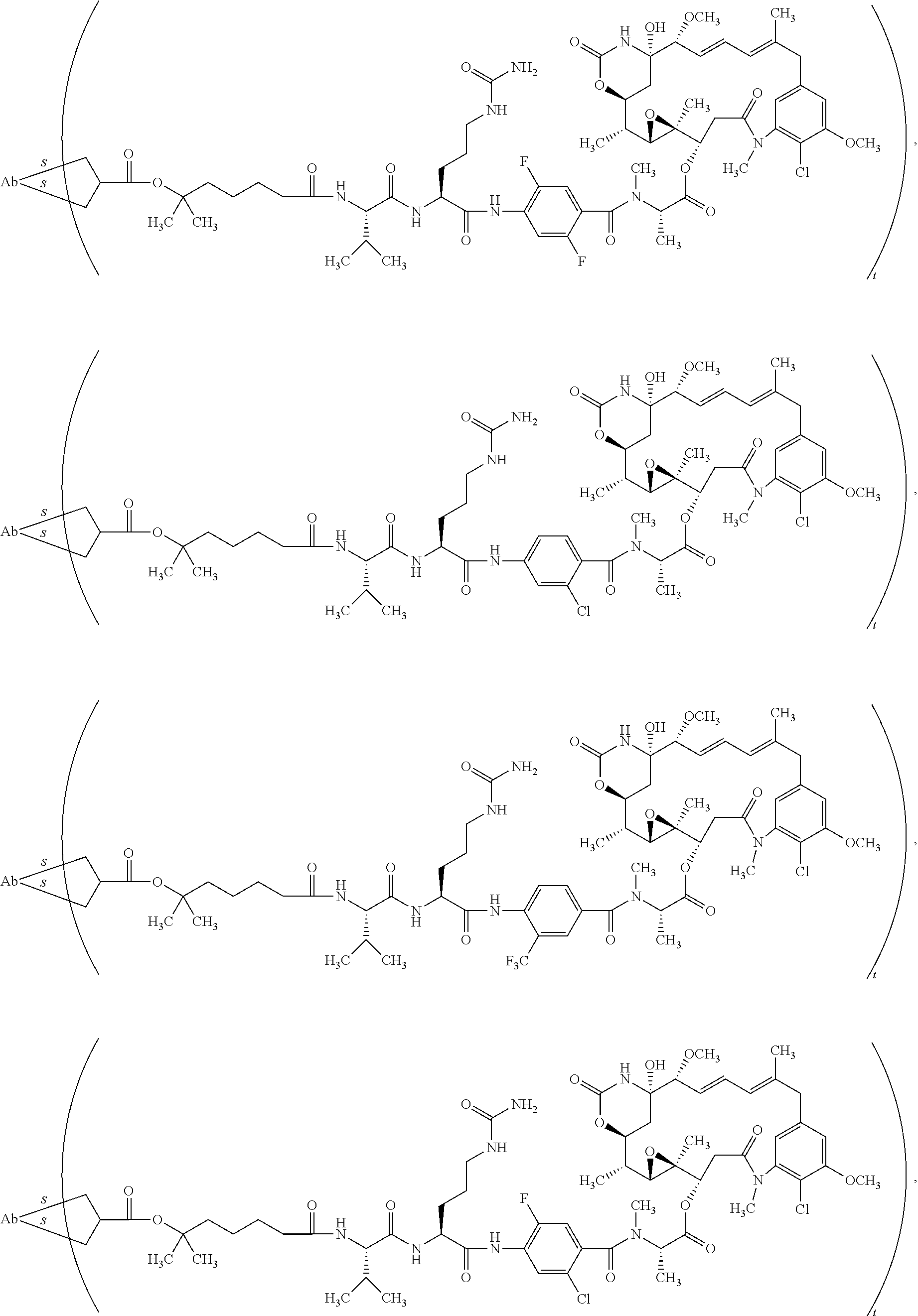

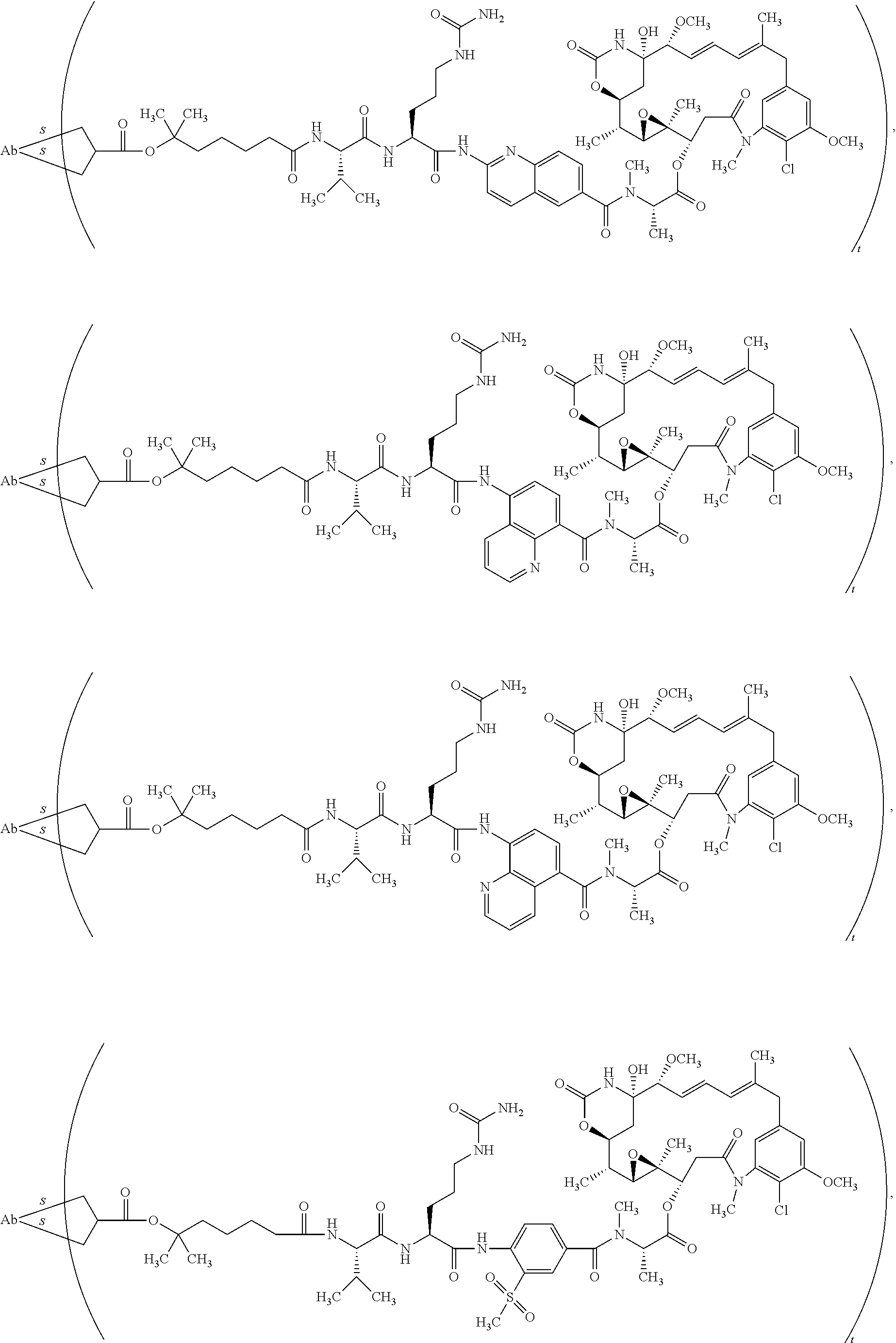

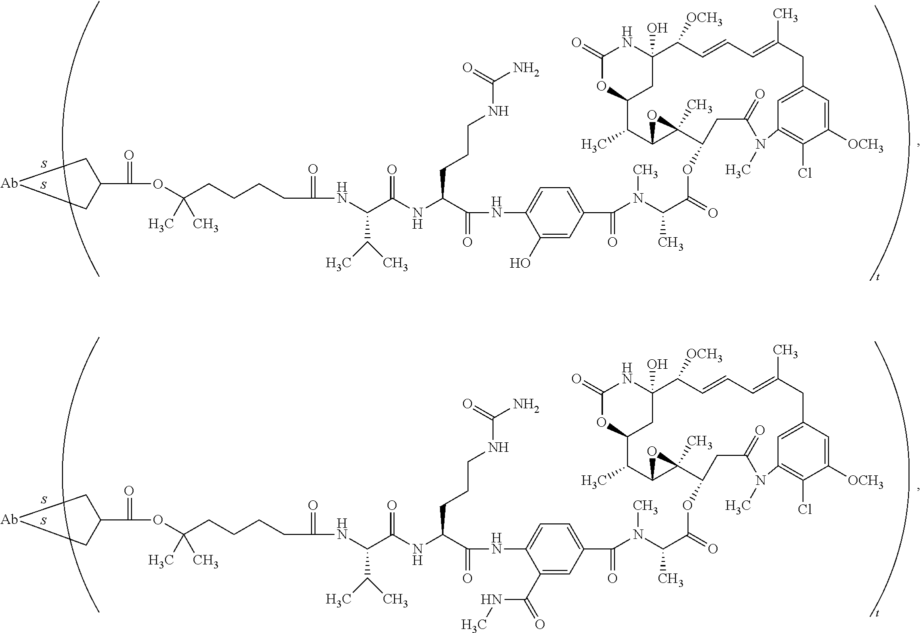

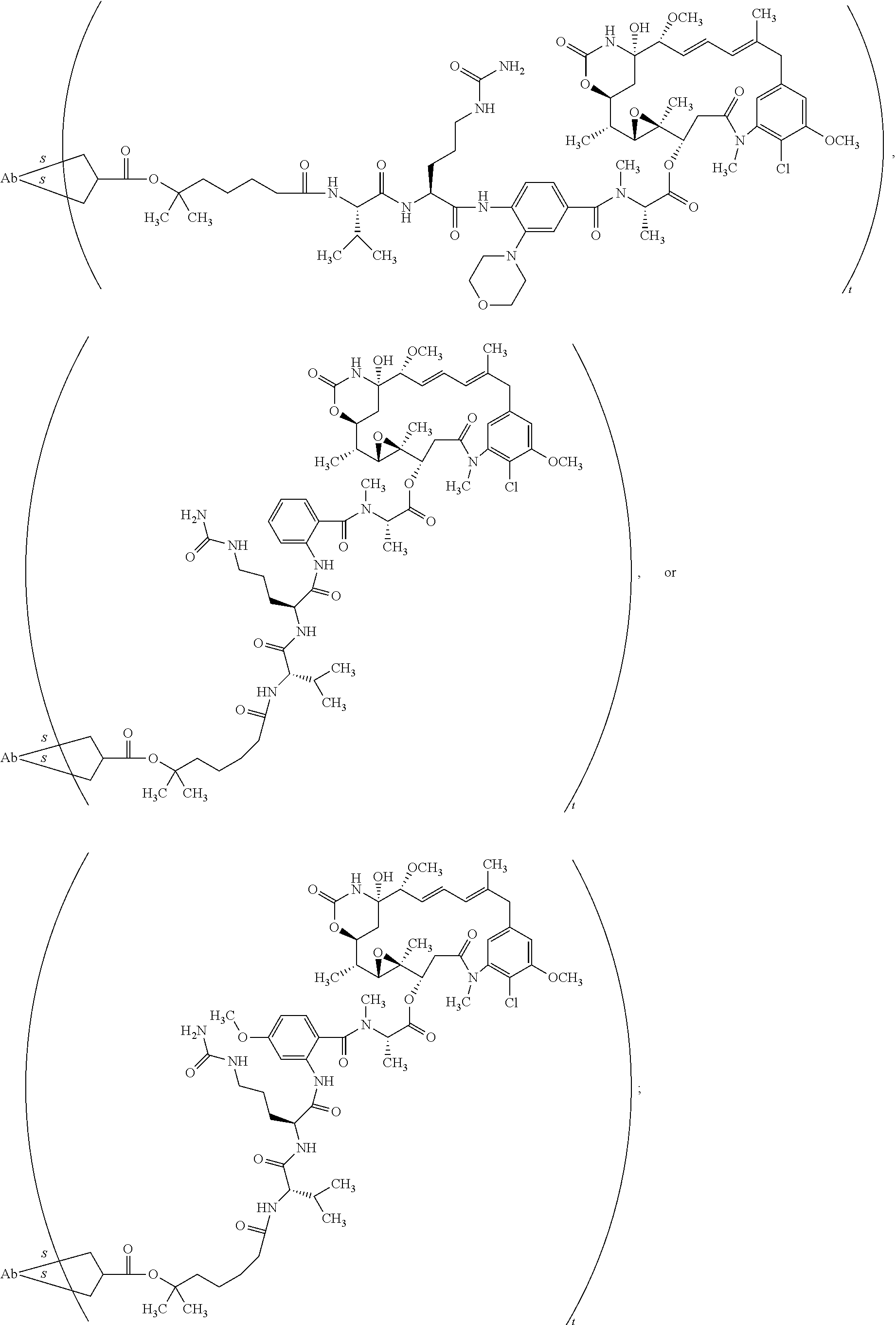

wherein:
Ab is an antibody;
[symbol] is a bond to a cysteine of the antibody;
[symbol] is a bond to a lysine of the antibody;
k is an integer from 1 to 30; and
t is an integer from 1 to 8. In some examples, k is an integer from 1 to 8. In some examples, t is an integer from 1 to 4. In some examples, when [symbol] is a bond to a cysteine of the antibody, up to 8 conjugates set forth herein may be bonded to the antibody. In some examples, when [symbol] is a bond to a lysine of the antibody, up to 30 conjugates set forth herein may be bonded to the antibody.

In some embodiments, the compound of Formula I is:

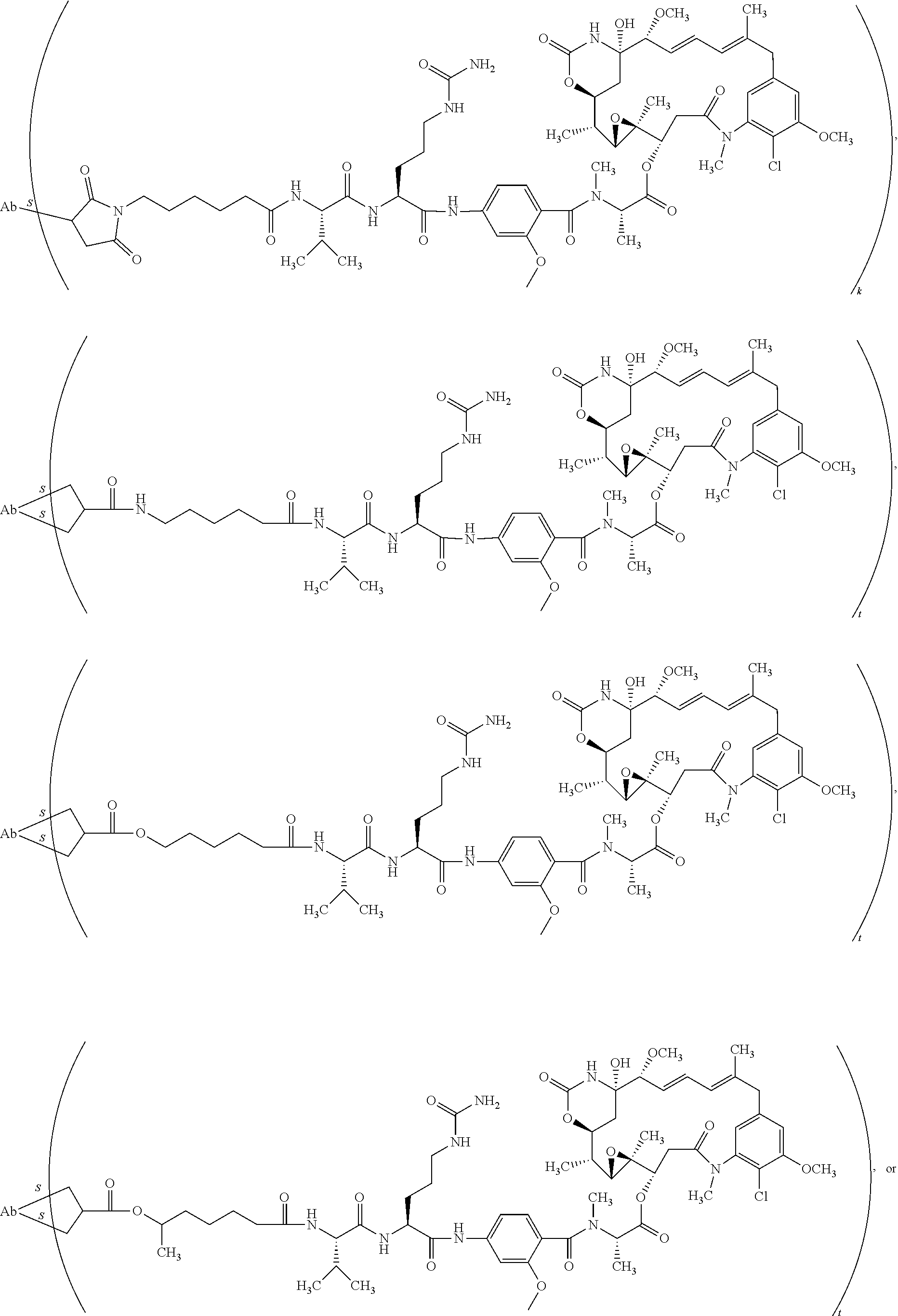

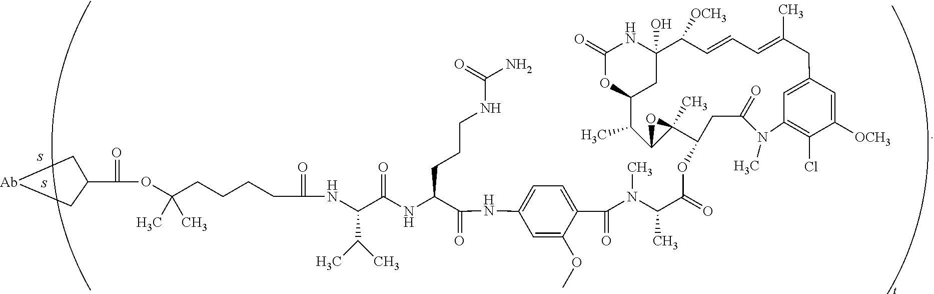

In some embodiment, k is an integer from 1 to 30. In some embodiment, k is an integer from 1 to 8. In some embodiment, k is an integer from 1 to 6. In some embodiments, k is an integer from 1 to 4. In some embodiments, k is an integer from 1 to 3. In some embodiments, the drug-antibody ratio (DAR) of the conjugate is from 1.0 to 3.0.

### C. Maytansinoid Derivatives

Provided herein are compounds of Formula (II):

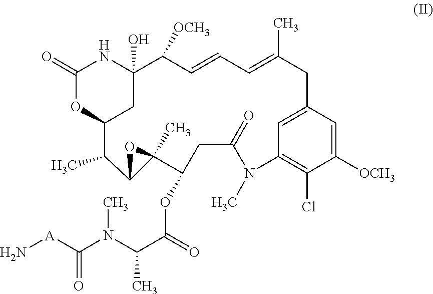

or a pharmaceutically acceptable salt thereof,
wherein A is arylene or heteroarylene.

In certain embodiments, these compounds represent the payload portion of the conjugates described herein and are released, e.g., by enzyme proteolysis, following internalization of the conjugate into a cell. The methods provided herein include methods of treating a proliferative disease, e.g., cancer, comprising administering to a patient a therapeutically effective amount of a conjugate, e.g., antibody-drug conjugate that releases a compound of Formula (II) following internalization of said conjugate into a cell in said patient.

In some embodiments, these compounds represent the metabolic product of the conjugates described herein, e.g., enzyme proteolysis product. In some embodiments, these compounds represent the catabolic product of the conjugates described herein. In some embodiments, these compounds represent the cellular product of the conjugates described herein.

In some embodiments, A is a divalent radical of benzene, of pyridine, of naphthalene, or of quinolone, which are optionally substituted.

In some embodiments, A is arylene.

In some embodiments, A is:

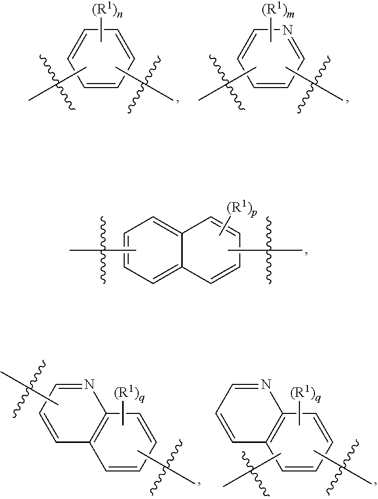

wherein:
   R¹ is, independently at each occurrence, alkyl, alkenyl, alkynyl, aryl, alkaryl, aralkyl, halo, heteroaryl, heterocycloalkyl, hydroxyl, cyano, nitro,

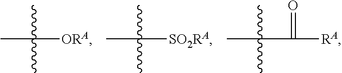

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, the compound of Formula (II) is a compound of the Formula (IIA):

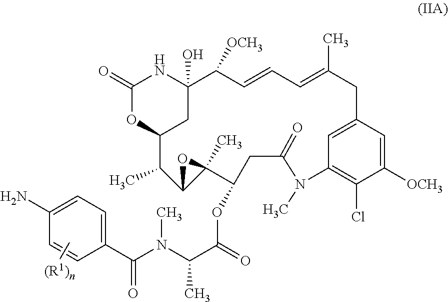

wherein R¹ and n are as defined herein.

In some embodiments, the compound of Formula (II) is a compound of the Formula (IIB):

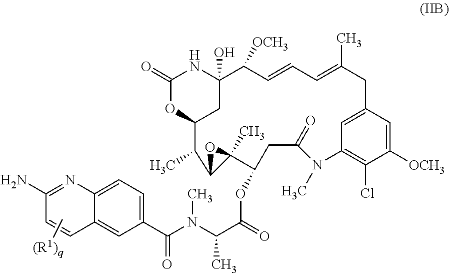

wherein R¹ and q are as defined herein.

In some embodiments, the compound of Formula (II) is a compound of the Formula (IIB2):

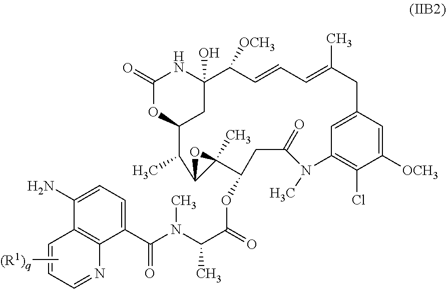

wherein R¹ and q are as defined herein.

In some embodiments, the compound of Formula (II) is a compound of the Formula (IIB3):

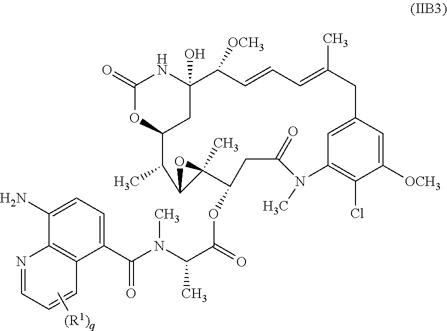

wherein R¹ and q are as defined herein.

In some embodiments, the compound of Formula (II) is a compound of the Formula (IIC):

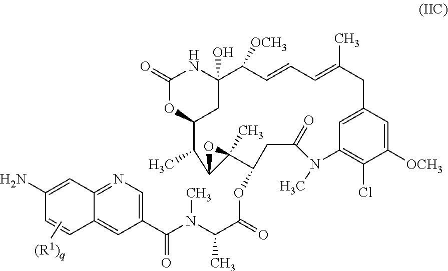

wherein R¹ and q are as defined herein.

In some embodiments, the compound of Formula (II) is a compound of the Formula (IID):

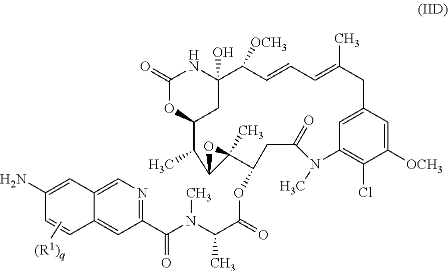

wherein R¹ and q are as defined herein.

In some embodiments, the compound of Formula (II) is a compound of the Formula (IIE):

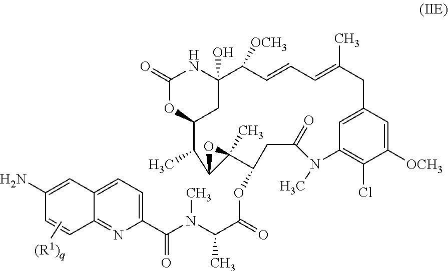

wherein R¹ and q are as defined herein.

In some embodiments, the compound of Formula (II) is a compound of the Formula (IIF):

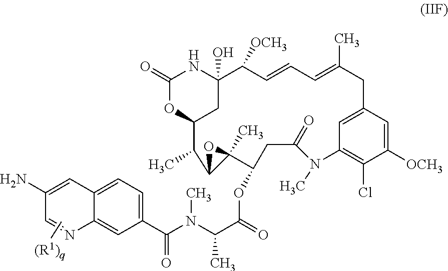

wherein R¹ and q are as defined herein.

In some embodiments, the compound of Formula (II) is a compound of the Formula (IIG):

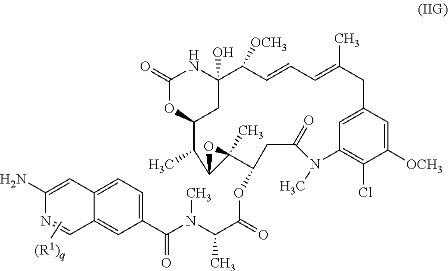

wherein R¹ and q are as defined herein.

In some embodiments, R¹ is, independently, alkyl or halo. In some embodiments, R¹ is, independently, C₁₋₆ alkyl, C₁₋₆ haloalkyl, or halo. In some embodiments, R¹ is, independently, C₁₋₆ haloalkyl or halo. In some embodiments, R¹ is, independently, halo. In some embodiments, R¹ is, independently, fluoro, chloro, bromo, iodo, or trifluoromethyl. In some embodiments, n, m, p, or q is 0, 1 or 2. In some embodiments, n, m, p, or q is 0 or 1. In some embodiments, n, m, p, or q is 0.

In some embodiments, R¹ is, independently, alkyl, alkoxy, heteroalkyl, halo, haloalkyl, or haloalkoxy. In some embodiments, R¹ is, independently, C₁₋₆ alkyl, C₁₋₆ alkoxy, C₁₋₆ haloalkyl, C₁₋₆ haloalkoxy, or halo. In some embodiments, R¹ is, independently, C₁₋₆ alkyl or C₁₋₆ alkoxy. In some embodiments, R¹ is, independently, alkoxy. In some embodiments, R¹ is, independently, methoxy, ethoxy, propoxy. In some embodiments, n, m, p, or q is 0, 1 or 2. In some embodiments, n, m, p, or q is 0 or 1. In some embodiments, n, m, p, or q is 0.In some embodiments, the compound of Formula (II) is a compound of Formula (IIA):

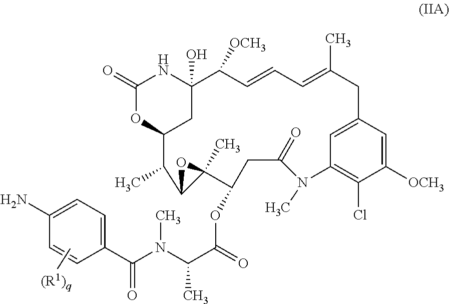

wherein:
   R¹ is, independently at each occurrence, halo or trifluoromethyl; and
   n is 0, 1, or 2.

In some embodiments, the compound of Formula (II) is a compound of Formula (IIB):

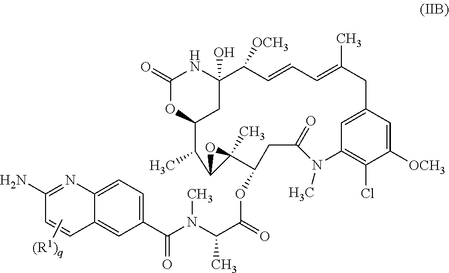

wherein:
   R¹ is, independently at each occurrence, halo or trifluoromethyl; and
   q is 0, 1, or 2.

In some embodiments, the compound of Formula (II) is:

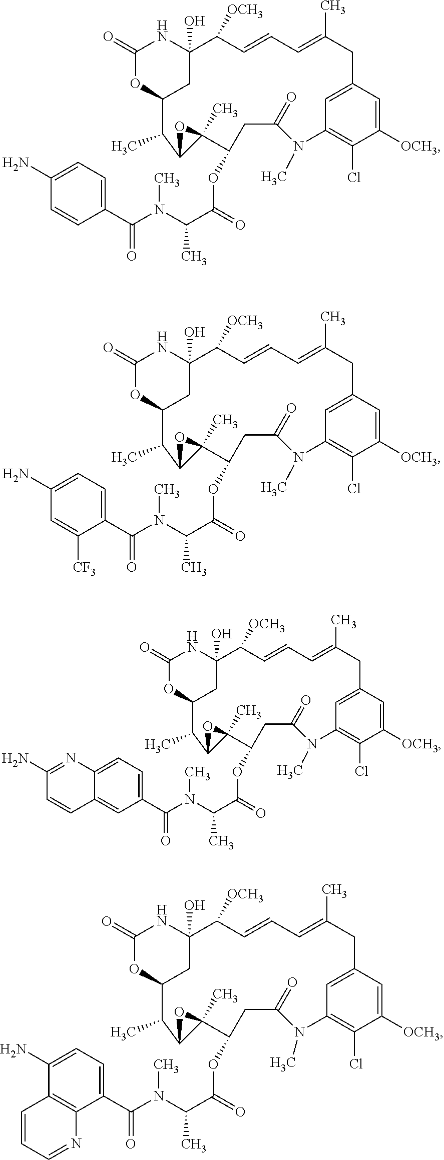

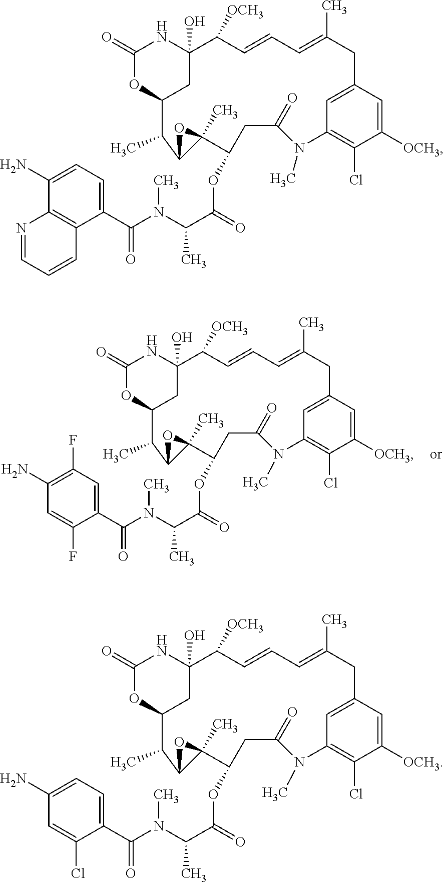 [SMILES: COc1cc2cc(c1Cl)N(C)C(=O)C[C@H](OC(=O)[C@H](C)N(C)C(=O)c1ccc(N)c3ncccc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cc(F)c(N)cc1F)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)cc1Cl)[C@]1(C)O[C@H]1[C@H](C)[C@@H]1C[C@@](O)(NC(=O)O1)[C@H](OC)/C=C/C=C(\C)C2]

In some embodiments, the compound of Formula (II) is a compound selected from

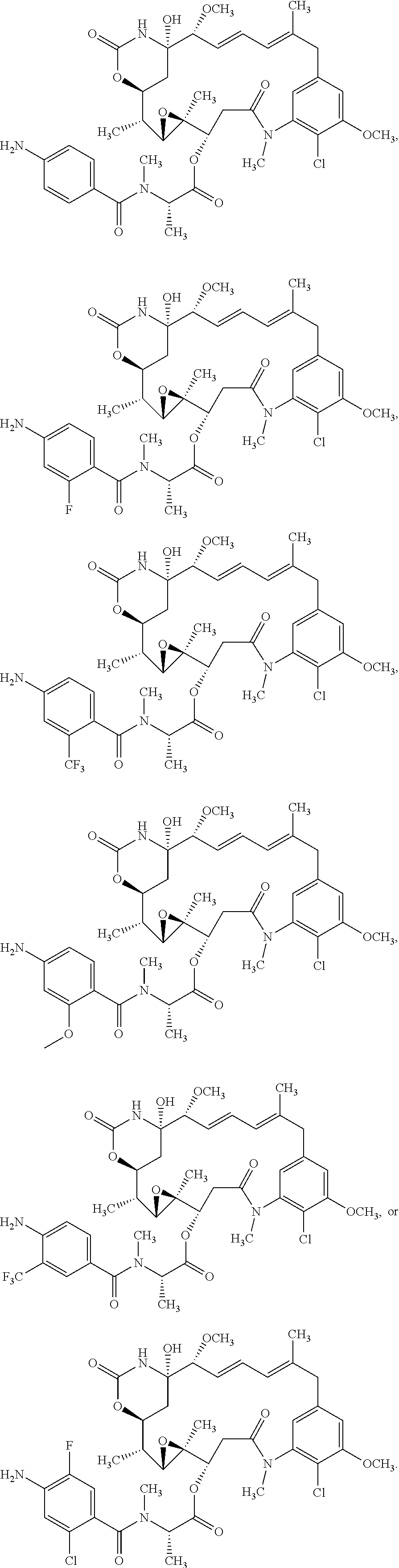

In some embodiments, the compound of Formula (II) is:

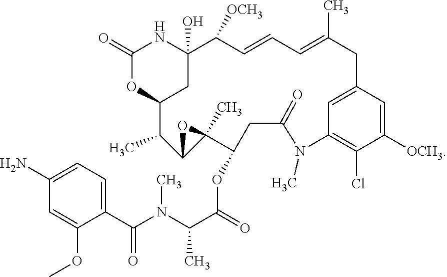 [SMILES: COc1cc(N)ccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

In certain embodiments, these compounds represent the payload portion of the conjugates described herein and are released, e.g., by enzyme proteolysis, following internalization of the conjugate into a cell. The methods provided herein include methods of treating a proliferative disease, e.g., cancer, comprising administering to a patient a therapeutically effective amount of a conjugate, e.g., antibody-drug conjugate that releases a compound of Formula (II) following internalization of said conjugate into a cell in said patient.

In some embodiments, these compounds represent the metabolic product of the conjugates described herein, e.g., enzyme proteolysis product.

In some embodiments, A is a divalent radical of benzene, of pyridine, of naphthalene, or of quinolone, which are optionally substituted.

In some embodiments, A is arylene.

In some embodiments, A is:

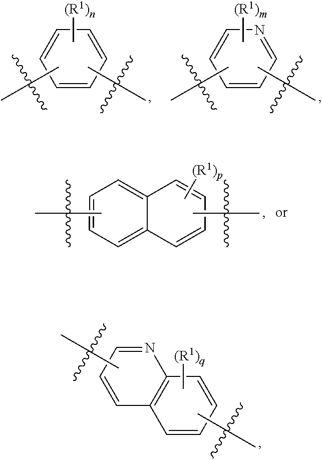

wherein:
   R¹ is, independently at each occurrence, alkyl, alkenyl, alkynyl, aryl, alkaryl, aralkyl, halo, haloalkoxy, heteroaryl, heterocycloalkyl, hydroxyl, cyano, nitro,

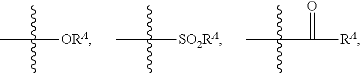

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, the compound of Formula (II) is a compound of the Formula (IIA):

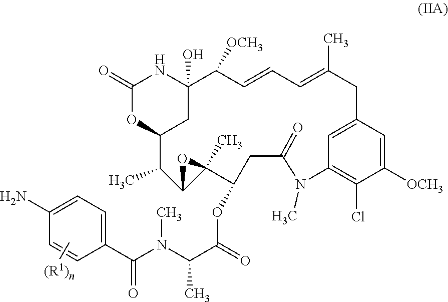

wherein R¹ is, independently at each occurrence, methoxy, halo or trifluoromethyl; and
n is 0, 1, or 2.

In some embodiments, the compound of Formula (II) is a compound of the Formula (IIB):

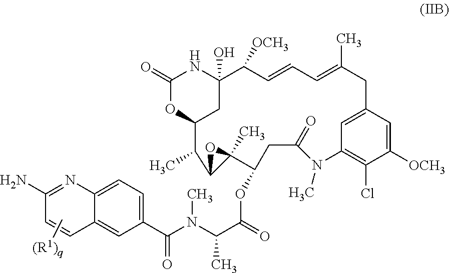

wherein R¹ is, independently at each occurrence, methoxy, halo or trifluoromethyl; and q is 0, 1, or 2.

In some embodiments, the compound of Formula (II) is a compound of the Formula (IIB2):

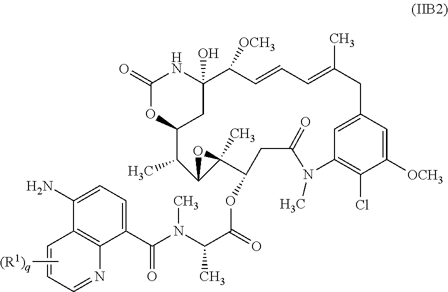

wherein R¹ is, independently at each occurrence, methoxy, halo or trifluoromethyl; and q is 0, 1, or 2.

In some embodiments, the compound of Formula (II) is a compound of the Formula (IIB3):

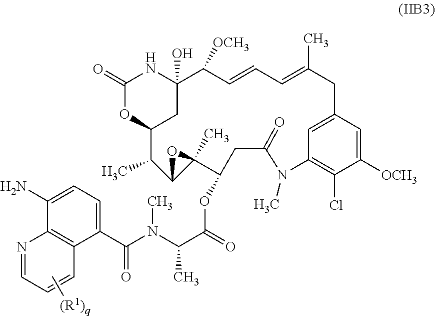

wherein R¹ is, independently at each occurrence, methoxy, halo or trifluoromethyl; and q is 0, 1, or 2. In some embodiments, R¹ is, independently, alkyl or halo. In some embodiments, R¹ is, independently, C₁₋₆ alkyl, C₁₋₆ haloalkyl, or halo. In some embodiments, R¹ is, independently, C₁₋₆ haloalkyl or halo. In some embodiments, R¹ is, independently, halo. In some embodiments, R¹ is, independently, fluoro, chloro, bromo, iodo, or trifluoromethyl. In some embodiments, n, m, p, or q is 0, 1 or 2. In some embodiments, n, m, p, or q is 0 or 1. In some embodiments, n, m, p, or q is 0.

In some embodiments, R¹ is, independently, alkyl, alkoxy, heteroalkyl, halo, haloalkyl, or haloalkoxy. In some embodiments, R¹ is, independently, C₁₋₆ alkyl, C₁₋₆ alkoxy, C₁₋₆ haloalkyl, C₁₋₆ haloalkoxy, or halo. In some embodiments, R¹ is, independently, C₁₋₆ alkyl or C₁₋₆ alkoxy. In some embodiments, R¹ is, independently, alkoxy. In some embodiments, R¹ is, independently, methoxy, ethoxy, propoxy. In some embodiments, n, m, p, or q is 0, 1 or 2. In some embodiments, n, m, p, or q is 0 or 1. In some embodiments, n, m, p, or q is 0.

In some embodiments, the compound of Formula (II) is:

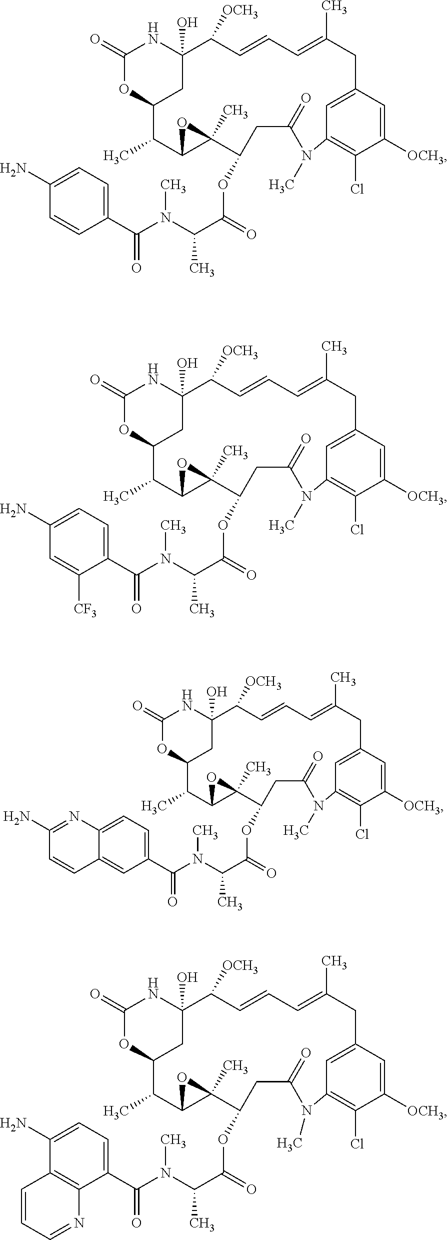

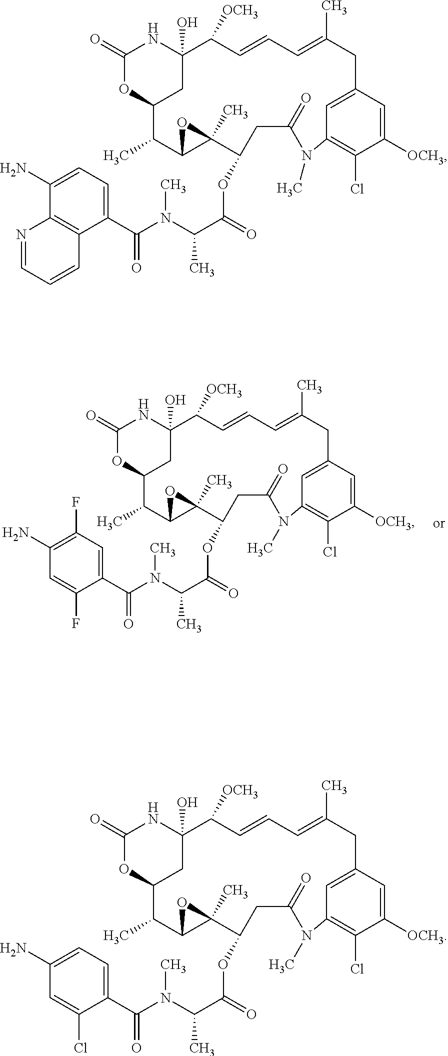 [SMILES: COc1cc2cc(c1Cl)N(C)C(=O)C[C@H](OC(=O)[C@H](C)N(C)C(=O)c1ccc(N)c3ncccc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cc(F)c(N)cc1F)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)cc1Cl)[C@]1(C)O[C@H]1[C@H](C)[C@@H]1C[C@@](O)(NC(=O)O1)[C@H](OC)/C=C/C=C(\C)C2]

In some embodiments, the compound of Formula (II) is a compound of the Formula (IIH):

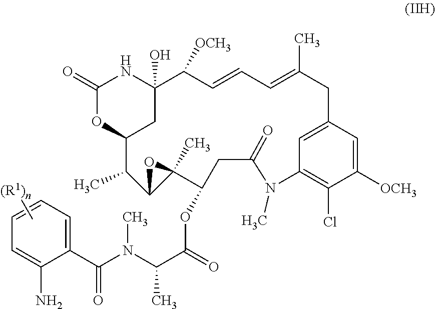

wherein R¹ and n are as defined herein.

In some embodiments, the compound of Formula (II) is a compound selected from

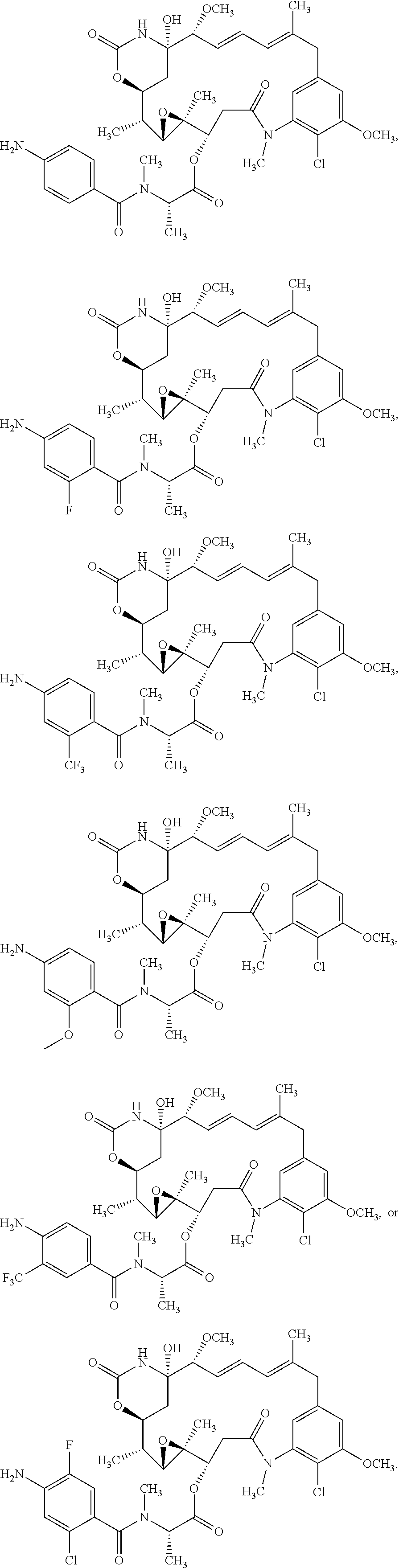

In some embodiments, the compound of Formula (II) is a compound selected fro

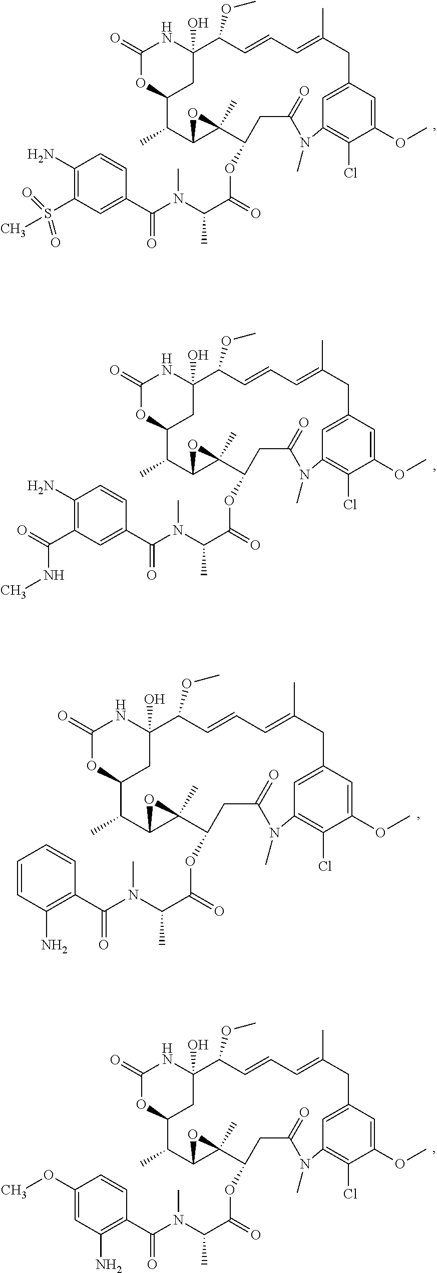

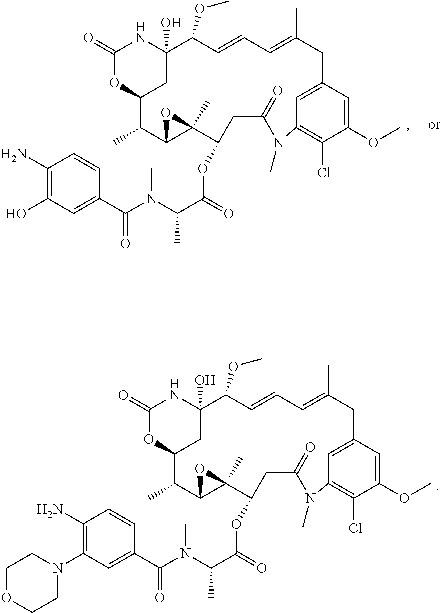 [SMILES: COc1cc2cc(c1Cl)N(C)C(=O)C[C@H](OC(=O)[C@H](C)N(C)C(=O)c1ccc(N)c(O)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(N)c(N3CCOCC3)c1)[C@]1(C)O[C@H]1[C@H](C)[C@@H]1C[C@@](O)(NC(=O)O1)[C@H](OC)/C=C/C=C(\C)C2]

### D. Preparation of Compounds

Compounds of Formula I can be synthesized by coupling compounds of Formula P1 with a binding agent, e.g., antibody under standard conjugation conditions (see, e.g, Doronina et al., Nature Biotechnology 2003, 21, 7, 778, which is incorporated herein by reference). When the binding agent is an antibody, the antibody can be coupled to a compound of Formula P1 via one or more cysteine or lysine residues of the antibody. Compounds of Formula P1 can be coupled to cysteine residues, for example, by subjecting the antibody to a reducing agent, e.g., dithiotheritol, to cleave the disulfide bonds of the antibody, purifying the reduced antibody, e.g., by gel filtration, and subsequently reacting the antibody with a compound of formula P1 containing a reactive moiety, e.g., a maleimido group. Suitable solvents include, but are not limited to water, DMA, DMF, and DMSO. Compounds of formula P1 containing a reactive moiety, e.g., activated ester or acid halide group, can be coupled to lysine residues. Suitable solvents include, but are not limited to water, DMA, DMF, and DMSO. The compounds of Formula I can be purified using known protein techniques, including, for example, size exclusion chromatography, dialysis, and ultrafiltration/diafiltration.

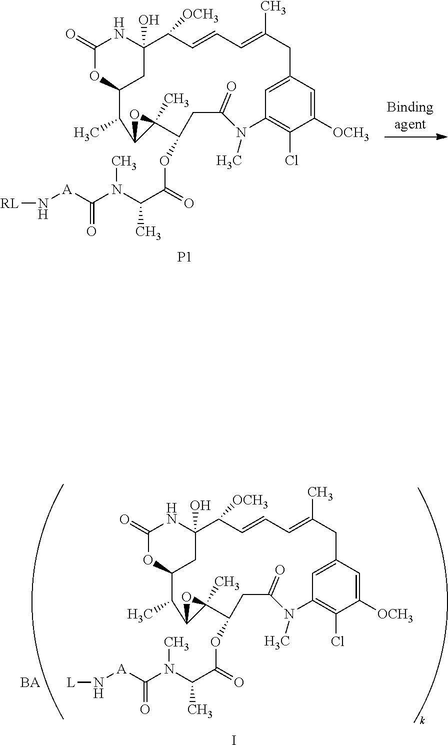

wherein RL is a reactive linker, A is arylene or heteroarylene, L is a linker, and BA is a binding agent.

In some embodiments, the compound of formula P1 includes A, wherein A is:

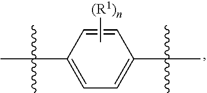

wherein n is 0 or 1; and R¹ is alkoxy, halo, or haloalkyl.

In some embodiments, the compound of formula P1 includes A, wherein A is:

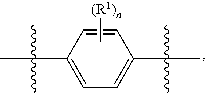

wherein n is 0 or 1; and R¹ is C₁₋₆ alkoxy, halo, or C₁₋₆ haloalkyl.

In some embodiments, the compound of formula P1 includes A, wherein A is:

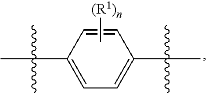

wherein n is 0 or 1; R¹ is C₁₋₆ alkoxy, halo, or C₁₋₆ haloalkyl; and RL is

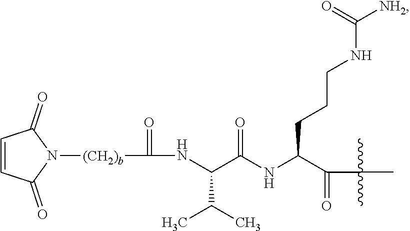

wherein b is an integer from 2 to 8 and

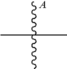

is a bond to the binding agent.

The reactive linker is a moiety comprising a portion in its structure that is capable of reacting with the binding agent (e.g., reacting with an antibody at its cysteine or lysine residues) to form the compound of Formula I. Following conjugation to the binding agent, the reactive linker becomes the linker (L) moiety of the compound of Formula I. Illustrative reactive linkers include, but are not limited to, those that comprise haloacetyl, isothiocyanate, or maleimide portions that are capable of reacting with the binding agent. Reactive portions also include moieties having the following structure:

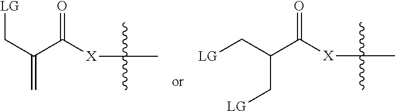

wherein X is —O— or —NH— and LG is a leaving group, e.g., Br.

In some embodiments, the reactive linker is:

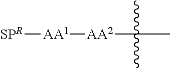

wherein:
   SP^{R} is a reactive spacer;
   AA¹ is an amino acid; and
   AA² is an amino acid.

The reactive spacer is a moiety that contains the above-described reactive linker portion that is capable of reacting with the binding agent and connects this portion to AA¹. Suitable spacers include, but are not limited to, those comprising alkylene or polyethylene glycol connecting the AA¹ to the portion capable of reacting with binding agent (e.g., haloacetyl, isothiocyanate, or maleimide).

In some embodiments, the reactive spacer comprises a non-cleavable moiety selected from

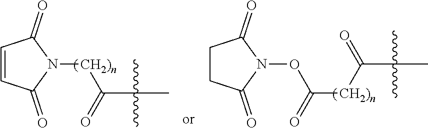

wherein

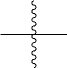

represents one or more bonds to the maytansinoid derivative; and wherein n is an integer from 4 to 10. In some examples, n is 4, 5, 6, 7, 8, 9, or 10. In some embodiments, the reactive spacer is

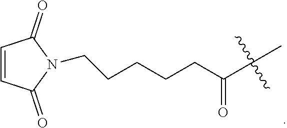 [SMILES: *C(=O)CCCCCN1C(=O)C=CC1=O]

In some embodiments, the reactive spacer is

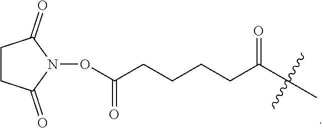 [SMILES: *C(=O)CCCCC(=O)ON1C(=O)CCC1=O]

In some embodiments, the reactive spacer is:

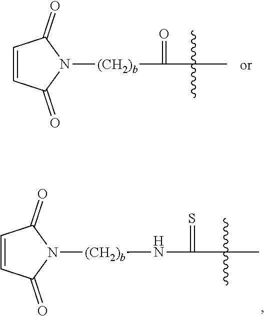

wherein b is an integer from 2 to 8.

In some embodiments, the reactive spacer is:

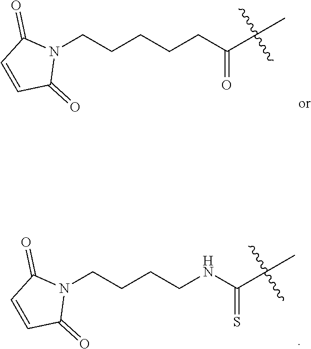 [SMILES: *C(=O)CCCCCN1C(=O)C=CC1=O ; *C(=S)NCCCCN1C(=O)C=CC1=O]

In some embodiments, the spacer is

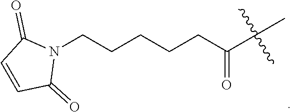 [SMILES: *C(=O)CCCCCN1C(=O)C=CC1=O]

In some embodiments, the spacer is

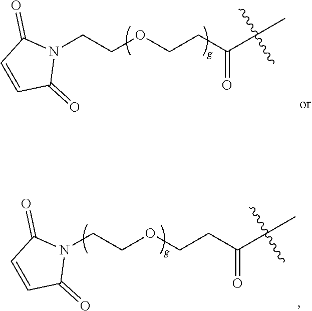

wherein g is an integer from 1 to 24.

In some embodiments, the reactive spacer is:

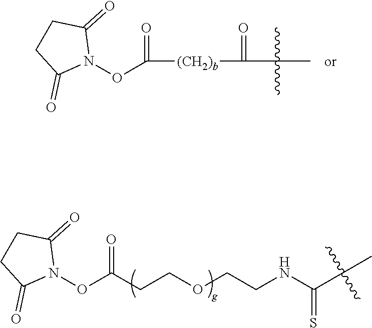

wherein b is an integer from 2 to 8 and g is an integer from 2 to 20.

In some embodiments, the reactive spacer is:

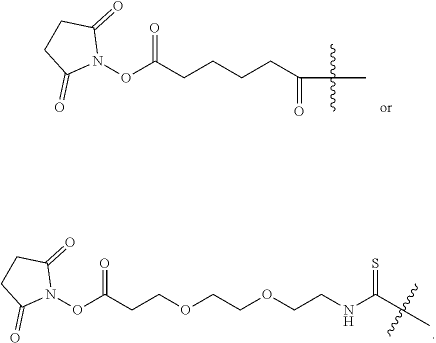 [SMILES: *C(=O)CCCCC(=O)ON1C(=O)CCC1=O ; *C(=S)NCCOCCOCCC(=O)ON1C(=O)CCC1=O]

In some embodiments, AA¹-AA² is: valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some embodiments, AA¹-AA² is: valine-citrulline or citrulline-valine. In some embodiments, AA¹-AA² is: valine-citrulline.

In some embodiments, the reactive linker is:

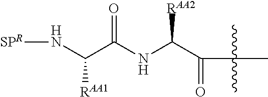

wherein:
   SP^{R} is a reactive spacer;
   R^{AA1} is an amino acid side chain; and
   R^{AA2} is an amino acid side chain.

In some embodiments, the reactive linker is:

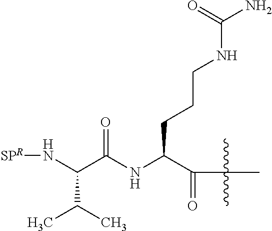

wherein:
   SP is a reactive spacer.

In some embodiments, the reactive linker is:

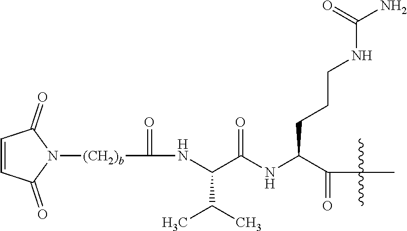

wherein b is an integer from 2 to 8.

In some embodiments, the reactive linker is:

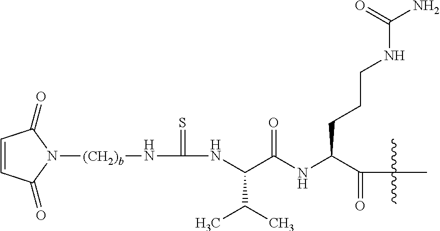

wherein b is an integer from 2 to 8.

In some embodiments, the reactive linker is:

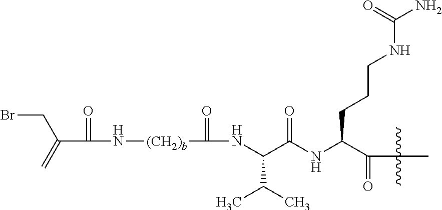

wherein b is an integer from 2 to 8.

In some embodiments, the reactive linker is:

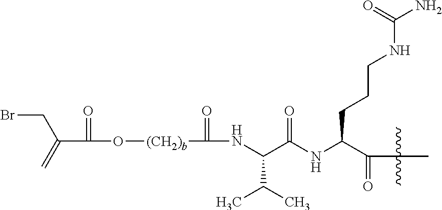

wherein b is an integer from 2 to 8.

In some embodiments, the reactive linker is:

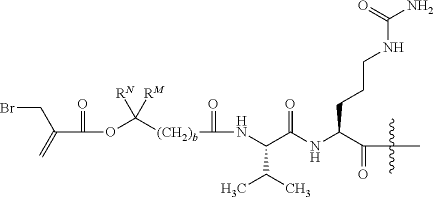

wherein b is an integer from 2 to 8, R^{N} is a hydrogen atom or alkyl, and R^{M} is alkyl.

In some embodiments, the reactive linker is:

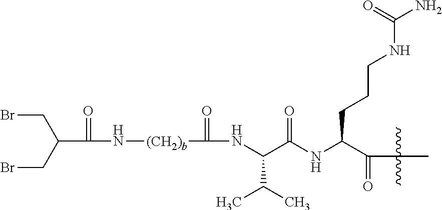

wherein b is an integer form 2 to 8.

In some embodiments, the reactive linker is:

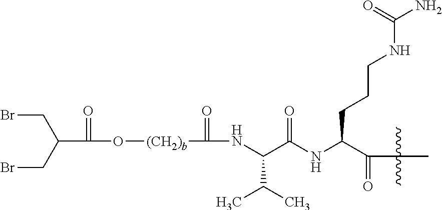

wherein b is an integer from 2 to 8.

In some embodiments, the reactive linker is:

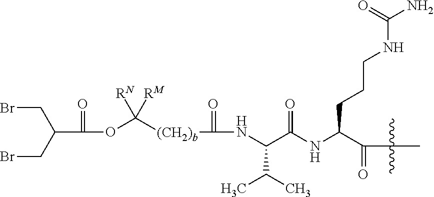

wherein b is an integer from 2 to 8; R^{N} is a hydrogen atom or alkyl; and R^{M} is alkyl.

In some embodiments, the reactive linker is:

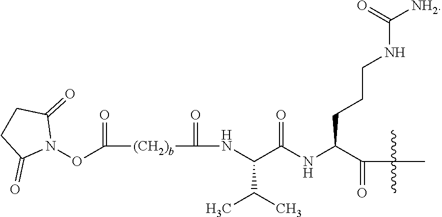

wherein b is an integer from 2 to 8.

In some embodiments, the reactive linker is:

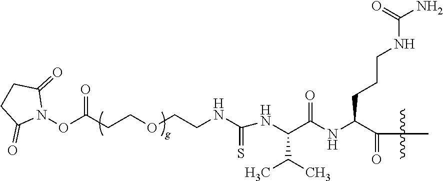

wherein g is an integer from 2 to 8.

In some embodiments, the reactive linker is:

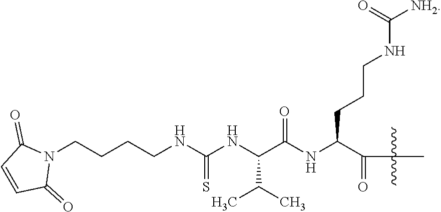 [SMILES: *C(=O)[C@H](CCCNC(N)=O)NC(=O)[C@@H](NC(=S)NCCCCN1C(=O)C=CC1=O)C(C)C]

In some embodiments, the reactive linker is:

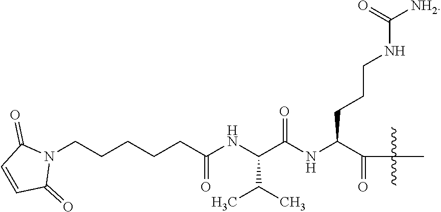 [SMILES: *C(=O)[C@H](CCCNC(N)=O)NC(=O)[C@@H](NC(=O)CCCCCN1C(=O)C=CC1=O)C(C)C]

In some embodiments, the reactive linker is:

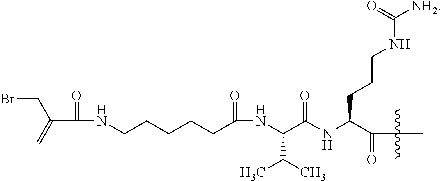 [SMILES: *C(=O)[C@H](CCCNC(N)=O)NC(=O)[C@@H](NC(=O)CCCCCNC(=O)C(=C)CBr)C(C)C]

In some embodiments, the reactive linker is:

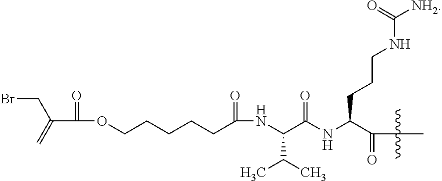 [SMILES: *C(=O)[C@H](CCCNC(N)=O)NC(=O)[C@@H](NC(=O)CCCCCOC(=O)C(=C)CBr)C(C)C]

In some embodiments, the reactive linker is:

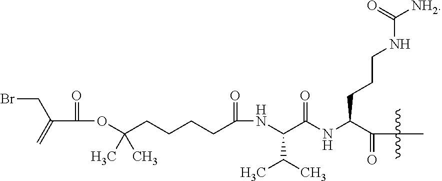 [SMILES: *C(=O)[C@H](CCCNC(N)=O)NC(=O)[C@@H](NC(=O)CCCCC(C)(C)OC(=O)C(=C)CBr)C(C)C]

In some embodiments, the reactive linker is:

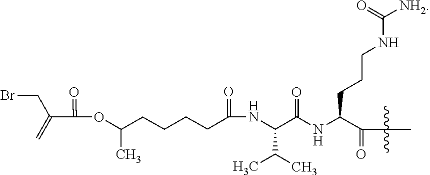 [SMILES: *C(=O)[C@H](CCCNC(N)=O)NC(=O)[C@@H](NC(=O)CCCCC(C)OC(=O)C(=C)CBr)C(C)C]

In some embodiments, the reactive linker is:

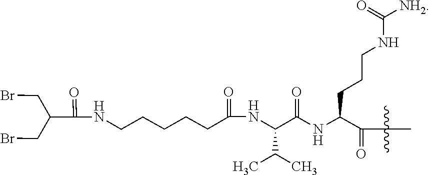 [SMILES: *C(=O)[C@H](CCCNC(N)=O)NC(=O)[C@@H](NC(=O)CCCCCNC(=O)C(CBr)CBr)C(C)C]

In some embodiments, the reactive linker is:

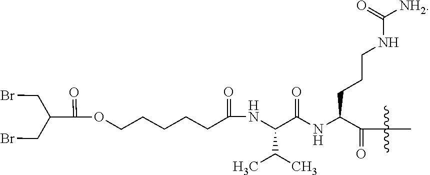 [SMILES: *C(=O)[C@H](CCCNC(N)=O)NC(=O)[C@@H](NC(=O)CCCCCOC(=O)C(CBr)CBr)C(C)C]

In some embodiments, the reactive linker is:

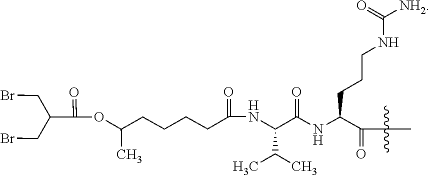 [SMILES: *C(=O)[C@H](CCCNC(N)=O)NC(=O)[C@@H](NC(=O)CCCCC(C)OC(=O)C(CBr)CBr)C(C)C]

In some embodiments, the reactive linker is:

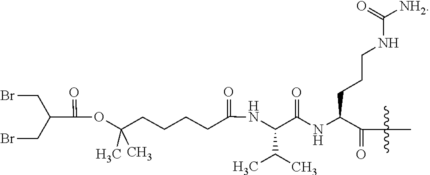 [SMILES: *C(=O)[C@H](CCCNC(N)=O)NC(=O)[C@@H](NC(=O)CCCCC(C)(C)OC(=O)C(CBr)CBr)C(C)C]

In some embodiments, the reactive linker is:

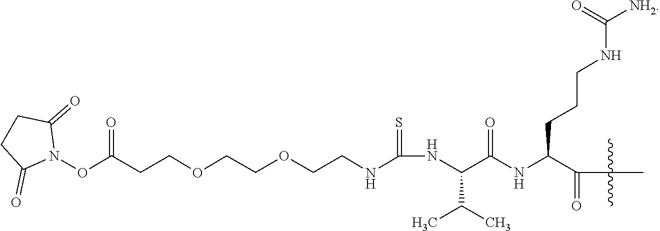 [SMILES: *C(=O)[C@H](CCCNC(N)=O)NC(=O)[C@@H](NC(=S)NCCOCCOCCC(=O)ON1C(=O)CCC1=O)C(C)C]

In some embodiments, the reactive linker is:

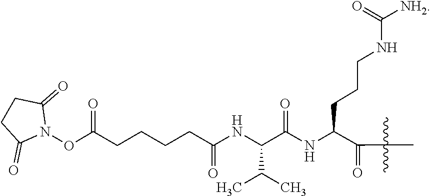 [SMILES: *C(=O)[C@H](CCCNC(N)=O)NC(=O)[C@@H](NC(=O)CCCCC(=O)ON1C(=O)CCC1=O)C(C)C]

In some embodiments, the compound of Formula P1 is a compound of Formula P1A:

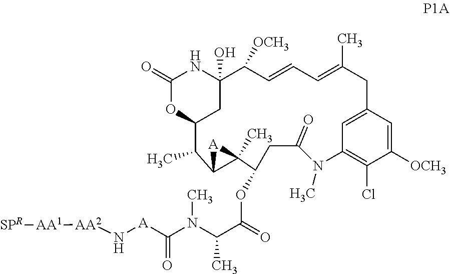

wherein:
   A is:

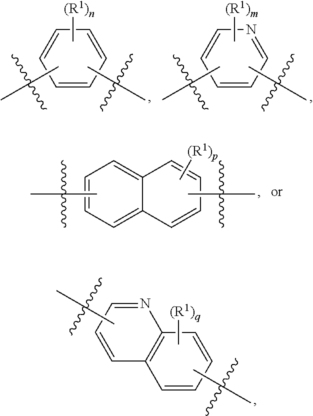

wherein:
   R¹ is alkyl, alkenyl, alkynyl, alkoxy, aryl, alkaryl, aralkyl, halo, haloalkoxy, haloalkyl, heteroaryl, heterocycloalkyl, hydroxyl, cyano, nitro,

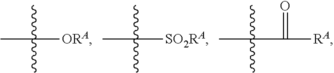

or azido, wherein R^{A} is alkyl or heteroalkyl;
n is an integer from 0 to 4;
m is an integer from 0 to 3;
p is an integer from 0 to 6; and
q is an integer from 0 to 5;
SP^{R} is a reactive spacer;
AA¹ is an amino acid; and
AA² is an amino acid.

In some embodiments, the compound of Formula P1A is a compound which includes A wherein A is:

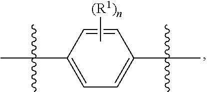

wherein n is 0 or 1; and R¹ is alkoxy, halo, or haloalkyl. In some examples, R¹ is methylsulfonyl, N-methylformamide, hydroxyl, or morpholinyl.

In some embodiments, the compound of Formula P1A is a compound which includes A wherein A is:

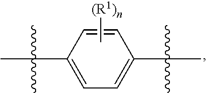

wherein n is 0 or 1; and R¹ is C₁₋₆ alkoxy, halo, or C₁₋₆ haloalkyl.

In some embodiments, the compound of Formula P1A is a compound which includes A wherein A is:

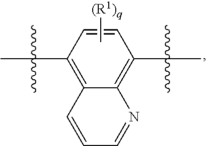

wherein q is an integer from 0 to 5; and R¹ is C₁₋₆ alkoxy, halo, or C₁₋₆ haloalkyl.

In some embodiments, the compound of Formula P1A is a compound which includes A wherein A is:

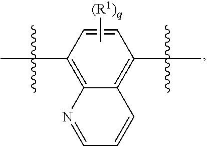

wherein q is an integer from 0 to 5; and R¹ is C₁₋₆ alkoxy, halo, or C₁₋₆ haloalkyl.

In some embodiments, the compound of Formula P1A is a compound which includes A wherein A is:

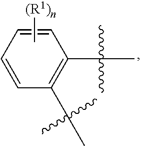

wherein n is 0 or 1; and R¹ is alkoxy, halo, or haloalkyl. In some examples, R¹ is methylsulfonyl, N-methylformamide, hydroxyl, or morpholinyl.

In some embodiments, the compound of Formula P1 is a compound of Formula P1B:

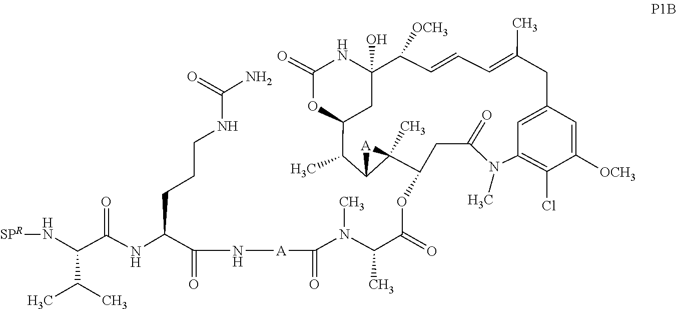

wherein
   A is:

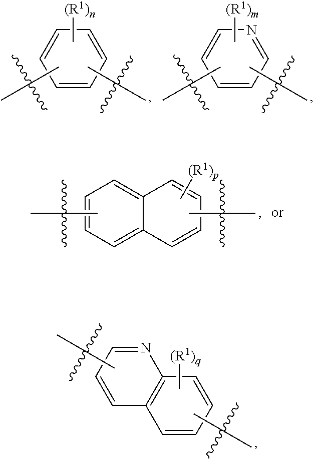

wherein:
   R¹ is alkyl, alkenyl, alkynyl, alkoxy, aryl, alkaryl, aralkyl, halo, haloalkoxy, haloalkyl, heteroaryl, heterocycloalkyl, hydroxyl, cyano, nitro,

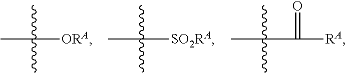

or azido, wherein R^{A} is alkyl or heteroalkyl;
n is an integer from 0 to 4;
m is an integer from 0 to 3;
p is an integer from 0 to 6; and
q is an integer from 0 to 5; and
SP^{R} is a reactive spacer.

In some embodiments, the compound of Formula P1 is a compound of Formula P1C:

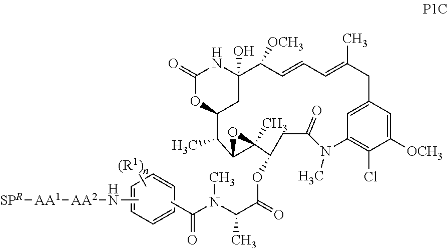

wherein:
   SP^{R} is a reactive spacer;
   AA¹ is an amino acid;
   AA² is an amino acid;
   R¹ is, independently at each occurrence, alkyl, alkoxy, halo, haloalkoxy, haloalkyl, or trifluoromethyl, and
   n is 0, 1, or 2.

In some embodiments, the compound of Formula P1 is a compound of Formula P1D:

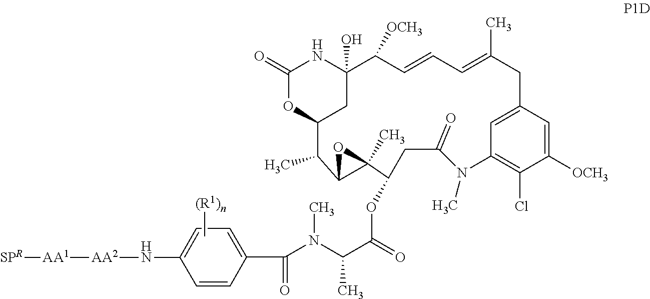

wherein:
   SP^{R} is a reactive spacer;
   AA¹ is an amino acid;
   AA² is an amino acid;
   R¹ is, independently at each occurrence, alkyl, alkoxy, halo, haloalkoxy, haloalkyl, or trifluoromethyl, and
   n is 0, 1, or 2. In some embodiments, n is 0. In some embodiments, n is 1. In some embodiments, n is 2.

In some embodiments, the compound of Formula P1 is a compound of Formula P1D, wherein R¹ is alkoxy, halo, or haloalkyl. In some embodiments, R¹ is C₁₋₆ alkoxy, halo, or C₁₋₆ haloalkyl. In some embodiments, n is 0. In some embodiments, n is 1. In some embodiments, n is 2.

In some embodiments, the compound of Formula P1 is a compound of Formula P1D, wherein R¹ is C₁₋₆ alkoxy, halo, or C₁₋₆ haloalkyl; and SP^{R}-AA¹-AA² is

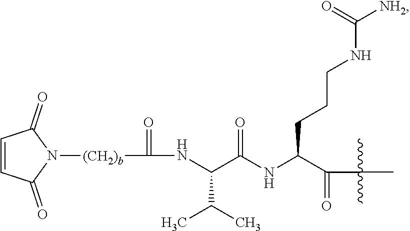

wherein b is an integer from 2 to 8 and

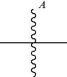

is a bond to the binding agent. In some embodiments, n is 0. In some embodiments, n is 1. In some embodiments, n is 2. In some embodiments b is 2. In some embodiments b is 3. In some embodiments b is 4. In some embodiments b is 5. In some embodiments b is 6. In some embodiments b is 7. In some embodiments b is 8.

In some embodiments, the compound of Formula P1 is a compound of Formula P1E:

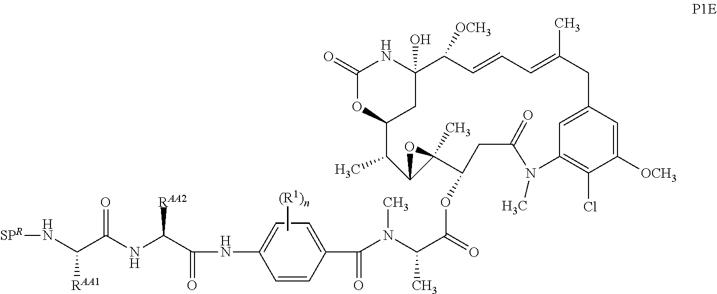

wherein:
   SP^{R} is a reactive spacer;
   R^{AA1} is an amino acid side chain;
   R^{AA2} is an amino acid side chain;
   R¹ is, independently at each occurrence, alkyl, alkoxy, halo, haloalkoxy, haloalkyl, or trifluoromethyl, and
   n is 0, 1, or 2.

In some embodiments, the compound of Formula P1 is a compound of Formula P1F:

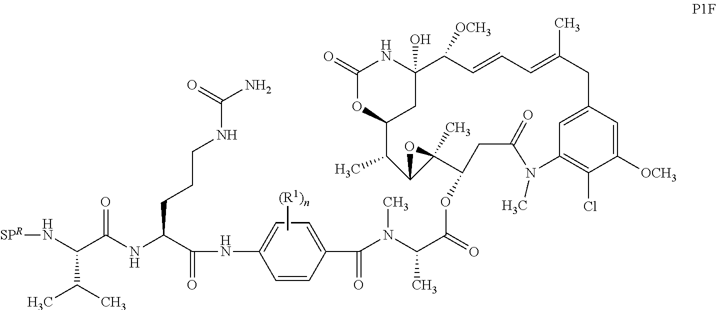

wherein:
   SP^{R} is a reactive spacer;
   R¹ is, independently at each occurrence, alkyl, alkoxy, halo, haloalkoxy, haloalkyl, or trifluoromethyl, and
   n is 0, 1, or 2.

In some embodiments, the compound of Formula P1 is a compound of Formula P1G:

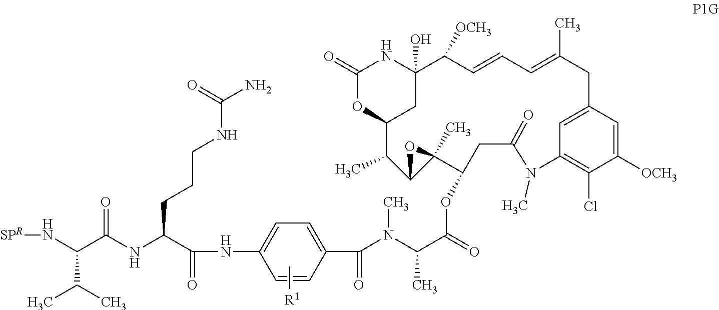

wherein:
   SP^{R} is a reactive spacer; and
   R¹ is a hydrogen atom, alkyl, alkoxy, halo, haloalkoxy, haloalkyl, or trifluoromethyl.

In some embodiments, the compound of Formula P1 is a compound of Formula P1H:

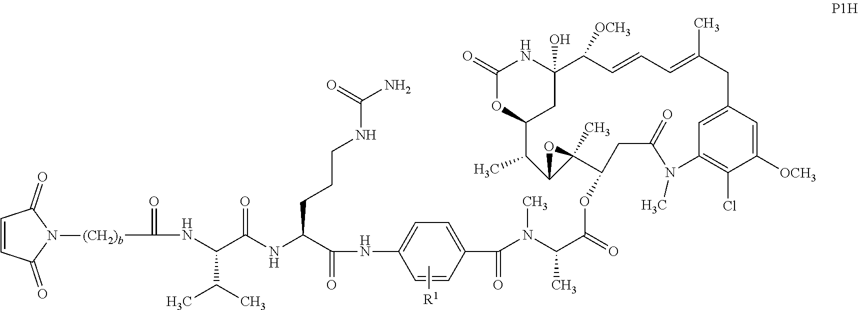

wherein:
   R¹ is hydrogen atom, alkyl, alkoxy, halo, haloalkoxy, haloalkyl, or trifluoromethyl; and
   b is an integer from 2 to 8.

In some embodiments, the compound of Formula P1 is a compound of Formula P1I:

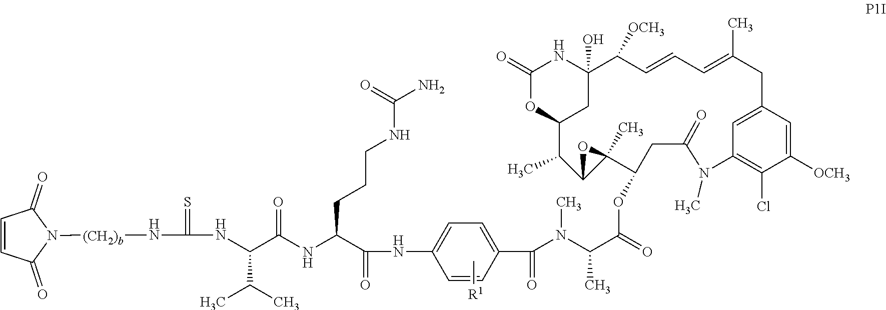

wherein:
   R¹ is a hydrogen atom, alkyl, alkoxy, halo, haloalkoxy, haloalkyl, or trifluoromethyl; and
   b is an integer from 2 to 8.

In some embodiments, the compound of Formula P1 is a compound of Formula P1J:

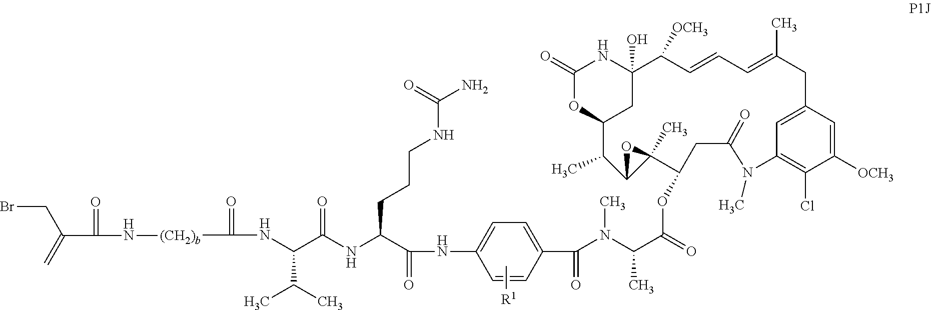

wherein:
   R¹ is a hydrogen atom, alkyl, alkoxy, halo, haloalkoxy, haloalkyl, or trifluoromethyl; and
   b is an integer from 2 to 8.

In some embodiments, the compound of Formula P1 is a compound of Formula P1K:

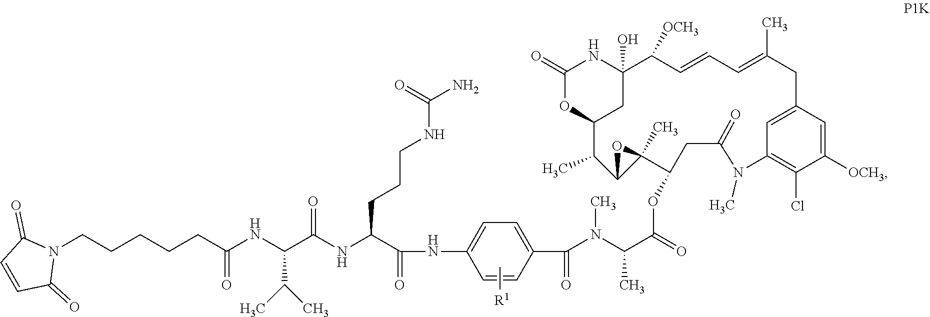

wherein R¹ is a hydrogen atom, alkyl, alkoxy, halo, haloalkoxy, haloalkyl, or trifluoromethyl.

In some embodiments, the compound of Formula P1 is a compound of Formula P1L:

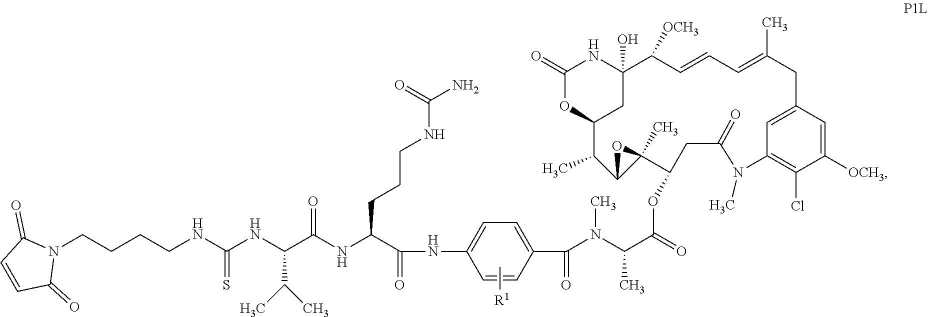

wherein R¹ is a hydrogen atom, alkyl, alkoxy, halo, haloalkoxy, haloalkyl, or trifluoromethyl.

In some embodiments, the compound of Formula P1 is a compound of Formula P1M:

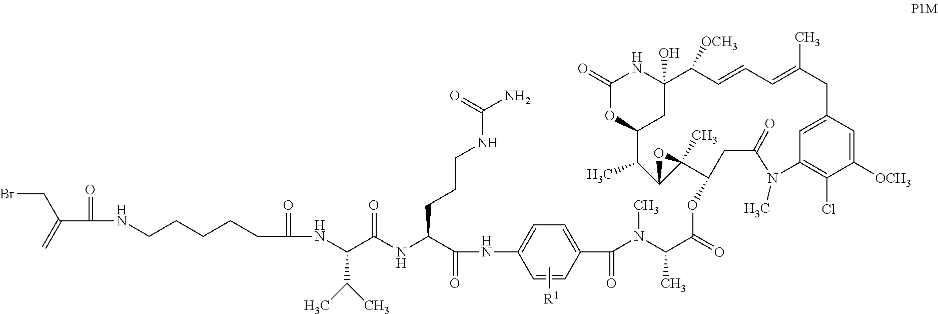

wherein R¹ is a hydrogen atom, alkyl, alkoxy, halo, haloalkoxy, haloalkyl, or trifluoromethyl.

In some embodiments, the compound of Formula P1 is a compound of Formula P1N:

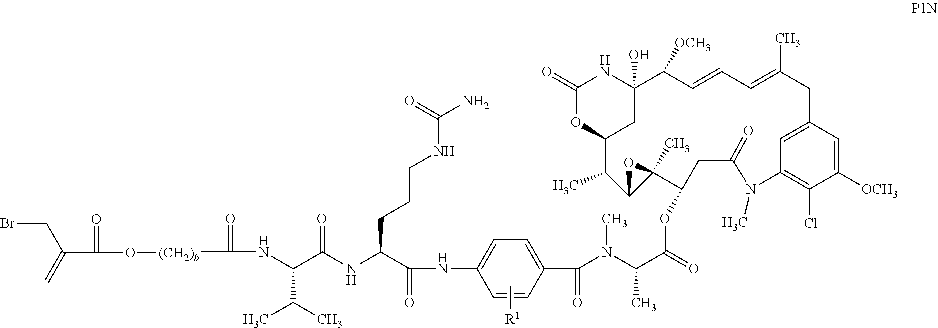

wherein R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1O:

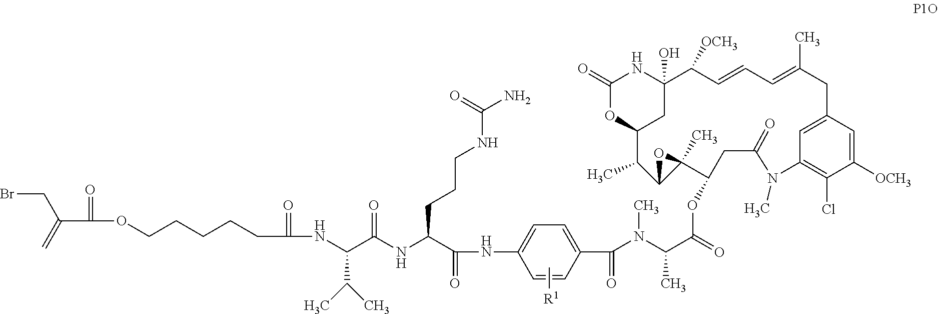

wherein R¹ is a hydrogen atom, alkyl, alkoxy, halo, haloalkoxy, haloalkyl, or trifluoromethyl.

In some embodiments, the compound of Formula P1 is a compound of Formula P1P:

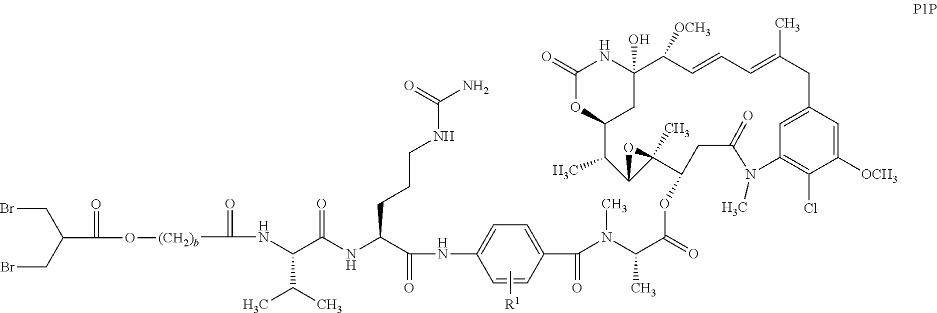

wherein R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1Q:

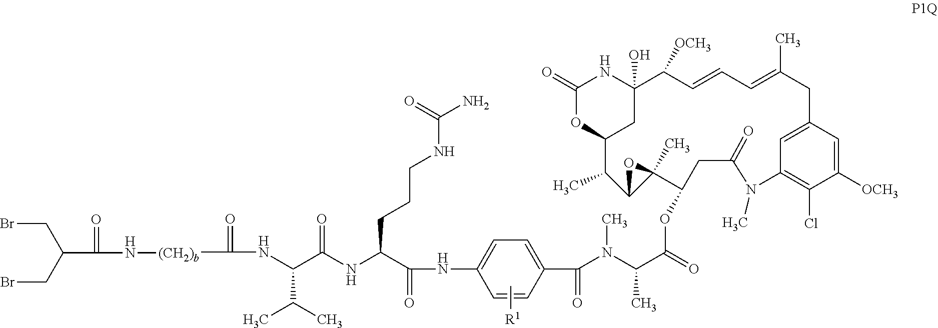

wherein R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1R:

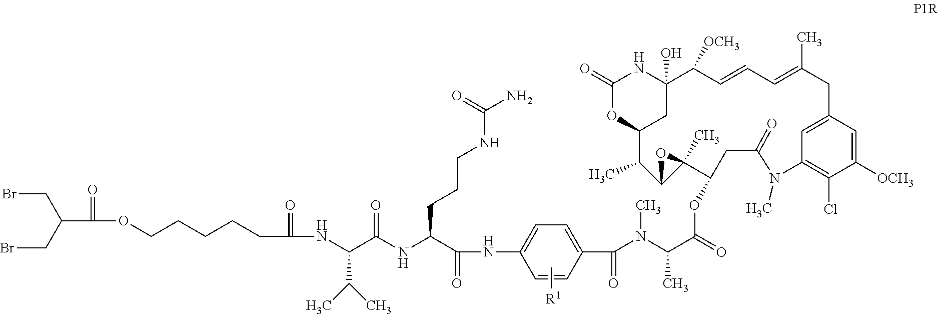

wherein R¹ is a hydrogen atom, alkyl, alkoxy, halo, haloalkoxy, haloalkyl, or trifluoromethyl.

In some embodiments, the compound of Formula P1 is a compound of Formula P1S:

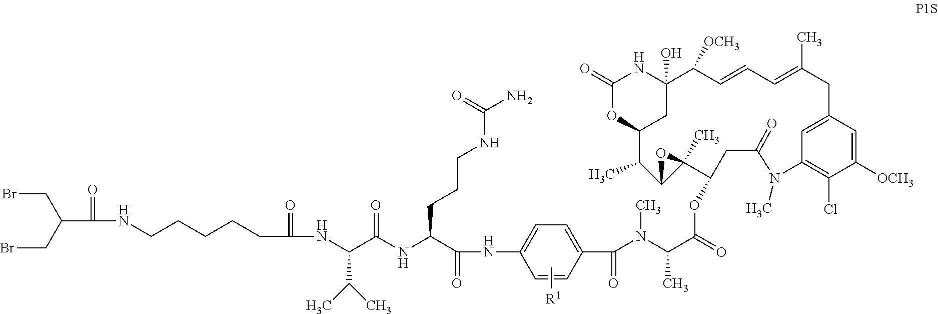

wherein R¹ is a hydrogen atom, alkyl, alkoxy, halo, haloalkoxy, haloalkyl, or trifluoromethyl.

In some embodiments, the compound of Formula P1 is a compound of Formula P1T:

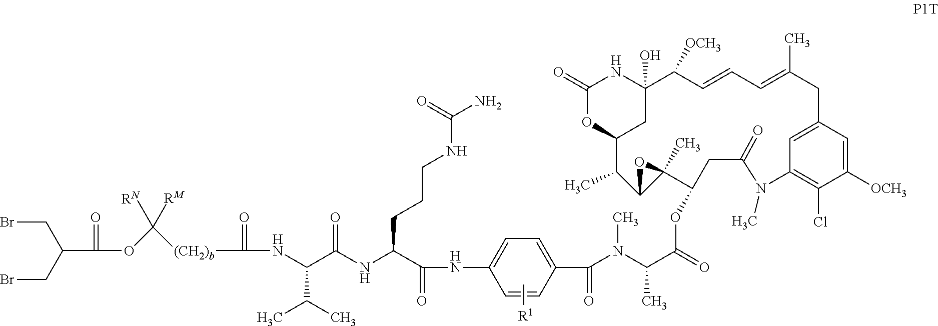

wherein R^{N} is a hydrogen atom or alkyl, R^{M} is alkyl, R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1U:

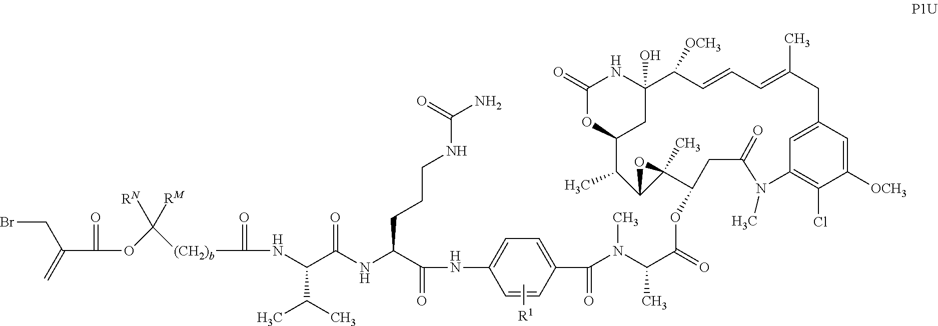

wherein R^{N} is a hydrogen atom or alkyl, R^{M} is alkyl, R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1V:

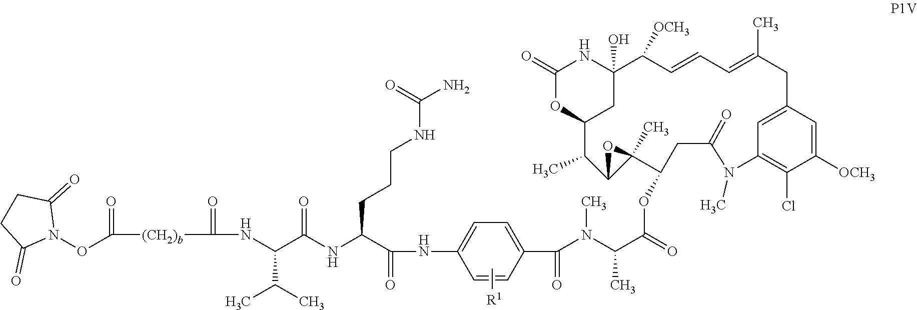

wherein R¹ is a hydrogen atom, alkyl, alkoxy, halo, haloalkoxy, haloalkyl, or trifluoromethyl, and b is an integer from 2 to 8.

In some embodiments, the compound of Formula P1 is a compound of Formula P1W:

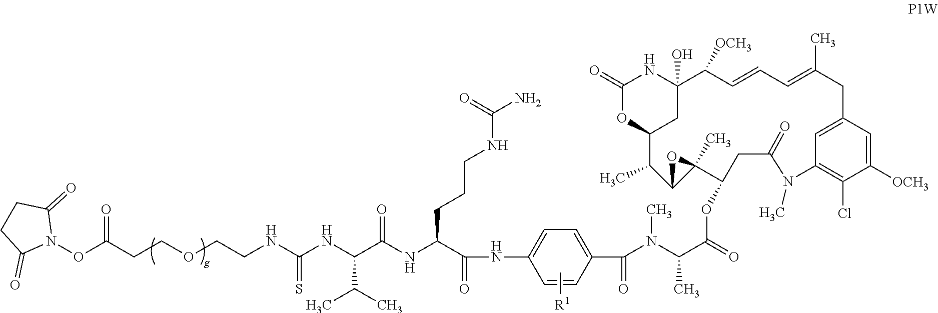

wherein R¹ is a hydrogen atom, alkyl, alkoxy, halo, haloalkoxy, haloalkyl, or trifluoromethyl, g is an integer from 2 to 20; and b is an integer from 2 to 8.

In some embodiments, the compound of Formula P1 is a compound of Formula P1X:

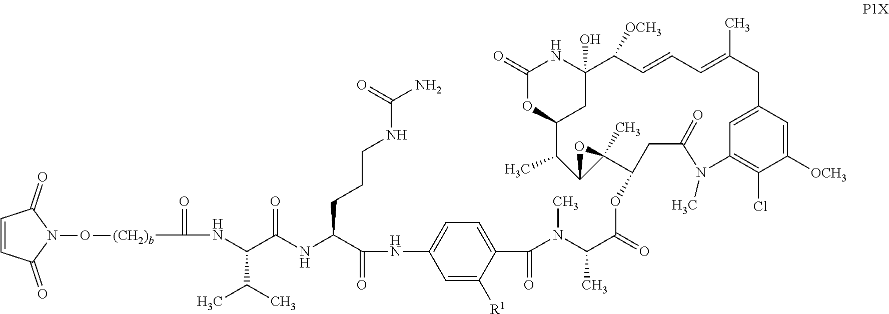

wherein:
R¹ is alkyl, alkenyl, alkynyl, alkoxy, heteroalkyl, halo, haloalkyl, or haloalkoxy; and b is an integer from 2 to 8. In some embodiments, R¹ is methyl, ethyl, methoxy, or ethoxy. In some of these embodiments, R¹ is methoxy. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is fluoro. In some embodiments, R¹ is hydrogen.

In some embodiments, the compound of Formula P1 is a compound of Formula P1Y:

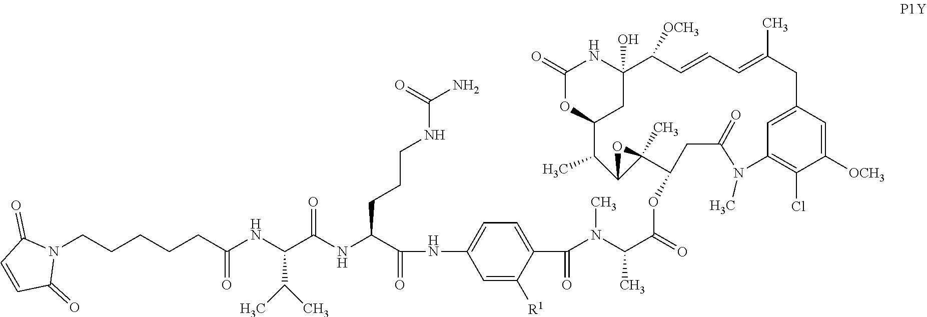

wherein R¹ is alkyl, alkenyl, alkynyl, alkoxy, heteroalkyl, halo, haloalkyl, haloalkoxy. In some embodiments, R¹ is methyl, ethyl, methoxy, or ethoxy. In some of these embodiments, R¹ is methoxy. In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is fluoro. In some embodiments, R¹ is hydrogen.

In some embodiments, the compound of Formula P1 is a compound of Formula P1Z:

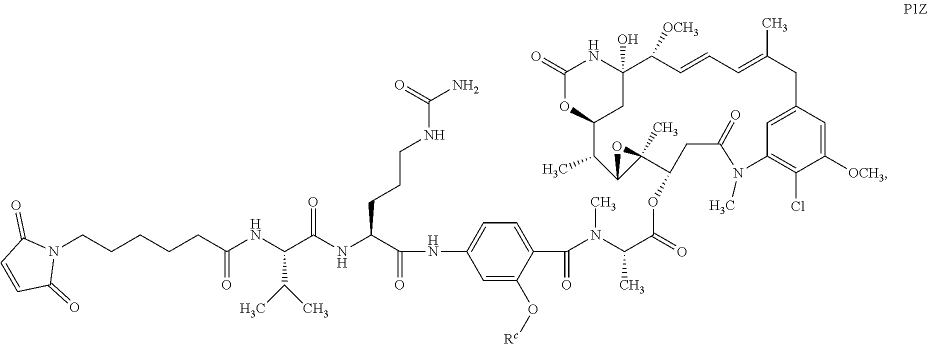

wherein R^{c} is selected from alkyl or haloalkyl and wherein the alkyl or haloalkyl is linear, branched, or cyclic.

In some embodiments, the compound of Formula P1 is a compound having one of the following structures:

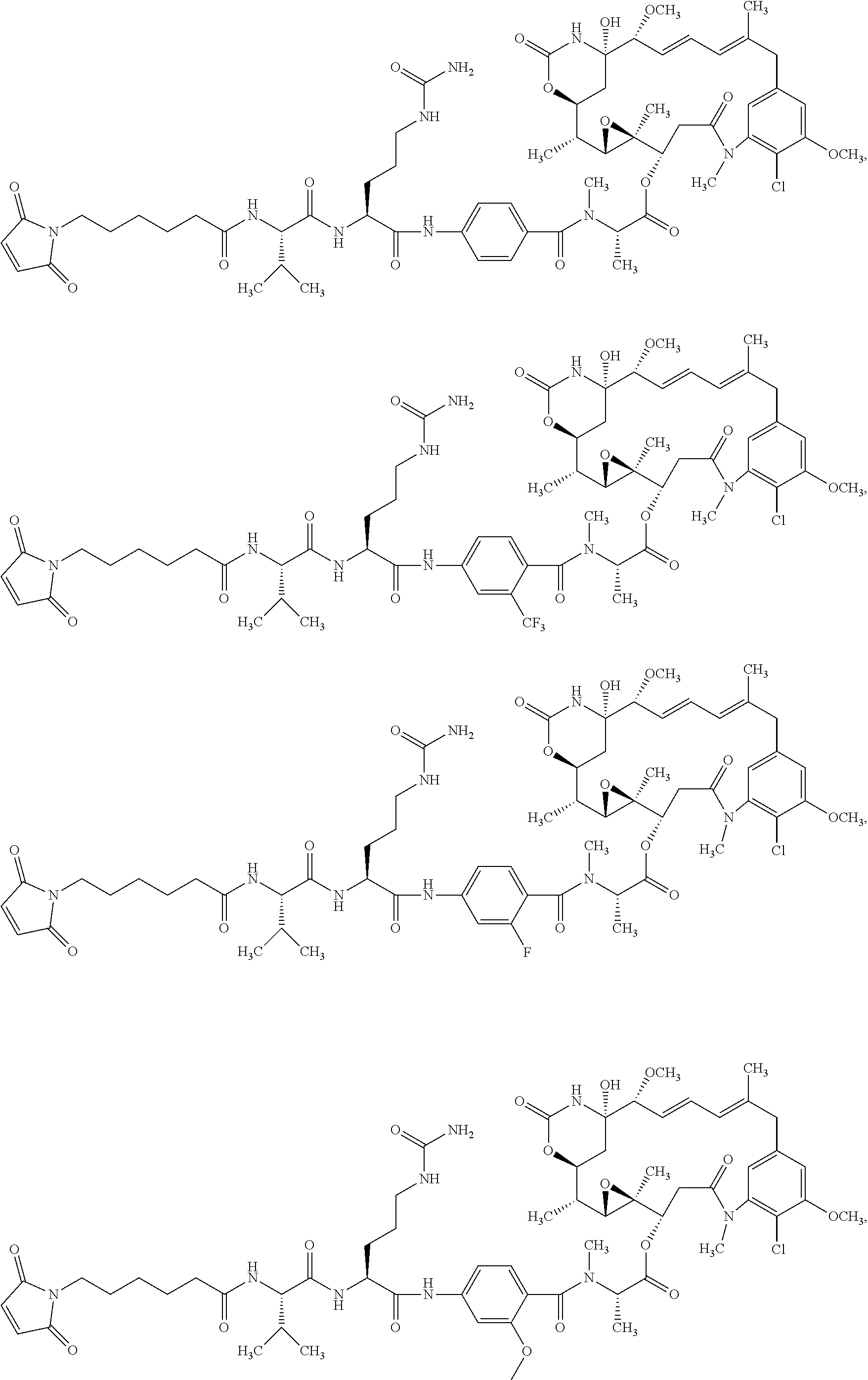

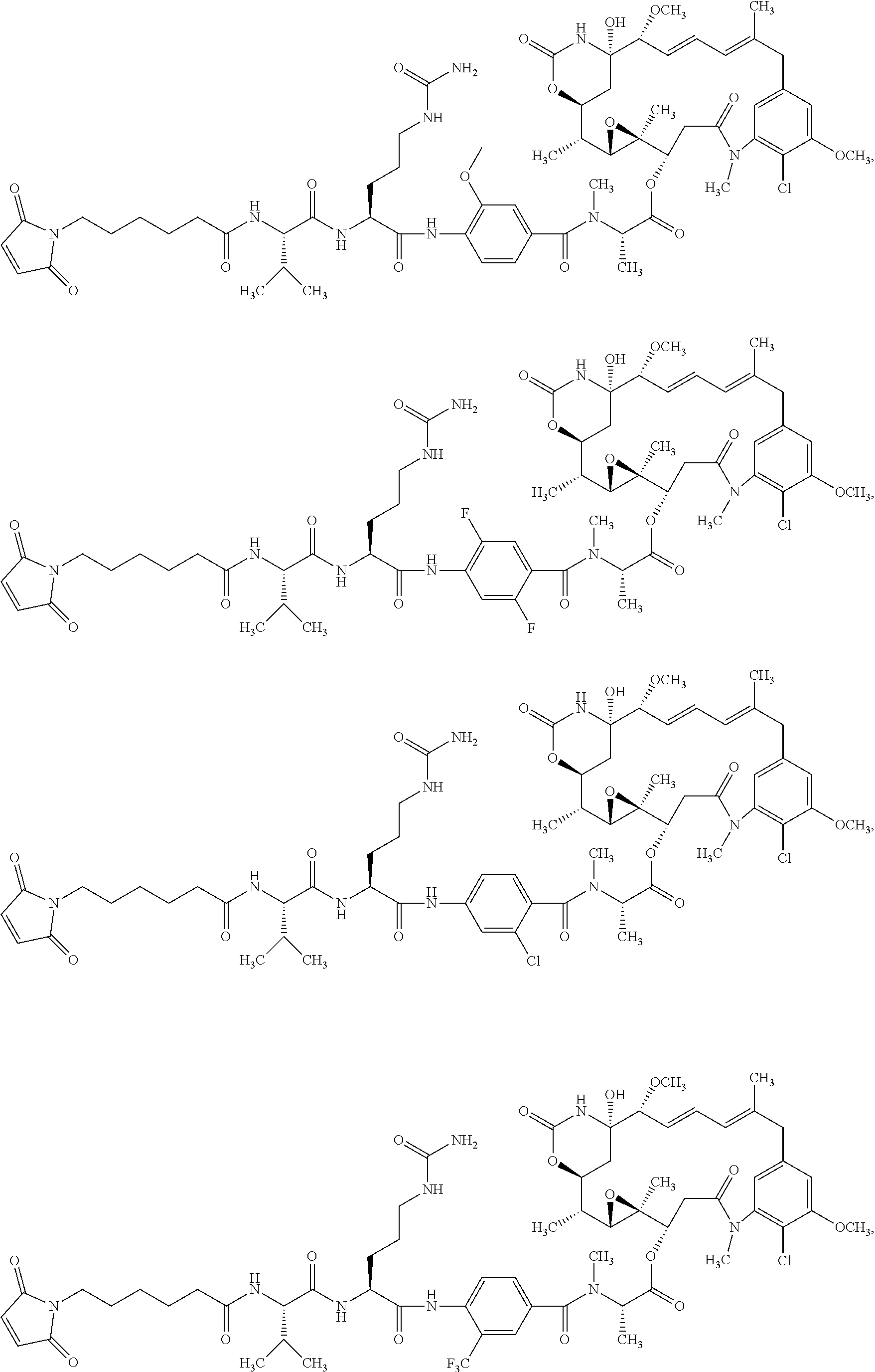

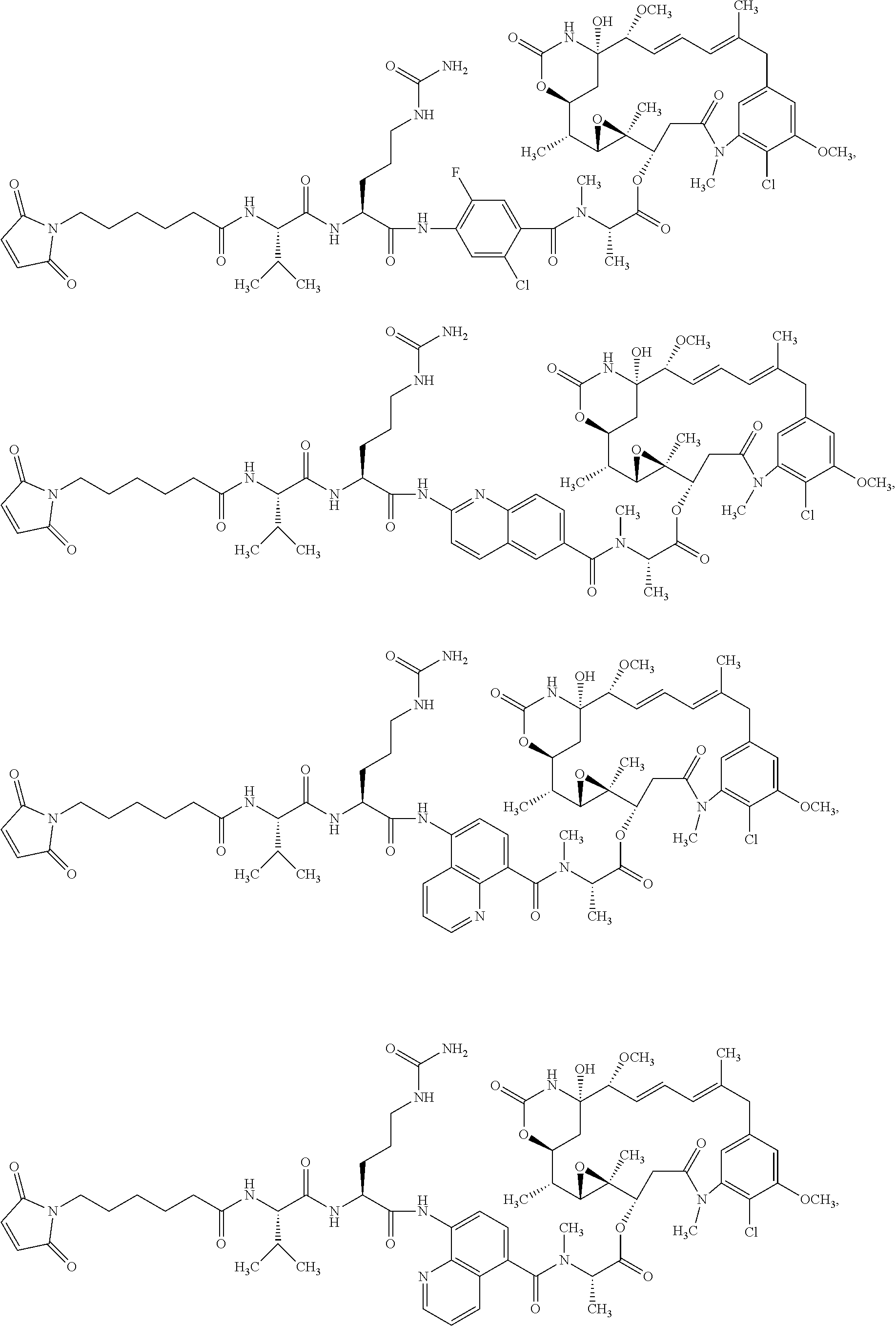

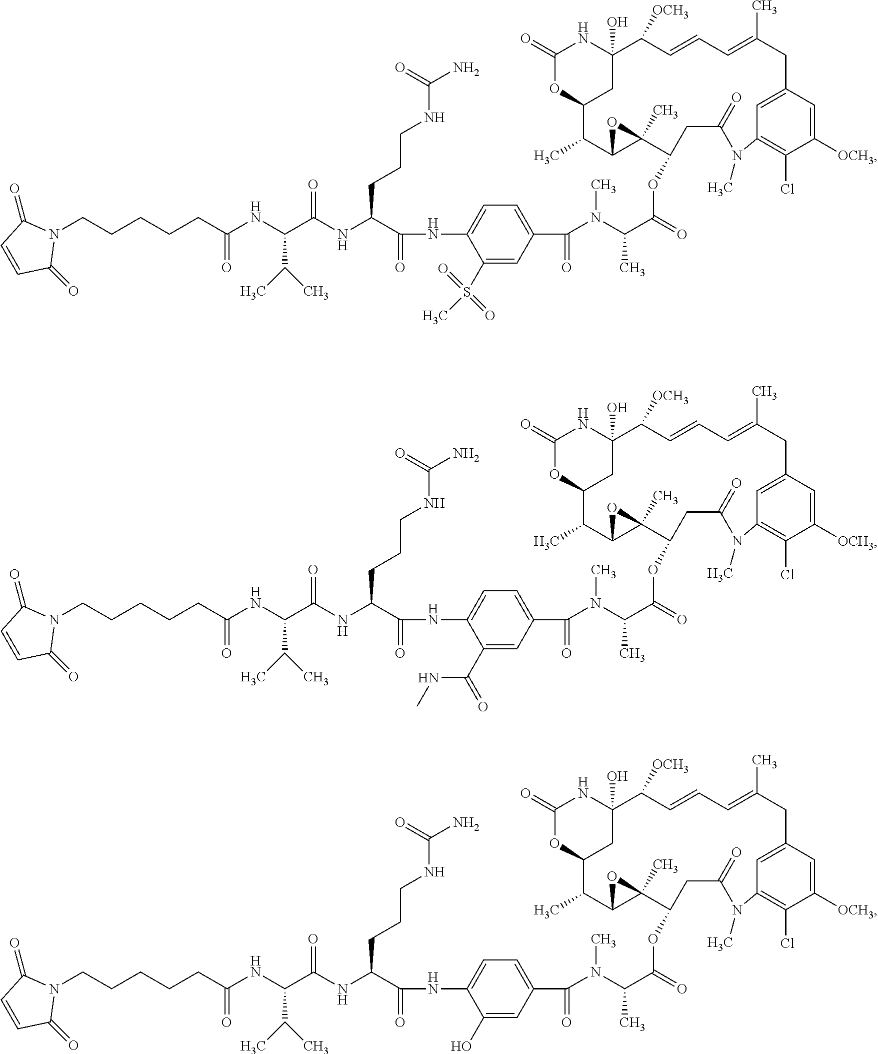

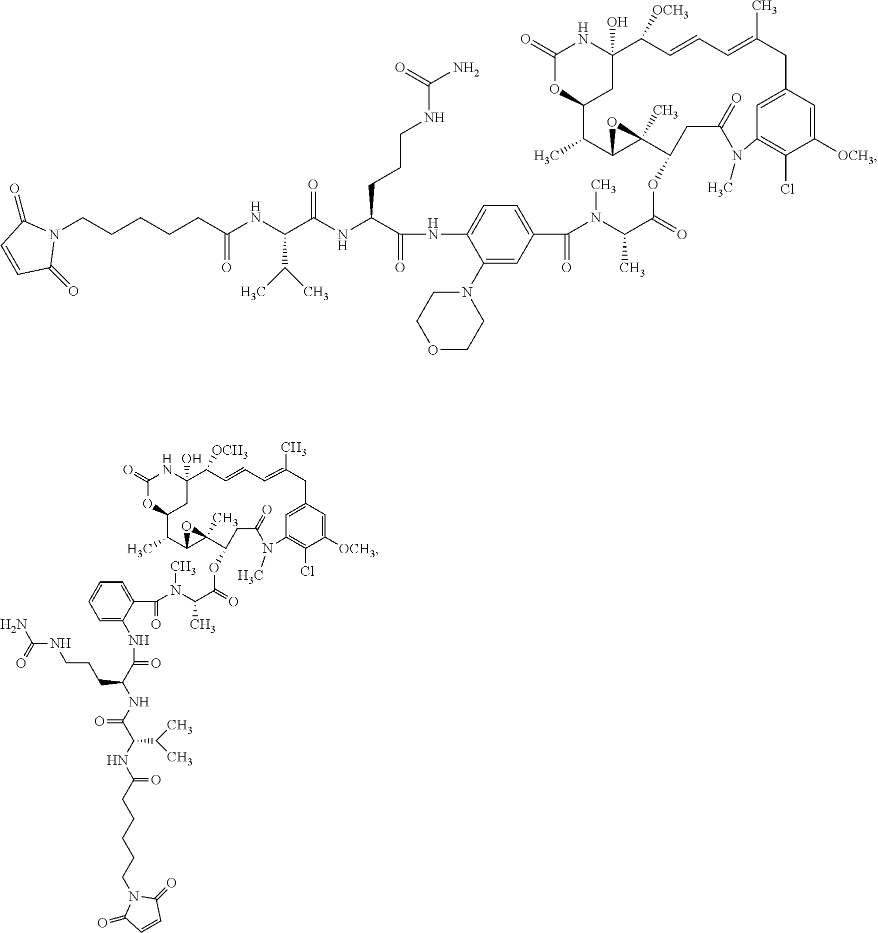 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCN3C(=O)C=CC3=O)C(C)C)c(N3CCOCC3)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=O)CCCCCN1C(=O)C=CC1=O)C(C)C)[C@]1(C)O[C@H]1[C@H](C)[C@@H]1C[C@@](O)(NC(=O)O1)[C@H](OC)/C=C/C=C(\C)C2]

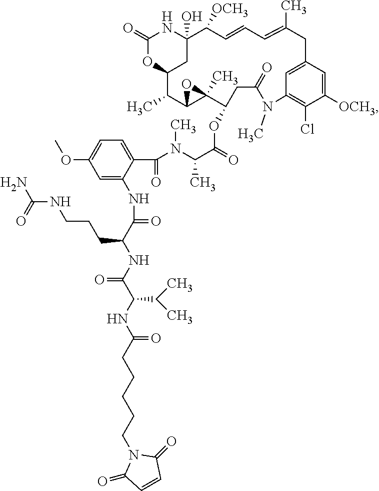 [SMILES: COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=O)CCCCCN2C(=O)C=CC2=O)C(C)C)c1]

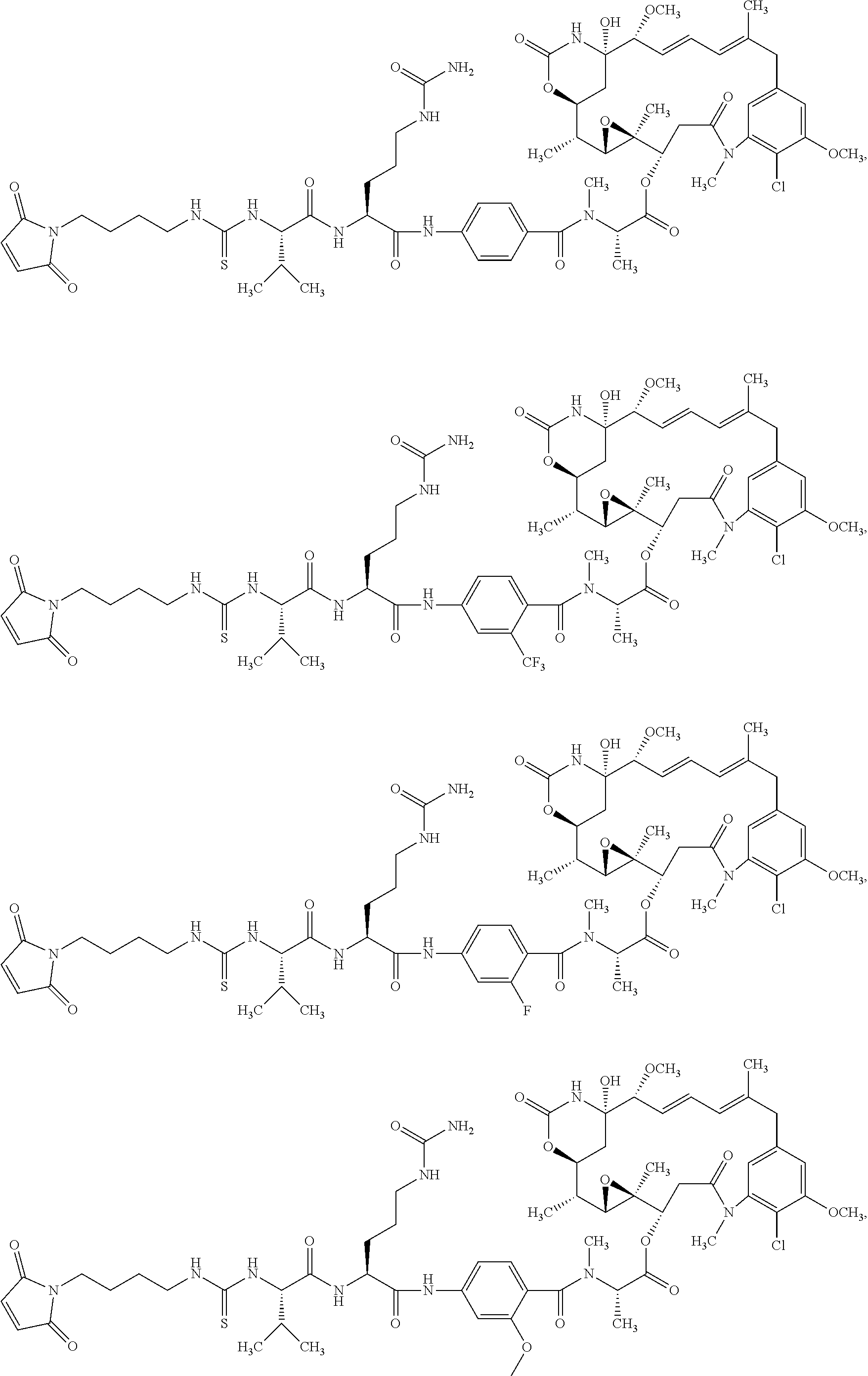

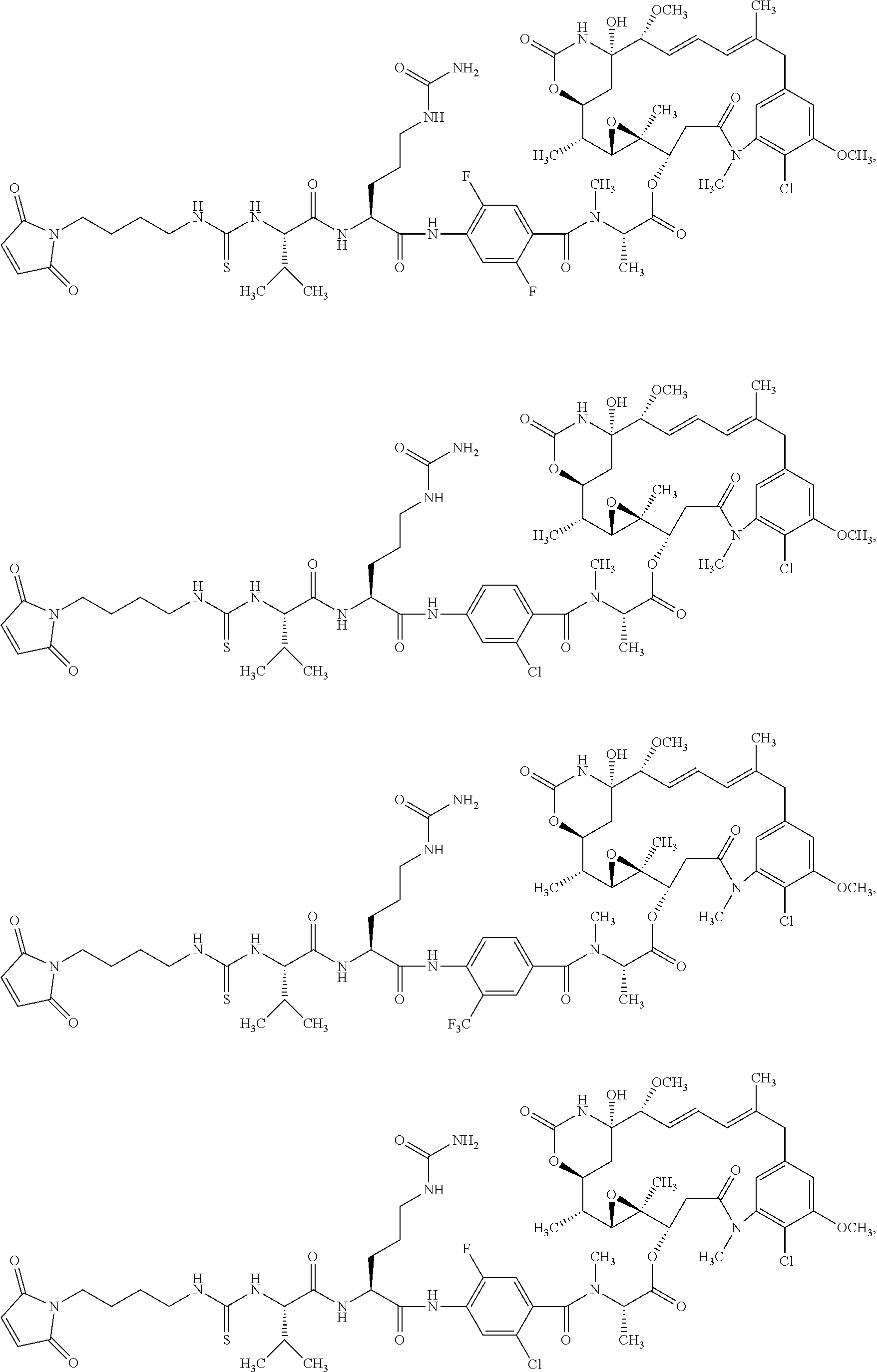

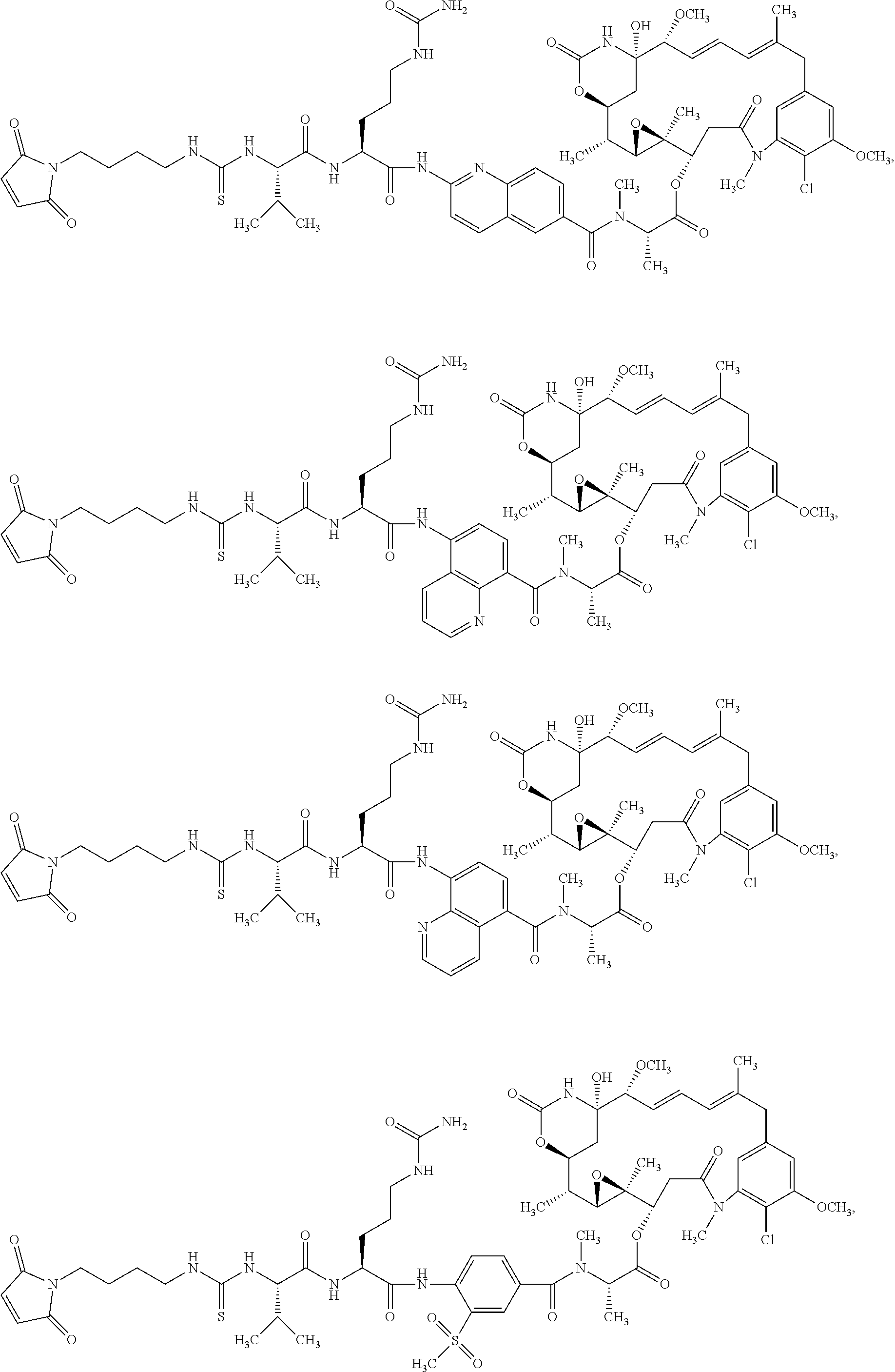

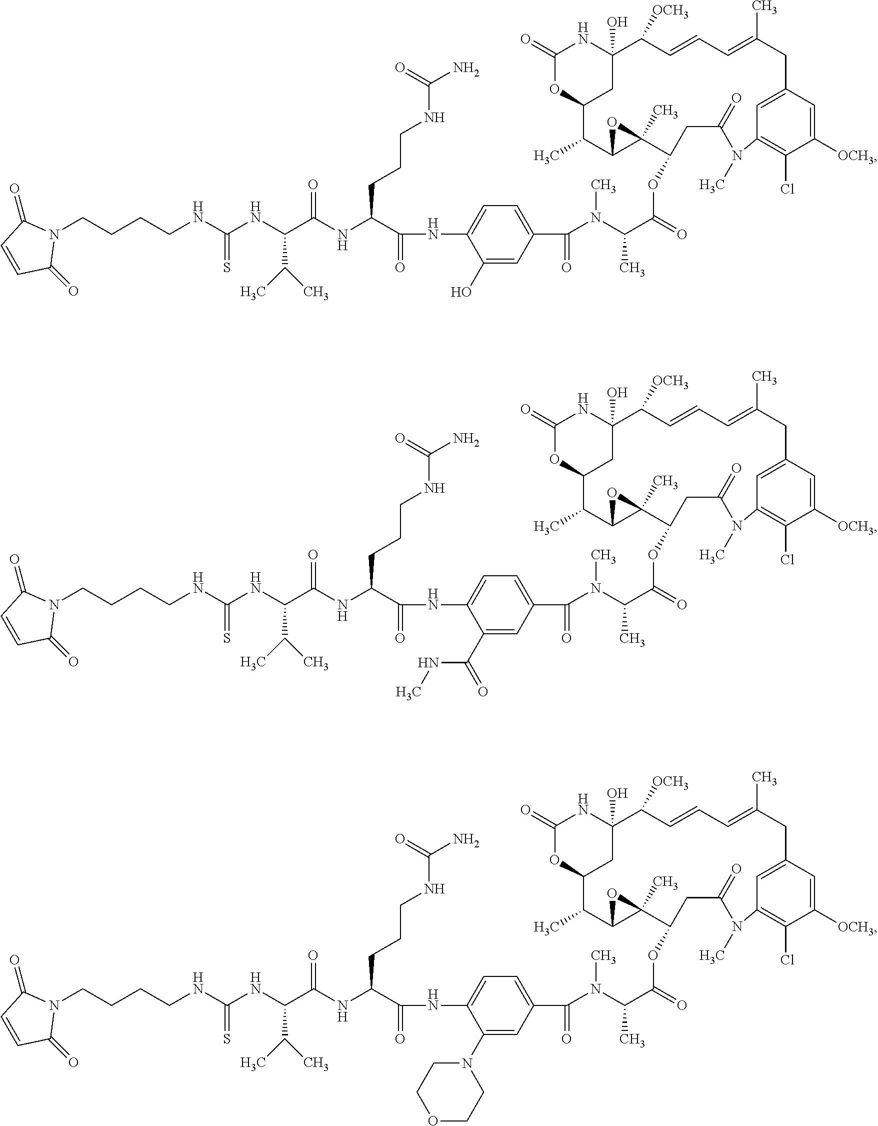

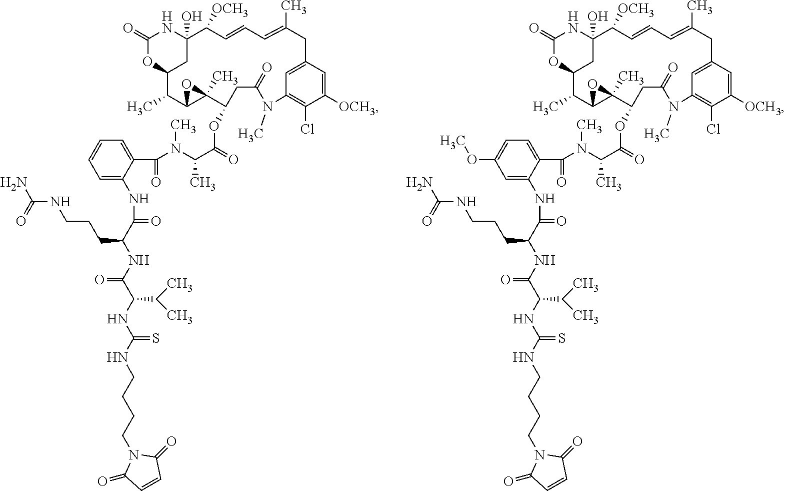 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=S)NCCCCN1C(=O)C=CC1=O)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=S)NCCCCN2C(=O)C=CC2=O)C(C)C)c1]

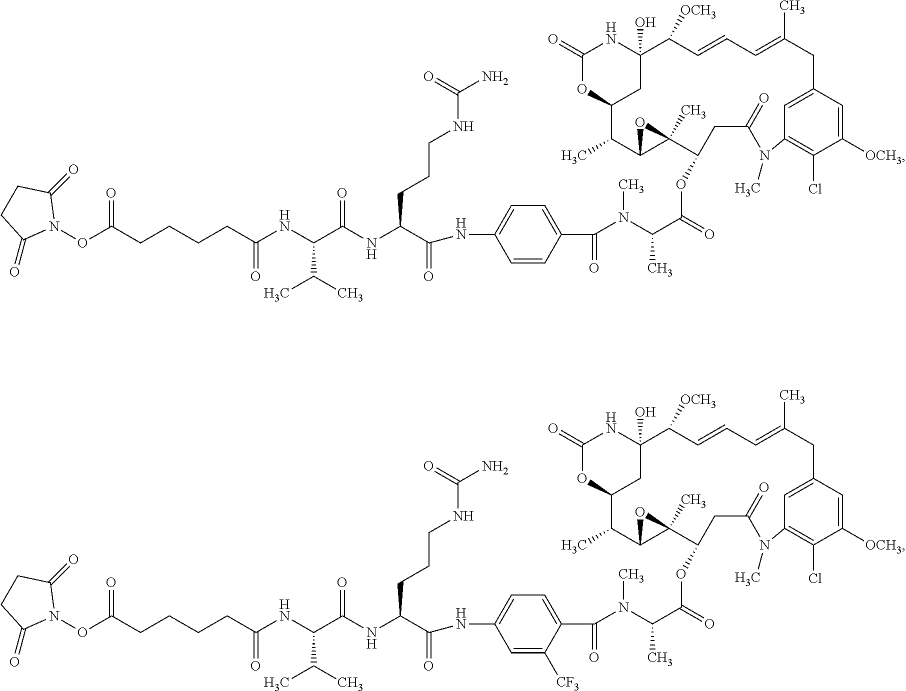 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(=O)ON3C(=O)CCC3=O)C(C)C)cc1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(=O)ON3C(=O)CCC3=O)C(C)C)cc1C(F)(F)F)[C@]1(C)O[C@H]1[C@H](C)[C@@H]1C[C@@](O)(NC(=O)O1)[C@H](OC)/C=C/C=C(\C)C2]

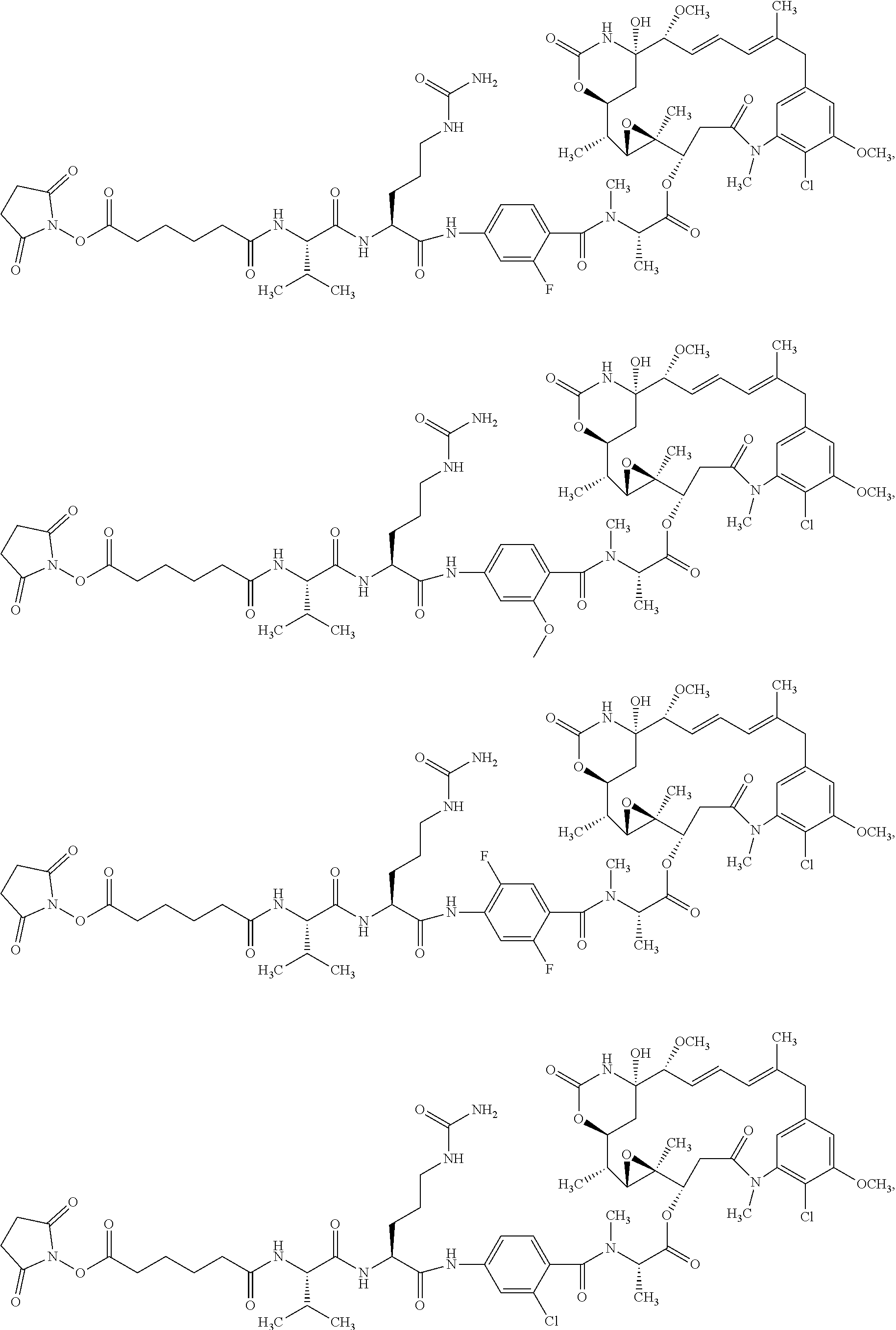

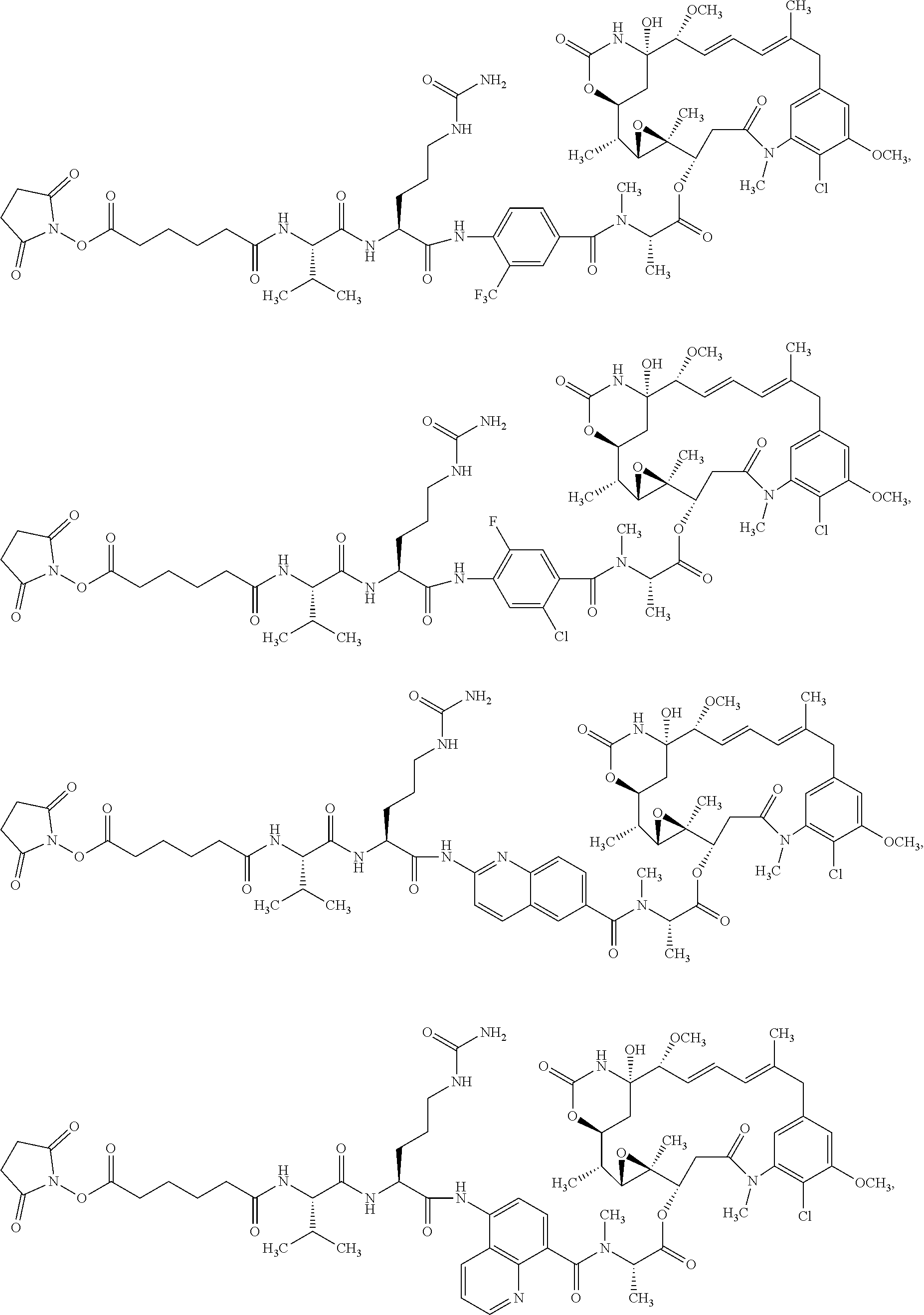

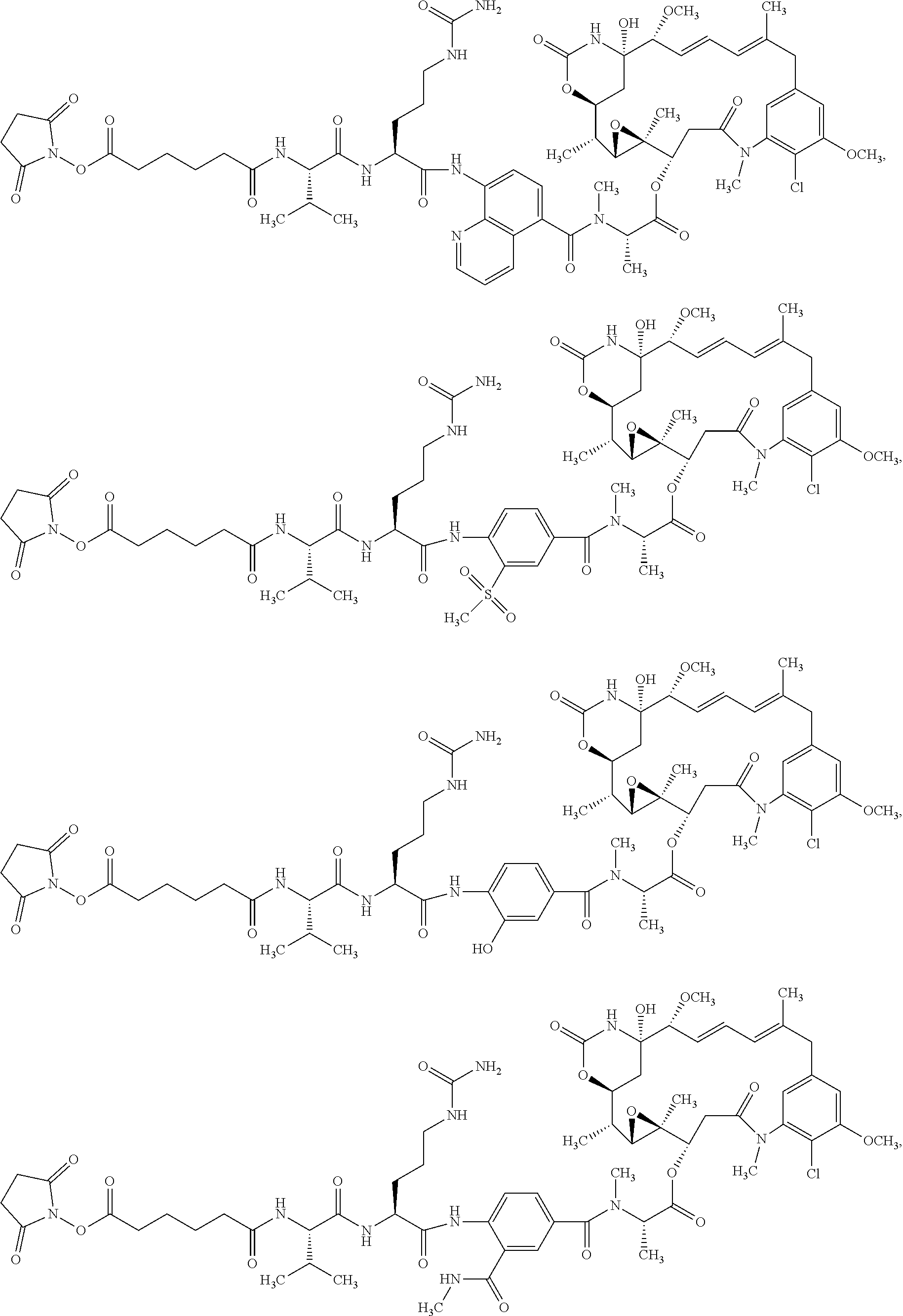

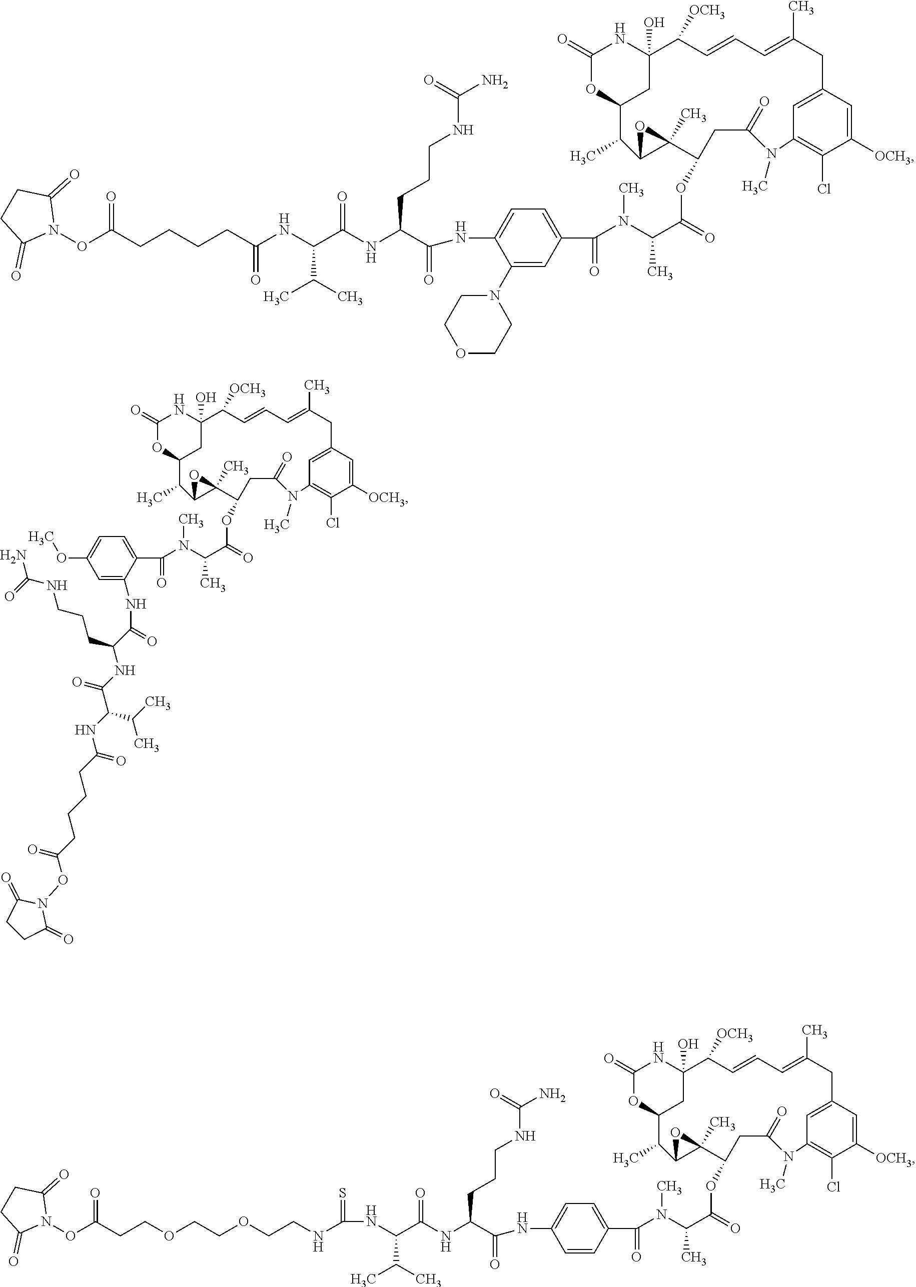

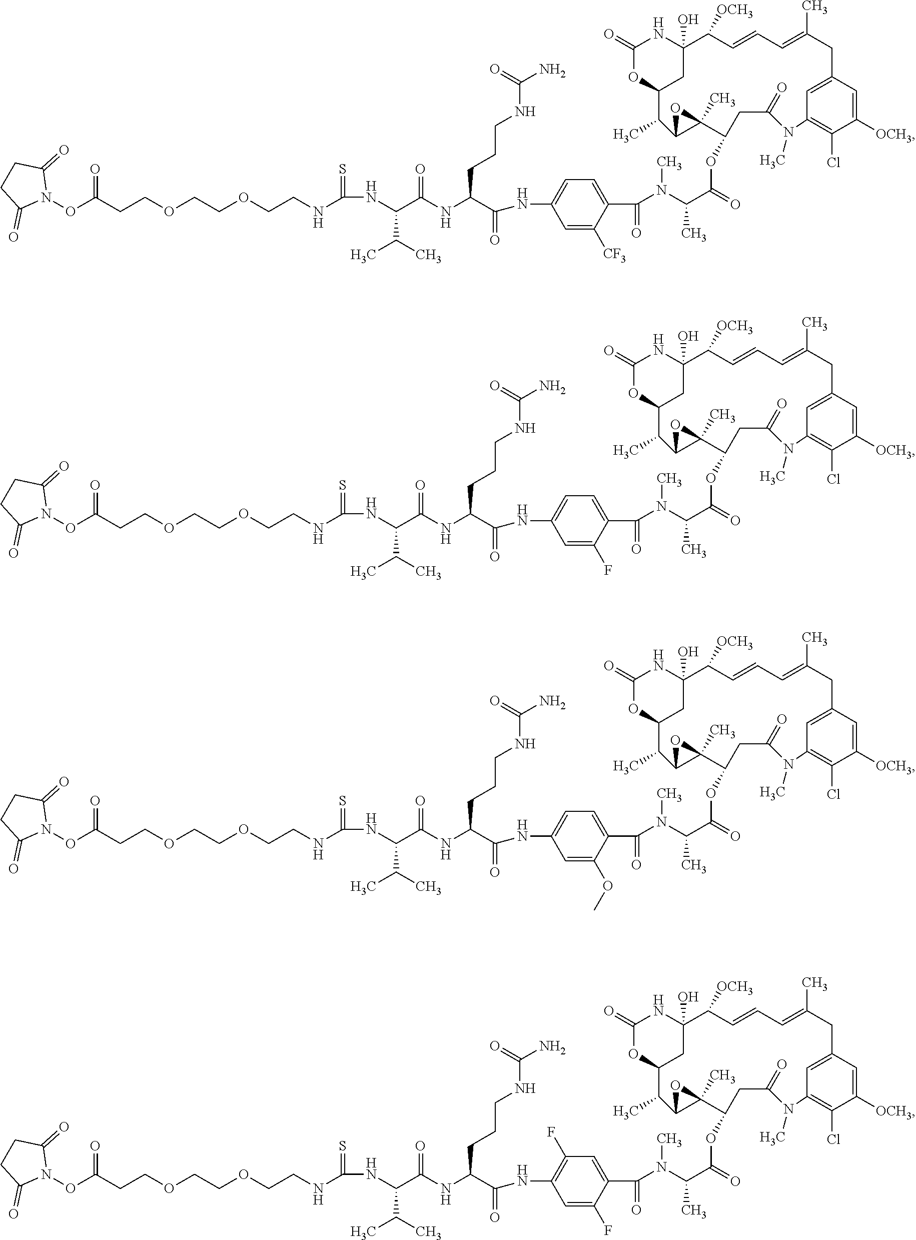

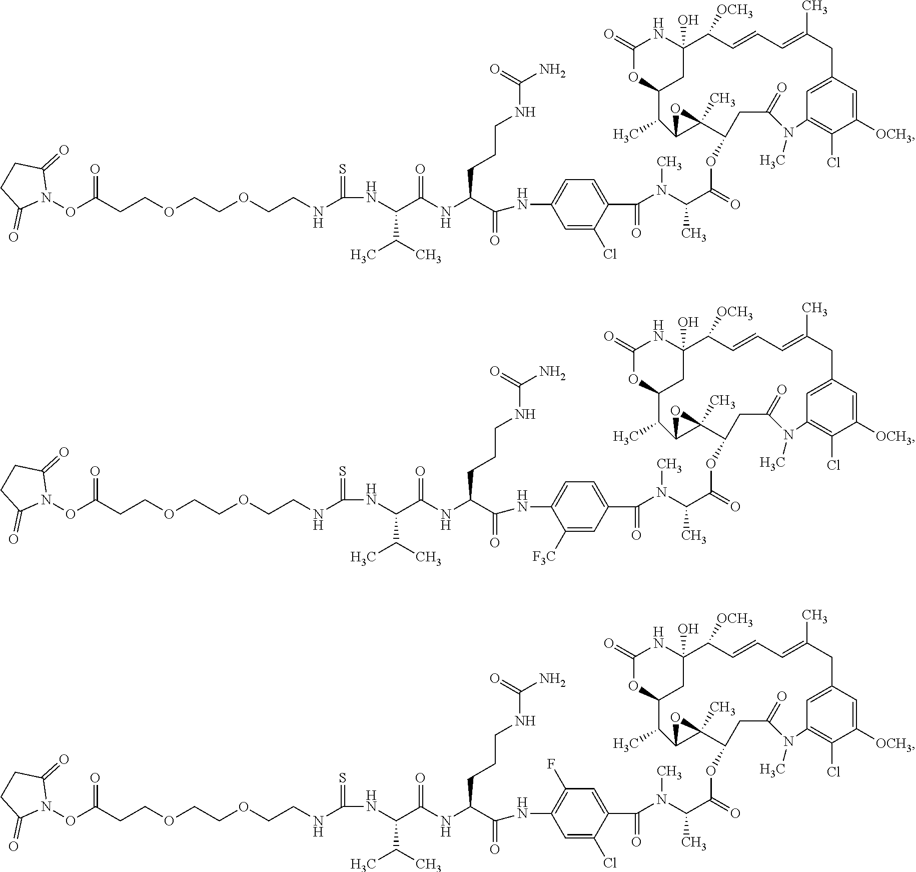

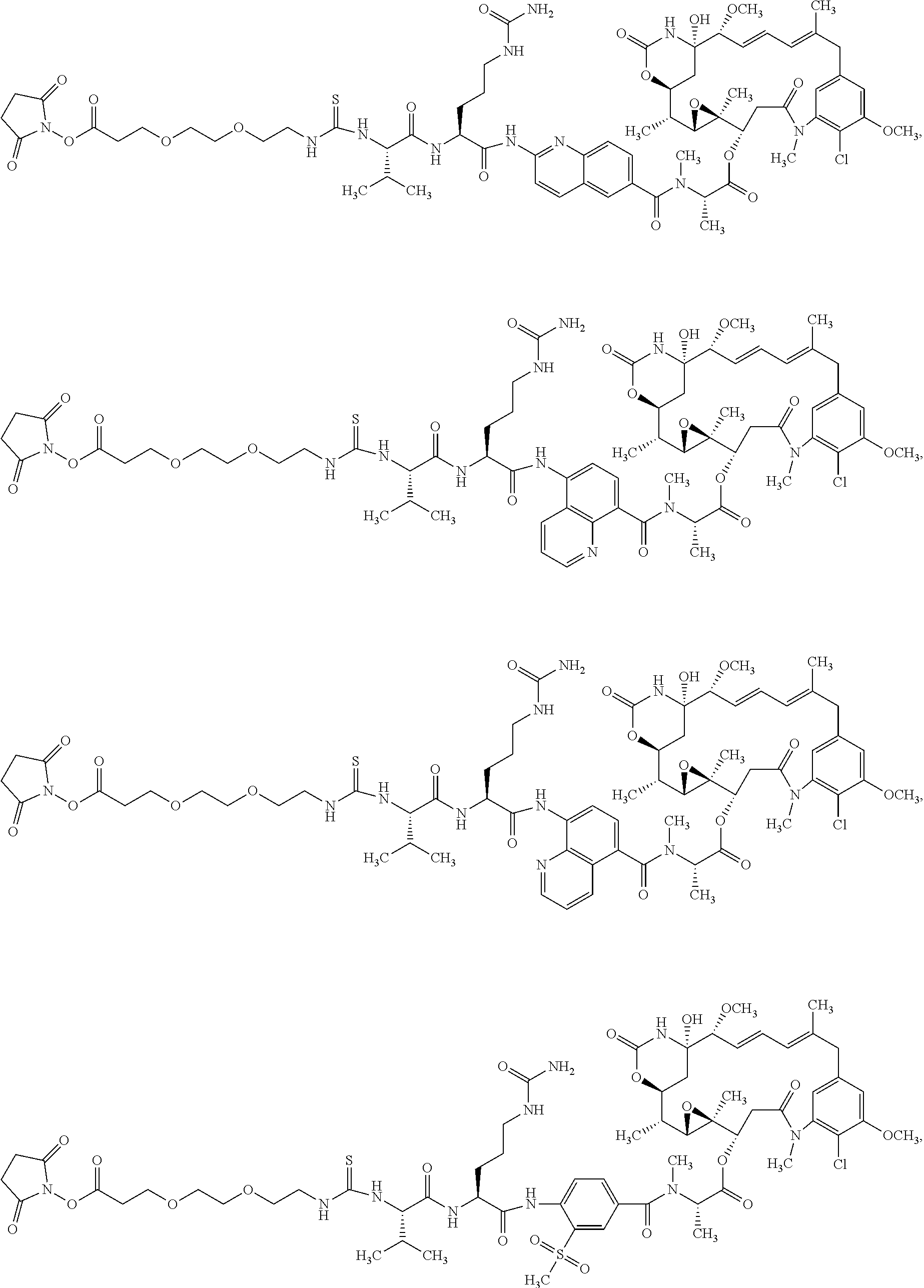

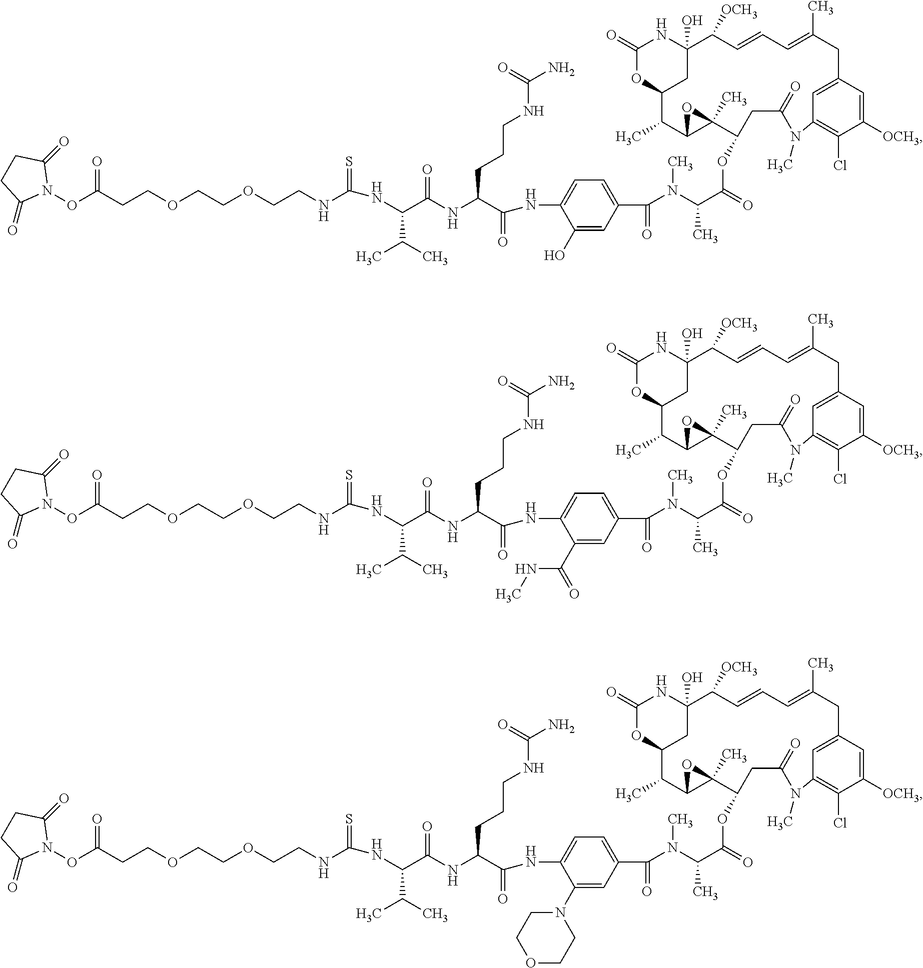

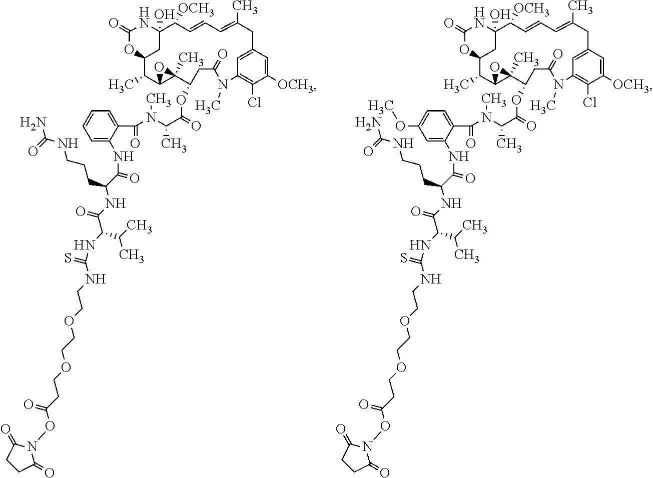 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=S)NCCOCCOCCC(=O)ON1C(=O)CCC1=O)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=S)NCCOCCOCCC(=O)ON2C(=O)CCC2=O)C(C)C)c1]

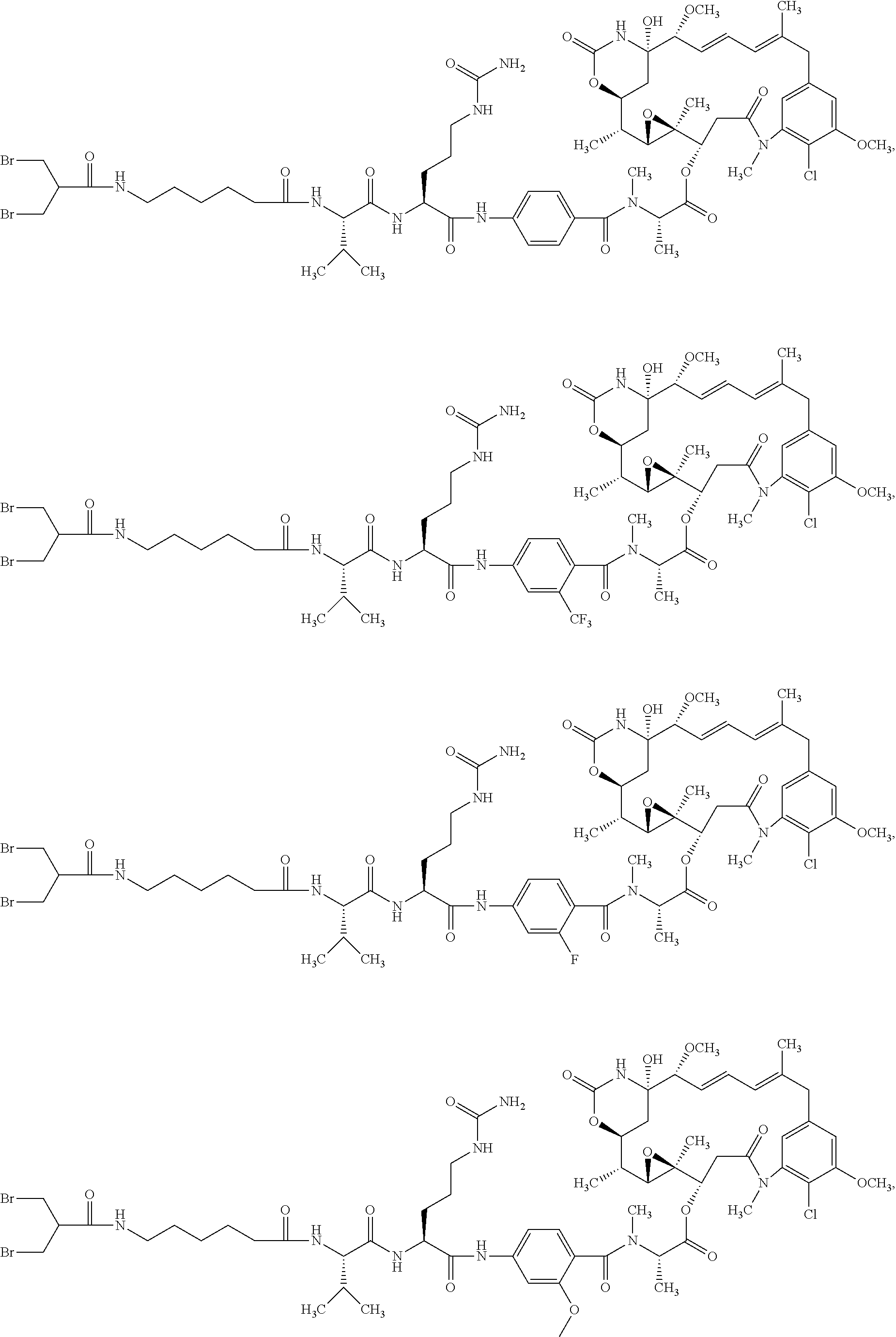

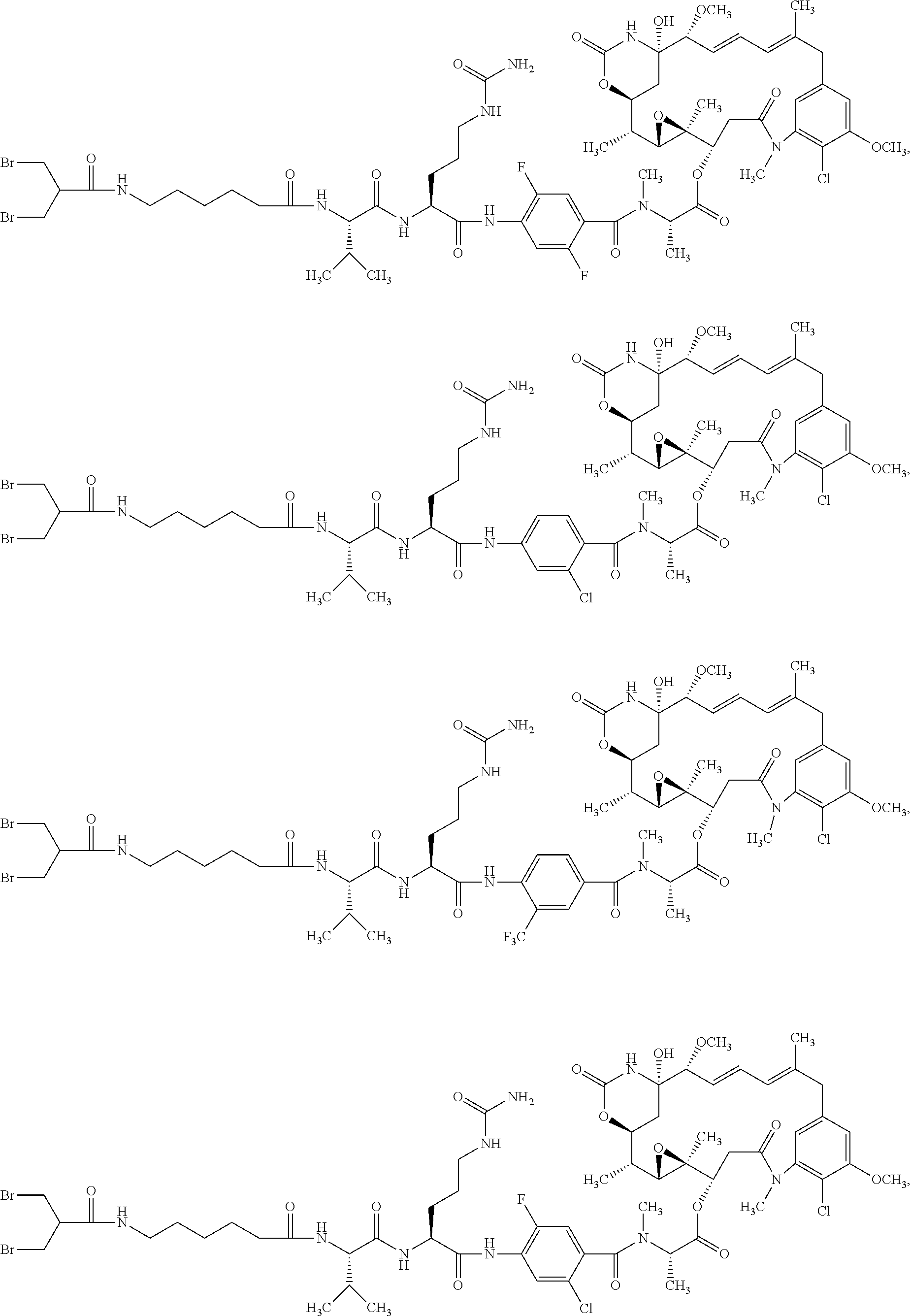

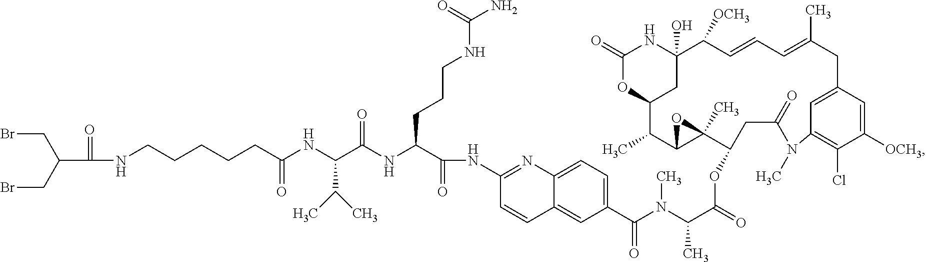 [SMILES: COc1cc2cc(c1Cl)N(C)C(=O)C[C@H](OC(=O)[C@H](C)N(C)C(=O)c1ccc3nc(NC(=O)[C@H](CCCNC(N)=O)NC(=O)[C@@H](NC(=O)CCCCCNC(=O)C(CBr)CBr)C(C)C)ccc3c1)[C@]1(C)O[C@H]1[C@H](C)[C@@H]1C[C@@](O)(NC(=O)O1)[C@H](OC)/C=C/C=C(\C)C2]

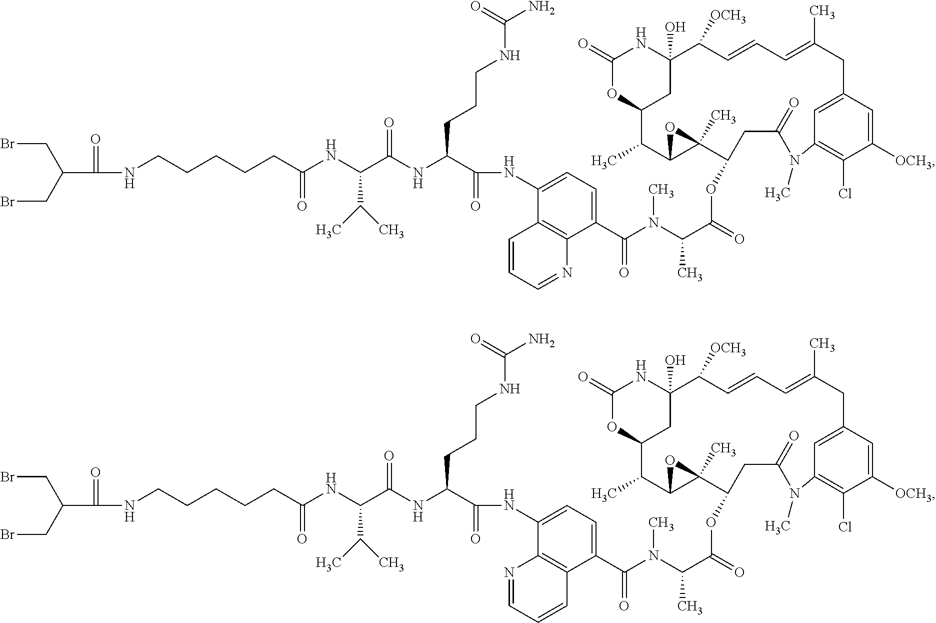 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCNC(=O)C(CBr)CBr)C(C)C)c3cccnc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCNC(=O)C(CBr)CBr)C(C)C)c3ncccc13)[C@]1(C)O[C@H]1[C@H](C)[C@@H]1C[C@@](O)(NC(=O)O1)[C@H](OC)/C=C/C=C(\C)C2]

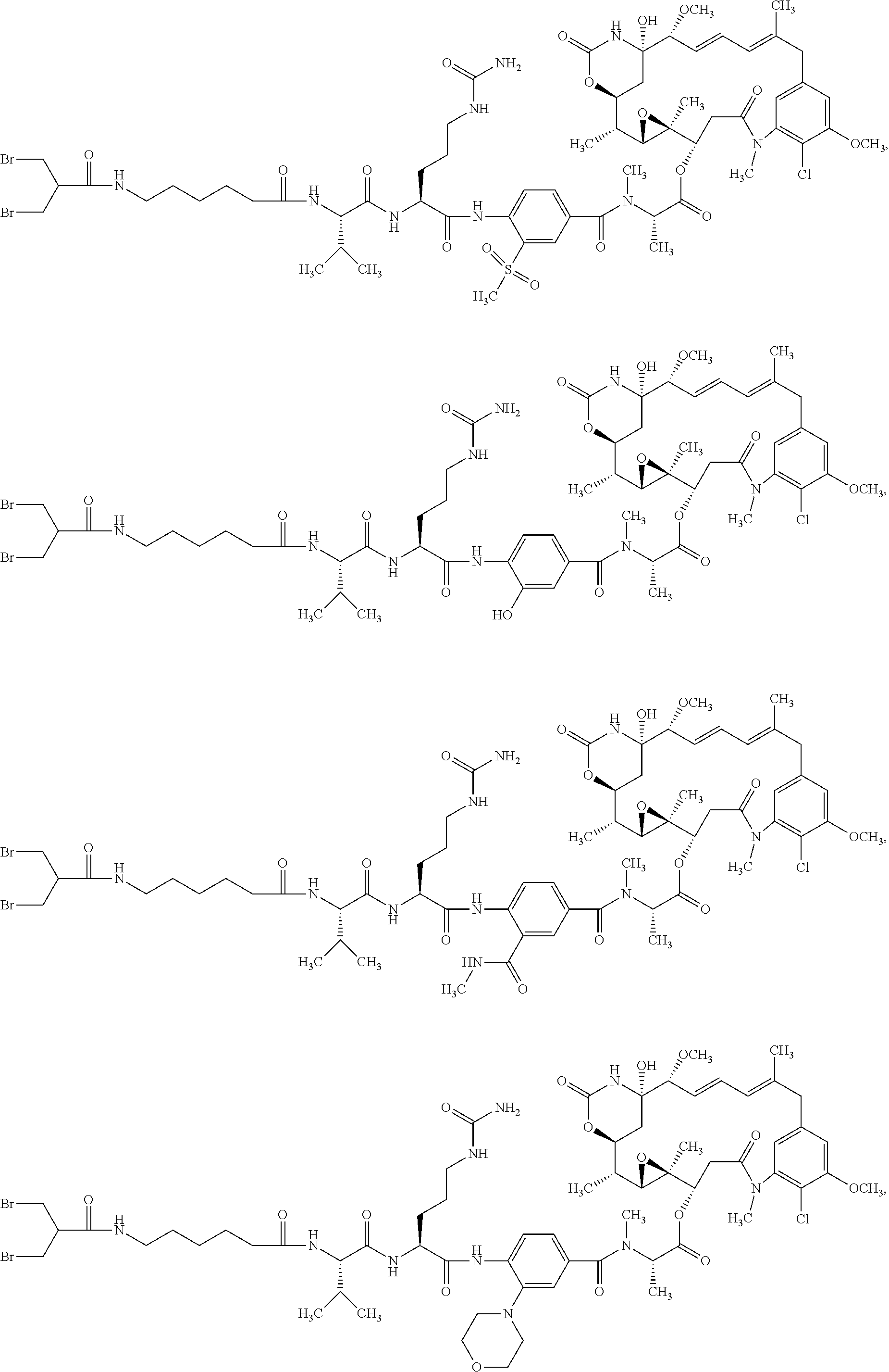

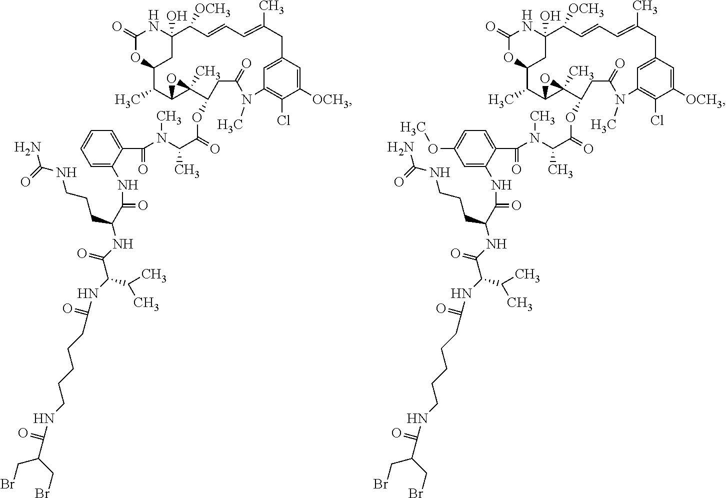 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=O)CCCCCNC(=O)C(CBr)CBr)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=O)CCCCCNC(=O)C(CBr)CBr)C(C)C)c1]

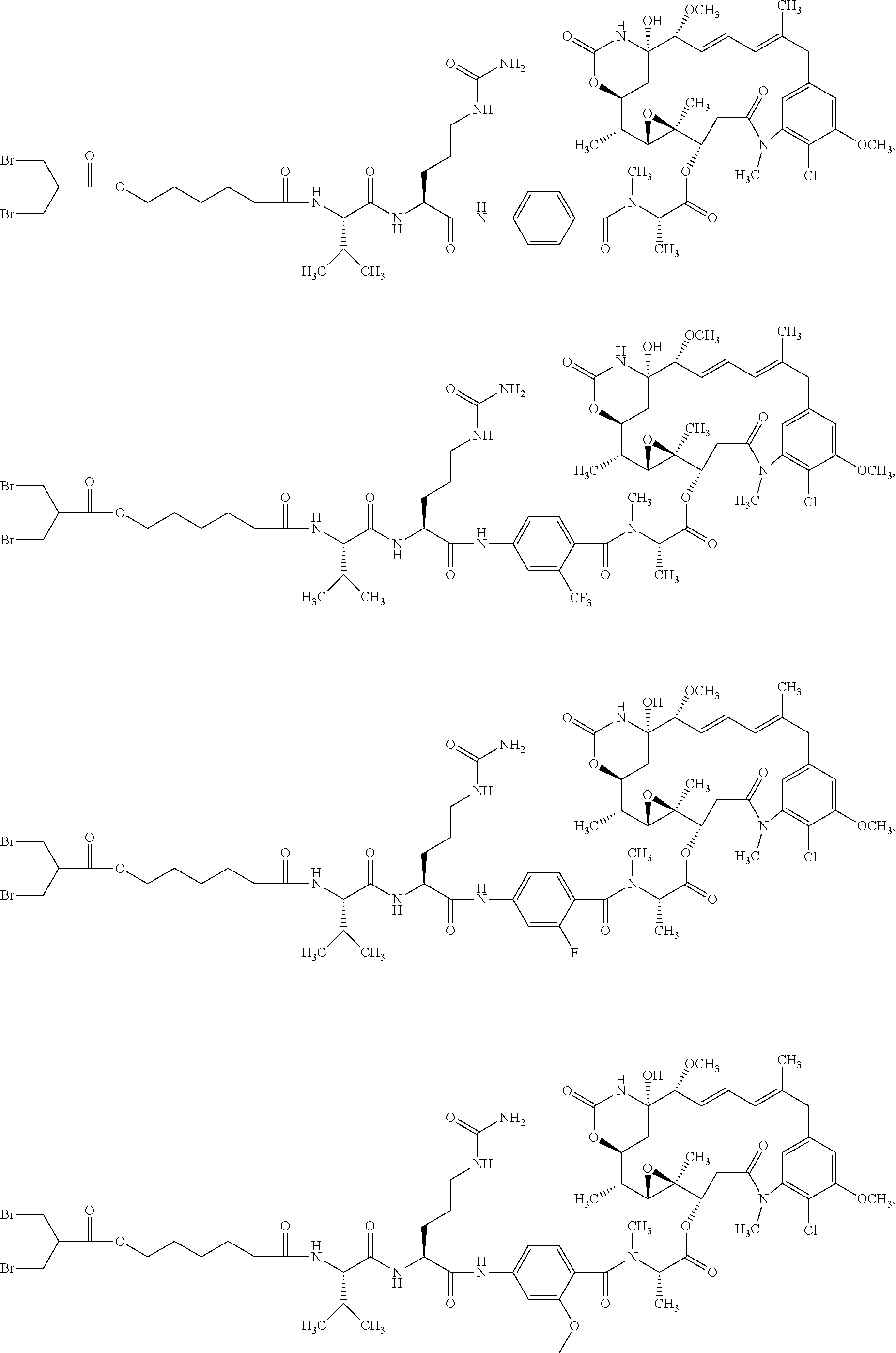

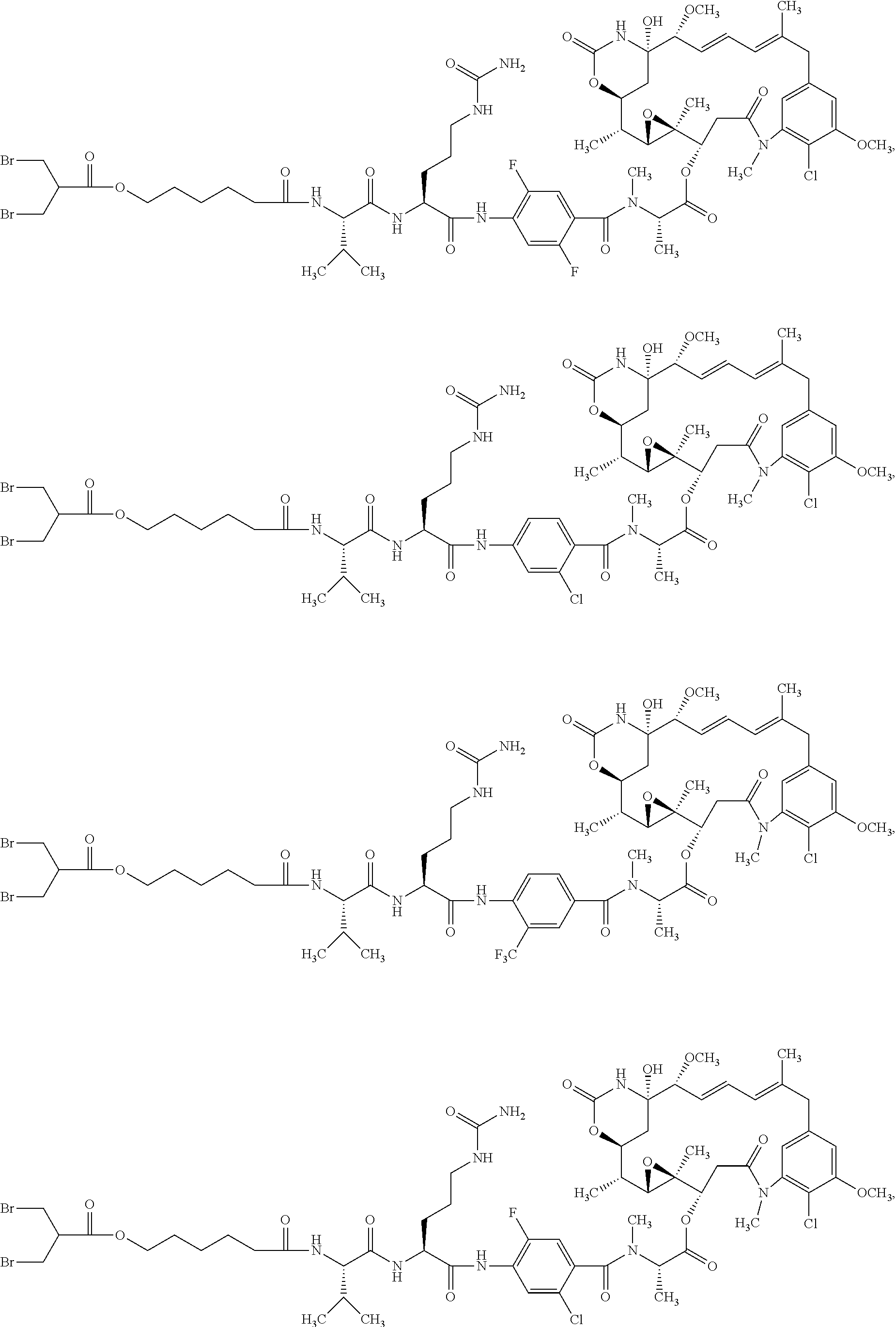

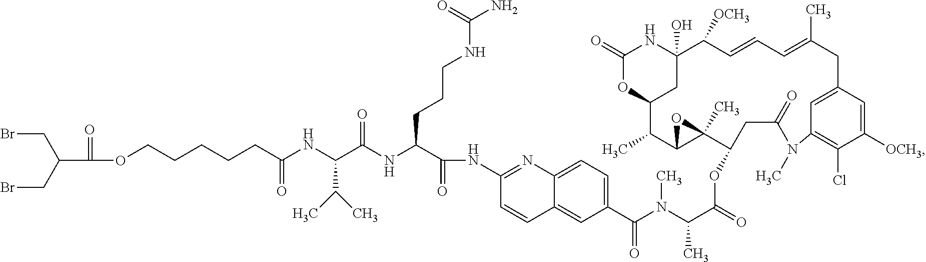 [SMILES: COc1cc2cc(c1Cl)N(C)C(=O)C[C@H](OC(=O)[C@H](C)N(C)C(=O)c1ccc3nc(NC(=O)[C@H](CCCNC(N)=O)NC(=O)[C@@H](NC(=O)CCCCCOC(=O)C(CBr)CBr)C(C)C)ccc3c1)[C@]1(C)O[C@H]1[C@H](C)[C@@H]1C[C@@](O)(NC(=O)O1)[C@H](OC)/C=C/C=C(\C)C2]

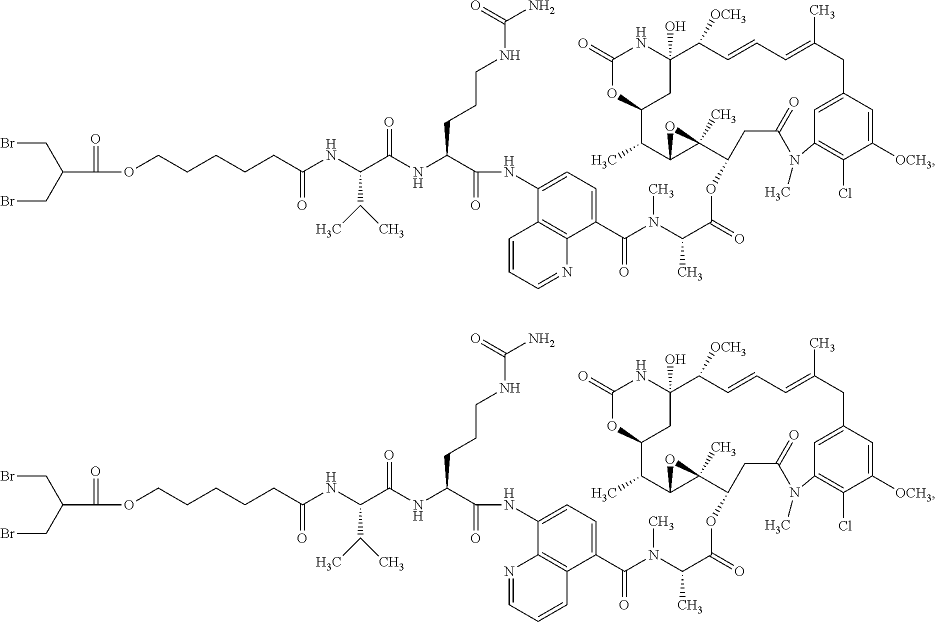 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCOC(=O)C(CBr)CBr)C(C)C)c3cccnc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCOC(=O)C(CBr)CBr)C(C)C)c3ncccc13)[C@]1(C)O[C@H]1[C@H](C)[C@@H]1C[C@@](O)(NC(=O)O1)[C@H](OC)/C=C/C=C(\C)C2]

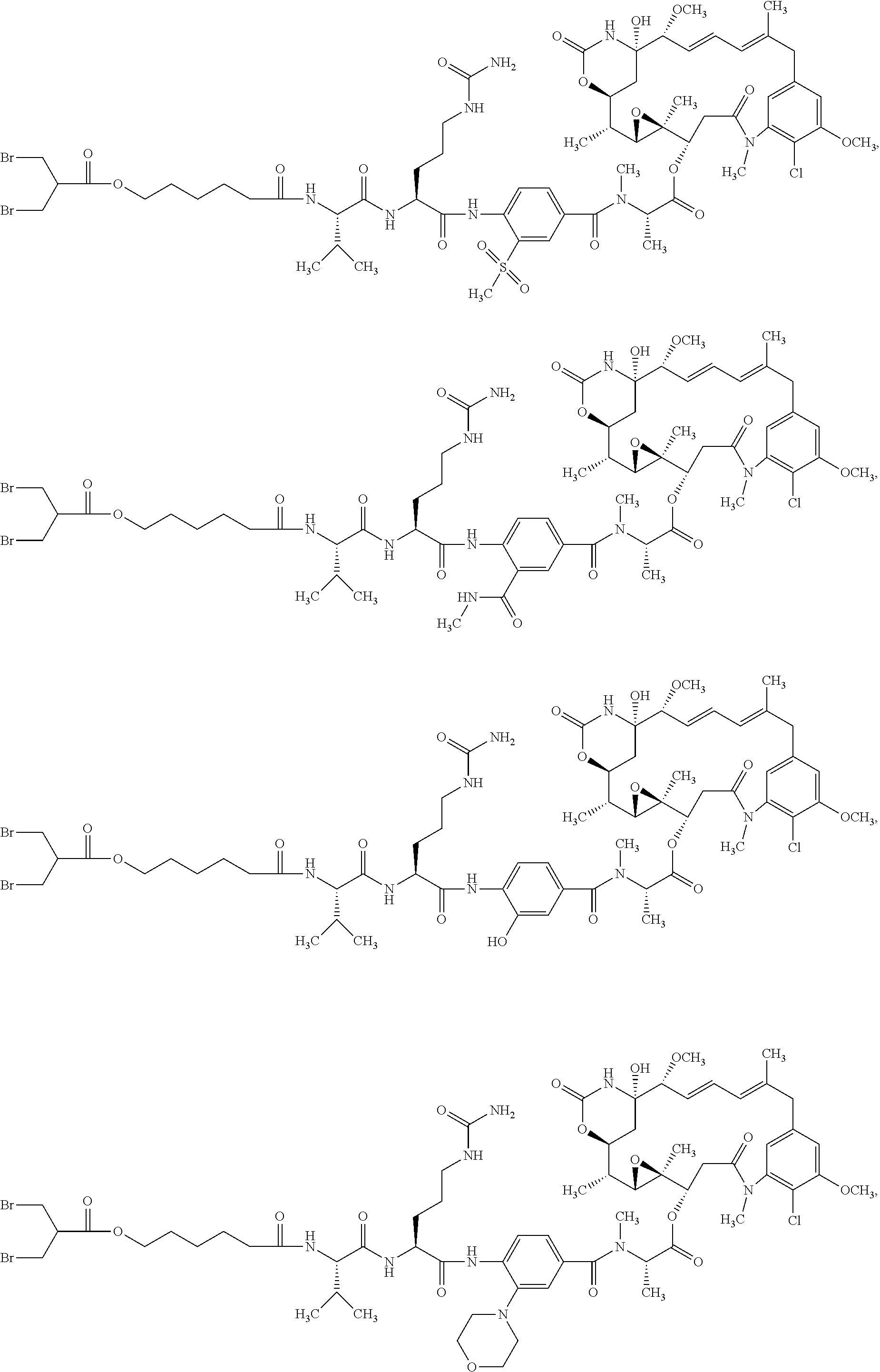

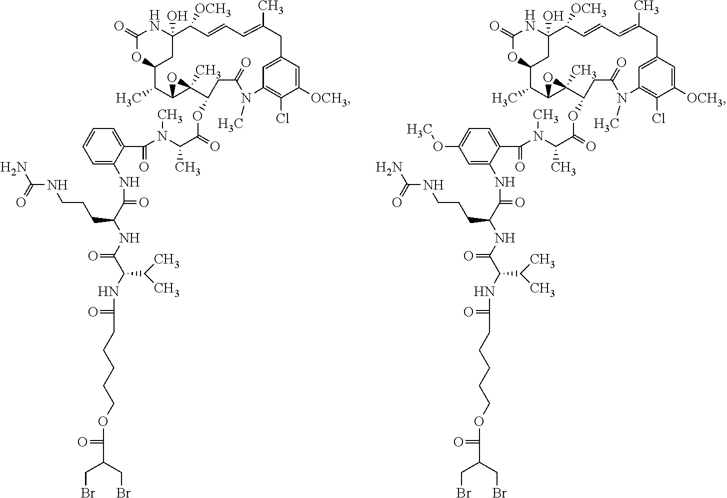 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=O)CCCCCOC(=O)C(CBr)CBr)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=O)CCCCCOC(=O)C(CBr)CBr)C(C)C)c1]

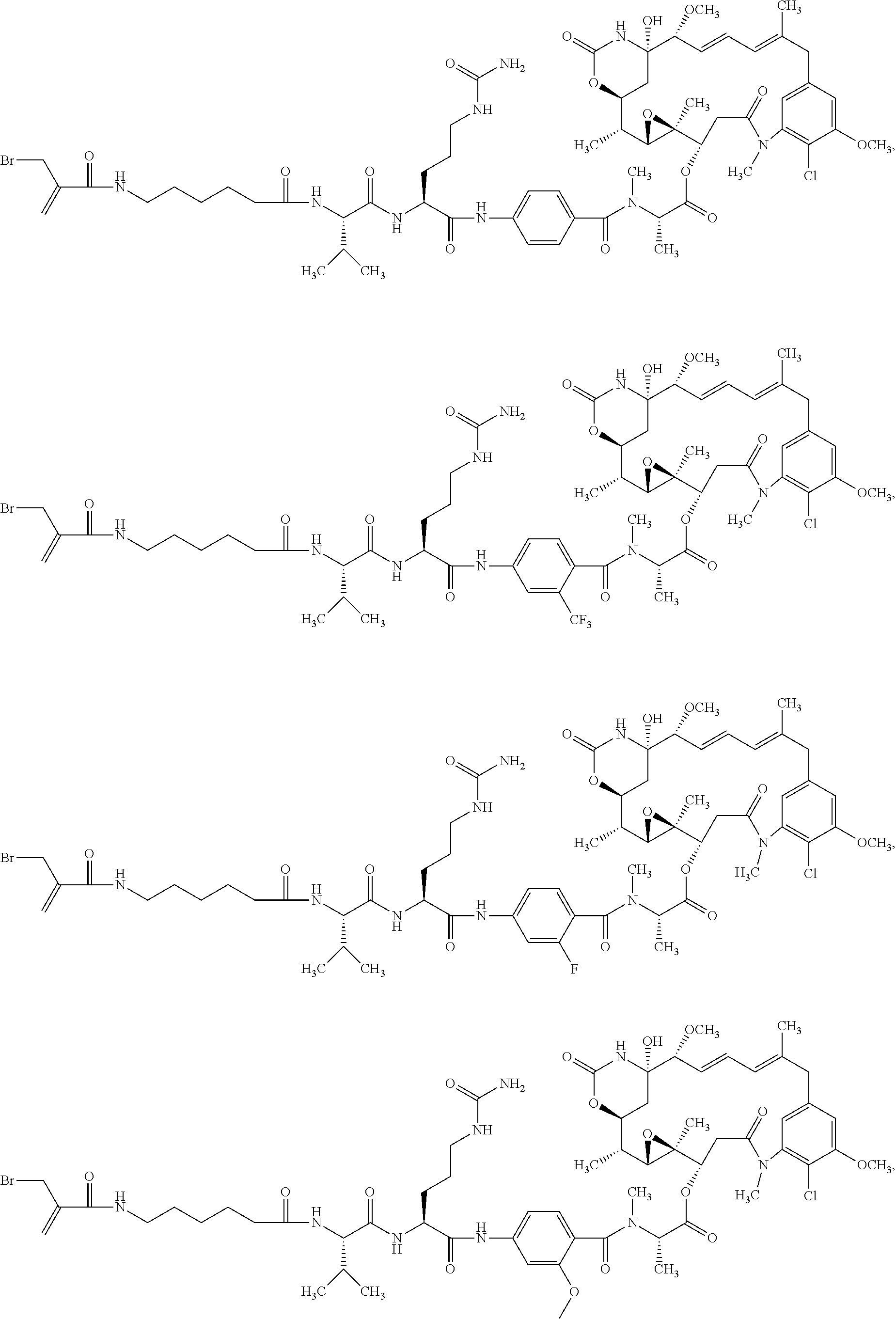

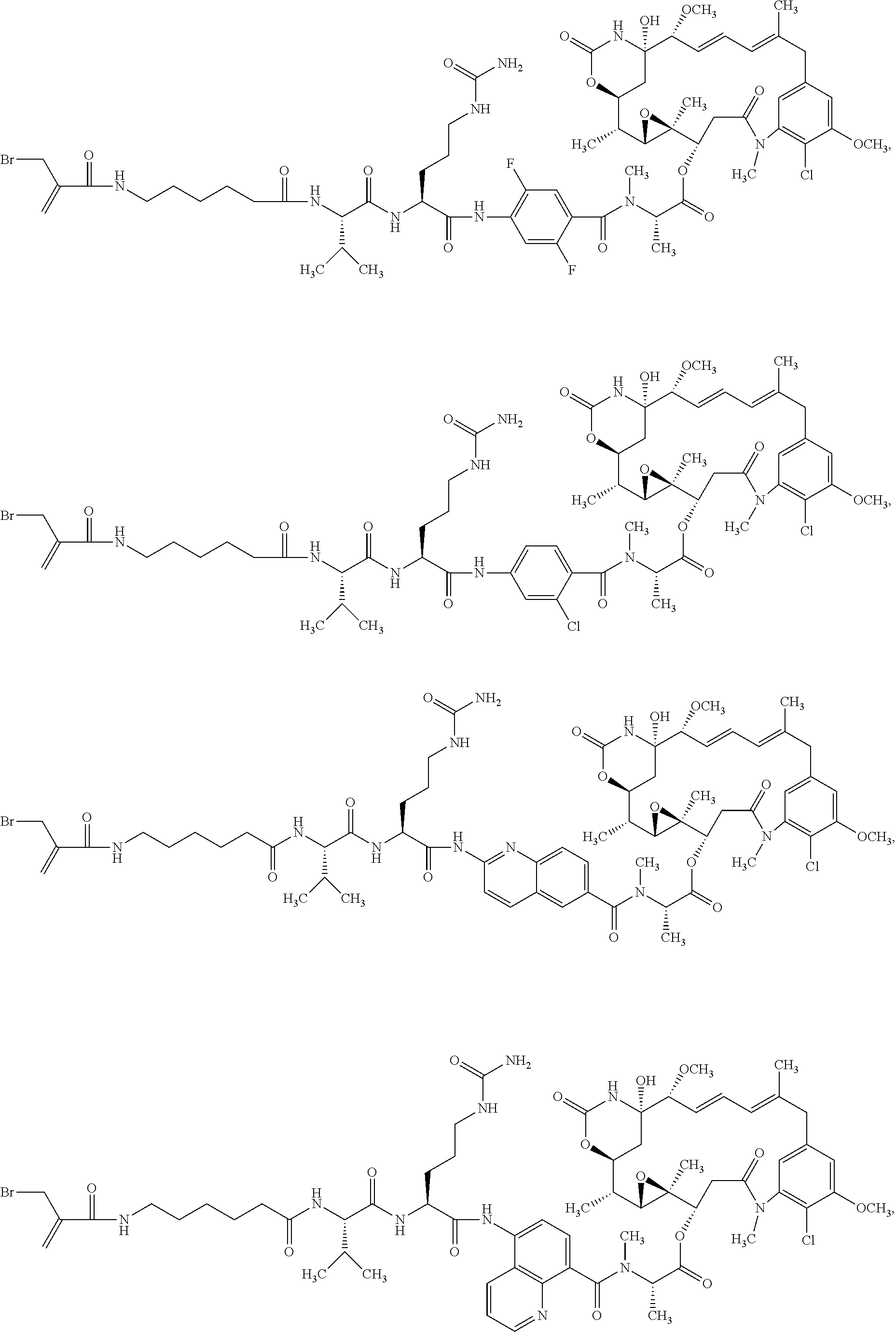

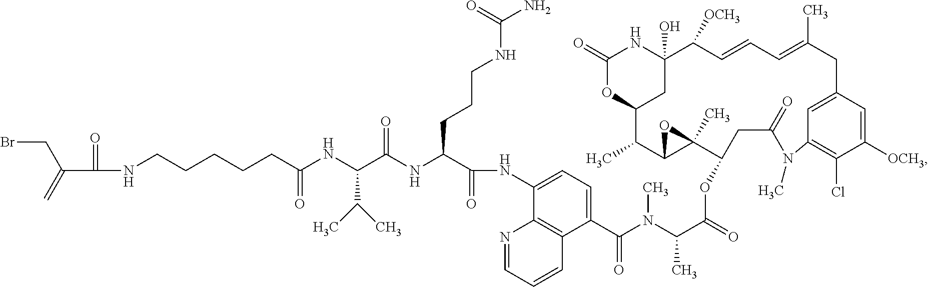 [SMILES: C=C(CBr)C(=O)NCCCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c2cccnc12)C(C)C]

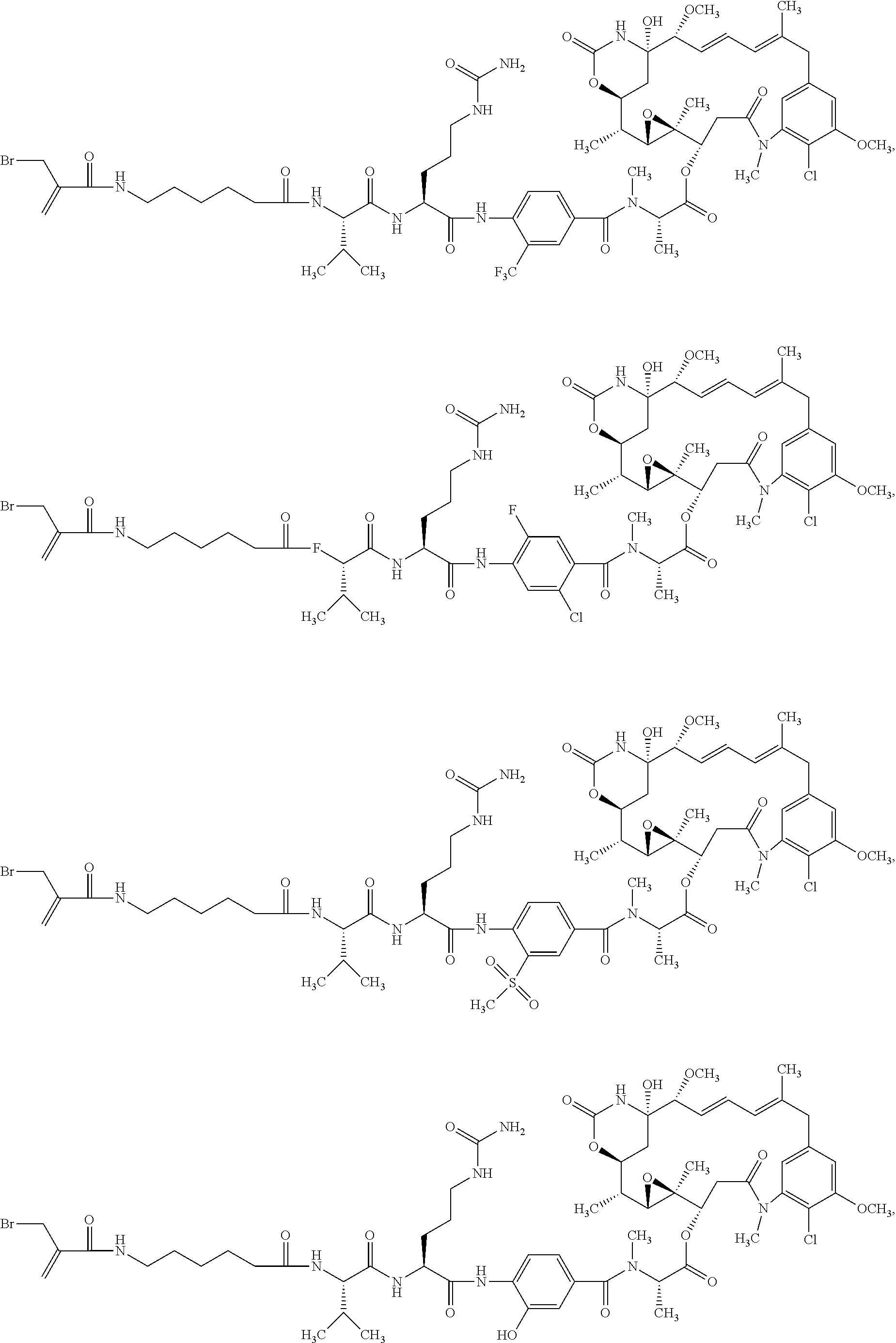

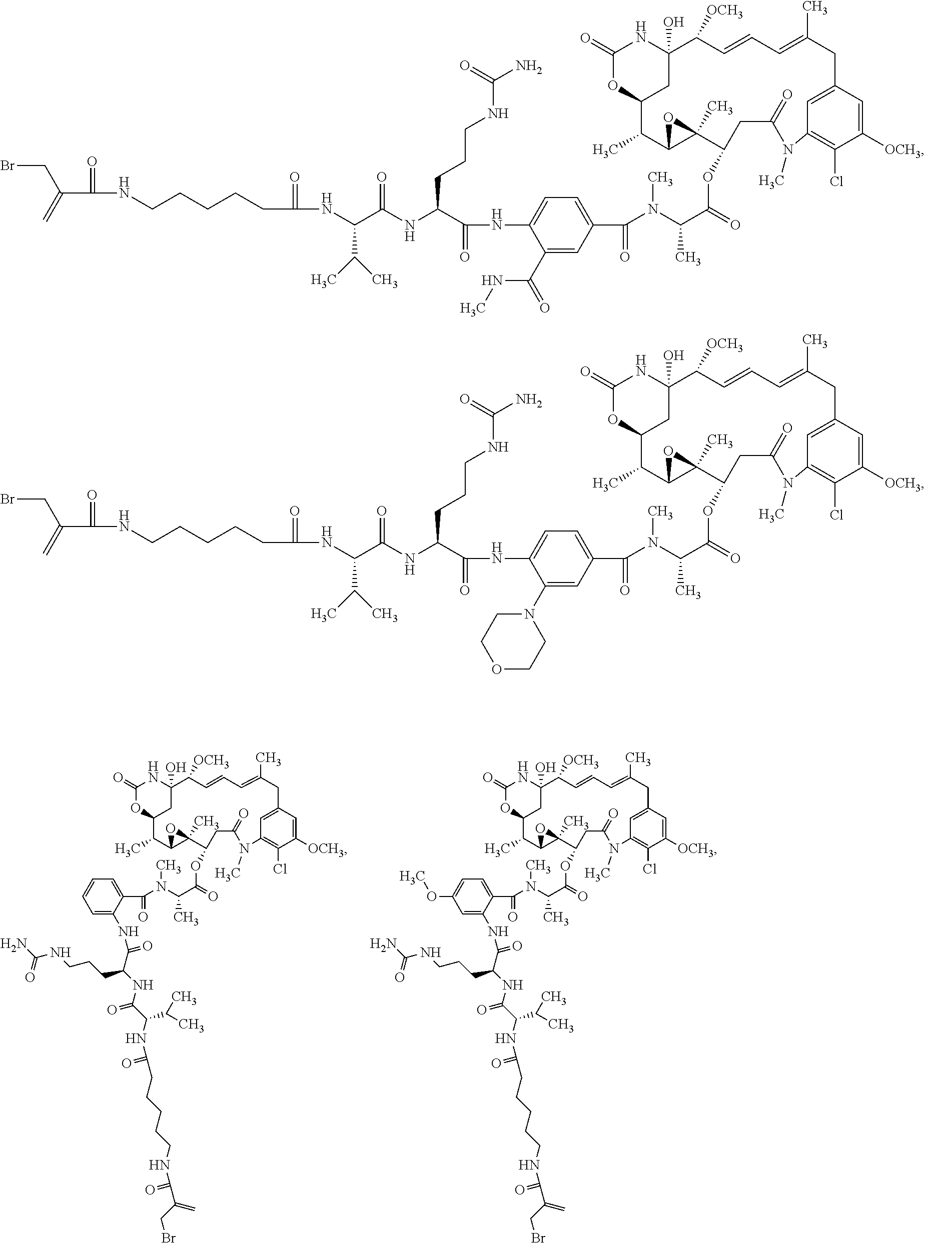

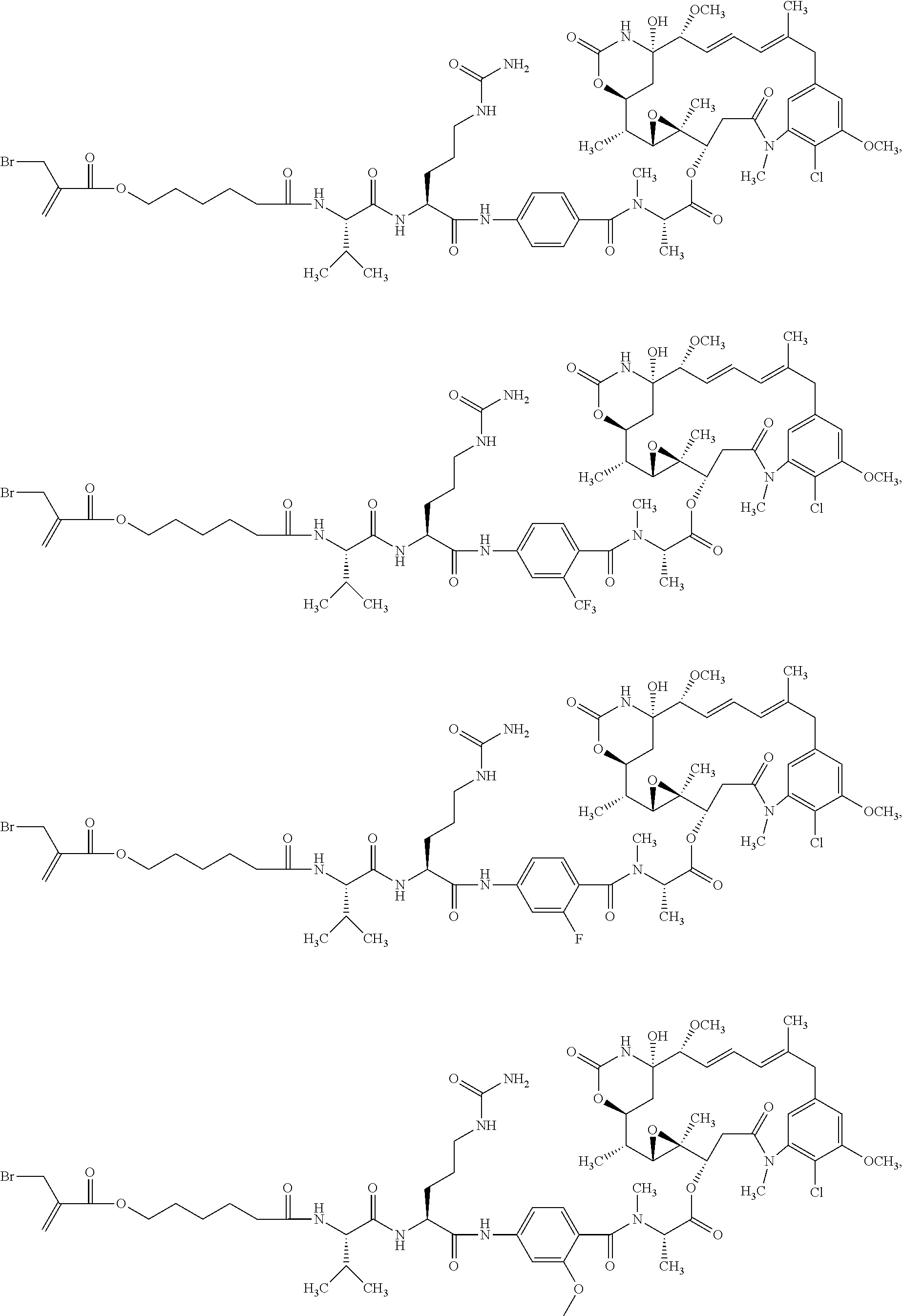

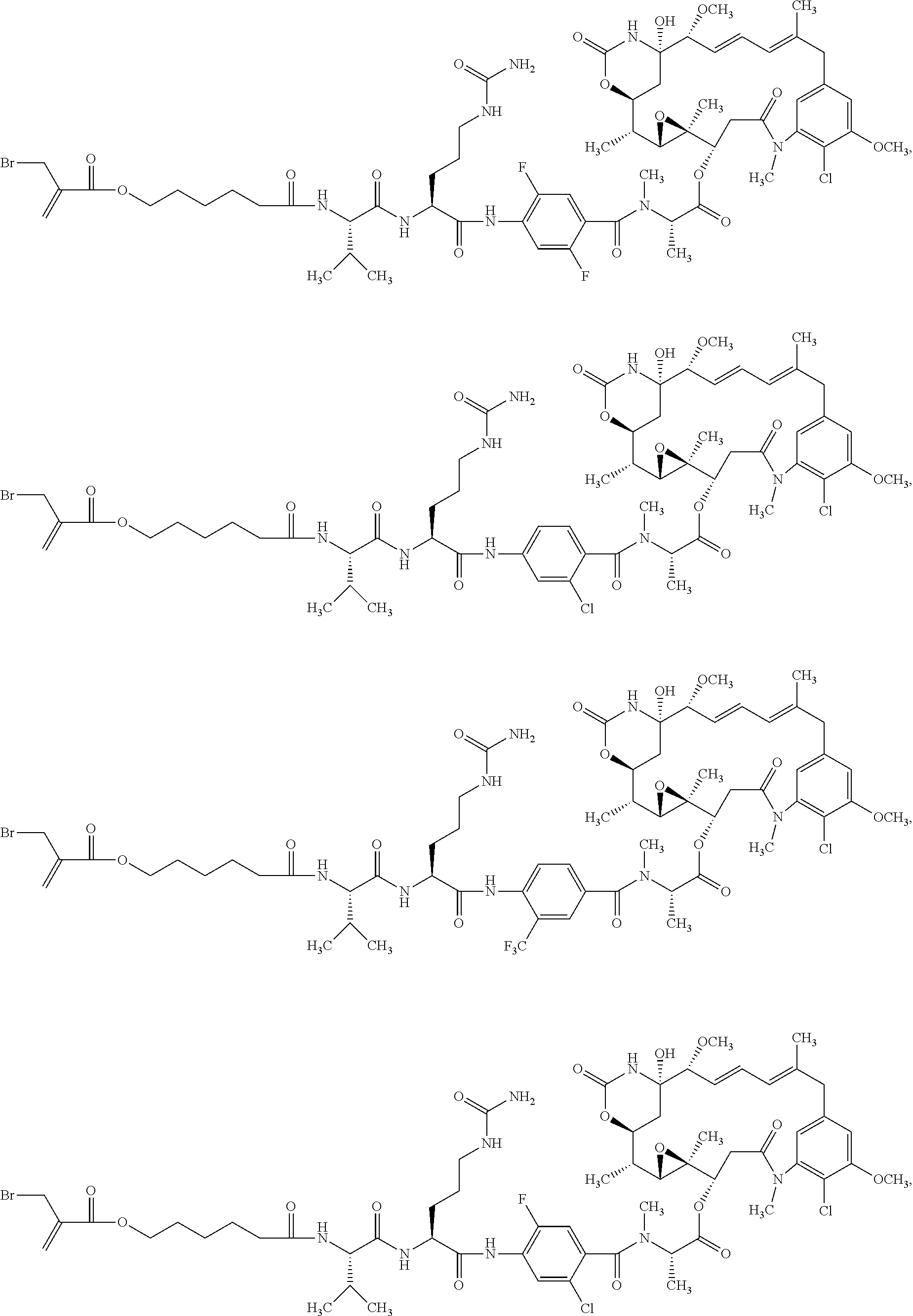

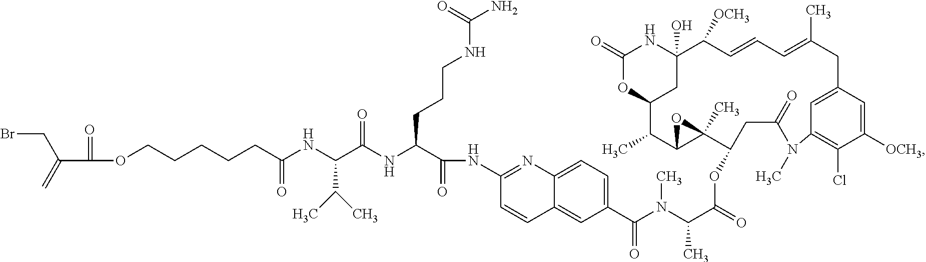 [SMILES: C=C(CBr)C(=O)OCCCCCC(=O)N[C@H](C(=O)N[C@@H](CCCNC(N)=O)C(=O)Nc1ccc2cc(C(=O)N(C)[C@@H](C)C(=O)O[C@H]3CC(=O)N(C)c4cc(cc(OC)c4Cl)C/C(C)=C/C=C/[C@@H](OC)[C@@]4(O)C[C@H](OC(=O)N4)[C@@H](C)[C@@H]4O[C@@]34C)ccc2n1)C(C)C]

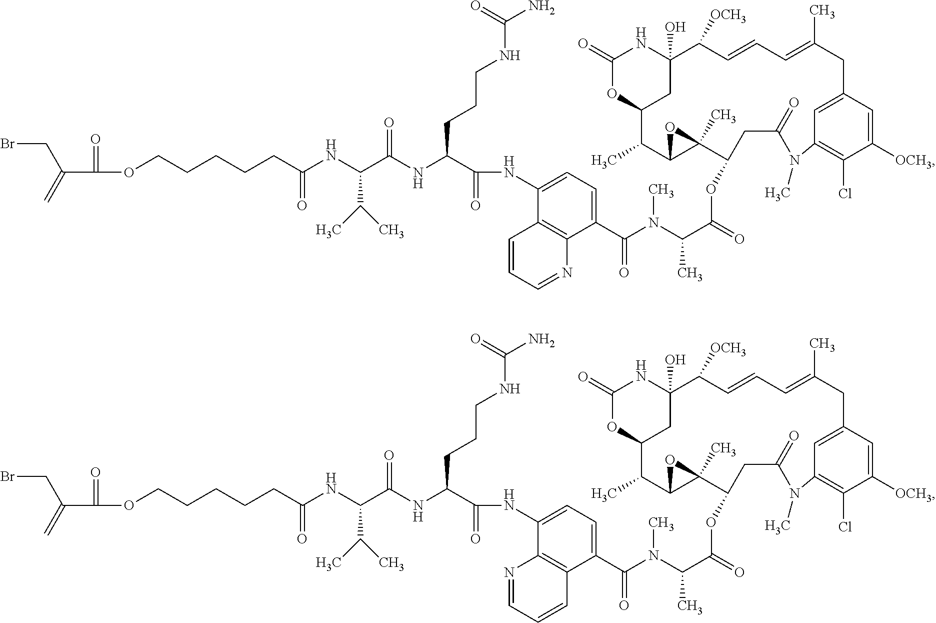 [SMILES: C=C(CBr)C(=O)OCCCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c2ncccc12)C(C)C ; C=C(CBr)C(=O)OCCCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c2cccnc12)C(C)C]

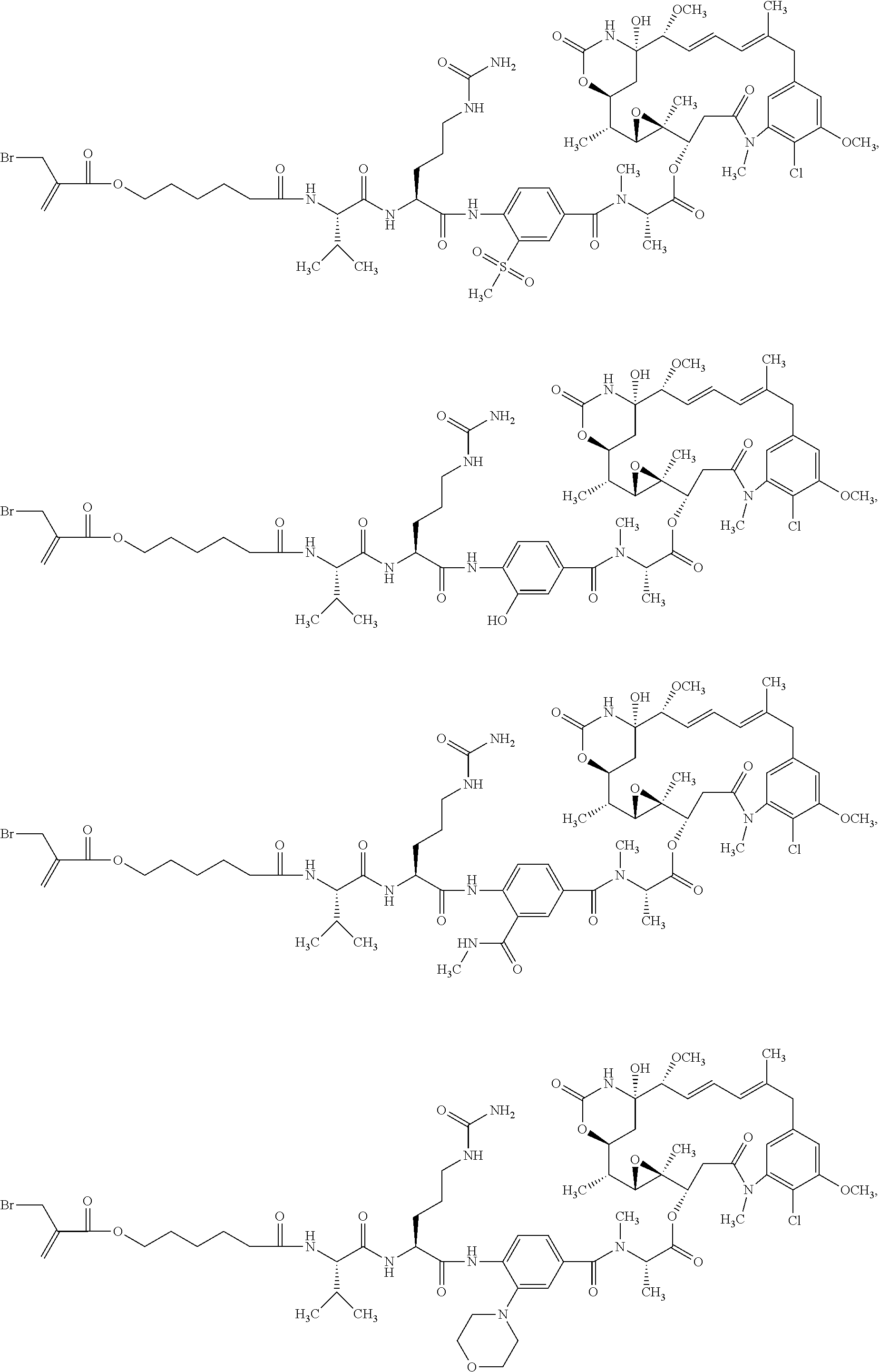

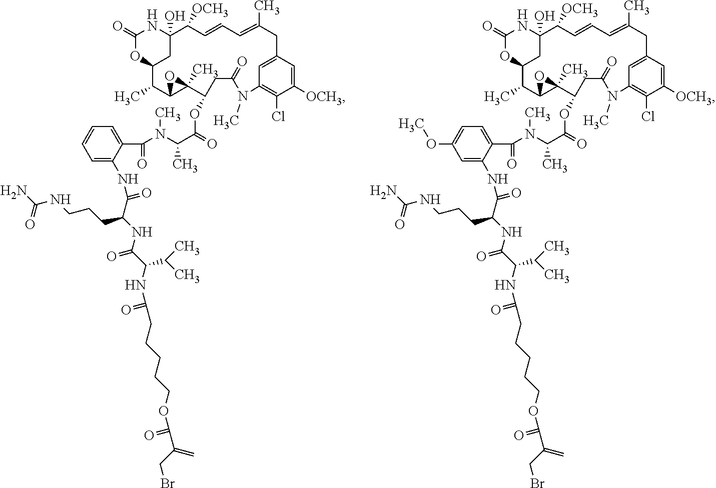 [SMILES: C=C(CBr)C(=O)OCCCCCC(=O)N[C@H](C(=O)N[C@@H](CCCNC(N)=O)C(=O)Nc1ccccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)C(C)C ; C=C(CBr)C(=O)OCCCCCC(=O)N[C@H](C(=O)N[C@@H](CCCNC(N)=O)C(=O)Nc1cc(OC)ccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)C(C)C]

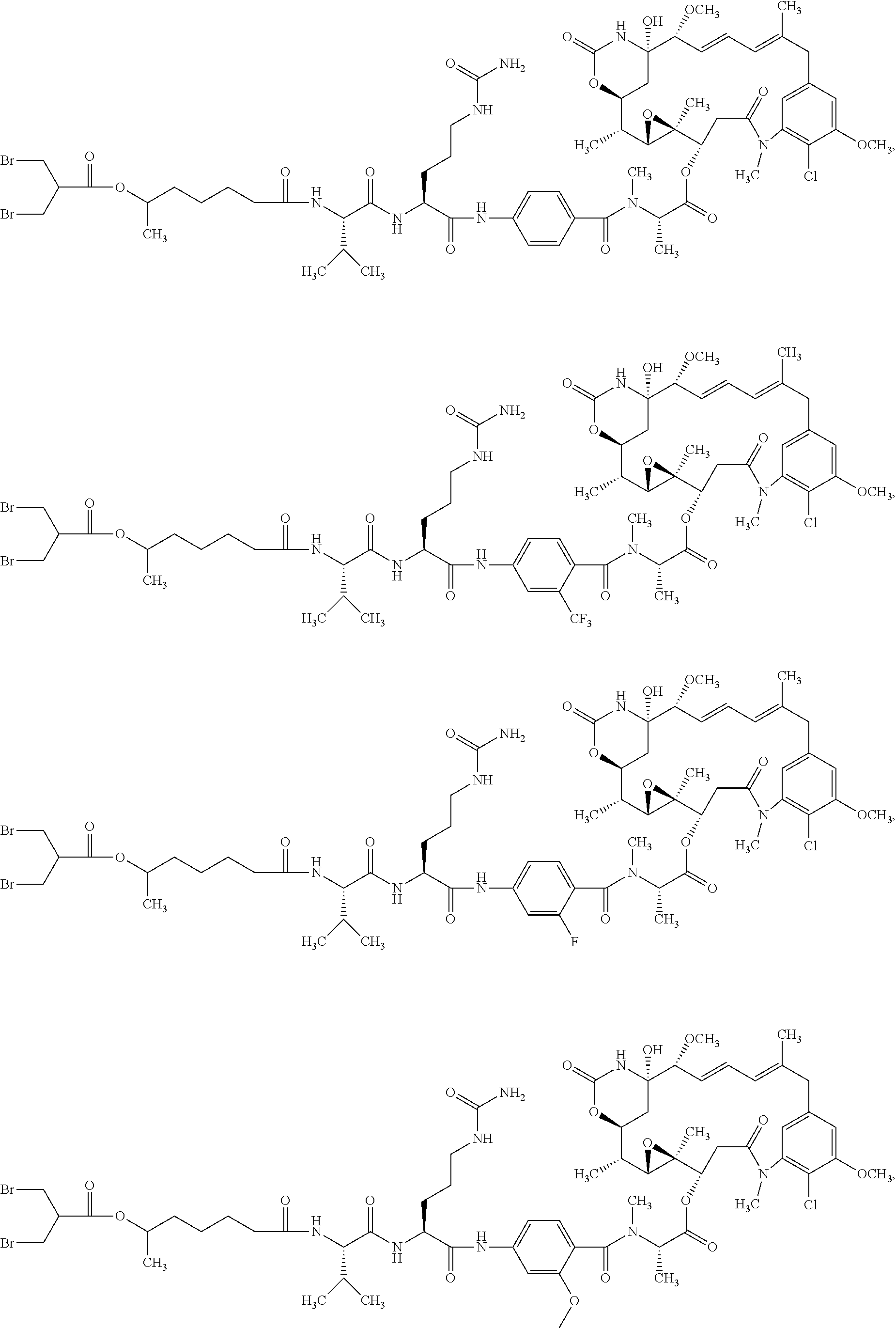

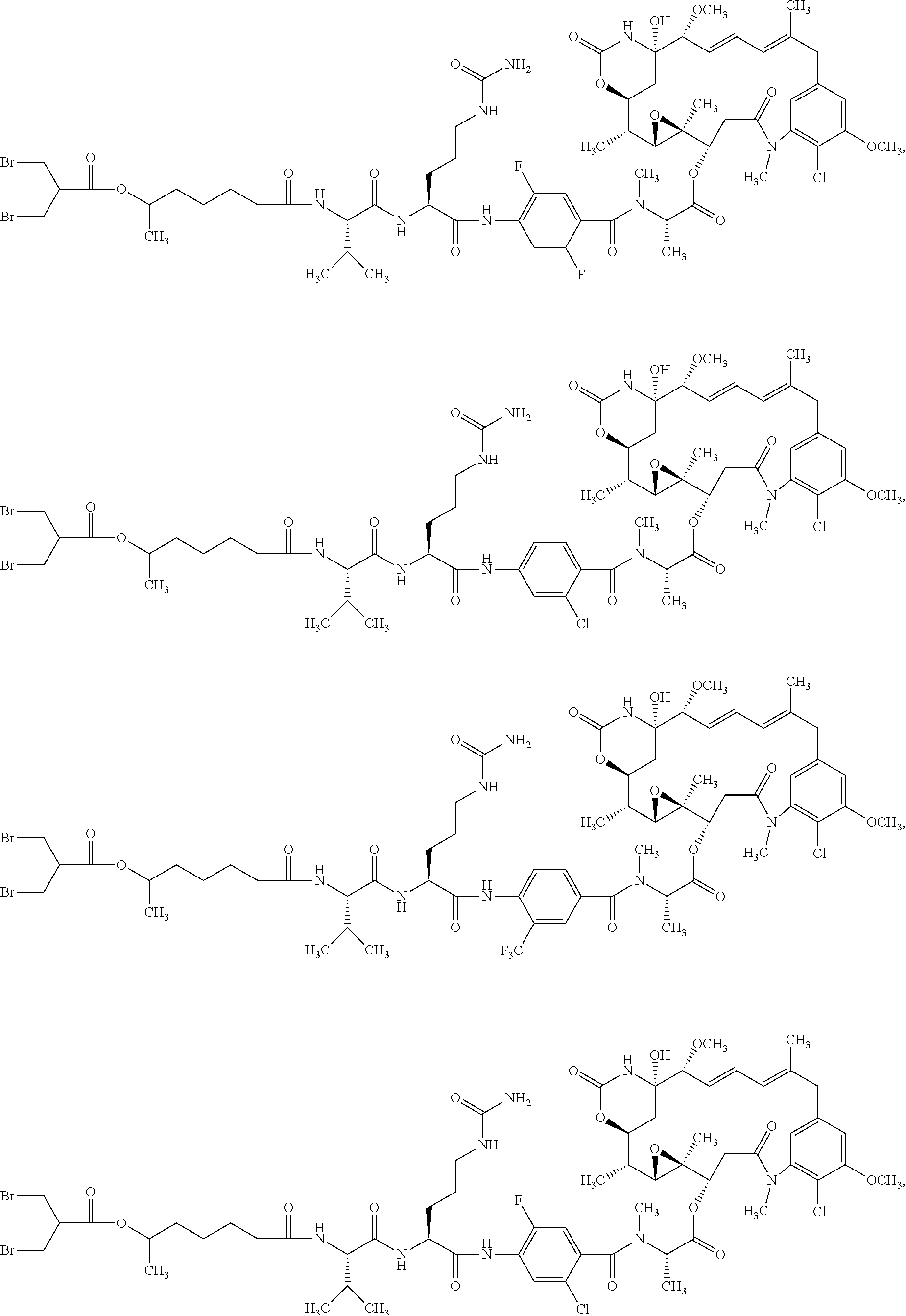

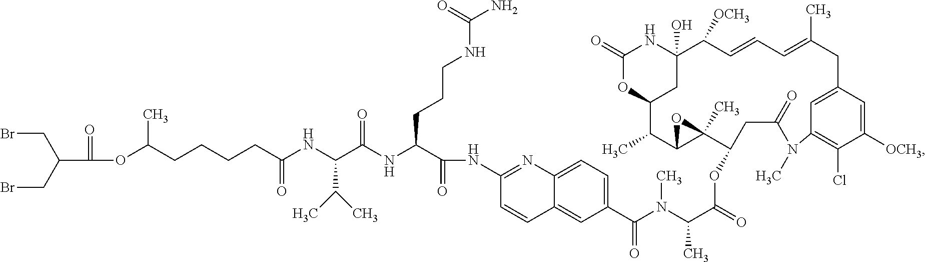 [SMILES: COc1cc2cc(c1Cl)N(C)C(=O)C[C@H](OC(=O)[C@H](C)N(C)C(=O)c1ccc3nc(NC(=O)[C@H](CCCNC(N)=O)NC(=O)[C@@H](NC(=O)CCCCC(C)OC(=O)C(CBr)CBr)C(C)C)ccc3c1)[C@]1(C)O[C@H]1[C@H](C)[C@@H]1C[C@@](O)(NC(=O)O1)[C@H](OC)/C=C/C=C(\C)C2]

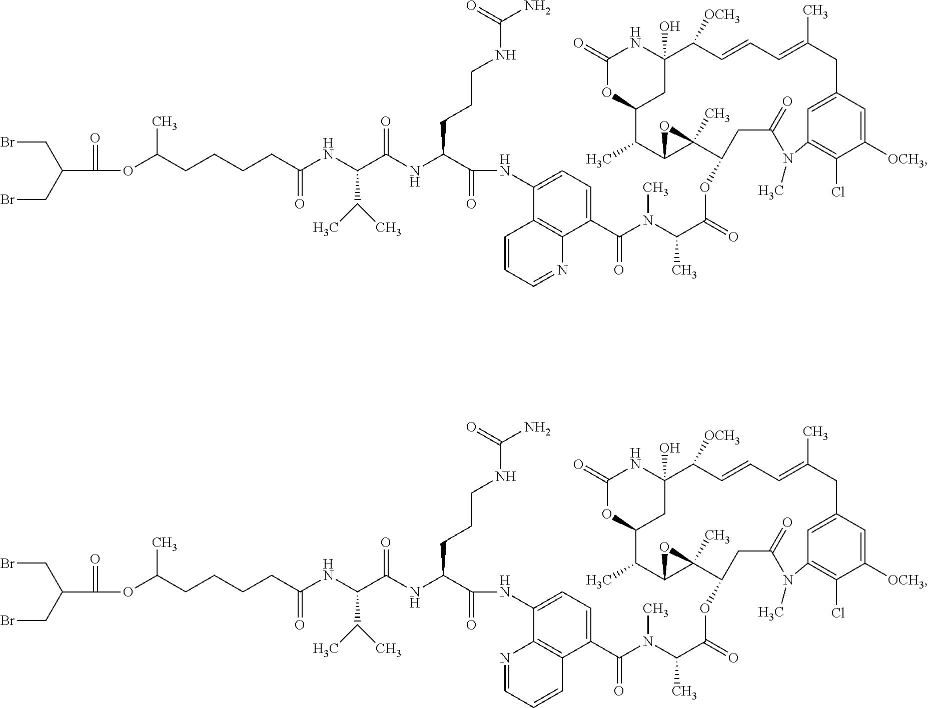 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(C)OC(=O)C(CBr)CBr)C(C)C)c3cccnc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(C)OC(=O)C(CBr)CBr)C(C)C)c3ncccc13)[C@]1(C)O[C@H]1[C@H](C)[C@@H]1C[C@@](O)(NC(=O)O1)[C@H](OC)/C=C/C=C(\C)C2]

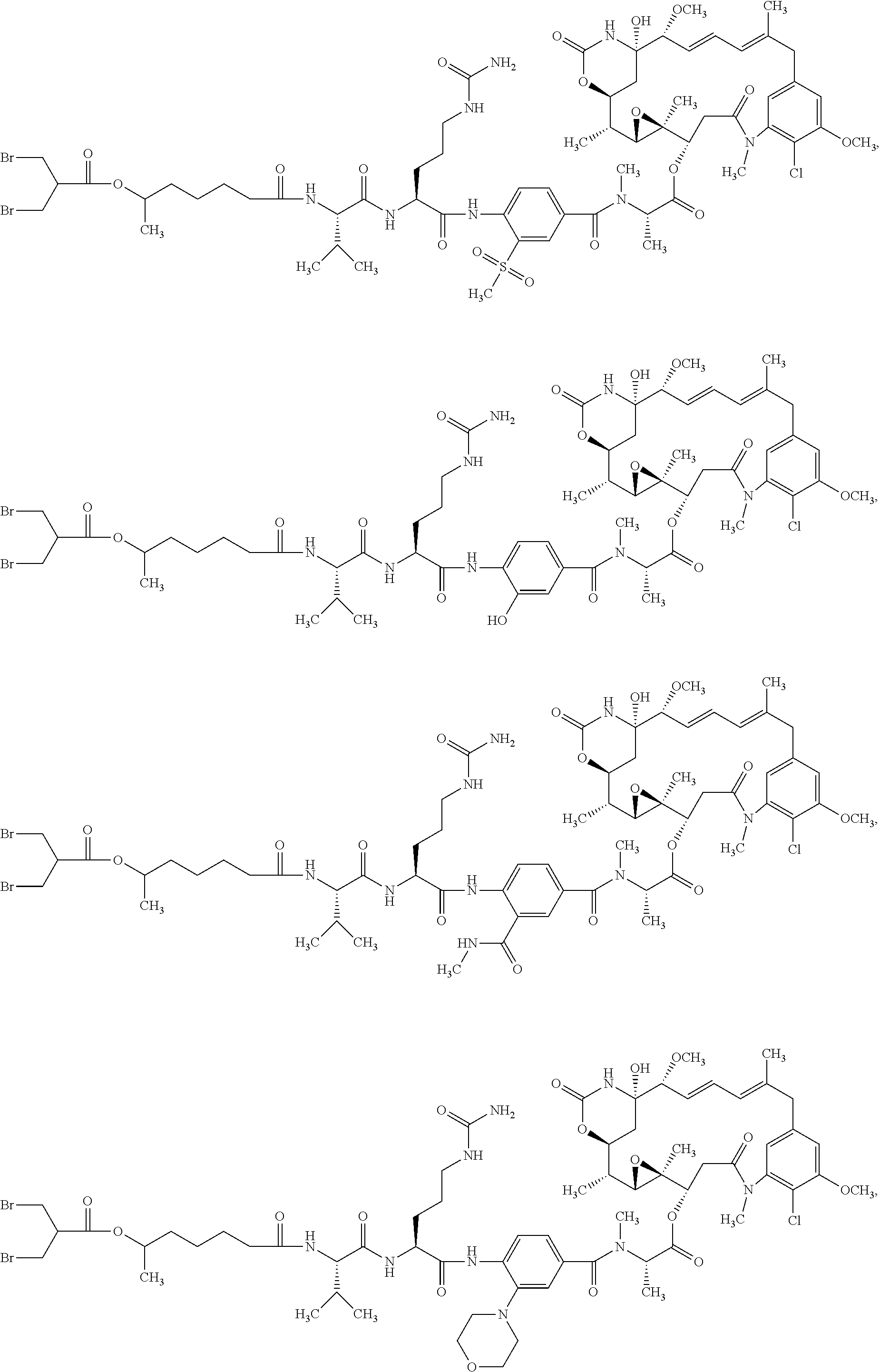

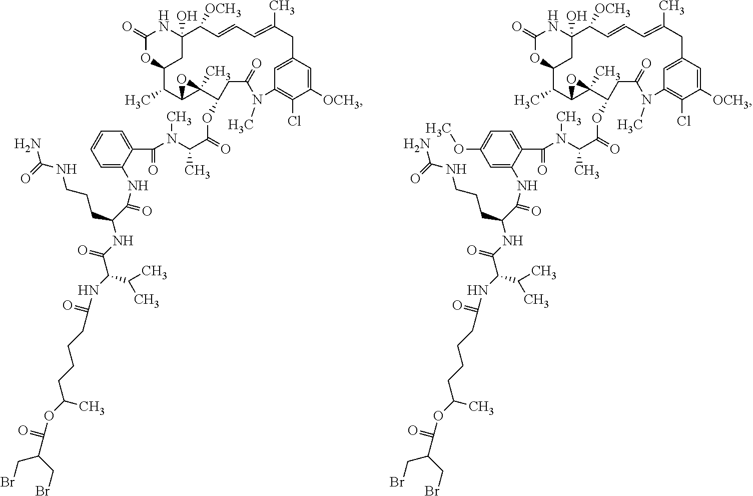 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=O)CCCCC(C)OC(=O)C(CBr)CBr)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=O)CCCCC(C)OC(=O)C(CBr)CBr)C(C)C)c1]

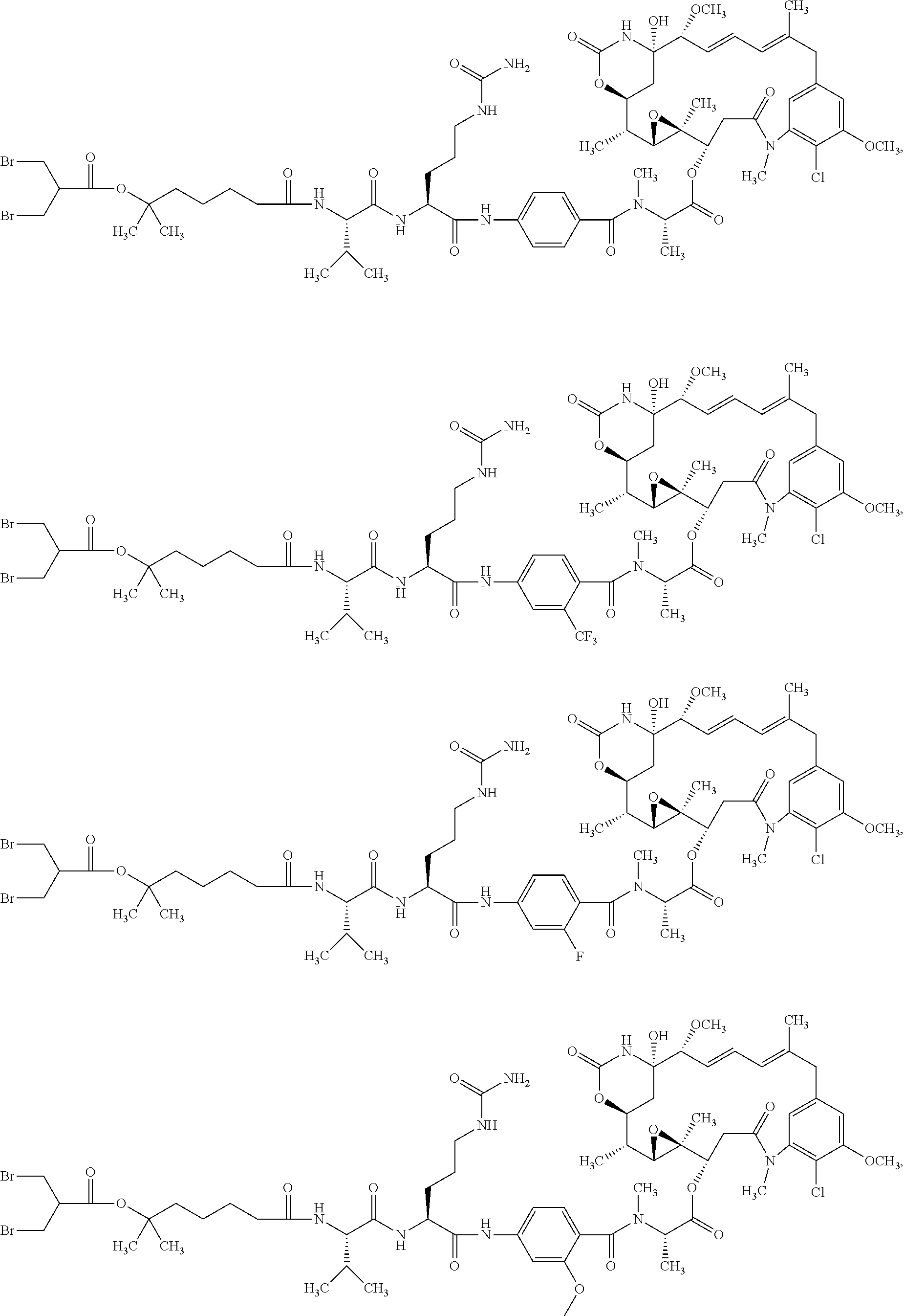

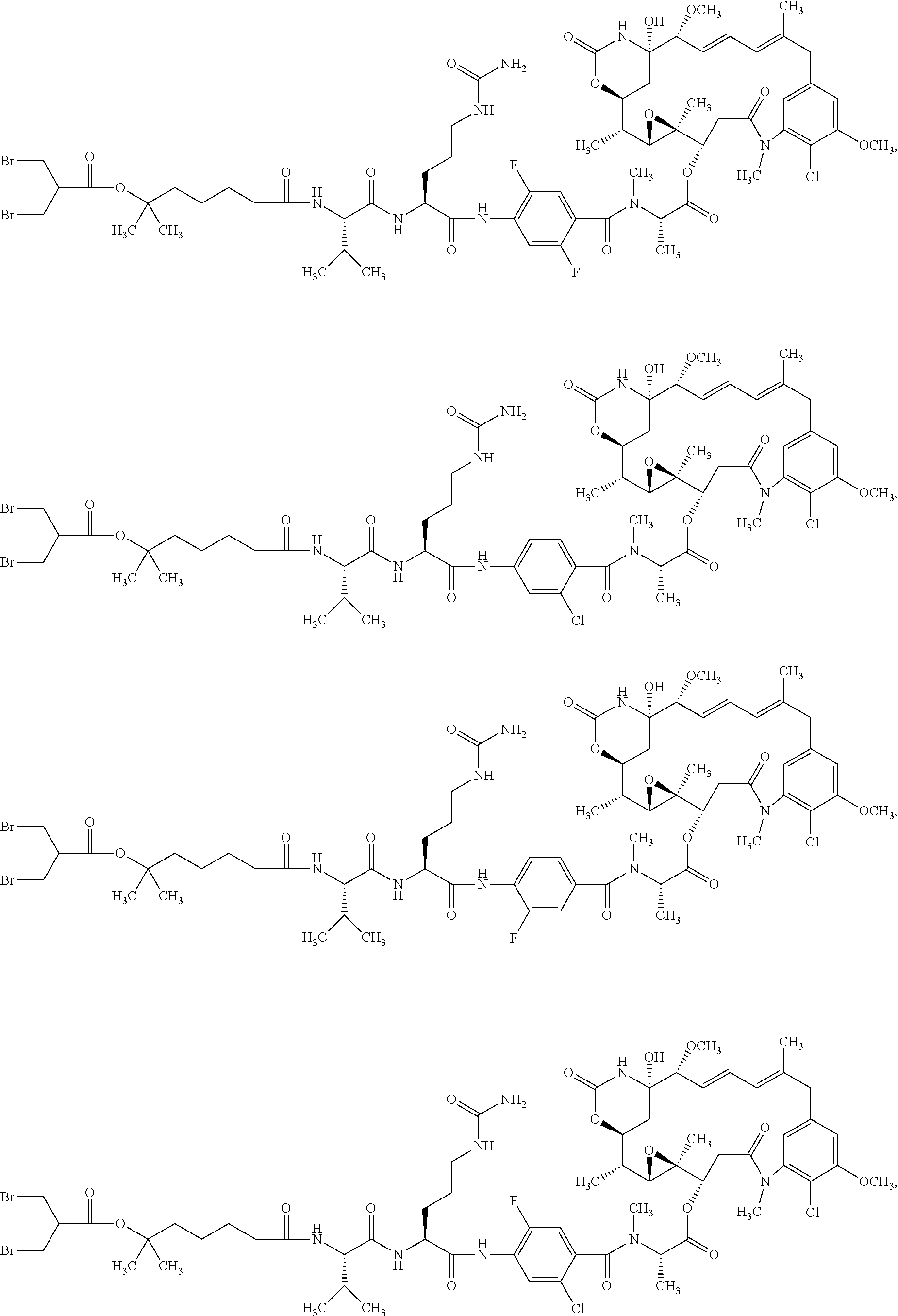

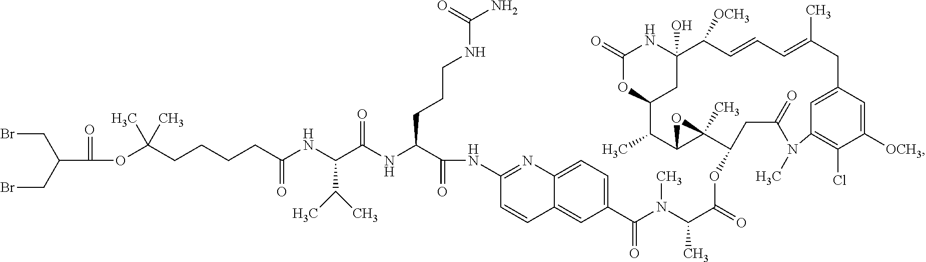 [SMILES: COc1cc2cc(c1Cl)N(C)C(=O)C[C@H](OC(=O)[C@H](C)N(C)C(=O)c1ccc3nc(NC(=O)[C@H](CCCNC(N)=O)NC(=O)[C@@H](NC(=O)CCCCC(C)(C)OC(=O)C(CBr)CBr)C(C)C)ccc3c1)[C@]1(C)O[C@H]1[C@H](C)[C@@H]1C[C@@](O)(NC(=O)O1)[C@H](OC)/C=C/C=C(\C)C2]

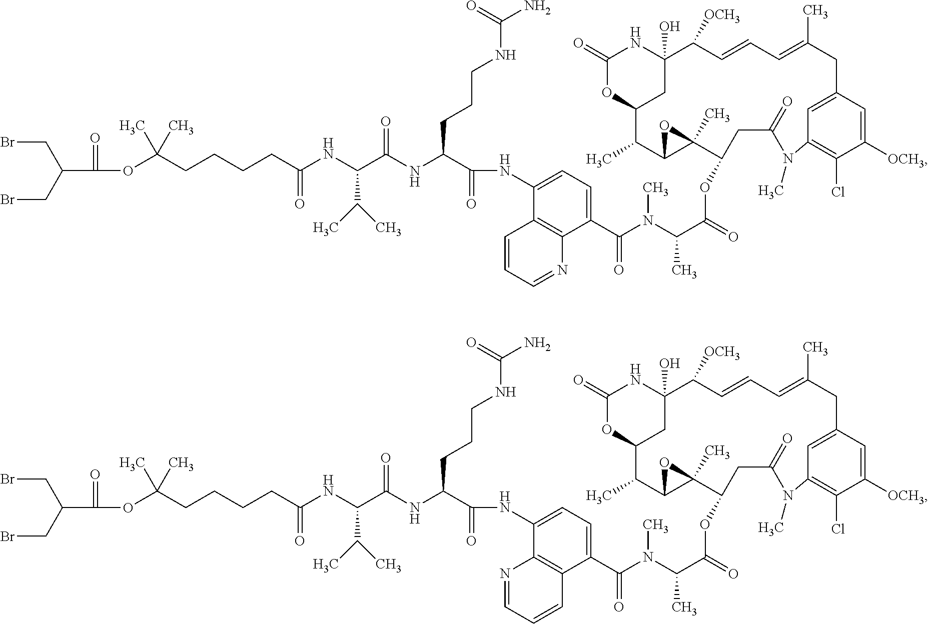 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(C)(C)OC(=O)C(CBr)CBr)C(C)C)c3cccnc13)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCC(C)(C)OC(=O)C(CBr)CBr)C(C)C)c3ncccc13)[C@]1(C)O[C@H]1[C@H](C)[C@@H]1C[C@@](O)(NC(=O)O1)[C@H](OC)/C=C/C=C(\C)C2]

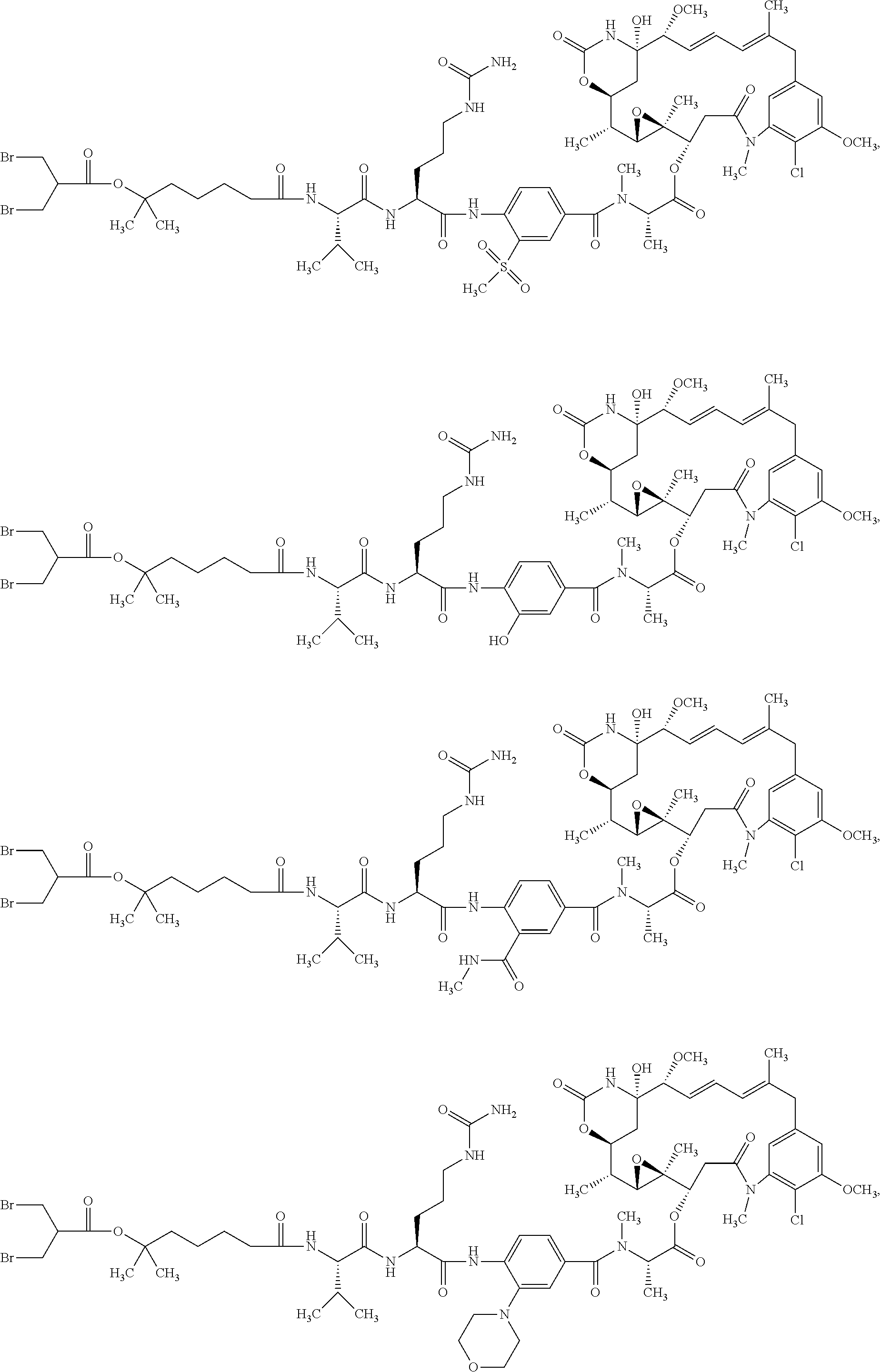

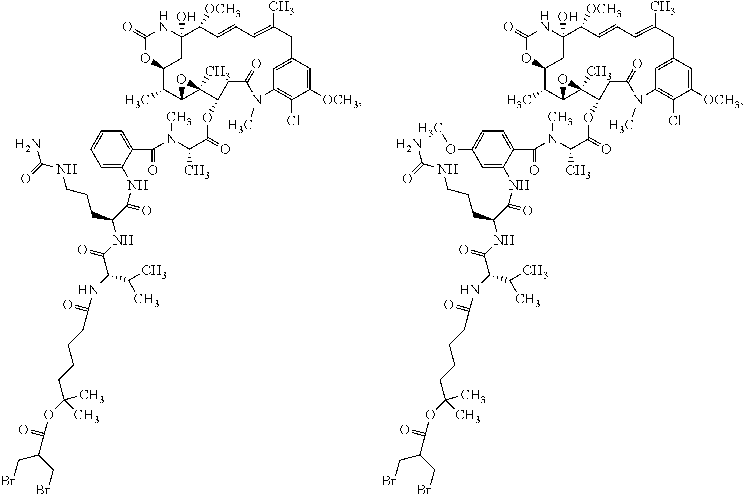 [SMILES: COc1cc2cc(c1Cl)N(C)C(=O)C[C@H](OC(=O)[C@H](C)N(C)C(=O)c1ccccc1NC(=O)[C@H](CCCNC(N)=O)NC(=O)[C@@H](NC(=O)CCCCC(C)(C)OC(=O)C(CBr)CBr)C(C)C)[C@]1(C)O[C@H]1[C@H](C)[C@@H]1C[C@@](O)(NC(=O)O1)[C@H](OC)/C=C/C=C(\C)C2 ; COc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c(NC(=O)[C@H](CCCNC(N)=O)NC(=O)[C@@H](NC(=O)CCCCC(C)(C)OC(=O)C(CBr)CBr)C(C)C)c1]

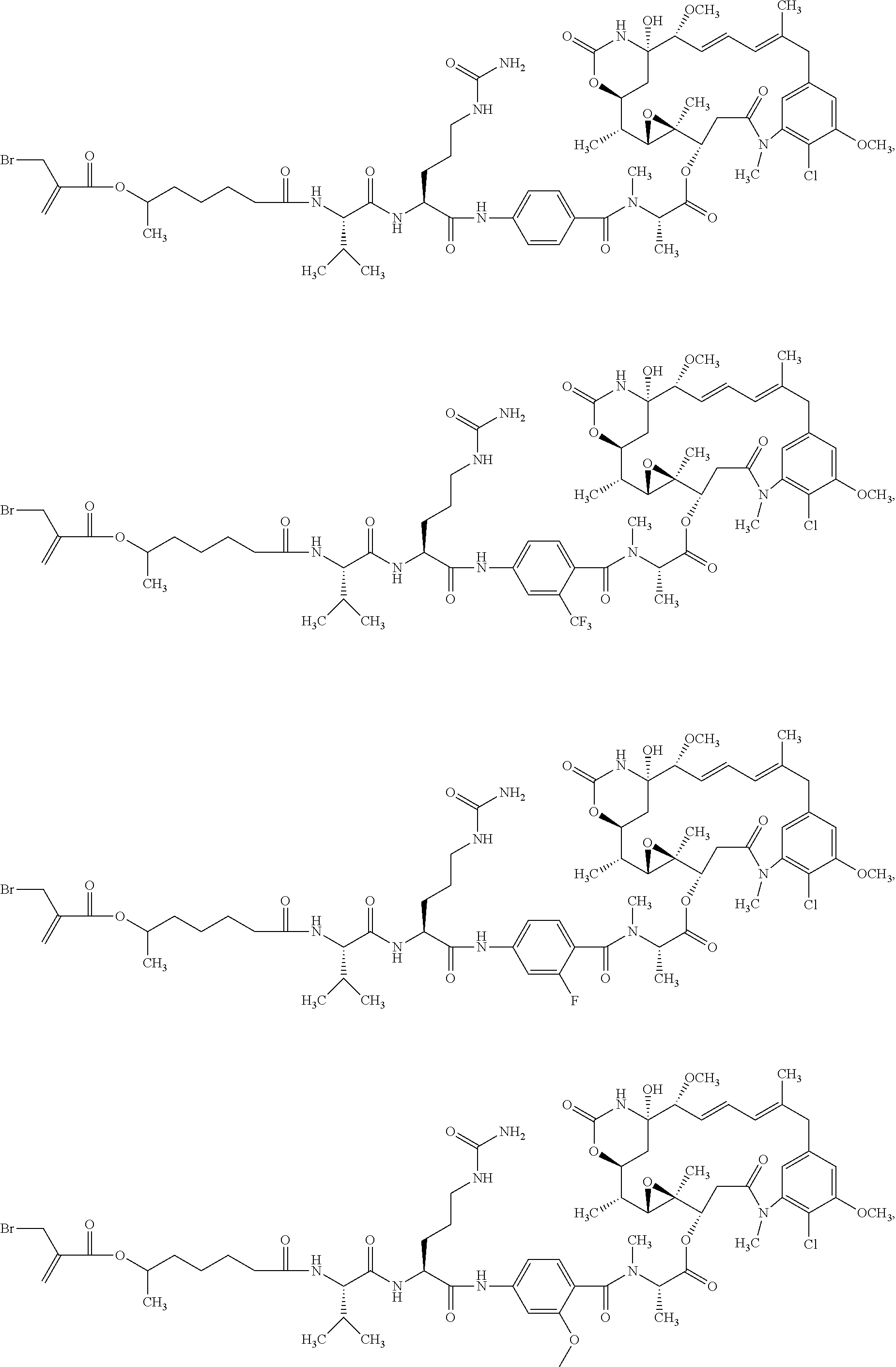

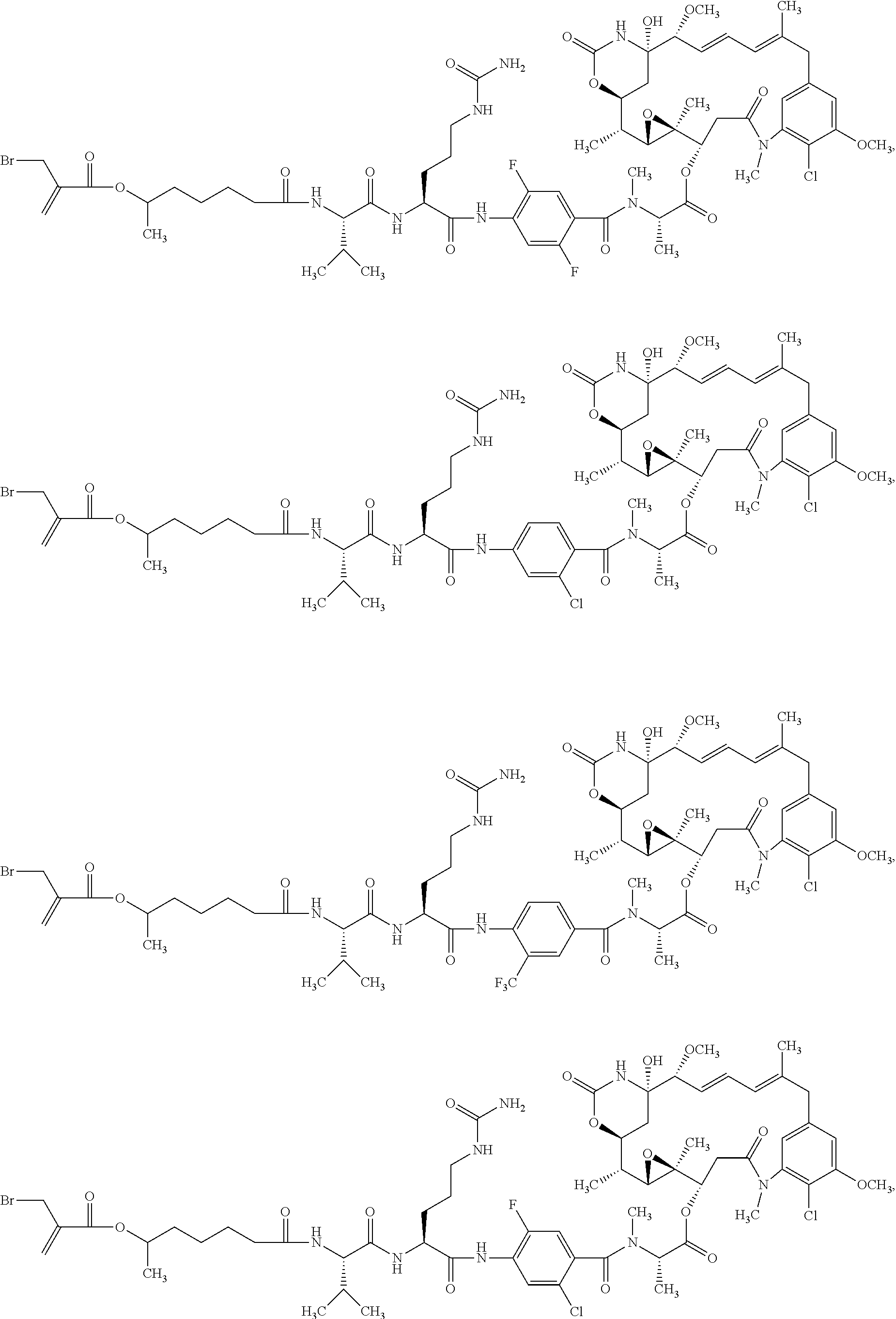

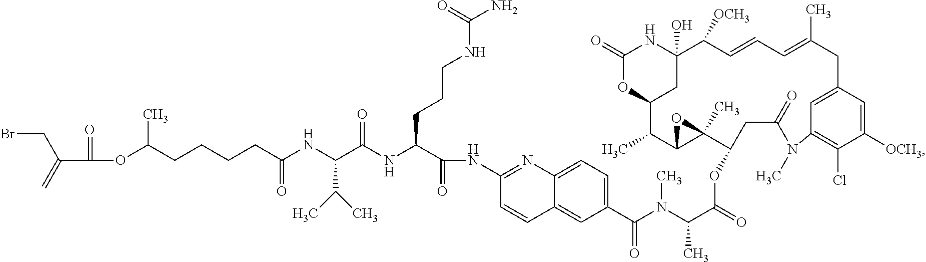 [SMILES: C=C(CBr)C(=O)OC(C)CCCCC(=O)N[C@H](C(=O)N[C@@H](CCCNC(N)=O)C(=O)Nc1ccc2cc(C(=O)N(C)[C@@H](C)C(=O)O[C@H]3CC(=O)N(C)c4cc(cc(OC)c4Cl)C/C(C)=C/C=C/[C@@H](OC)[C@@]4(O)C[C@H](OC(=O)N4)[C@@H](C)[C@@H]4O[C@@]34C)ccc2n1)C(C)C]

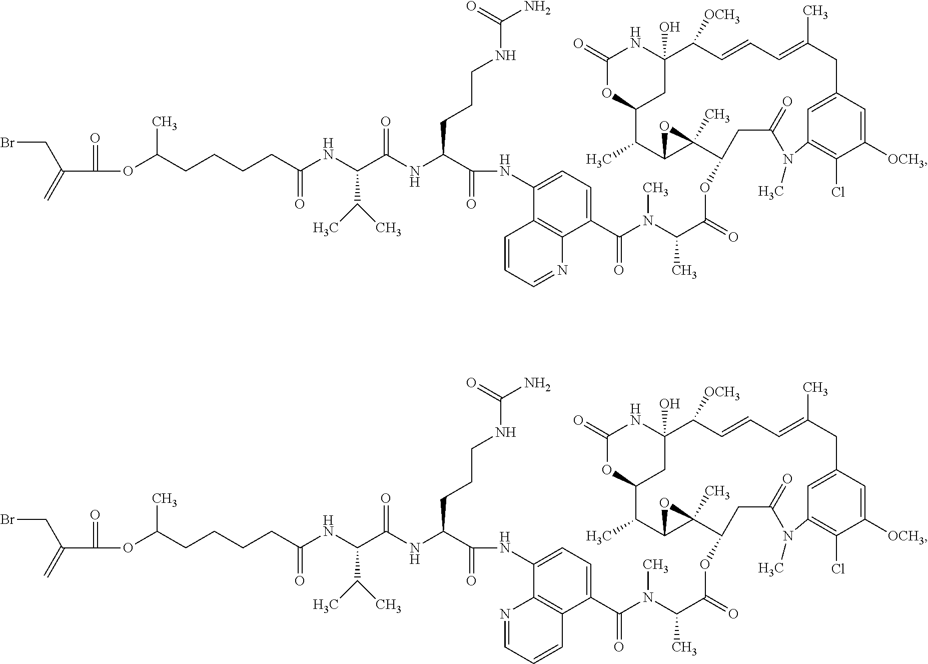 [SMILES: C=C(CBr)C(=O)OC(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c2ncccc12)C(C)C ; C=C(CBr)C(=O)OC(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)c2cccnc12)C(C)C]

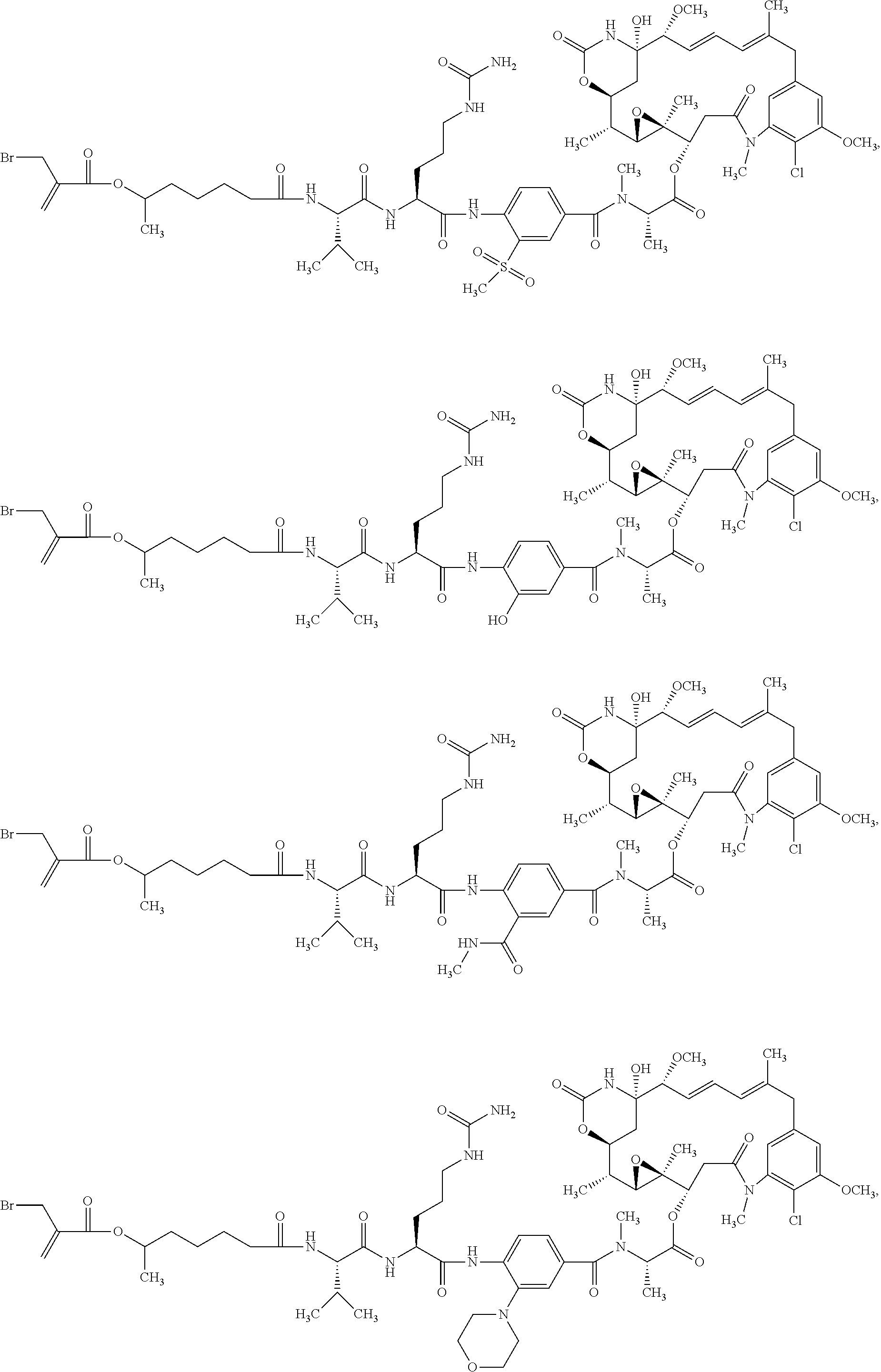

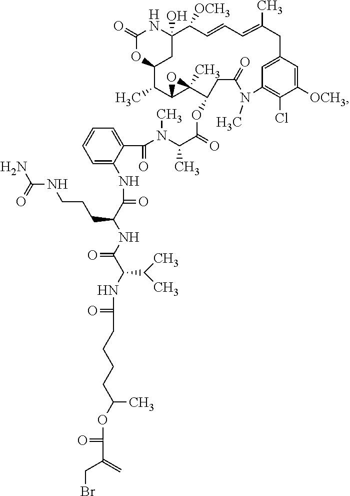 [SMILES: C=C(CBr)C(=O)OC(C)CCCCC(=O)N[C@H](C(=O)N[C@@H](CCCNC(N)=O)C(=O)Nc1ccccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)C(C)C]

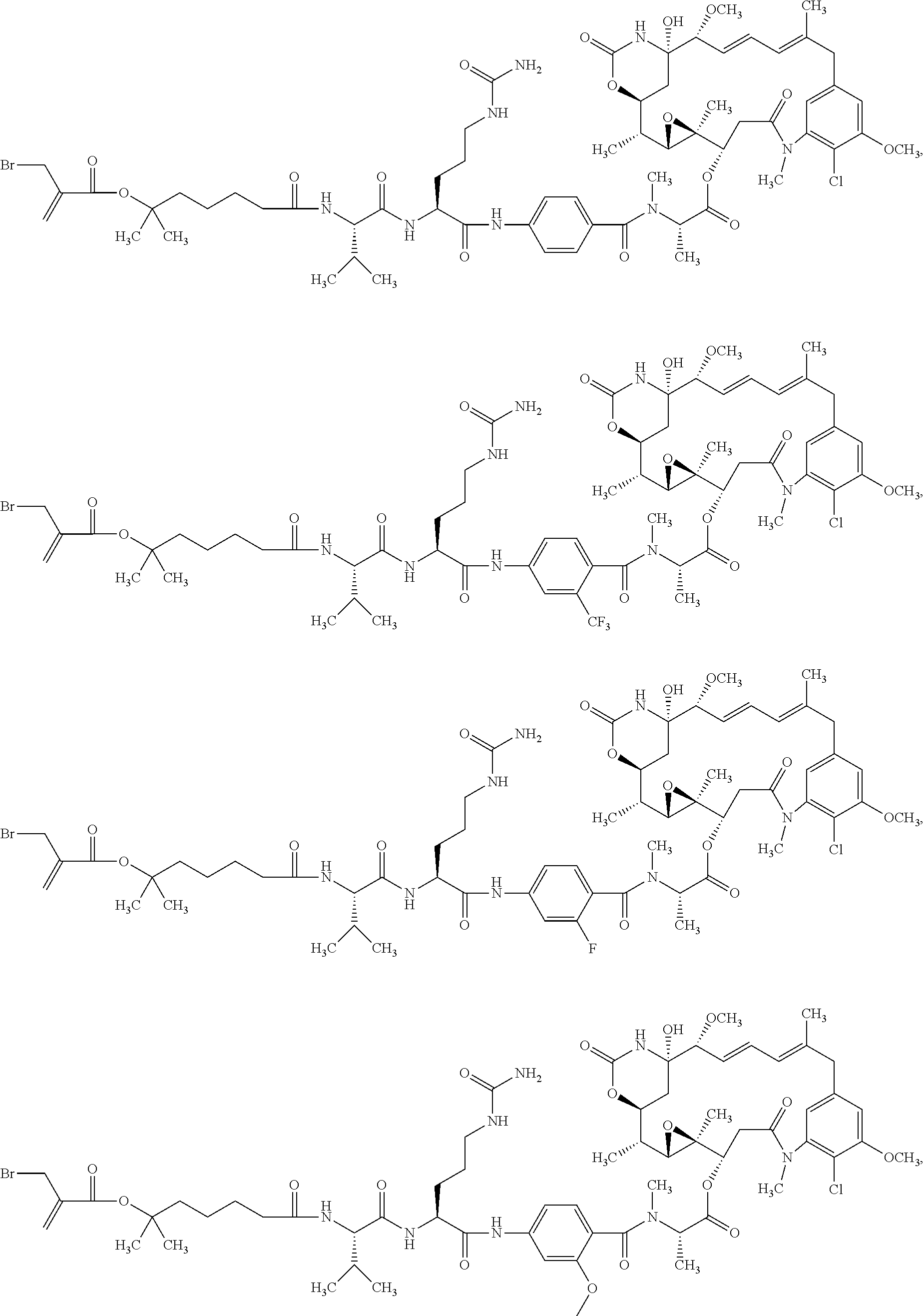

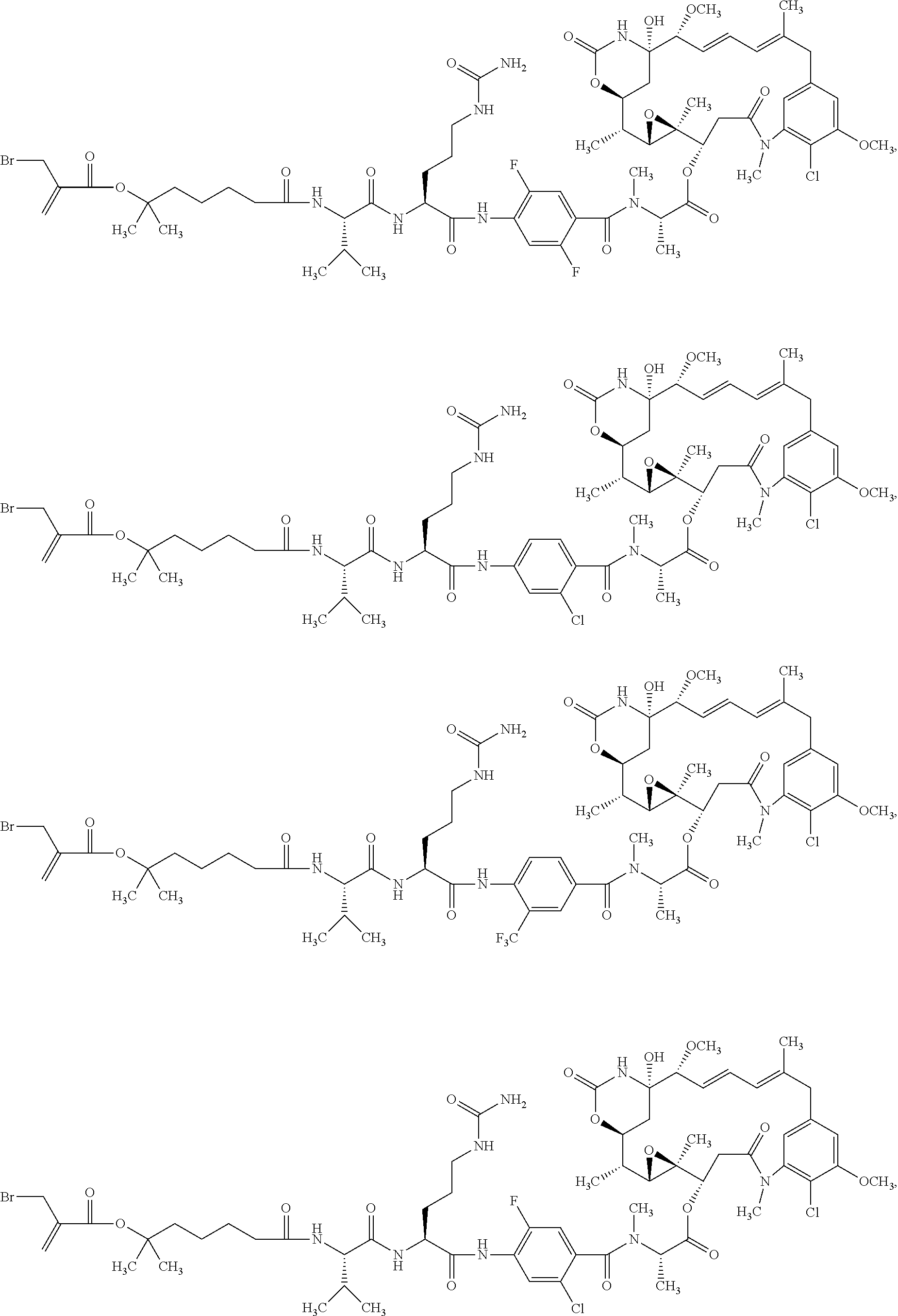

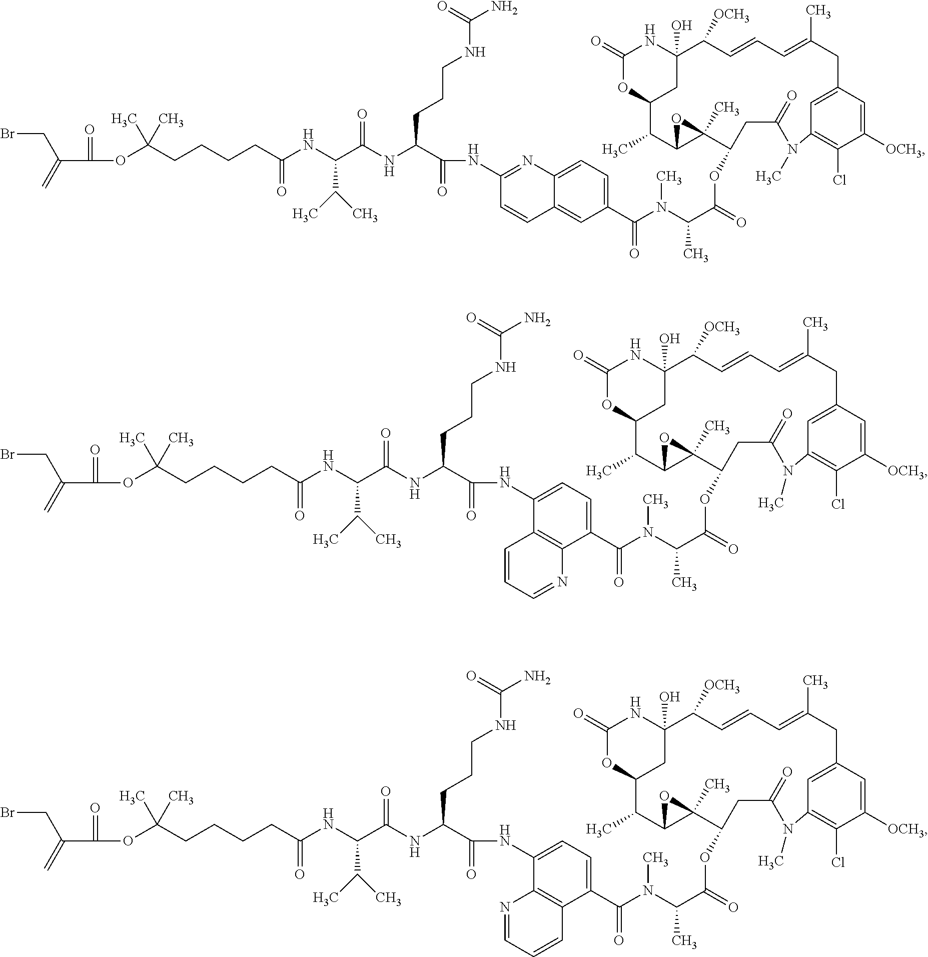

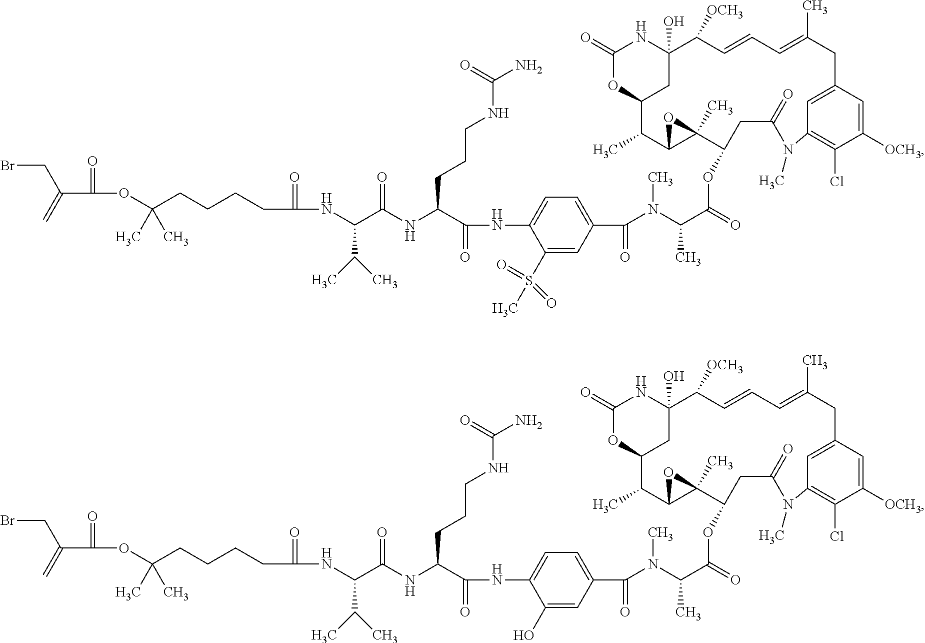 [SMILES: C=C(CBr)C(=O)OC(C)(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)cc1S(C)(=O)=O)C(C)C ; C=C(CBr)C(=O)OC(C)(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)cc1O)C(C)C]

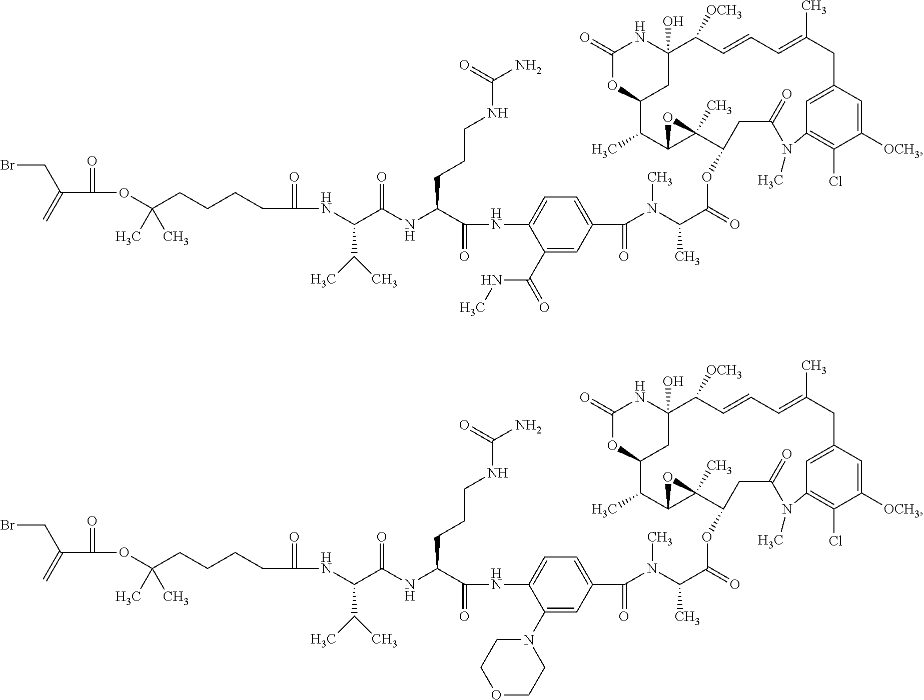 [SMILES: C=C(CBr)C(=O)OC(C)(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)cc1C(=O)NC)C(C)C ; C=C(CBr)C(=O)OC(C)(C)CCCCC(=O)N[C@H](C(=O)N[C@@H](CCCNC(N)=O)C(=O)Nc1ccc(C(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)cc1N1CCOCC1)C(C)C]

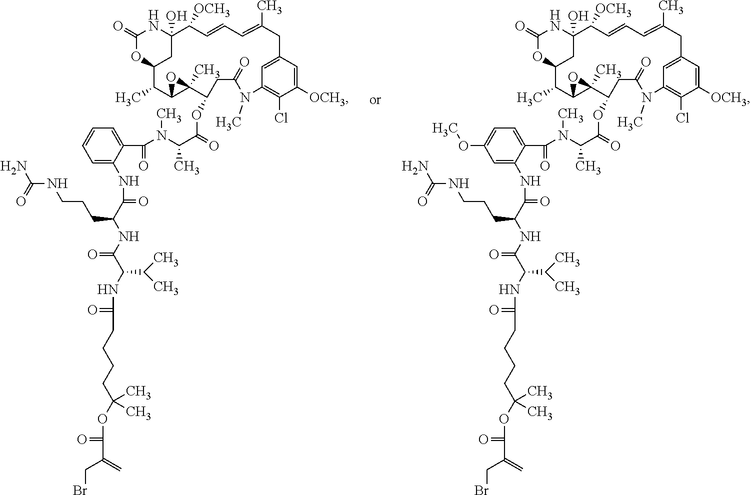 [SMILES: C=C(CBr)C(=O)OC(C)(C)CCCCC(=O)N[C@H](C(=O)N[C@@H](CCCNC(N)=O)C(=O)Nc1ccccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)C(C)C ; C=C(CBr)C(=O)OC(C)(C)CCCCC(=O)N[C@H](C(=O)N[C@@H](CCCNC(N)=O)C(=O)Nc1cc(OC)ccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C)C(C)C]

In some embodiments, the compound of Formula P1 is a compound having one of the following structures:

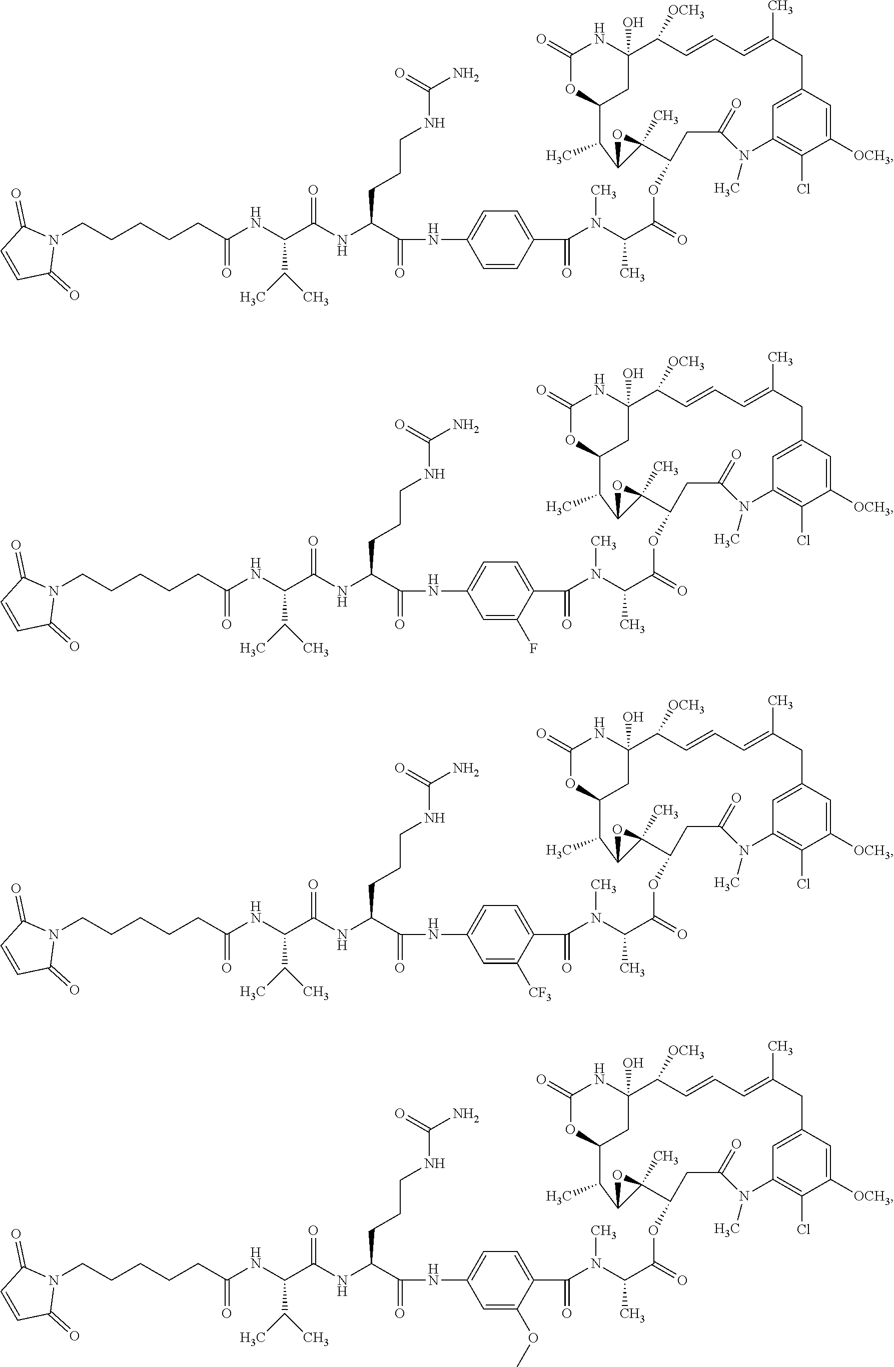

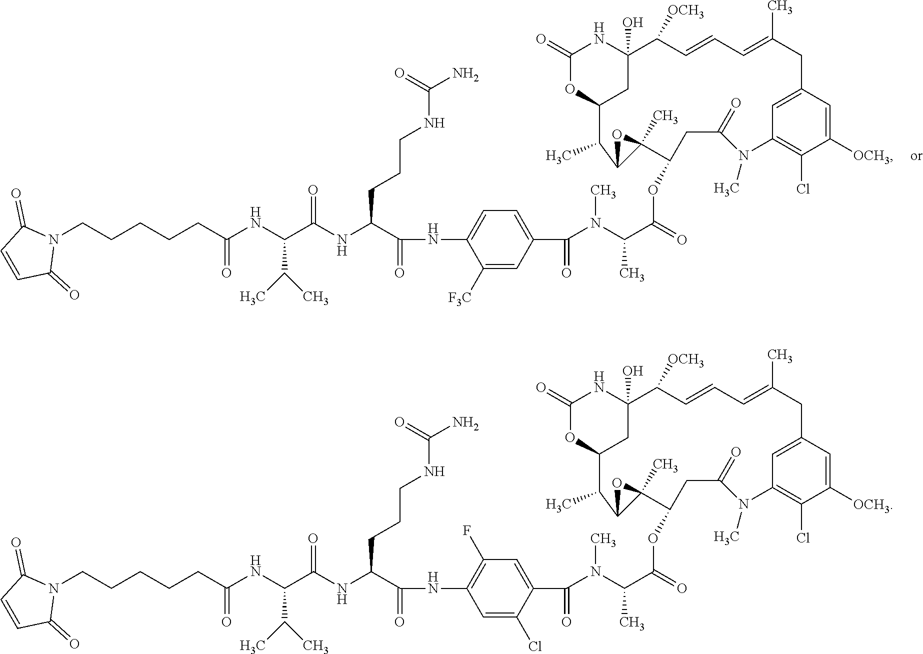 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCN3C(=O)C=CC3=O)C(C)C)c(C(F)(F)F)c1)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)c1cc(F)c(NC(=O)[C@H](CCCNC(N)=O)NC(=O)[C@@H](NC(=O)CCCCCN3C(=O)C=CC3=O)C(C)C)cc1Cl)[C@]1(C)O[C@H]1[C@H](C)[C@@H]1C[C@@](O)(NC(=O)O1)[C@H](OC)/C=C/C=C(\C)C2]

Compounds of Formula P1 can by synthesized by reacting compounds of Formula P2 with the compound of Formula P3 under amide synthesis conditions. Suitable amide synthesis conditions include, but are not limited to, contacting the compound of Formula P2 in the presence of a carboxylic acid activating agent and base. Suitable activating agents include, but are not limited to EDC, HATU, HBTU, DCC, BOP, and EEDQ. Suitable bases include, but are not limited to DIEA, DBU, Tributylamine, and 2,6-Lutidine.

The compound of Formula P2 can be synthesized directly from maytansinol and alanine using known techniques (see, e.g., U.S. Pat. No. 4,308,269, which is incorporated herein by reference).

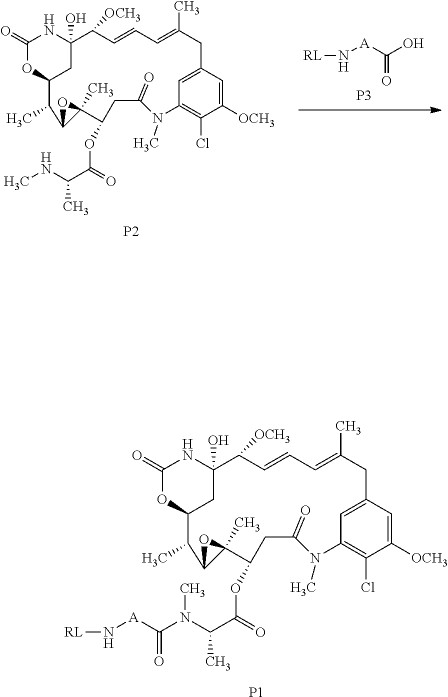

Compounds of Formula I can be synthesized by coupling compounds of Formula PP3:

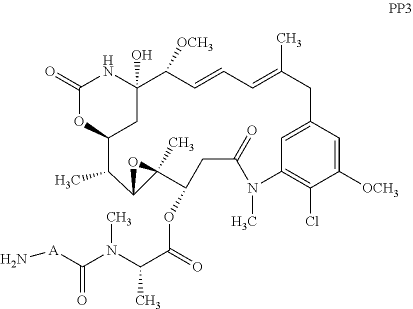

with compounds of Formula PP4 under amide synthesis conditions:

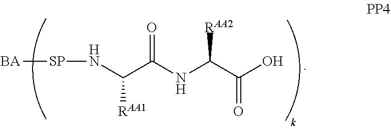

wherein:
   BA is a binding agent;
   SP is a spacer;
   R^{AA1} is an amino acid side chain;
   R^{AA2} is an amino acid side chain;
   A is arylene or heteroarylene; and
   k is an integer from 1 to 10.

Compounds of Formula PP3 can be synthesized by contacting compounds of Formula PP5 with a suitable reducing agent:

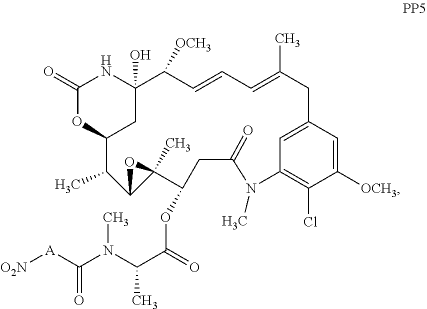

wherein A is arylene or heteroarylene.

In some embodiments, the suitable reducing agent includes a metal, a metal foil, a metal powder, a metal amalgam, or metal filings. In certain embodiments, the metal is selected from zinc, iron, aluminum, palladium, or Raney nickel.

For example, in some embodiments, the following reducing agent conditions are employed. With respect to the amount of compound PP5, for example, in some of the methods herein about twenty (20) equivalents of zinc dust and forty (40) equivalents of acetic acid were combined. In some examples, the reducing reaction was conducted at room temperature for about from 1 to 20 hours. In some of these examples, the aforementioned acetic acid is substituted with another suitable mild acid or proton donor. Examples of suitable mild acids or proton donors include, but are not limited to formic acid, pTsOH, and NH₄Cl. In some of these examples, the reducing metal is substituted with a suitable reducing agent selected from iron, aluminum, palladium, or Raney nickel. In some of these examples, suitable solvents includes those solvents having 10-50% water (by volume) in a miscible organic solvent. Example miscible organic solvents include, but are not limited to THF, Dioxane, and diethyl ether. In some examples, the reducing reactions set forth herein are conducted at reaction temperatures which range from 0 to 50° C. In some examples, the reducing reactions set forth herein are conducted at reaction times which range from 1 to 40 hours.

Suitable acids include, but are not limited to, acetic acid.

In some embodiments, A is:

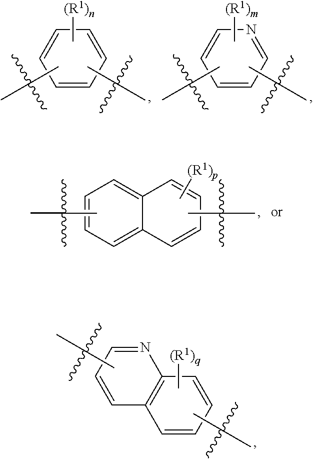

wherein:
   R¹ is, independently at each occurrence, alkyl, alkenyl, alkynyl, aryl, alkaryl, aralkyl, halo, heteroaryl, heterocycloalkyl, hydroxyl, cyano, nitro,

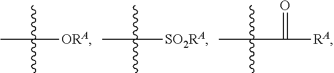

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, the compound of Formula PP5 is a compound of the Formula PP5A:

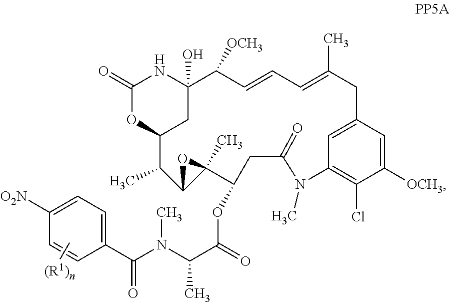

wherein R¹ and n are as defined herein.

In some embodiments, R¹ is, independently, alkyl, alkoxy, heteroalkyl, halo, haloalkyl, or haloalkoxy. In some embodiments, R¹ is, independently, C₁₋₆ alkyl, C₁₋₆ alkoxy, C₁₋₆ haloalkyl, C₁₋₆ haloalkoxy, or halo. In some embodiments, R¹ is, independently, C₁₋₆ alkyl or C₁₋₆ alkoxy. In some embodiments, R¹ is, independently, alkoxy. In some embodiments, R¹ is, independently, methoxy, ethoxy, propoxy. In some embodiments, n, m, p, or q is 0, 1 or 2. In some embodiments, n, m, p, or q is 0 or 1. In some embodiments, n, m, p, or q is 0.

In some embodiments, the compound of Formula PP5 is a compound of the Formula PP5A:

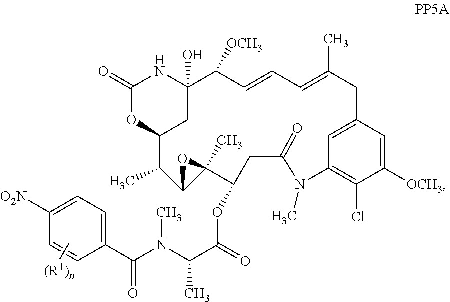

wherein:
   R¹ is, independently at each occurrence, halo or trifluoromethyl; and
   n is 0, 1, or 2.

In some embodiments, the compound of Formula PP5 is a compound of the Formula PP5A2:

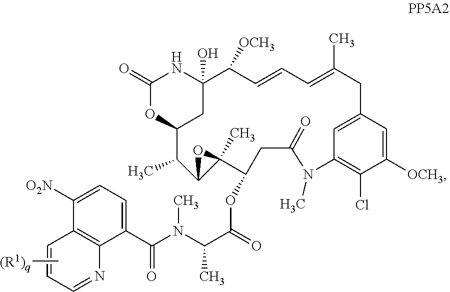

wherein:
   R¹ is, independently at each occurrence, halo or trifluoromethyl; and
   q is an integer from 0 to 5

In some embodiments, the compound of Formula PP5 is a compound of the Formula PP5A3:

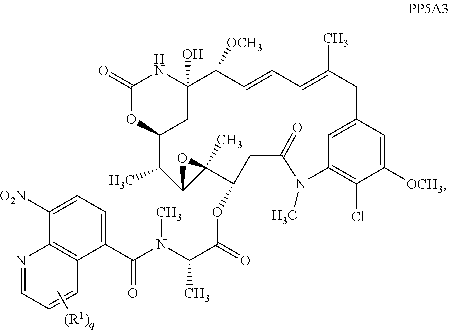

wherein:
   R¹ is, independently at each occurrence, halo or trifluoromethyl; and q is an integer from 0 to 5.In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen.

In some embodiments, the compound of Formula PP5 is a compound of the Formula PP5A4:

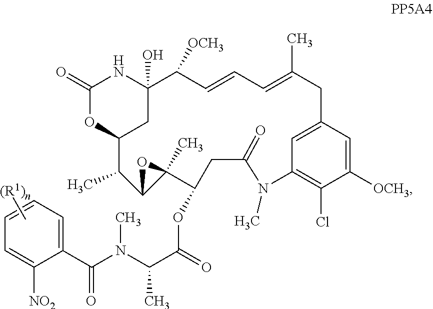

wherein R¹ and n are as defined herein.

Compounds of Formula PP5 can be synthesized by contacting compounds of Formula P2 with compounds of Formula PP6 under amide synthesis conditions:

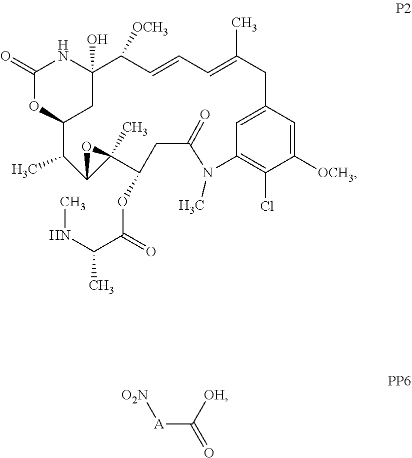

Suitable compounds of Formula PP6 include, but are not limited to, 3-nitro-benzoic acid, 3-chloro-5-nitro-benzoic acid, 3-fluoro-5-nitro-benzoic acid, 3-nitro-1-naphthalenecarboxylic acid, 2-fluoro-5-nitro-benzoic acid, 3-(dimethylamino)-5-nitro-benzoic acid, 3-ethoxy-5-nitro-benzoic acid, 2-methoxy-5-nitro-benzoic acid, 4-methoxy-3-nitro-benzoic acid, 2,6-difluoro-3-nitro-benzoic acid, 2-chloro-6-fluoro-3-nitro-benzoic acid, 6-chloro-2-fluoro-3-nitro-benzoic acid, 2-chloro-4-fluoro-5-nitro-benzoic acid, 4-chloro-2-fluoro-5-nitro-benzoic acid, 2-ethoxy-5-nitro-benzoic acid, 2-(methylamino)-3-nitro-benzoic acid, 6-nitro-8-quinolinecarboxylic acid, 4-(dimethylamino)-3-nitro-benzoic acid hydrochloride (1:1), 2-methyl-nitro-benzoic acid, 3-methyl-4-nitro-benzoic acid, 4-nitro-1-naphthalenecarboxylic acid, 4-nitro-1-naphthalenecarboxylic acid, 2,6-dimethyl-4-nitro-benzoic acid, 3-fluoro-4-nitro-benzoic acid, 3-chloro-4-nitro-benzoic acid, 3-bromo-4-nitro-benzoic acid, 3-cyano-4-nitro-benzoic acid, 3-cyclopropyl-4-nitro-benzoic acid, 3-methoxy-4-nitro-benzoic acid, 2-methoxy-4-nitro-benzoic acid, 5-chloro-2-methyl-4-nitro-benzoic acid, 8-nitro-5-isoquinolinecarboxylic acid, 5-nitro-8-quinolinecarboxylic acid, 8-nitro-5-quinolinecarboxylic acid, 2,5-difluoro-4-nitro-benzoic acid, 2-(dimethylamino)-4-nitro-benzoic acid, 2-chloro-5-fluoro-4-nitro-benzoic acid, 3-(dimethylamino)-4-nitro-benzoic acid, 2-[(1-methylethyl)thio]-4-nitro-benzoic acid, 4-nitro-3-(trifluoromethyl)-benzoic acid, 4-nitro-2-(trifluoromethyl)-benzoic acid, 3,5-dimethoxy-4-nitro-benzoic acid, 4-nitro-2-(propylamino)-benzoic acid, 3-(difluoromethoxy)-4-nitro-benzoic acid, 2-(2-fluoro-phenyl)-4-nitro-benzoic acid, 4-nitro-2-(4-pyridinyl)-benzoic acid, 4-nitro-3-(4-pyridinyl)-benzoic acid, or 4-nitro-2-(1-pyrrolidinyl)-benzoic acid.

Suitable compounds of Formula PP6 include compounds having any one of the following formula:

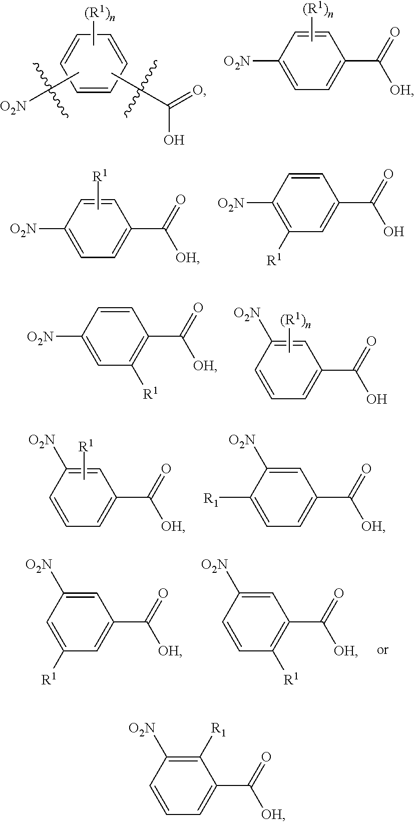

wherein R¹ is, independently at each occurrence, C₁₋₆ alkyl, C₁₋₆ alkoxy, halo, C₁₋₆ haloalkyl, or C₁₋₆ haloalkoxy, wherein n is 0, 1, 2, 3, or 4. In certain of these embodiments, R¹ is methoxy or methyl. In some specific embodiments, R¹ is methoxy, fluoro, or trifluoromethyl. In certain embodiments, n is 1 or 2. In some of these embodiments, n is 1. In some embodiments, R¹ is fluoro, chloro, bromo, or iodo.

In some embodiments, R¹ is 1-methylethyl-thiol, phenyl, 2-fluorophenyl, pyridinyl, 4-pyridinyl, pyrrolidinyl, or 1-pyrrolidinyl. In some embodiments, R¹ is trifluoromethyl. In some embodiments, R¹ is methoxy. In some embodiments, R¹ is fluoro. In some embodiments, R¹ is hydrogen.

In some embodiments, provided herein are compounds of Formula PP5:

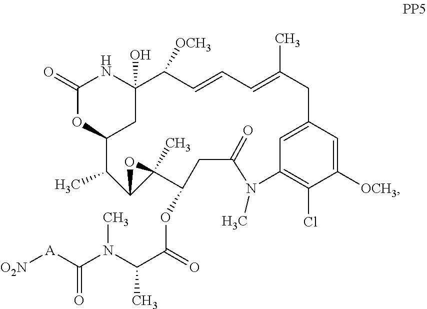

wherein A is arylene or heteroarylene.

In some embodiments, the compound of Formula PP5 is a compound selected from

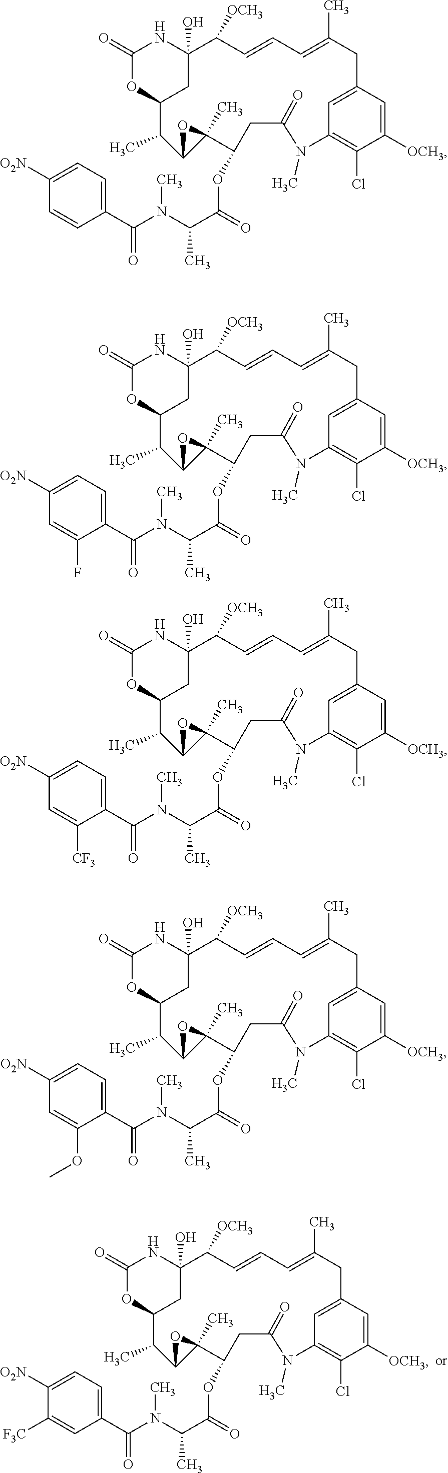

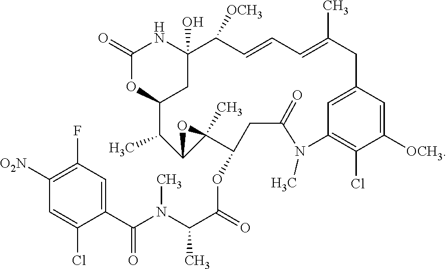 [SMILES: COc1cc2cc(c1Cl)N(C)C(=O)C[C@H](OC(=O)[C@H](C)N(C)C(=O)c1cc(F)c([N+](=O)[O-])cc1Cl)[C@]1(C)O[C@H]1[C@H](C)[C@@H]1C[C@@](O)(NC(=O)O1)[C@H](OC)/C=C/C=C(\C)C2]

In some embodiments, the compound of Formula PP5 is:

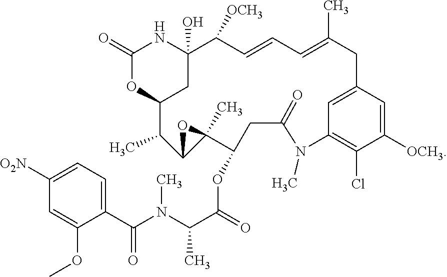 [SMILES: COc1cc([N+](=O)[O-])ccc1C(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C]

Compounds of Formula III:

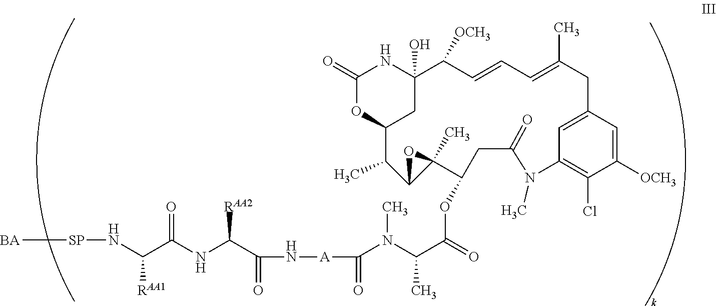

can be synthesized by contacting compounds of Formula PP:

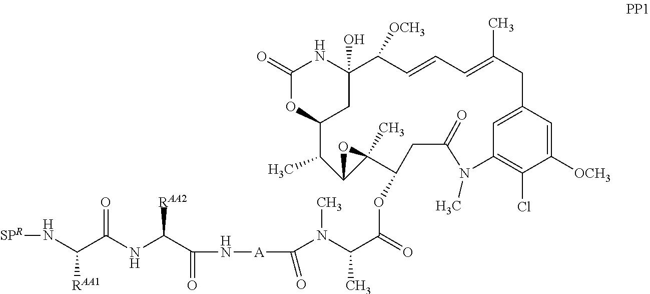

with a binding agent under conjugation conditions,
wherein:
   BA is a binding agent;
   SP is a spacer;
   SP^{R} is a reactive spacer;
   R^{AA1} is an amino acid side chain;
   R^{AA2} is an amino acid side chain;
   A is arylene or heteroarylene; and
   k is an integer from 1 to 30.

Compounds of Formula PP1 can be prepared by contacting a compound of Formula PP2 with the compound of Formula P2:

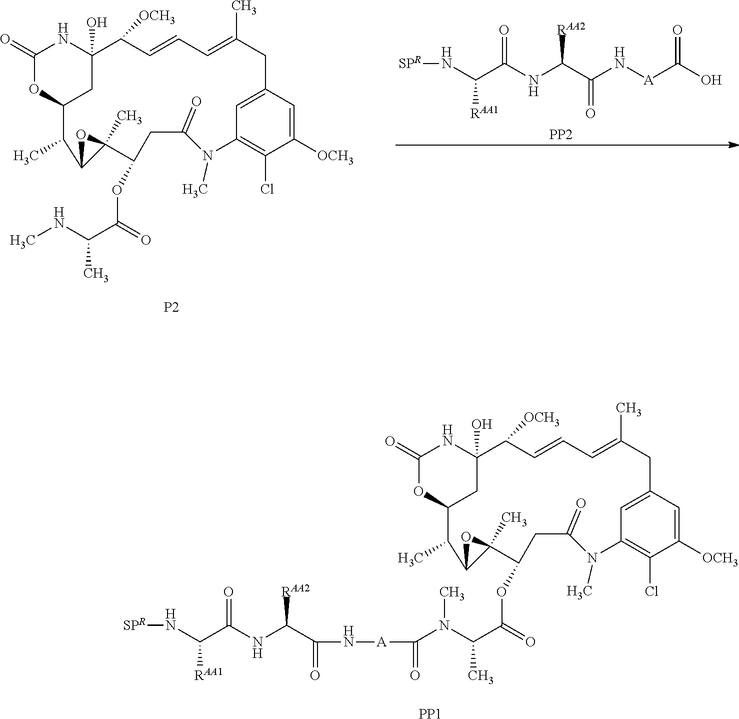

wherein:
   SP^{R} is a reactive linker;
   R^{AA1} is an amino acid side chain;
   R^{AA1} is an amino acid side chain; and
   A is arylene or heteroarylene.

Compounds of Formula PP1 can be prepared by contacting a compound of Formula PP3 with the compound of Formula PP7:

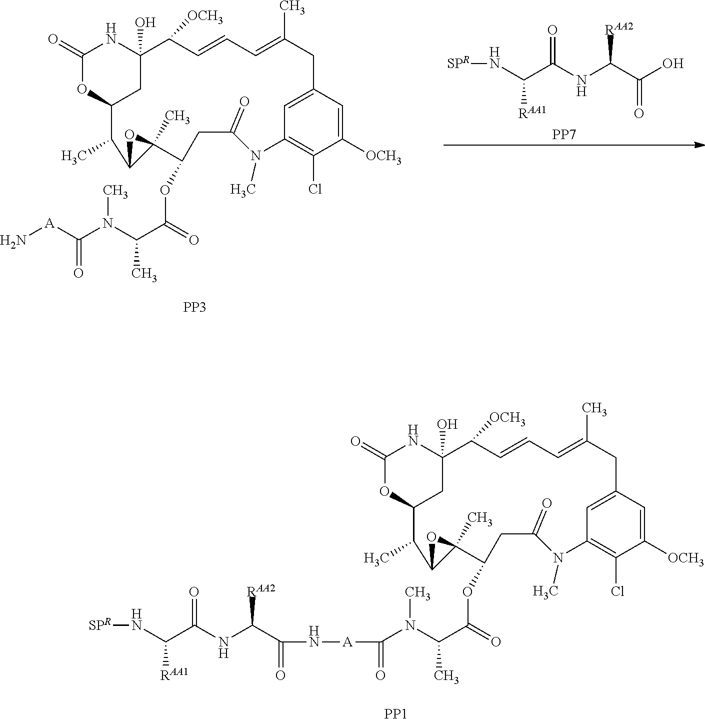

wherein:
   SP^{R} is a reactive linker;
   R^{AA1} is an amino acid side chain;
   R^{AA1} is an amino acid side chain; and
   A is arylene or heteroarylene.

Compounds of Formula PP2 can be prepared by contacting a compound of Formula PP8 with a bifunctional spacer:

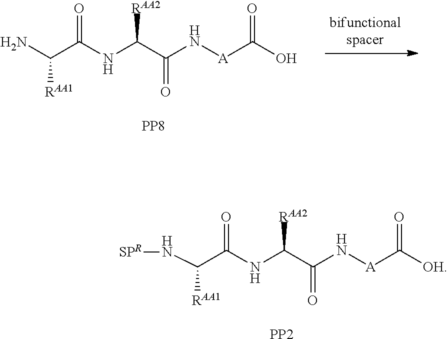

Compounds of Formula PP7 can be prepared by contacting a compound of Formula PP9 with a bifunctional spacer:

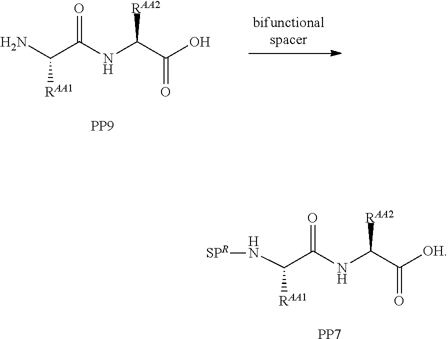

Bifunctional spacers are compounds that react with the compound of Formula PP3 to append the SP^{R} moiety present in the compounds of Formula PP2. Illustrative bifunctional spacers include, but are not limited to:

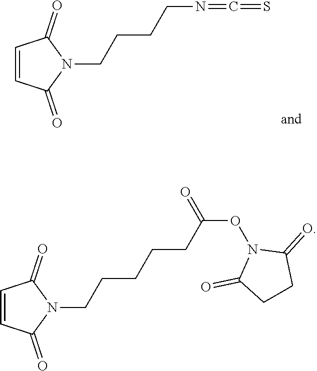 [SMILES: O=C1C=CC(=O)N1CCCCN=C=S ; O=C(CCCCCN1C(=O)C=CC1=O)ON1C(=O)CCC1=O]

Compounds of Formula PP8 can be prepared by contacting a compound of Formula PP10 with a compound of Formula PP11, following by removal of the protecting group:

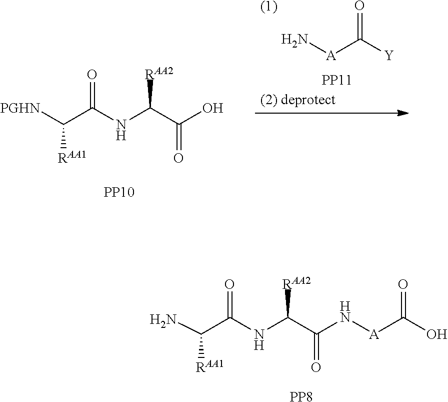

wherein PG is an amine protecting group and Y is a moiety that renders the carbonyl to which it is attached electrophilic. Compound of Formula PP10 can be prepared by coupling its corresponding amino acids using standard amino acid coupling techniques, including, for example, active ester formation using HATU, BOP/HOBt, or EDC/N-hydroxysuccinamide in the presence of DIEA, DBU, or tributylamine.

Bifunctional spacers are compounds that react with the compound of Formula PP9 to append the SP^{R} moiety present in the compounds of Formula PP7. Illustrative bifunctional spacers include, but are not limited to:

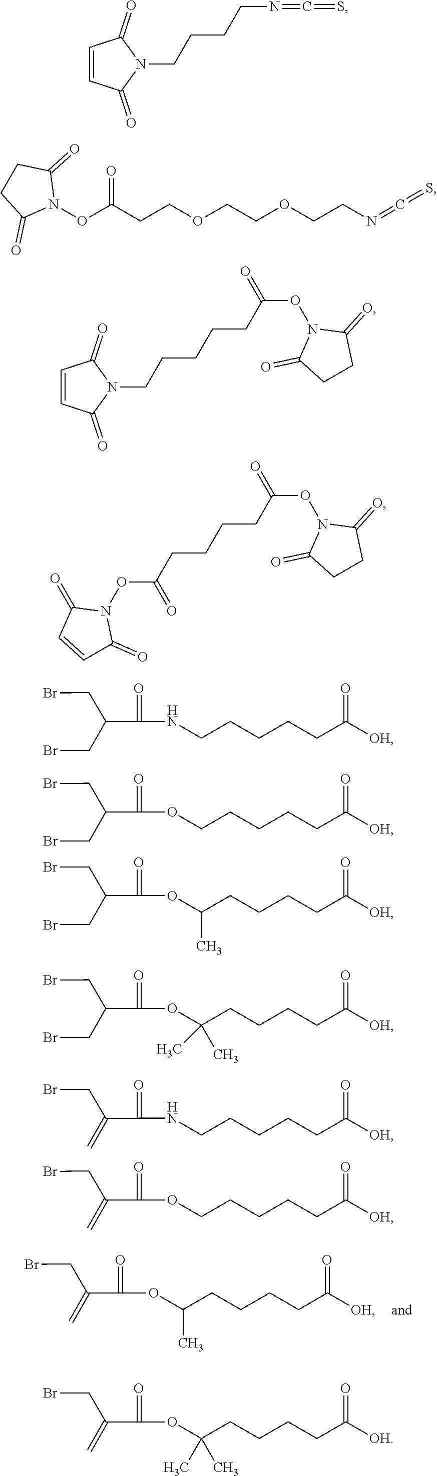

Antibody drug conjugate compounds of Formula (I) can also be prepared by reacting a suitable antibody, e.g., deglycosylated antibody or aglycosylated antibody with a compound of Formula (PT1) in the presence of transglutaminase:

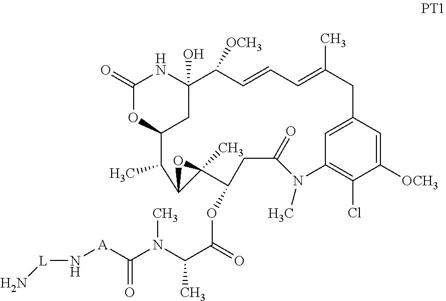

wherein:
   A is arylene or heteroarylene; and
   L is a linker.

In some embodiments, A is:

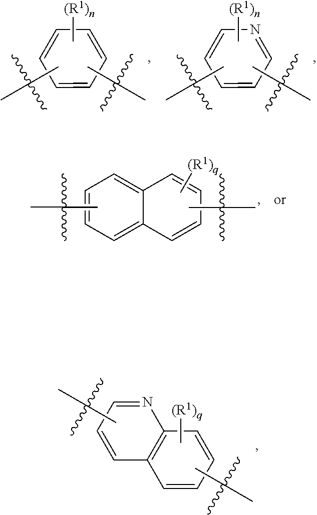

wherein:
   R¹ is, independently at each occurrence, halo, haloalkyl, haloalkoxy, hydroxyl, alkyl, alkenyl, alkynyl, alkoxy, haloalkoxy, aryl, alkaryl, aralkyl, heteroaryl, heteroalkyl, heterocycloalkyl, cyano, nitro,

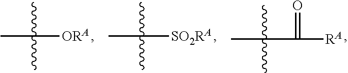

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, A is:

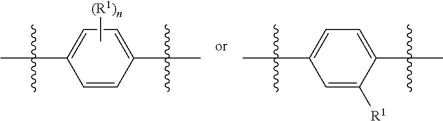

wherein:
   R¹ is, independently at each occurrence, halo, haloalkyl, hydroxyl, alkyl, alkenyl, alkynyl, alkoxy, aryl, alkaryl, aralkyl, heteroaryl, heteroalkyl, heterocycloalkyl, cyano, nitro,

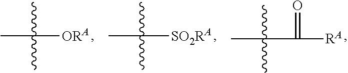

or azido,
   wherein R^{A} is alkyl or heteroalkyl;
n is an integer from 0 to 4;
m is an integer from 0 to 3;
p is an integer from 0 to 6; and
q is an integer from 0 to 5.

In some embodiments, R¹ is, independently, alkyl or halo. In some embodiments, R¹ is, independently, C₁₋₆ alkyl, C₁₋₆ haloalkyl, or halo. In some embodiments, R¹ is, independently, halo. In some embodiments, R¹ is, independently, fluoro, chloro, bromo, iodo, or trifluoromethyl. In some embodiments, n, m, p, or q is 0, 1 or 2. In some embodiments, n, m, p, or q is 0 or 1. In some embodiments, n, m, p, or q is 0.

In some embodiments, R¹ is

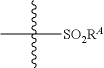

In some embodiments, R¹ is

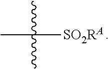

wherein R^{A} is methyl. In some embodiments, R¹ is hydroxyl. In some embodiments, R¹ is N-methylformamide. In some embodiments, R¹ is morpholinyl.

In some embodiments, the linker comprises one or more amino acids. Suitable amino acids include natural, non-natural, standard, non-standard, proteinogenic, non-proteinogenic, and L-, or D-α-amino acids. In some embodiments, the linker comprises alanine, valine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, or derivative thereof.

In some embodiments, the linker comprises valine and citrulline.

In some embodiments, the linker is:

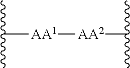

wherein:
   one

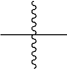

is one or more bonds to the payload;
the other

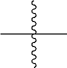

is one or more bonds to the —NH₂ of PT1;
AA¹ is an amino acid; and
AA² is an amino acid.

The linker may further comprise a divalent moiety that connects the AA¹-AA² moiety to the —NH₂ of PT1. Suitable divalent moieties include, but are not limited to, those comprising alkylene or polyethylene glycol. The divalent moiety may comprise one or more reactive groups to facilitate bonding to the rest of the compound, or one or more residues of such reactive groups.

PT1 includes a primary amine-terminated alkylene or a primary amine-terminated polyethylene glycol. The primary amine-terminating moiety can be directly bonded to a deglycosylated antibody or aglycosylated antibody in the presence of transglutaminase.

In some embodiments, the compound comprises a primary amine-terminated alkylene. In some embodiments, the compound comprises a NH₂—C₅₋₇ alkylene. In some embodiments, the compound comprises:

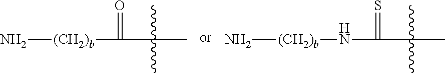

wherein:

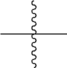

is a bond to the payload; and
b is an integer from 2 to 8.

In some embodiments, the compound comprises:

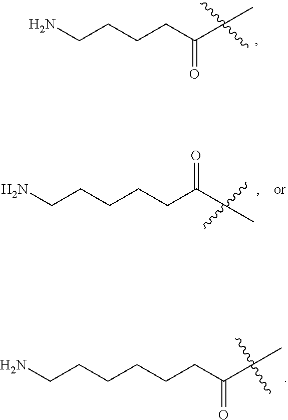 [SMILES: *C(=O)CCCCN ; *C(=O)CCCCCN ; *C(=O)CCCCCCN]

wherein:

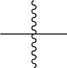

is a bond to the payload.

In some embodiments, the compound of PT1 is

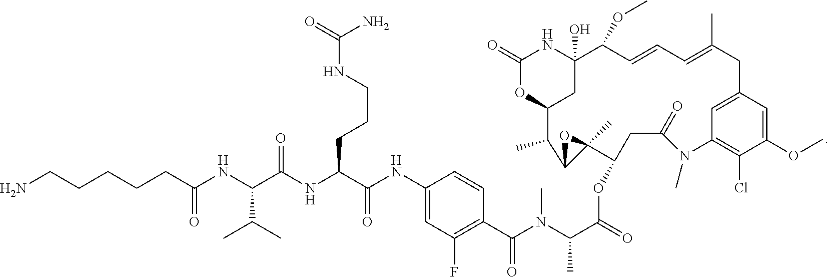 [SMILES: COc1cc2cc(c1Cl)N(C)C(=O)C[C@H](OC(=O)[C@H](C)N(C)C(=O)c1ccc(NC(=O)[C@H](CCCNC(N)=O)NC(=O)[C@@H](NC(=O)CCCCCN)C(C)C)cc1F)[C@]1(C)O[C@H]1[C@H](C)[C@@H]1C[C@@](O)(NC(=O)O1)[C@H](OC)/C=C/C=C(\C)C2]

In some embodiments, the compound of Formula (I) is prepared by contacting a binding agent with PT1 in the presence of transglutaminase under conditions suitable for a transglutamination reaction. In some embodiments, the transglutaminase reaction is at a pH between about 7 and about 8 for at least 4 hr. In some examples, the pH is 7.2, 7.3, 7.4, 7.5, 7.6, 7.8, or 8.

In some embodiments, the compound of Formula (I) is prepared by a transglutaminase reaction wherein the concentration of the compound of Formula (PT1) is at a concentration of at least 30 molar equivalents compared to the deglycosylated antibody or aglycosylated antibody. In some embodiments, the compound of Formula (I) is prepared by a transglutaminase reaction wherein the concentration of the compound of Formula (PT1) is at a concentration of 30 to 150 molar equivalents compared to the deglycosylated antibody or aglycosylated antibody.

In some embodiments, the compound of Formula (I) is prepared by a transglutaminase reaction wherein the concentration of the compound of Formula (PT1) is 1 to 30 U per milligram of deglycosylated antibody or aglycosylated antibody.

In some embodiments, the antibody is deglycosylated with peptide N-glycosidase F (PNGaseF) prior to the transglutaminase reaction.

In some embodiments, the antibody is aglycosylated. An aglycosylated antibody can be prepared by mutagenesis techniques to remove one or more amino acid sequences that are necessary for glycosylation of the antibody. In certain embodiments the antibody comprises a heavy chain with a mutation that substitutes another amino acid for N180. In certain embodiments, the aglycosylated antibody comprises one or more N180Q heavy chain polypeptides.

In some embodiments, the compound of Formula (I) is prepared by a transglutaminase reaction which is conducted in one or more solvent(s) selected from the group consisting of water, buffered water, saline water, buffered saline water, and an organic.

In some embodiments, the compound of Formula (I) is prepared by a transglutaminase reaction which is conducted in water buffered with phosphate, HEPES, or MOPS.

In some embodiments, the compound of Formula (I) is prepared by a transglutaminase reaction which includes reacting the glutaminyl-modified antibody with a reactive spacer compound to form an antibody-spacer conjugate; and then reacting the antibody-spacer conjugate with a reactive payload compound to form an antibody-spacer-payload conjugate.

In some embodiments, provided herein is a glutaminyl-modified antibody produced by a method set forth herein.

In some embodiments, provided herein is a pharmaceutical composition comprising a glutaminyl-modified antibody produced by a method set forth herein.

In some embodiments, provided herein is a method of treating a condition in a subject in need thereof comprising administering to the subject a pharmaceutically acceptable amount of the antibody or antibody-drug-conjugate provided herein.

In some embodiments, provided herein is an antibody or antibody-drug-conjugate described herein for therapy.

In some embodiments, provided herein is an antibody or antibody-drug-conjugate described herein for the treatment of cancer.

### E. Methods of Use and Pharmaceutical Compositions

The present disclosure includes methods of treating or preventing diseases, conditions, or disorders e.g., proliferative diseases such as cancer, comprising administering a therapeutically effective amount or one or more of the compounds disclosed herein, e.g., one or more of the compounds of Formula (I) or (II). Diseases, disorders, and/or conditions include, but are not limited to, those associated with the antigens listed herein. In some embodiments, the antigen is PSMA, MUC16, or EGFRvIII.

The compounds disclosed herein can be used for treating primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the compounds provided herein are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, or melanoma. In some embodiments, the cancer is breast cancer.

The compounds described herein can be administered alone or together with one or more additional therapeutic agents. The one or more additional therapeutic agents can be administered just prior to, concurrent with, or shortly after the administration of the compounds described herein. The present disclosure also includes pharmaceutical compositions comprising any of the compounds described herein in combination with one or more additional therapeutic agents, and methods of treatment comprising administering such combinations to subjects in need thereof.

Suitable additional therapeutic agents include, but are not limited to: an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2 [e.g., trastuzumab or T-DM1 {KADCYLA®}], anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET antagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-a inhibitor (e.g., an anti-PDGFR-a antibody), a PDGFR-P inhibitor (e.g., an anti-PDGFR-P antibody or small molecule kinase inhibitor such as, e.g., imatinib mesylate or sunitinib malate), a PDGF ligand inhibitor (e.g., anti-PDGF-A, —B, —C, or -D antibody, aptamer, siRNA, etc.), a VEGF antagonist (e.g., a VEGF-Trap such as aflibercept, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 antagonist (e.g., an anti-FOLH1 antibody), a STEAPI or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin [e.g., anti-UPK3A] antibody), a MUC16 antagonist (e.g., an anti-MUC16 antibody), a Tn antigen antagonist (e.g., an anti-Tn antibody), a CLEC12A antagonist (e.g., an anti-CLEC12A antibody), a TNFRSF17 antagonist (e.g., an anti-TNFRSF17 antibody), a LGR5 antagonist (e.g., an anti-LGR5 antibody), a monovalent CD20 antagonist (e.g., a monovalent anti-CD20 antibody such as rituximab), etc. Other agents that may be beneficially administered in combination with compounds of the disclosure include, e.g., tamoxifen, aromatase inhibitors, and cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors.

Suitable therapeutic agents also include, but are not limited to chemotherapeutic agents, including alkylating agents such as thiotepa and cyclosphosphamide (Cytoxan™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

The compounds described herein can also be administered and/or co-formulated in combination with antivirals, antibiotics, analgesics, corticosteroids, steroids, oxygen, antioxidants, COX inhibitors, cardioprotectants, metal chelators, IFN-gamma, and/or NSAIDs.

In some embodiments of the methods described herein, multiple doses of a compound described herein (or a pharmaceutical composition comprising a combination of an compound described herein and any of the additional therapeutic agents mentioned herein) may be administered to a subject over a defined time course. The methods according to this aspect of the disclosure comprise sequentially administering to a subject multiple doses of a compound described herein. As used herein, “sequentially administering” means that each dose of the compound is administered to the subject at a different point in time, e.g., on different days separated by a predetermined interval (e.g., hours, days, weeks or months). The present disclosure includes methods which comprise sequentially administering to the patient a single initial dose of a compound described herein, followed by one or more secondary doses of the compound, and optionally followed by one or more tertiary doses of the compound.

The terms “initial dose,” “secondary doses,” and “tertiary doses,” refer to the temporal sequence of administration of the compounds described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all contain the same amount the compound described herein, but generally can differ from one another in terms of frequency of administration. In certain embodiments, the amount of the compound contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose the compound which is administered to a patient prior to the administration of the very next dose in the sequence with no intervening doses.

The methods according to this aspect of the disclosure may comprise administering to a patient any number of secondary and/or tertiary doses of the compound. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of once a month, then the maintenance doses may be administered to the patient once every six weeks, once every two months, once every three months, etc.

The present disclosure includes pharmaceutical compositions of the compounds and/or conjugates described herein, e.g., the compounds of Formula (I) and (II), e.g., compositions comprising a compound described herein, a salt, stereoisomer, polymorph thereof, and a pharmaceutically acceptable carrier, diluent, and/or excipient. Examples of suitable carriers, diluents and excipients include, but are not limited to: buffers for maintenance of proper

1-62. (canceled)

63. A method for treating cancer in a patient comprising administering to the patient a compound of Formula (I) or a pharmaceutically acceptable salt thereof and/or a pharmaceutical composition thereof or a method for killing tumor cells comprising contacting the tumor cells with Formula (I) or a pharmaceutically acceptable salt thereof and/or a pharmaceutical composition thereof wherein Formula (I) is of the structure: 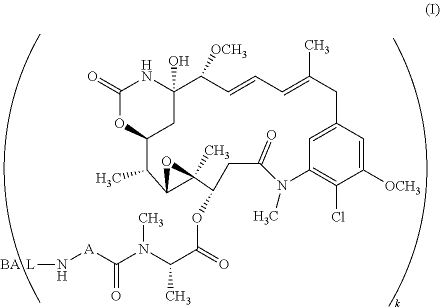
wherein:
A is arylene or heteroarylene;
L is a linker;
BA is a binding agent; and
k is an integer from 1 to 30.

64. The method of claim 63, wherein
BA is an antibody or antigen binding fragment thereof, and
k is an integer from 1-6.

65. The method of claim 64, wherein
A is 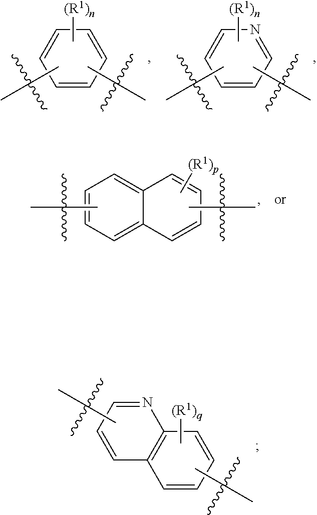
R¹, independently at each occurrence, is alkyl, alkenyl, alkynyl, alkoxy, aryl, alkaryl, arylalkyl, halo, haloalkyl, haloalkoxy, heteroaryl, heterocycloalkyl, hydroxyl, cyano, nitro, 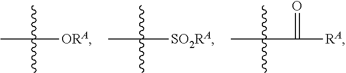
or azido;
R^{A} is alkyl or heteroalkyl;
n is an integer from 0 to 4;
m is an integer from 0 to 3;
p is an integer from 0 to 6; and
q is an integer from 0 to 5.

66. The method of claim 65, wherein
A is: 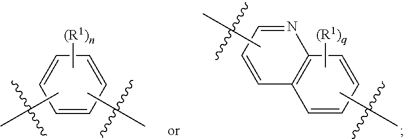
R¹ is halo, haloalkyl, alkoxy, alkyl, 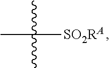
—OH, heterocycloalkyl or 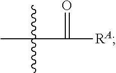
and
R^{A} is alkyl.

67. The method of claim 66, wherein A is

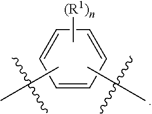

68. The method of claim 66, wherein A is: 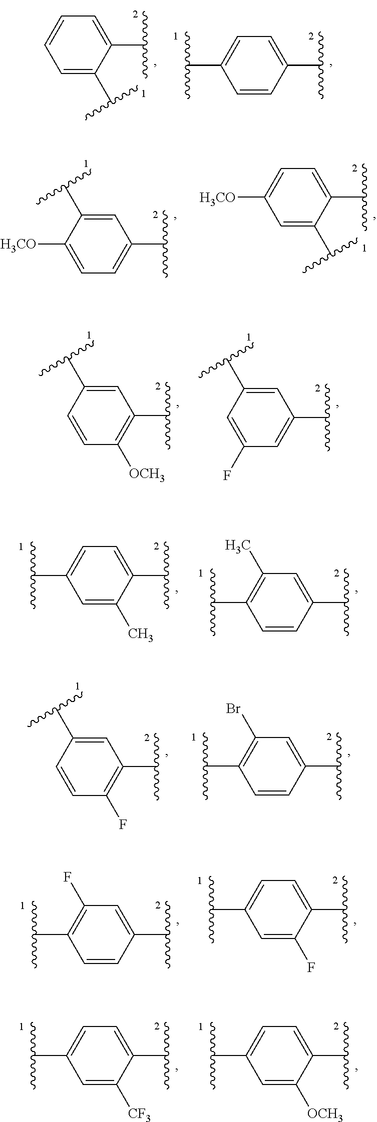 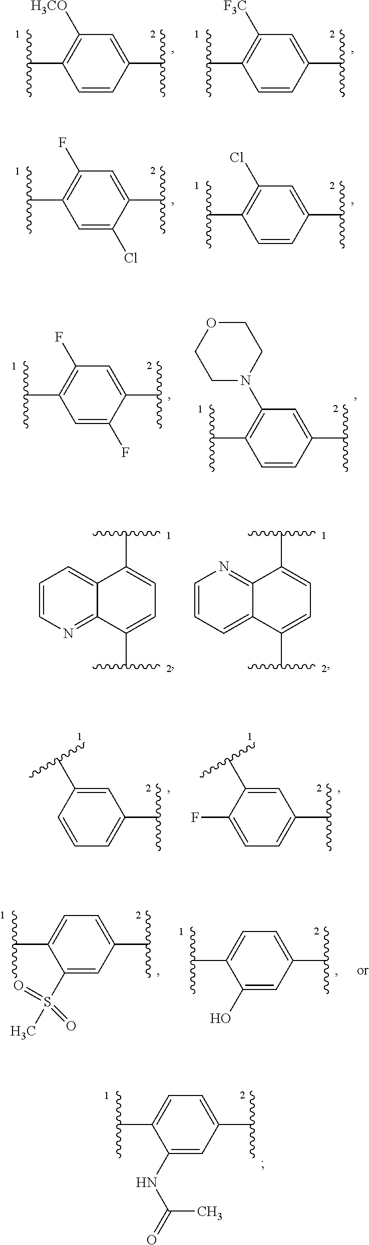 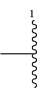
is the bond linking A to the N atom, and 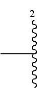
is the bond linking A to the carbonyl.

69. The method of claim 64, wherein
L is 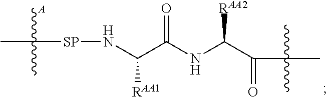
SP is a spacer; 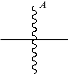
is one or more bonds to the binding agent;
R^{AA}1 is an amino acid side chain; and
R^{AA} is an amino acid side chain.

70. The method of claim 69, wherein R^{AA1} is 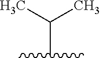 [SMILES: *C(C)C]
and R^{AA2} is

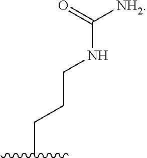 [SMILES: *CCCNC(N)=O]

71. The method of claim 69, wherein
SP is 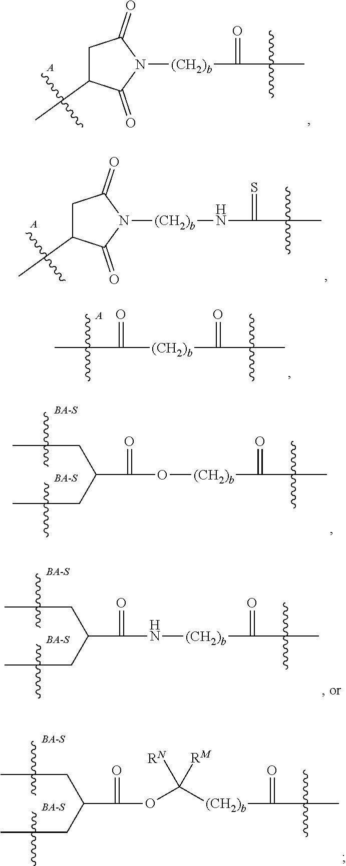
is a bond to the antibody or antigen binding fragment thereof, 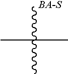
is a bond to a cysteine on the antibody or antigen binding fragment thereof,
b is an integer from 2 to 8;
R^{N} is a hydrogen atom or alkyl; and
R^{M} is alkyl.

72. The method of claim 64, wherein
L is 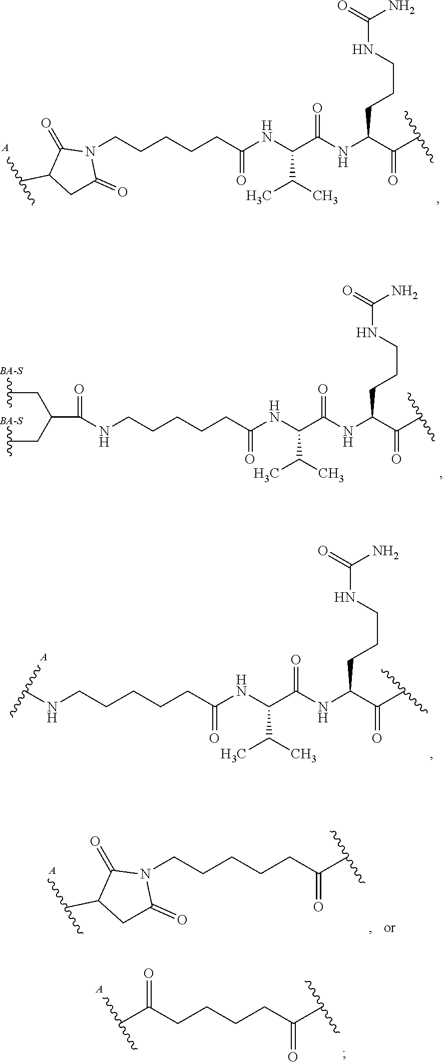
wherein: 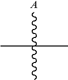
is a bond to the antibody or antigen binding fragment thereof, and 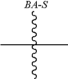
is a bond to a cysteine on the antibody or antigen binding fragment thereof.

73. The method of claim 64, wherein the compound is 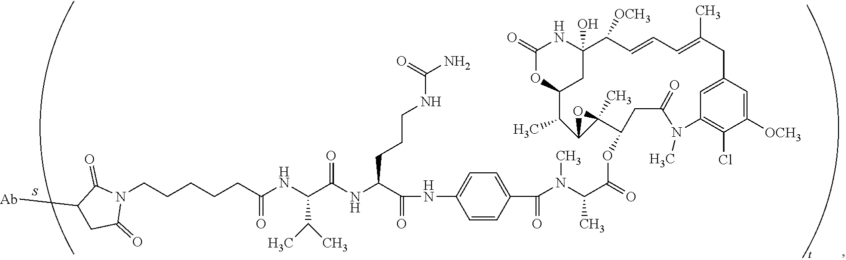
wherein t is an integer from 1 to 6;
Ab is an antibody or antigen binding fragment thereof; and [symbol]
is a bond to a cysteine on said antibody or antigen binding fragment thereof.

74. The method of claim 64, wherein the antibody, or antigen binding fragment thereof, binds PSMA, MUC16, or PRLR.

75. The method of claim 64, wherein the antibody is an antibody or antigen binding fragment thereof that binds a tumor associated antigen.

76. The method of claim 66, wherein A is 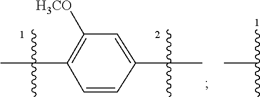
is the bond linking A to the N atom, and 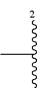
is the bond linking A to the carbonyl.

77. The method of claim 64, wherein the compound is 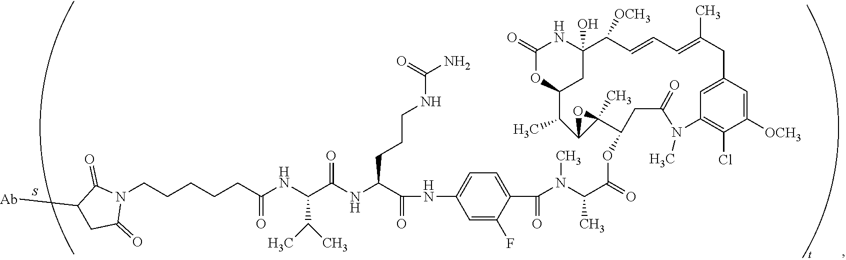
wherein t is an integer from 1 to 6;
Ab is an antibody or antigen binding fragment thereof, and [symbol]
is a bond to a cysteine on said antibody or antigen binding fragment thereof.

78. The method of claim 64, wherein the compound is 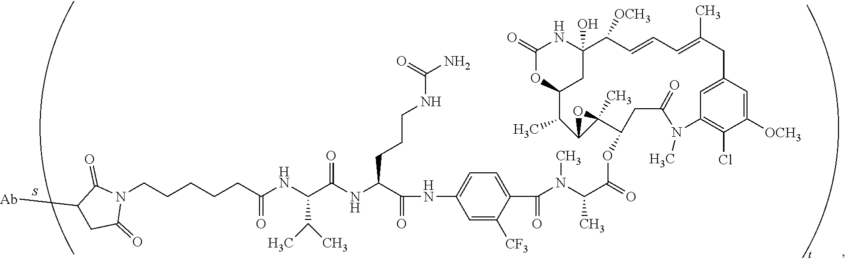
wherein t is an integer from 1 to 6;
Ab is an antibody or antigen binding fragment thereof, and [symbol]
is a bond to a cysteine on said antibody or antigen binding fragment thereof.

79. The method of claim 64, wherein the compound is 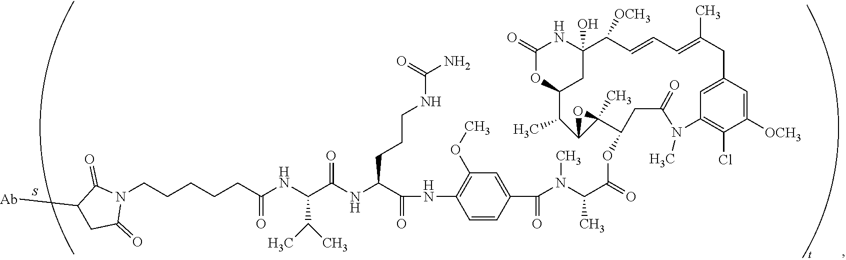
wherein t is an integer from 1 to 6;
Ab is an antibody or antigen binding fragment thereof, and [symbol]
is a bond to a cysteine on said antibody or antigen binding fragment thereof.

80. The method of claim 64, wherein the compound is 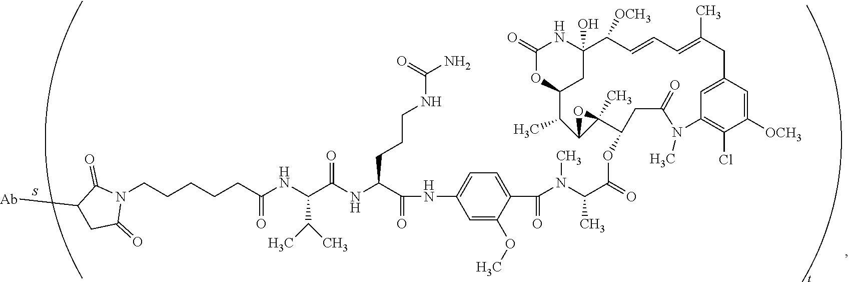
wherein t is an integer from 1 to 6;
Ab is an antibody or antigen binding fragment thereof, and [symbol]
is a bond to a cysteine on said antibody or antigen binding fragment thereof.

81. The method of claim 64, wherein the compound is 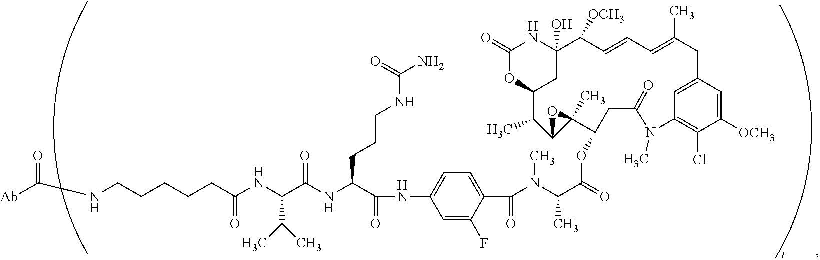
wherein t is an integer from 1 to 6; and
Ab is an antibody or antigen binding fragment thereof.

82. The method of claim 64, wherein the compound is 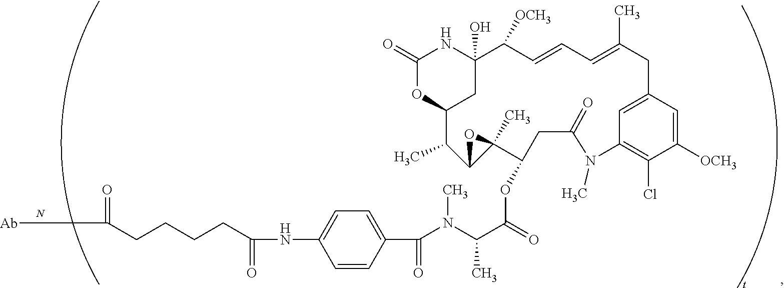
wherein t is an integer from 1 to 6;
Ab is an antibody or antigen binding fragment thereof, and [symbol]
is a bond to a lysine on said antibody or antigen binding fragment thereof.

83. The method of claim 64, wherein the compound is 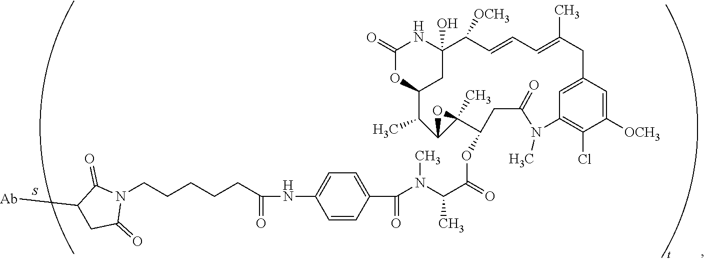
wherein t is an integer from 1 to 6;
Ab is an antibody or antigen binding fragment thereof, and [symbol]
is a bond to a cysteine on said antibody or antigen binding fragment thereof.

84. The method of claim 63, wherein the cancer is selected from renal cell carcinoma, pancreatic carcinoma, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, breast cancer, and melanoma.

85. A method for treating cancer in a patient comprising administering to the patient a compound of Formula II or a pharmaceutically acceptable salt thereof and/or a pharmaceutical composition thereof or a method for killing tumor cells comprising contacting the tumor cells with a compound of Formula (II) or a pharmaceutically acceptable salt thereof and/or a pharmaceutical composition thereof wherein Formula (II) is of the structure: 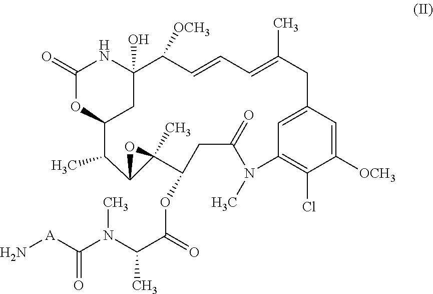
wherein A is arylene or heteroarylene.

86. The method of claim 85, wherein the cancer is selected from renal cell carcinoma, pancreatic carcinoma, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, breast cancer, and melanoma.
